(19) United States
(12) Patent Application Publication
(10) Pub. No.: US 2007/0082007 A1
(43) Pub. Date: Apr. 12, 2007

(54) Novel immunogenic compositions for the prevention and treatment of meningococcal disease

(76) Inventors: Gary W. Zlotnick, New Windor, NY (US); Leah Diane Fletcher, Geneseo, NY (US); John Erwin Farley, Chapel Hill, NC (US); Liesel A. Bernfield, Pittsford, NY (US); Robert J. Zagursky, Victor, NY (US); Benjamin J. Metcalf, Rochester, NY (US)

Correspondence Address: HUNTON & WILLIAMS LLP;INTELLECTUAL PROPERTY DEPARTMENT, 1900 K STREET, N.W., SUITE 1200, WASHINGTON, DC, 20006-1109, US

(21) Appl. No.: 11/638,520

(22) Filed: Dec. 14, 2006

Related U.S. Application Data
(62) Division of application No. 10/798,894, filed on Mar. 12, 2004.
(60) Provisional application No. 60/463,161, filed on Apr. 16, 2003.

(51) Int. Cl. A61K 39/095; A61K 39/02; C07K 14/22

(52) U.S. Cl. 424/190.1; 424/250.1; 530/350

(57) ABSTRACT

The present invention relates to Neisseria ORF2086 proteins, crossreactive immunogenic proteins which can be isolated from nesserial strains or prepared recombinantly, including immunogenic portions thereof, biological equivalents thereof, antibodies that immunospecifically bind to the foregoing and nucleic acid sequences encoding each of the foregoing, as well as the use of same in immunogenic compositions that are effective against infection by Neisseria meningitidis serogroup B.

### PRIORITY DATA

This is a divisional of U.S. patent application Ser. No. 10/798,894 filed Mar. 12, 2004, which claims priority under 35 U.S.C. § 119(e) from U.S. Provisional Patent Application No. 60/463,161 filed Apr. 16, 2003, which are incorporated by reference in their entirety.

### FIELD OF THE INVENTION

The present invention relates to Neisseria ORF2086 proteins (Subfamily A and Subfamily B), which may be isolated from bacterial strains such as those of Neisseria species, including strains of Neisseria meningitidis (serogroups A, B, C, D, W-135, X, Y, Z and 29E), Neisseria gonorrhoeae, and Neisseria lactamica, as well as immunogenic portions and/or biological equivalents of said proteins. The present invention also relates to antibodies that immunospecifically bind to said proteins, immunogenic portions and/or biological equivalents. Further, the present invention relates to isolated polynucleotides comprising nucleic acid sequences encoding any of the foregoing proteins, immunogenic portions, biological equivalents and/or antibodies. Additionally, the present invention relates to immunogenic compositions and their use in preventing, treating and/or diagnosing meningococcal infection caused by N. meningitidis, and in particular meningococcal disease caused by N. meningitidis serogroup B, as well as methods for preparing said compositions. This invention relates to both recombinant forms and forms isolated from a natural source, as well as both lipidated and non-lipidated forms.

### BACKGROUND OF THE INVENTION

Meningococcal meningitis is a devastating disease that can kill children and young adults within hours despite the availability of antibiotics. Pizza et al., 2000, Science 287:1816-1820. Meningitis is characterized as an inflammation of the meninges resulting in an intense headache, fever, loss of appetite, intolerance to light and sound, rigidity of muscles, especially in the neck, and in severe cases convulsions, vomiting and delirium leading to death. The symptoms of meningococcal meningitis appear suddenly and culminate in meningococcal septicemia with its characteristic hemorrhagic rash. A rapid diagnosis and immediate treatment with large doses of antibiotics is critical if there is to be any chance of survival. 2000. Bantam Medical Dictionary, Third Edition 302.

Meningococcal meningitis is caused by Neisseria meningitidis (the meningococcus), a Gram-negative, capsulated bacterium that has been classified into several pathogenic serogroups including A, B, C, D, W-135, X, Y, Z and 29E. Serogroup B strains of N. meningitidis are a major cause of meningococcal disease throughout the world. For example, it is reported in the medical literature that serogroup B is responsible for about 50% of bacterial meningitis in infants and children residing in the United States and Europe. No vaccine currently exists to prevent meningococcal disease caused by N. meningitidis serogroup B.

Developing an immunogenic composition for the prevention of serogroup B meningococcal disease has been a challenge to researchers since the work of Goldschneider et al. over thirty years ago. Goldschneider et al., 1969, J. Exp. Med 129(6):1307-26; Goldschneider et al, 1969, J. Exp. Med 129(6):1327-48; Gotschlich et al., 1969, J. Exp. Med. 129(6):1385-95; and Gotschlich et al., 1969, J. Exp. Med. 129(6):1367-84. Unlike serogroup A disease, which virtually disappeared from North America after World War II, Achtman, M., 1995, Trends in Microbiology 3(5):186-92, disease caused by serogroup B and C organisms remains endemic throughout much of the economically developed world. The incidence of disease varies from <1/100,000 where endemic disease is rare to 200/100,000 in high risk populations during epidemics.

Vaccines based on polysaccharide conjugates have been developed against N. meningitidis serogroups A and C and appear to be effective in preventing disease. Currently, an immunogenic composition made of capsular polysaccharide from serogroups A, C, Y, & W-135 is available. Ambrosch et al., 1983, limunogenicity and side-effects of a new tetravalent. Bulletin of the World Health Organization 61(2):317-23. However, this immunogenic composition elicits a T-cell independent immune response, is not effective in young children, and provides no coverage for serogroup B strains, which cause upwards of 50% of meningococcal disease.

Others have also attempted to develop immunogenic compositions using capsular polysaccharides. Recently, immunogenic compositions for serogroup C disease prepared by conjugating the serogroup C capsular material to proteins have been licensed for use in Europe. However, the serogroup B capsule may be unsuitable as a vaccine candidate because the capsule polysaccharide is composed of polysialic acid which bears a similarity to carbohydrate moieties on developing human neural tissues. This sugar moiety is recognized as a self-antigen and is thus poorly immunogenic in humans.

Outer membrane proteins (OMP's) have been developed as alternative vaccine antigens for serogroup B disease. Monoclonal antibody binding to the two variable regions of PorA define the serosubtyping scheme for meningococci. PorA proteins thus serve as the serosubtyping antigens (Abdillahi et al., 1988, Microbial Pathogenesis 4(1):27-32) for meningococcal strains and are being actively investigated as components of a serogroup B immunogenic composition (Poolman, 1996, Adv. Exp. Med. Biol. 397:73-7), since they can elicit bactericidal antibodies (Saukkonen, 1987, Microbial Pathogenesis 3(4):261-7). Bactericidal antibodies are thought to be an indicator of protection and any new immunogenic composition candidate should elicit these functional antibodies.

Studies in humans as well as animals indicate that the serosubtyping antigen, PorA, elicits bactericidal antibodies. However, the immune response to Por A is generally serosubtype specific. In particular, serosubtyping data indicate that an immunogenic composition made of PorAs may require a PorA for each serosubtype to be covered by such an immunogenic composition, perhaps as many as six to nine. Therefore, 6-9 PorAs will be needed to cover 70-80% of serogroup B strains. Thus, the variable nature of this protein requires a multivalent vaccine composition to protect against a sufficient number of meningococcal serosubtype clinical isolates.

Developing an immunogenic composition for serogroup B meningococci has been so difficult that recently several groups have sequenced the genomes from strains representing both serogroups A and B to assist in identifying new immunogenic composition candidates. Tettelin, 2000, Science, 287(5459):1809-15; Pizza et al., 2000, Science 287:1816-1820. Identifying new immunogenic composition candidates, even with the knowledge of the neisserial genome, is a challenging process for which adequate mathematical algorithms do not currently exist. In fact, a recent report indicates that despite identifying hundreds of open reading frames (“ORFs”) containing theoretical membrane spanning domains, problems with expression, purification, and inducing surface reactive, and functionally active antibodies have led investigators to only seven candidates for a serogroup B meningococcal immunogenic composition. See Id. One of these was previously known.

Accordingly, there remains a need for immunogenic compositions that (1) elicit bactericidal antibodies to multiple neisserial strains; (2) react with the surface of multiple strains; (3) confer passive protection against a live challenge; and/or (4) prevent colonization.

### SUMMARY OF THE INVENTION

To meet these and other needs, and in view Qf its purposes, the present invention provides Neisseria ORF2086 proteins (“2086 proteins”), including 2086 Subfamily A proteins and 2086 Subfamily B proteins. Each of the 2086 proteins are proteins that can be isolated from native neisserial strains, including strains of Neisseria meningitidis (serogroups A, B, C, D, W-135, X, Y, Z and 29E), Neisseria gonorrhoeae, and Neisseria lactamica. The 2086 proteins may also be prepared using recombinant technology.

In particular, the present invention provides the 2086 proteins, immunogenic portions thereof, and/or biological equivalents thereof, antibodies that immunospecifically bind to any of the foregoing, and polynucleotides comprising nucleic acid sequences that encode any of the foregoing. The present invention includes compositions, immunogenic compositions and their use in preventing, treating and/or diagnosing meningococcal infection, and in particular meningococcal disease caused by N. meningitidis, as well as methods for preparing said compositions. The 2086 proteins herein include recombinant forms and forms isolated from a natural source, as well as both lipidated and non-lipidated forms.

The present invention unexpectedly and advantageously provides compositions that (1) elicit bactericidal antibodies to multiple neisserial strains, such as strains of N. meningitidis, N. gonorrhoeae, and/or N. lactamica; (2) react with the surface of multiple strains; (3) confer passive protection against a live challenge; and/or (4) prevent colonization, as well as methods of using said compositions and methods of preparing said compositions. Various embodiments of the invention are described below.

### BRIEF DESCRIPTION OF THE DRAWINGS

FIG. 1A depicts an SDS-PAGE gel that depicts the two major proteins of the protein fractions obtained from the experiments for identifying neisserial membrane protein extract that is capable of eliciting bactericidal antibodies against heterologous strains.

FIG. 1B depicts the results from the experiments from the identification of the two major proteins by analysis of TMAE Flow Through components by protease digestion and reverse Phase N-terminal sequencing.

FIG. 2 depicts the purification scheme and homogeneity as determined by SDS-PAGE of rLP2086.

FIG. 3 depicts the results from the experiments from the identification of the two major proteins and one minor protein by analysis of TMAE Flow Through components by LC-MS/MS and the corresponding SDS-PAGE.

FIG. 4 is an SDS-PAGE gel from the recombinant expression of 2086 protein.

FIG. 5 is a schematic diagram of plasmid pPX7340, as described in the examples herein.

FIG. 6 is a schematic diagram of plasmid pPX7328 as described in the examples herein.

FIG. 7 is a schematic diagram of plasmid pPX7343 as described in the examples herein.

FIG. 8 illustrates N-terminal regions of 2086 gene from various strains.

FIG. 9A is a flow chart showing the preliminary steps in the identification of an immunogenic component in a nesserial strain.

FIG. 9B is a flow chart showing the final steps in the identification of an immunogenic component in a nesserial strain.

FIG. 10A is a schematic diagram of the pBAD arabinose inducible promoter which drives the expression of the P4 signal/ORF2086 fusion protein to express a lipidated form of rP2086 as described in the examples herein.

FIG. 10B is a schematic diagram of the pET9a-T7 vector for recombinant expression of nonlipidated form of ORF2086.

FIG. 11A is a photograph representing whole cell lysates of E. coli B expressing the rLP2086 protein.

FIG. 11B shows whole cell lysates of E. coli B expressing the rP2086 protein.

FIG. 12 is a phylogenetic tree showing an organization of the subfamilies and groups of ORF2086 proteins according an implementation of the present invention.

FIG. 13 is a graphic illustration of whole cell ELISA data for the rLP2086 Subfamily A antisera.

FIG. 14 is a graphical illustration of whole cell ELISA data for the rLP2086 Subfamily B antisera.

FIG. 15 is a graphical illustration of the results of the rLP2086 mixing study—WCE Titers.

FIG. 16 is a graphical illustration of the results of the rLP2086/rPorA mixing study—WCE Titers.

FIG. 17 is a Western Blot showing reactivity of rLP2086 mouse antisera to P2086 Subfamily B N. meningitidis whole cell lysates.

FIG. 18 is a Western Blot showing reactivity of rLP2086 mouse antisera to P2086 Subfamily A N. meningitidis and N. lactamica whole cell lysates.

FIG. 19 is a phylogenetic tree showing an organization of subfamilies and groups of ORF2086 proteins according to an implementation of the invention.

FIG. 20 is a sequence alignment comparing polypeptides of the present invention.

### SEQUENCE SUMMARY

SEQ ID NOS. for Studied Sequences:

SEQ ID NO:1 nucleic acid sequence encoding amino acid sequence for mature 2086 protein from L3 6275 strain when combined with a native leader sequence.
SEQ ID NO:2 amino acid sequence for mature 2086 protein from L3 6275 strain prepared using a native leader sequence.
SEQ ID NO:3 nucleic acid sequence for encoding amino acid sequence for mature 2086 protein from L3 6275 when combined with a P4 leader sequence.
SEQ ID NO:4 amino acid sequence for mature 2086 protein from L3 6275 strain prepared using a P4 leader sequence.
SEQ ID NO:5 nucleic acid sequence encoding amino acid sequence for mature 2086 protein from L3 6275 strain.
SEQ ID NO:6 amino acid sequence for mature 2086 protein from L3 6275 strain.
SEQ ID NO:7 nucleic acid sequence encoding amino acid sequence for mature 2086 protein from CDC2369 strain when combined with a native leader sequence.
SEQ ID NO:8 amino acid sequence for mature 2086 protein from CDC2369 strain prepared using a native leader sequence.
SEQ ID NO:9 nucleic acid sequence for encoding amino acid sequence for mature 2086 protein from CDC2369 when combined with a P4 leader sequence.
SEQ ID NO:10 amino acid sequence for mature 2086 protein from CDC2369 strain prepared using a P4 leader sequence.
SEQ ID NO:11 nucleic acid sequence encoding amino acid sequence for mature 2086 protein from CDC2369 strain.
SEQ ID NO:12 amino acid sequence for mature 2086 protein from CDC2369 strain.
SEQ ID NO:13 nucleic acid sequence encoding amino acid sequence for mature 2086 protein from CDC1034 strain when combined with a native leader sequence.
SEQ ID NO:14 amino acid sequence for mature 2086 protein from CDC1034 strain prepared using a native leader sequence.
SEQ ID NO:15 nucleic acid sequence for encoding amino acid sequence for mature 2086 protein from CDC1034 when combined with a P4 leader sequence.
SEQ ID NO:16 amino acid sequence for mature 2086 protein from CDC1034 strain prepared using a P4 leader sequence.
SEQ ID NO:17 nucleic acid sequence encoding amino acid sequence for mature 2086 protein from CDC1034 strain.
SEQ ID NO:18 amino acid sequence for mature 2086 protein from CDC 1034 strain.
SEQ ID NO:19 nucleic acid sequence encoding amino acid sequence for mature 2086 protein from L4 891 strain when combined with a native leader sequence.
SEQ ID NO:20 amino acid sequence for mature 2086 protein from L4 891 strain prepared using a native leader sequence.
SEQ ID NO:21 nucleic acid sequence for encoding amino acid sequence for mature 2086 protein from L4 891 when combined with a P4 leader sequence.
SEQ ID NO:22 amino acid sequence for mature 2086 protein from L4 891 strain prepared using a P4 leader sequence.
SEQ ID NO:23 nucleic acid sequence encoding amino acid sequence for mature 2086 protein from L4 891 strain.
SEQ ID NO:24 amino acid sequence for mature 2086 protein from L4 891 strain.
SEQ ID NO:25 nucleic acid sequence encoding amino acid sequence for mature 2086 protein from B16B6 strain when combined with a native leader sequence.
SEQ ID NO:26 amino acid sequence for mature 2086 protein from B16B6 strain prepared using a native leader sequence.
SEQ ID NO:27 nucleic acid sequence for encoding amino acid sequence for mature 2086 protein from B16B6 when combined with a P4 leader sequence.
SEQ ID NO:28 amino acid sequence for mature 2086 protein from B16B6 strain prepared using a P4 leader sequence.
SEQ ID NO:29 nucleic acid sequence encoding amino acid sequence for mature 2086 protein from B16B6 strain.
SEQ ID NO:30 amino acid sequence for mature 2086 protein from B16B6 strain.
SEQ ID NO:31 nucleic acid sequence encoding amino acid sequence for mature 2086 protein from W135 (ATCC35559) strain when combined with a native leader.sequence.
SEQ ID NO:32 amino acid sequence for mature 2086 protein from W135 (ATCC35559) strain prepared using a native leader sequence.
SEQ ID NO:33 nucleic acid sequence for encoding amino acid sequence for mature 2086 protein from W135 (ATCC35559) when combined with a P4 leader sequence.
SEQ ID NO:34 amino acid sequence for mature 2086 protein from W135 (ATCC35559) strain prepared using a P4 leader sequence.
SEQ ID NO:35 nucleic acid sequence encoding amino acid sequence for mature 2086 protein from W135 (ATCC35559) strain.
SEQ ID NO:36 amino acid sequence for mature 2086 protein from W135 (ATCC35559) strain.
SEQ ID NO:37 nucleic acid sequence encoding amino acid sequence for mature 2086 protein from C11 strain when combined with a native leader sequence.
SEQ ID NO:38 amino acid sequence for mature 2086 protein from C11 strain prepared using a native leader sequence.
SEQ ID NO:39 nucleic acid sequence for encoding amino acid sequence for mature 2086 protein from C11 when combined with a P4 leader sequence.
SEQ ID NO:40 amino acid sequence for mature 2086 protein from C11 strain prepared using a P4 leader sequence.
SEQ ID NO:41 nucleic acid sequence encoding amino acid sequence for mature 2086 protein from C11 strain.
SEQ ID NO:42 amino acid sequence for mature 2086 protein from C11 strain.
SEQ ID NO:43 nucleic acid sequence encoding amino acid sequence for mature 2086 protein from Y (ATCC35561) strain when combined with a native leader sequence.
SEQ ID NO:44 amino acid sequence for mature 2086 protein from Y (ATCC35561) strain prepared using a native leader sequence.
SEQ ID NO:45 nucleic acid sequence for encoding amino acid sequence for mature 2086 protein from Y (ATCC35561) when combined with a P4 leader sequence.
SEQ ID NO:46 amino acid sequence for mature 2086 protein from Y (ATCC35561) strain prepared using a P4 leader sequence.
SEQ ID NO:47 nucleic acid sequence encoding amino acid sequence for mature 2086 protein from Y (ATCC35561) strain.
SEQ ID NO:48 amino acid sequence for mature 2086 protein from Y (ATCC35561) strain.
SEQ ID NO:49 nucleic acid sequence encoding amino acid sequence for mature 2086 protein from M98 250732 strain when combined with a native leader sequence.
SEQ ID NO:50 amino acid sequence for mature 2086 protein from M98 250732 strain prepared using a native leader sequence.
SEQ ID NO:51 nucleic acid sequence for encoding amino acid sequence for mature 2086 protein from M98 250732 when combined with a P4 leader sequence.
SEQ ID NO:52 amino acid sequence for mature 2086 protein from M98 250732 strain prepared using a P4 leader sequence.
SEQ ID NO:53 nucleic acid sequence encoding amino acid sequence for mature 2086 protein from M98 250732 strain.
SEQ ID NO:54 amino acid sequence for mature 2086 protein from M98 250732 strain.
SEQ ID NO:55 nucleic acid sequence encoding amino acid sequence for mature 2086 protein from M98 250771 strain when combined with a native leader sequence.
SEQ ID NO:56 amino acid sequence for mature 2086 protein from M98 250771 strain prepared using a native leader sequence.
SEQ ID NO:57 nucleic acid sequence for encoding amino acid sequence for mature 2086 protein from M98 250771 when combined with a P4 leader sequence.
SEQ ID NO:58 amino acid sequence for mature 2086 protein from M98 250771 strain prepared using a P4 leader sequence.
SEQ ID NO:59 nucleic acid sequence encoding amino acid sequence for mature 2086 protein from M98 250771 strain.
SEQ ID NO:60 amino acid sequence for mature 2086 protein from M98 250771 strain.
SEQ ID NO:61 nucleic acid sequence encoding amino acid sequence for mature 2086 protein from CDC1135 strain when combined with a native leader sequence.
SEQ ID NO:62 amino acid sequence for mature 2086 protein from CDC1135 strain prepared using a native leader sequence.
SEQ ID NO:63 nucleic acid sequence for encoding amino acid sequence for mature 2086 protein from CDC1135 when combined with a P4 leader sequence.
SEQ ID NO:64 amino acid sequence for mature 2086 protein from CDC1135 strain prepared using a P4 leader sequence.
SEQ ID NO:65 nucleic acid sequence encoding amino acid sequence for mature 2086 protein from CDC1135 strain.
SEQ ID NO:66 amino acid sequence for mature 2086 protein from CDC1135 strain.
SEQ ID NO:67 nucleic acid sequence encoding amino acid sequence for mature 2086 protein from M97 252153 strain when combined with a native leader sequence.
SEQ ID NO:68 amino acid sequence for mature 2086 protein from M97 252153 strain prepared using a native leader sequence.
SEQ ID NO:69 nucleic acid sequence for encoding amino acid sequence for mature 2086 protein from M97 252153 when combined with a P4 leader sequence.
SEQ ID NO:70 amino acid sequence for mature 2086 protein from M97 252153 strain prepared using a P4 leader sequence.
SEQ ID NO:71 nucleic acid sequence encoding amino acid sequence for mature 2086 protein from M97 252153 strain.
SEQ ID NO:72 amino acid sequence for mature 2086 protein from M97 252153 strain.
SEQ ID NO:73 nucleic acid sequence encoding amino acid sequence for mature 2086 protein from CDC1610 strain when combined with a native leader sequence.
SEQ ID NO:74 amino acid sequence for mature 2086 protein from CDC1610 strain prepared using a native leader sequence.
SEQ ID NO:75 nucleic acid sequence for encoding amino acid sequence for mature 2086 protein from CDC1610 when combined with a P4 leader sequence.
SEQ ID NO:76 amino acid sequence for mature 2086 protein from CDC1610 strain prepared using a P4 leader sequence.
SEQ ID NO:77 nucleic acid sequence encoding amino acid sequence for mature 2086 protein from CDC1610 strain.
SEQ ID NO:78 amino acid sequence for mature 2086 protein from CDC1610 strain.
SEQ ID NO:79 nucleic acid sequence encoding amino acid sequence for mature 2086 protein from CDC1492 strain when combined with a native leader sequence.
SEQ ID NO:80 amino acid sequence for mature 2086 protein from CDC1492 strain prepared using a native leader sequence.
SEQ ID NO:81 nucleic acid sequence for encoding amino acid sequence for mature 2086 protein from CDC1492 when combined with a P4 leader sequence.
SEQ ID NO:82 amino acid sequence for mature 2086 protein from CDC1492 strain prepared using a P4 leader sequence.
SEQ ID NO:83 nucleic acid sequence encoding amino acid sequence for mature 2086 protein from CDC1492 strain.
SEQ ID NO:84 amino acid sequence for mature 2086 protein from CDC1492 strain.
SEQ ID NO:85 nucleic acid sequence encoding amino acid sequence for mature 2086 protein from L8 M978 strain when combined with a native leader sequence.
SEQ ID NO:86 amino acid sequence for mature 2086 protein from L8 M978 strain prepared using a native leader sequence.
SEQ ID NO:87 nucleic acid sequence for encoding amino acid sequence for mature 2086 protein from L8 M978 when combined with a P4 leader sequence.
SEQ ID NO:88 amino acid sequence for mature 2086 protein from L8 M978 strain prepared using a P4 leader sequence.
SEQ ID NO:89 nucleic acid sequence encoding amino acid sequence for mature 2086 protein from L8 M978 strain.
SEQ ID NO:90 amino acid sequence for mature 2086 protein from L8 M978 strain.
SEQ ID NO:91 nucleic acid sequence encoding amino acid sequence for mature 2086 protein from M97 252988 strain when combined with a native leader sequence.
SEQ ID NO:92 amino acid sequence for mature 2086 protein from M97 252988 strain prepared using a native leader sequence.
SEQ ID NO:93 nucleic acid sequence for encoding amino acid sequence for mature 2086 protein from M97 252988 when combined with a P4 leader sequence.
SEQ ID NO:94 amino acid sequence for mature 2086 protein from M97 252988 strain prepared using a P4 leader sequence.
SEQ ID NO:95 nucleic acid sequence encoding amino acid sequence for mature 2086 protein from M97 252988 strain.
SEQ ID NO:96 amino acid sequence for mature 2086 protein from M97 252988 strain.
SEQ ID NO:97 nucleic acid sequence encoding amino acid sequence for mature 2086 protein from M97 252697 strain when combined with a native leader sequence.
SEQ ID NO:98 amino acid sequence for mature 2086 protein from M97 252697 strain prepared using a native leader sequence.
SEQ ID NO:99 nucleic acid sequence for encoding amino acid sequence for mature 2086 protein from M97 252697 when combined with a P4 leader sequence.
SEQ ID NO:100 amino acid sequence for mature 2086 protein from M97 252697 strain prepared using a P4 leader sequence.
SEQ ID NO:101 nucleic acid sequence encoding amino acid sequence for mature 2086 protein from M97 252697 strain.
SEQ ID NO:102 amino acid sequence for mature 2086 protein from M97 252697 strain.
SEQ ID NO:103 nucleic acid sequence encoding amino acid sequence for mature 2086 protein from 6557 strain when combined with a native leader sequence.
SEQ ID NO:104 amino acid sequence for mature 2086 protein from 6557 strain prepared using a native leader sequence.
SEQ ID NO:105 nucleic acid sequence for encoding amino acid sequence for mature 2086 protein from 6557 when combined with a P4 leader sequence.
SEQ ID NO:106 amino acid sequence for mature 2086 protein from 6557 strain prepared using a P4 leader sequence.
SEQ ID NO:107 nucleic acid sequence encoding amino acid sequence for mature 2086 protein from 6557 strain.
SEQ ID NO:108 amino acid sequence for mature 2086 protein from 6557 strain.
SEQ ID NO:109 nucleic acid sequence encoding amino acid sequence for mature 2086 protein from 2996 strain when combined with a native leader sequence.
SEQ ID NO:110 amino acid sequence for mature 2086 protein from 2996 strain prepared using a native leader sequence.
SEQ ID NO:111 nucleic acid sequence for encoding amino acid sequence for mature 2086 protein from 2996 when combined with a P4 leader sequence.
SEQ ID NO:112 amino acid sequence for mature 2086 protein from 2996 strain prepared using a P4 leader sequence.
SEQ ID NO:113 nucleic acid sequence encoding amino acid sequence for mature 2086 protein from 2996 strain.
SEQ ID NO:114 amino acid sequence for mature 2086 protein from 2996 strain.
SEQ iD NO:115 nucleic acid sequence encoding amino acid sequence for mature 2086 protein from M97 252976 strain when combined with a native leader sequence.
SEQ ID NO:116 amino acid sequence for mature 2086 protein from M97 252976 strain prepared using a native leader sequence.
SEQ ID NO:117 nucleic acid sequence for encoding amino acid sequence for mature 2086 protein from M97 252976 when combined with a P4 leader sequence.
SEQ ID NO:118 amino acid sequence for mature 2086 protein from M97 252976 strain prepared using a P4 leader sequence.
SEQ ID NO:119 nucleic acid sequence encoding amino acid sequence for mature 2086 protein from M97 252976 strain.
SEQ ID NO:120 amino acid sequence for mature 2086 protein from M97 252976 strain.
SEQ ID NO:121 nucleic acid sequence encoding amino acid sequence for mature 2086 protein from M97 251854 strain when combined with a native leader sequence.
SEQ ID NO:122 amino acid sequence for mature 2086 protein from M97 251854 strain prepared using a native leader sequence.
SEQ ID NO:123 nucleic acid sequence for encoding amino acid sequence for mature 2086 protein from M97 251854 when combined with a P4 leader sequence.
SEQ ID NO:124 amino acid sequence for mature 2086 protein from M97 251854 strain prepared using a P4 leader sequence.
SEQ ID NO:125 nucleic acid sequence encoding amino acid sequence for mature 2086 protein from M97 251854 strain.
SEQ ID NO:126 amino acid sequence for mature 2086 protein from M97 251854 strain.
SEQ ID NO:127 nucleic acid sequence encoding amino acid sequence for mature 2086 protein from CDC1521 strain when combined with a native leader sequence.
SEQ ID NO:128 amino acid sequence for mature 2086 protein from CDC1521 strain prepared using a native leader sequence.
SEQ ID NO:129 nucleic acid sequence for encoding amino acid sequence for mature 2086 protein from CDC1521 when combined with a P4 leader sequence.
SEQ ID NO:130 amino acid sequence for mature 2086 protein from CDC1521 strain prepared using a P4 leader sequence.
SEQ ID NO:131 nucleic acid sequence encoding amino acid sequence for mature 2086 protein from CDC1521 strain.
SEQ ID NO:132 amino acid sequence for mature 2086 protein from CDC1521 strain.
SEQ ID NO:133 nucleic acid sequence encoding amino acid sequence for mature 2086 protein from M98 250622 strain when combined with a native leader sequence.
SEQ ID NO:134 amino acid sequence for mature 2086 protein from M98 250622 strain prepared using a native leader sequence.
SEQ ID NO:135 nucleic acid sequence for encoding amino acid sequence for mature 2086 protein from M98 250622 when combined with a P4 leader sequence.
SEQ ID NO:136 amino acid sequence for mature 2086 protein from M98 250622 strain prepared using a P4 leader sequence.
SEQ ID NO:137 nucleic acid sequence encoding amino acid sequence for mature 2086 protein from M98 250622 strain.
SEQ ID NO:138 amino acid sequence for mature 2086 protein from M98 250622 strain.
SEQ ID NO:139 nucleic acid sequence encoding amino acid sequence for mature 2086 protein from 870446 strain when combined with a native leader sequence.
SEQ ID NO:140 amino acid sequence for mature 2086 protein from 870446 strain prepared using a native leader sequence.
SEQ ID NO:141 nucleic acid sequence for encoding amino acid sequence for mature 2086 protein from 870446 when combined with a P4 leader sequence.
SEQ ID NO:142 amino acid sequence for mature 2086 protein from 870446 strain prepared using a P4 leader sequence.
SEQ ID NO:143 nucleic acid sequence encoding amino acid sequence for mature 2086 protein from 870446 strain.
SEQ ID NO:144 amino acid sequence for mature 2086 protein from 870446 strain.
SEQ ID NO:145 nucleic acid sequence encoding amino acid sequence for mature 2086 protein from M97 253248 strain when combined with a native leader sequence.
SEQ ID NO:146 amino acid sequence for mature 2086 protein from M97 253248 strain prepared using a native leader sequence.
SEQ ID NO:147 nucleic acid sequence for encoding amino acid sequence for mature 2086 protein from M97 253248 when combined with a P4 leader sequence.
SEQ ID NO:148 amino acid sequence for mature 2086 protein from M97 253248 strain prepared using a P4 leader sequence.
SEQ ID NO:149 nucleic acid sequence encoding amino acid sequence for mature 2086 protein from M97 253248 strain.
SEQ ID NO:150 amino acid sequence for mature 2086 protein from M97 253248 strain.
SEQ ID NO:151 nucleic acid sequence encoding amino acid sequence for mature 2086 protein from M98 250809 strain when combined with a native leader sequence.
SEQ ID NO:152 amino acid sequence for mature 2086 protein from M98 250809 strain prepared using a native leader sequence.
SEQ ID NO:153 nucleic acid sequence for encoding amino acid sequence for mature 2086 protein from M98 250809 when combined with a P4 leader sequence.
SEQ ID NO:154 amino acid sequence for mature 2086 protein from M98 250809 strain prepared using a P4 leader sequence.
SEQ ID NO:155 nucleic acid sequence encoding amino acid sequence for mature 2086 protein from M98 250809 strain.
SEQ ID NO:156 amino acid sequence for mature 2086 protein from M98 250809 strain.
SEQ ID NO:157 nucleic acid sequence encoding amino acid sequence for mature 2086 protein from L5 M981 strain when combined with a native leader sequence.
SEQ ID NO:158 amino acid sequence for mature 2086 protein from L5 M981 strain prepared using a native leader sequence.
SEQ ID NO:159 nucleic acid sequence for encoding amino acid sequence for mature 2086 protein from L5 M981 when combined with a P4 leader sequence.
SEQ ID NO:160 amino acid sequence for mature 2086 protein from L5 M981 strain prepared using a P4 leader sequence.
SEQ ID NO:161 nucleic acid sequence encoding amino acid sequence for mature 2086 protein from L5 M981 strain.
SEQ ID NO:162 amino acid sequence for mature 2086 protein from L5 M981 strain.
SEQ ID NO:163 nucleic acid sequence encoding amino acid sequence for mature 2086 protein from NMB strain when combined with a native leader sequence.
SEQ ID NO:164 amino acid sequence for mature 2086 protein from NMB strain prepared using a native leader sequence.
SEQ ID NO:165 nucleic acid sequence for encoding amino acid sequence for mature 2086 protein from NMB when combined with a P4 leader sequence.
SEQ ID NO:166 amino acid sequence for mature 2086 protein from NMB strain prepared using a P4 leader sequence.
SEQ ID NO:167 nucleic acid sequence encoding amino acid sequence for mature 2086 protein from NMB strain.
SEQ ID NO:168 amino acid sequence for mature 2086 protein from NMB strain.
SEQ ID NO:169 nucleic acid sequence encoding amino acid sequence for mature 2086 protein from M98 250572 strain when combined with a native leader sequence.
SEQ ID NO:170 amino acid sequence for mature 2086 protein from M98 250572 strain prepared using a native leader sequence.
SEQ ID NO:171 nucleic acid sequence for encoding amino acid sequence for mature 2086 protein from M98 250572 when combined with a P4 leader sequence.
SEQ ID NO:172 amino acid sequence for mature 2086 protein from M98 250572 strain prepared using a P4 leader sequence.
SEQ ID NO:173 nucleic acid sequence encoding amino acid sequence for mature 2086 protein from M98 250572 strain.
SEQ ID NO:174 amino acid sequence for mature 2086 protein from M98 250572 strain.
SEQ ID NO:175 nucleic acid sequence encoding amino acid sequence for mature 2086 protein from A4 Sanford; M97 251836 PART; M97 251957; M97 251985; M97 252060; M97 251870; M97 251994; M98 250024; M97 251905; M97 251876; M97 251898; or M97 251830 strain when combined with a native leader sequence.
SEQ ID NO:176 amino acid sequence for mature 2086 protein from A4 Sanford; M97 251836 PART; M97 251957; M97 251985; M97 252060; M97 251870; M97 251994; M98 250024; M97 251905; M97 251876; M97 251898; or M97 251830 strain prepared using a native leader sequence.
SEQ ID NO:177 nucleic acid sequence for encoding amino acid sequence for mature 2086 protein from A4 Sanford; M97 251836 PART; M97 251957; M97 251985; M97 252060; M97 251870; M97 251994; M98 250024; M97 251905; M97 251876; M97 251898; or M97 251830 when combined with a P4 leader sequence.
SEQ ID NO:178 amino acid sequence for mature 2086 protein from A4 Sanford; M97 251836 PART; M97 251957; M97 251985; M97 252060; M97 251870; M97 251994; M98 250024; M97 251905; M97 251876; M97 251898; or M97 251830 strain prepared using a P4 leader sequence.
SEQ ID NO:179 nucleic acid sequence encoding amino acid sequence for mature 2086 protein from A4 Sanford; M97 251836 part; M97 251957; M97 251985; M97 252060; M97 251870; M97 251994; M98 250024; M97 251905; M97 251876; M97 251898; or M97 251830 strain.
SEQ ID NO:180 amino acid sequence for mature 2086 protein from A4 Sanford; M97 251836 PART; M97 251957; M97 251985; M97 252060; M97 251870; M97 251994; M98 250024; M97 251905; M97 251876; M97 251898; or M97 251830 strain.
SEQ ID NO:181 nucleic acid sequence encoding amino acid sequence for mature 2086 protein from CDC937 strain when combined with a native leader sequence.
SEQ ID NO:182 amino acid sequence for mature 2086 protein from CDC937 strain prepared using a native leader sequence.
SEQ ID NO:183 nucleic acid sequence for encoding amino acid sequence for mature 2086 protein from CDC937 when combined with a P4 leader sequence.
SEQ ID NO:184 amino acid sequence for mature 2086 protein from CDC937 strain prepared using a P4 leader sequence.
SEQ ID NO:185 nucleic acid sequence encoding amino acid sequence for mature 2086 protein from CDC937 strain.
SEQ ID NO:186 amino acid sequence for mature 2086 protein from CDC937 strain.
SEQ ID NO:187 nucleic acid sequence encoding amino acid sequence for mature 2086 protein from M97 252097 strain when combined with a native leader sequence.
SEQ ID NO:188 amino acid sequence for mature 2086 protein from M97 252097 strain prepared using a native leader sequence.
SEQ ID NO:189 nucleic acid sequence for encoding amino acid sequence for mature 2086 protein from M97 252097 when combined with a P4 leader sequence.
SEQ ID NO:190 amino acid sequence for mature 2086 protein from M97 252097 strain prepared using a P4 leader sequence.
SEQ ID NO:191 nucleic acid sequence encoding amino acid sequence for mature 2086 protein from M97 252097 strain.
SEQ ID NO:192 amino acid sequence for mature 2086 protein from M97 252097 strain.
SEQ ID NO:193 nucleic acid sequence encoding amino acid sequence for mature 2086 protein from 870227 strain when combined with a native leader sequence.
SEQ ID NO:194 amino acid sequence for mature 2086 protein from 870227 strain prepared using a native leader sequence.
SEQ ID NO:195 nucleic acid sequence for encoding amino acid sequence for mature 2086 protein from 870227 when combined with a P4 leader sequence.
SEQ ID NO:196 amino acid sequence for mature 2086 protein from 870227 strain prepared using a P4 leader sequence.
SEQ ID NO:197 nucleic acid sequence encoding amino acid sequence for mature 2086 protein from 870227 strain.
SEQ ID NO:198 amino acid sequence for mature 2086 protein from 870227 strain.
SEQ ID NO:199 nucleic acid sequence encoding amino acid sequence for mature 2086 protein from H355 strain when combined with a native leader sequence.
SEQ ID NO:200 amino acid sequence for mature 2086 protein from H355 strain prepared using a native leader sequence.
SEQ ID NO:201 nucleic acid sequence for encoding amino acid sequence for mature 2086 protein from H355 when combined with a P4 leader sequence.
SEQ ID NO:202 amino acid sequence for mature 2086 protein from H355 strain prepared using a P4 leader sequence.
SEQ ID NO:203 nucleic acid sequence encoding amino acid sequence for mature 2086 protein from H355 strain.
SEQ ID NO:204 amino acid sequence for mature 2086 protein from H355 strain.
SEQ ID NO:205 nucleic acid sequence encoding amino acid sequence for mature 2086 protein from H44_{—}76 strain when combined with a native leader sequence.
SEQ ID NO:206 amino acid sequence for mature 2086 protein from H44_{—}76 strain prepared using a native leader sequence.
SEQ ID NO:207 amino acid sequence for mature 2086 protein from H44_{—}76 strain prepared using a P4 leader sequence.
SEQ ID NO:208 nucleic acid sequence encoding amino acid sequence for mature 2086 protein from H44_{—}76 strain.
SEQ ID NO:209 nucleic acid sequence for encoding amino acid sequence for mature 2086 protein from H44_{—}76 when combined with a P4 leader sequence.
SEQ ID NO:210 amino acid sequence for mature 2086 protein from H44_{—}76 strain.
SEQ ID NO:211 nucleic acid sequence encoding amino acid sequence for mature 2086 protein from 8529 strain when combined with a native leader sequence.
SEQ ID NO:212 amino acid sequence for mature 2086 protein from 8529 strain prepared using a native leader sequence.
SEQ ID NO:213 nucleic acid sequence for encoding amino acid sequence for mature 2086 protein from 8529 when combined with a P4 leader sequence.
SEQ ID NO:214 amino acid sequence for mature 2086 protein from 8529 strain prepared using a P4 leader sequence.
SEQ ID NO:215 nucleic acid sequence encoding amino acid sequence for mature 2086 protein from 8529 strain.
SEQ ID NO:216 amino acid sequence for mature 2086 protein from 8529 strain.
SEQ ID NO:217 nucleic acid sequence encoding amino acid sequence for mature 2086 protein from 6940 strain when combined with a native leader sequence.
SEQ ID NO:218 amino acid sequence for mature 2086 protein from 6940 strain prepared using a native leader sequence.
SEQ ID NO:219 nucleic acid sequence for encoding amino acid sequence for mature 2086 protein from 6940 when combined with a P4 leader sequence.
SEQ ID NO:220 amino acid sequence for mature 2086 protein from 6940 strain prepared using a P4 leader sequence.
SEQ ID NO:221 nucleic acid sequence encoding amino acid sequence for mature 2086 protein from 6940 strain.
SEQ ID NO:222 amino acid sequence for mature 2086 protein from 6940 strain.
SEQ ID NO:223 nucleic acid sequence encoding amino acid sequence for mature 2086 protein from M982 strain when combined with a native leader sequence.
SEQ ID NO:224 amino acid sequence for mature 2086 protein from M982 strain prepared using a native leader sequence.
SEQ ID NO:225 nucleic acid sequence for encoding amino acid sequence for mature 2086 protein from M982 when combined with a P4 leader sequence.
SEQ ID NO:226 amino acid sequence for mature 2086 protein from M982 strain prepared using a P4 leader sequence.
SEQ ID NO:227 nucleic acid sequence encoding amino acid sequence for mature 2086 protein from M982 strain.
SEQ ID NO:228 amino acid sequence for mature 2086 protein from M982 strain.
SEQ ID NO:229 nucleic acid sequence encoding amino acid sequence for mature 2086 protein from 880049 strain when combined with a native leader sequence.
SEQ ID NO:230 amino acid sequence for mature 2086 protein from 880049 strain prepared using a native leader sequence.
SEQ ID NO:231 nucleic acid sequence for encoding amino acid sequence for mature 2086 protein from 880049 when combined.with a P4 leader sequence.
SEQ ID NO:232 amino acid sequence for mature 2086 protein from 880049 strain prepared using a P4 leader sequence.
SEQ ID NO:233 nucleic acid sequence encoding amino acid sequence for mature 2086 protein from 880049 strain.
SEQ ID NO:234 amino acid sequence for mature 2086 protein from 880049 strain.
SEQ ID NO:235 nucleic acid sequence encoding amino acid sequence for mature 2086 protein from M97 253524, M97 251885, and M97 251926 strains when combined with a native leader sequence.
SEQ ID NO:236 amino acid sequence for mature 2086 protein from M97 253524, M97 251885, and M97 251926 strains prepared using a native leader sequence.
SEQ ID NO:237 nucleic acid sequence for encoding amino acid sequence for mature 2086 protein from M97 253524, M97 251885, and M97 251926 strains when combined with a P4 leader sequence.
SEQ ID NO:238 amino acid sequence for mature 2086 protein from M97 253524, M97 251885, and M97 251926 strains prepared using a P4 leader sequence.
SEQ ID NO:239 nucleic acid sequence encoding amino acid sequence for mature 2086 protein from M97 253524, M97 251885, and M97 251926 strains.
SEQ ID NO:240 amino acid sequence for mature 2086 protein from M97 253524, M97 251885, and M97 251926 strains.
SEQ ID NO:241 nucleic acid sequence encoding amino acid sequence for mature 2086 protein from M98 250670 strain when combined with a native leader sequence.
SEQ ID NO:242 amino acid sequence for mature 2086 protein from M98 250670 strain prepared using a native leader sequence.
SEQ ID NO:243 nucleic acid sequence for encoding amino acid sequence for mature 2086 protein from M98 250670 when combined with a P4 leader sequence.
SEQ ID NO:244 amino acid sequence for mature 2086 protein from M98 250670 strain prepared using a P4 leader sequence.
SEQ ID NO:245 nucleic acid sequence encoding amino acid sequence for mature 2086 protein from M98 250670 strain.
SEQ ID NO:246 amino acid sequence for mature 2086 protein from M98 250670 strain.
SEQ ID NO:247 nucleic acid sequence encoding amino acid sequence for mature 2086 protein from CDC1573 strain when combined with a native leader sequence.
SEQ ID NO:248 amino acid sequence for mature 2086 protein from CDC1573 strain prepared using a native leader sequence.
SEQ ID NO:249 nucleic acid sequence for encoding amino acid sequence for mature 2086 protein from CDC1573 when combined with a P4 leader sequence.
SEQ ID NO:250 amino acid sequence for mature 2086 protein from CDC1573 strain prepared using a P4 leader sequence.
SEQ ID NO:251 nucleic acid sequence encoding amino acid sequence for mature 2086 protein from CDC1573 strain.
SEQ ID NO:252 amino acid sequence for mature 2086 protein from CDC1573 strain.
SEQ ID NO:253 partial nucleic acid sequence encoding amino acid sequence for 2086 protein from a strain of Neisseria lactamica.
SEQ ID NOS:254 to 259 amino acid sequences associated with proteins of 2086 family of proteins.
SEQ ID NOS:260 to 278 amino acid sequences associated with proteins of 2086 Subfamily A.
SEQ ID NOS:279 to 299 amino acid sequences associated with proteins of 2086 Subfamily B.
SEQ ID NO:300 is the amino acid consensus sequence corresponding to the 2086 protein family (“2086 proteins”) according to an embodiment of the present invention.
SEQ ID NO:301 is the amino acid consensus sequence corresponding to the 2086 protein Subfamily A according to an embodiment of the present invention.
SEQ ID NO:302 is the amino acid consensus sequence corresponding to the 2086 protein Subfamily B according to an embodiment of the present invention.
SEQ ID NO:303 nucleic acid sequence for a reverse primer with BamHI restriction site (Compound No. 4623).
SEQ ID NO:304 nucleic acid sequence for a forward primer with NdeI restriction site (Compound No. 4624).
SEQ ID NO:305 nucleic acid sequence for a forward primer (Compound No. 4625).
SEQ ID NO:306 nucleic acid sequence for a forward primer (Compound No. 5005).
SEQ ID NO:307 nucleic acid sequence for a reverse primer (Compound No. 5007).
SEQ ID NO:308 nucleic acid sequence for a reverse primer with BglII restriction site (Compound No. 5135).
SEQ ID NO:309 nucleic acid sequence for a forward primer with BamHI restriction site (Compound No. 5658).
SEQ ID NO:3 10 nucleic acid sequence for a reverse primer with SphI restriction site (Compound No. 5660).
SEQ ID NO:311 nucleic acid sequence for a forward primer with BamHI restriction site (Compound No. 6385).
SEQ ID NO:312 nucleic acid sequence for a forward primer with BglII and NdeI restriction sites (Compound No. 6406).
SEQ ID NO:313 nucleic acid sequence for a forward primer (Compound No. 6470).
SEQ ID NO:314 nucleic acid sequence for a reverse primer (Compound No. 6472).
SEQ ID NO:315 nucleic acid sequence for a forward primer with BamHI restriction site (Compound 6473).
SEQ ID NO:316 nucleic acid sequence for a forward primer with BglII and NdeI restriction sites (Compound No. 6474).
SEQ ID NO:317 nucleic acid sequence for a forward primer (Compound No. 6495).
SEQ ID NO:318 nucleic acid sequence for a reverse primer (Compound No. 6496).
SEQ ID NO:319 nucleic acid sequence for a reverse primer with SphI restriction site (Compound No. 6543).
SEQ ID NO:320 nucleic acid sequence for a reverse primer with BglII restriction site (Compound No. 6605).
SEQ ID NO:321 nucleic acid sequence for a forward primer with BglII and NdeI restriction sites (Compound No. 6721).
SEQ ID NO:322 nucleic acid sequence for the P4 leader sequence.
SEQ ID NO:323 nucleic acid sequence for native 2086 leader variant 1.
SEQ ID NO:324 nucleic acid sequence for native 2086 leader variant 2.
SEQ ID NO:325 nucleic acid sequence for native 2086 leader variant 3.
SEQ ID NO:326 nucleic acid sequence for native 2086 leader variant 4.
SEQ ID NO:327 is the amino acid sequence of P4431.
SEQ ID NO:328 is the amino acid sequence of P5163.
SEQ ID NO:329 is an amino acid sequence according to an embodiment of the present invention.
SEQ ID NO:330 nucleic acid sequence encoding amino acid sequence for 2086 protein from 880049 strain when combined with a native leader sequence.
SEQ ID NO:331 amino acid sequence for 2086 protein from 880049 strain prepared using a native leader sequence.
SEQ ID NO:332 nucleic acid sequence for encoding amino acid sequence for 2086 protein from 880049 strain when combined with a P4 leader sequence.
SEQ ID NO:333 amino acid sequence for 2086 protein from 880049 strain prepared using a P4 leader sequence.
SEQ ID NO:334 nucleic acid sequence encoding amino acid sequence for 2086 protein from 880049 strain.
SEQ ID NO:335 amino acid sequence for 2086 protein from 880049 strain.
SEQ ID NO:336 nucleic acid sequence encoding amino acid sequence for 2086 protein from CDC-937 strain when combined with a native leader sequence.
SEQ ID NO:337 amino acid sequence for 2086 protein from CDC-937 strain prepared using a native leader sequence.
SEQ ID NO:338 nucleic acid sequence for encoding amino acid sequence for 2086 protein from CDC-937 strain when combined with a P4 leader sequence.
SEQ ID NO:339 amino acid sequence for 2086 protein from CDC-937 strain prepared using a P4 leader sequence.
SEQ ID NO:340 nucleic acid sequence encoding amino acid sequence for 2086 protein from CDC-937 strain.
SEQ ID NO:341 amino acid sequence for 2086 protein from CDC-937 strain.
SEQ ID NO:342 nucleic acid sequence encoding amino acid sequence for 2086 protein from M97 252097 strain when combined with a native leader sequence.
SEQ ID NO:343 amino acid sequence for 2086 protein from M97 252097 strain prepared using a native leader sequence.
SEQ ID NO:344 nucleic acid sequence for encoding amino acid sequence for 2086 protein from M97 252097 strain when combined with a P4 leader sequence.
SEQ ID NO:345 amino acid sequence for 2086 protein from M97 252097 strain prepared using a P4 leader sequence.
SEQ ID NO:346 nucleic acid sequence encoding amino acid sequence for 2086 protein from M97 252097 strain.
SEQ ID NO:347 amino acid sequence for 2086 protein from M97 252097 strain.
SEQ ID NO:348 nucleic acid sequence encoding amino acid sequence for 2086 protein from B40 strain when combined with a native leader sequence.
SEQ ID NO:349 amino acid sequence for 2086 protein from B40 strain prepared using a native leader sequence.
SEQ ID NO:350 nucleic acid sequence for encoding amino acid sequence for 2086 protein from B40 strain when combined with a P4 leader sequence.
SEQ ID NO:351 amino acid sequence for 2086 protein from B40 strain prepared using a P4 leader sequence.
SEQ ID NO:352 nucleic acid sequence encoding amino acid sequence for 2086 protein from B40 strain.
SEQ ID NO:353 amino acid sequence for 2086 protein from B40 strain.
SEQ ID NO:354 nucleic acid sequence encoding amino acid sequence for mature 2086 protein from B40 strain when combined with a native leader sequence.
SEQ ID NO:355 amino acid sequence for mature 2086 protein from B40 strain prepared using a native leader sequence.
SEQ ID NO:356 nucleic acid sequence for encoding amino acid sequence for mature 2086 protein from B40 strain when combined with a P4 leader sequence.
SEQ ID NO:357 amino acid sequence for mature 2086 protein from B40 strain prepared using a P4 leader sequence.
SEQ ID NO:358 nucleic acid sequence encoding amino acid sequence for mature 2086 protein from B40 strain.
SEQ ID NO:359 amino acid sequence for mature 2086 protein from B40 strain.
SEQ ID NO:360 nucleic acid sequence encoding amino acid sequence for 2086 protein from CDC-1343 strain when combined with a native leader sequence.
SEQ ID NO:361 amino acid sequence for 2086 protein from CDC-1343 strain prepared using a native leader sequence.
SEQ ID NO:362 nucleic acid sequence for encoding amino acid sequence for 2086 protein from CDC-1343 strain when combined with a P4 leader sequence.
SEQ ID NO:363 amino acid sequence for 2086 protein from CDC-1343 strain prepared using a P4 leader sequence.
SEQ ID NO:364 nucleic acid sequence encoding amino acid sequence for 2086 protein from CDC-1343 strain.
SEQ ID NO:365 amino acid sequence for 2086 protein from CDC-1343 strain.
SEQ ID NO:366 nucleic acid sequence encoding amino acid sequence for mature 2086 protein from CDC-1343 strain when combined with a native leader sequence.
SEQ ID NO:367 amino acid sequence for mature 2086 protein from CDC-1343 strain prepared using a native leader sequence.
SEQ ID NO:368 nucleic acid sequence for encoding amino acid sequence for mature 2086 protein from CDC-1343 strain when combined with a P4 leader sequence.
SEQ ID NO:369 amino acid sequence for mature 2086 protein from CDC-1343 strain prepared using a P4 leader sequence.
SEQ ID NO:370 nucleic acid sequence encoding amino acid sequence for mature 2086 protein from CDC-1343 strain.
SEQ ID NO:371 amino acid sequence for mature 2086 protein from CDC-1343 strain.
SEQ ID NO:372 nucleic acid sequence encoding amino acid sequence for 2086 protein from CDC-2367 strain when combined with a native leader sequence.
SEQ ID NO:373 amino acid sequence for 2086 protein from CDC-2367 strain prepared using a native leader sequence.
SEQ ID NO:374 nucleic acid sequence for encoding amino acid sequence for 2086 protein from CDC-2367 strain when combined with a P4 leader sequence.
SEQ ID NO:375 amino acid sequence for 2086 protein from CDC-2367 strain prepared using a P4 leader sequence.
SEQ ID NO:376 nucleic acid sequence encoding amino acid sequence for 2086 protein from CDC-2367 strain.
SEQ ID NO:377 amino acid sequence for 2086 protein from CDC-2367 strain.
SEQ ID NO:378 nucleic acid sequence encoding amino acid sequence for mature 2086 protein from CDC-2367 strain when combined with a native leader sequence.
SEQ ID NO:379 amino acid sequence for mature 2086 protein from CDC-2367 strain prepared using a native leader sequence.
SEQ ID NO:380 nucleic acid sequence for encoding amino acid sequence for mature 2086 protein from CDC-2367 strain when combined with a P4 leader sequence.
SEQ ID NO:381 amino acid sequence for mature 2086 protein from CDC-2367 strain prepared using a P4 leader sequence.
SEQ ID NO:382 nucleic acid sequence encoding amino acid sequence for mature 2086 protein from CDC-2367 strain.
SEQ ID NO:383 amino acid sequence for mature 2086 protein from CDC-2367 strain.
SEQ ID NO:384 nucleic acid sequence encoding amino acid sequence for 2086 protein from CDC-5315 strain when combined with a native leader sequence.
SEQ ID NO:385 amino acid sequence for 2086 protein from CDC-5315 strain prepared using a native leader sequence.
SEQ ID NO:386 nucleic acid sequence for encoding amino acid sequence for 2086 protein from CDC-5315 strain when combined with a P4 leader sequence.
SEQ ID NO:387 amino acid sequence for 2086 protein from CDC-5315 strain prepared using a P4 leader sequence.
SEQ ID NO:388 nucleic acid sequence encoding amino acid sequence for 2086 protein from CDC-5315 strain.
SEQ ID NO:389 amino acid sequence for 2086 protein from CDC-5315 strain.
SEQ ID NO:390 nucleic acid sequence encoding amino acid sequence for mature 2086 protein from CDC-5315 strain when combined with a native leader sequence.
SEQ ID NO:391 amino acid sequence for mature 2086 protein from CDC-5315 strain prepared using a native leader sequence.
SEQ ID NO:392 nucleic acid sequence for encoding amino acid sequence for mature 2086 protein from CDC-5315 strain when combined with a P4 leader sequence.
SEQ ID NO:393 amino acid sequence for mature 2086 protein from CDC-5315 strain prepared using a P4 leader sequence.
SEQ ID NO:394 nucleic acid sequence encoding amino acid sequence for mature 2086 protein from CDC-5315 strain.
SEQ ID NO:395 amino acid sequence for mature 2086 protein from CDC-5315 strain.
SEQ ID NO:396 nucleic acid sequence encoding amino acid sequence for 2086 protein from CDC-852 strain when combined with a native leader sequence.
SEQ ID NO:397 amino acid sequence for 2086 protein from CDC-852 strain prepared using a native leader sequence.
SEQ ID NO:398 nucleic acid sequence for encoding amino acid sequence for 2086 protein from CDC-852 strain when combined with a P4 leader sequence.
SEQ ID NO:399 amino acid sequence for 2086 protein from CDC-852 strain prepared using a P4 leader sequence.
SEQ ID NO:400 nucleic acid sequence encoding amino acid sequence for 2086 protein from CDC-852 strain.
SEQ ID NO:401 amino acid sequence for 2086 protein from CDC-852 strain.
SEQ ID NO:402 nucleic acid sequence encoding amino acid sequence for mature 2086 protein from CDC-852 strain when combined with a native leader sequence.
SEQ ID NO:403 amino acid sequence for mature 2086 protein from CDC-852 strain prepared using a native leader sequence.
SEQ ID NO:404 nucleic acid sequence for encoding amino acid sequence for mature 2086 protein from CDC-852 strain when combined with a P4 leader sequence.
SEQ ID NO:405 amino acid sequence for mature 2086 protein from CDC-852 strain prepared using a P4 leader sequence.
SEQ ID NO:406 nucleic acid sequence encoding amino acid sequence for mature 2086 protein from CDC-852 strain.
SEQ ID NO:407 amino acid sequence for mature 2086 protein from CDC-852 strain.
SEQ ID NO:408 nucleic acid sequence encoding amino acid sequence for 2086 protein from CDC-983 strain when combined with a native leader sequence.
SEQ ID NO:409 amino acid sequence for 2086 protein from CDC-983 strain prepared using a native leader sequence.
SEQ ID NO:410 nucleic acid sequence for encoding amino acid sequence for 2086 protein from CDC-983 strain when combined with a P4 leader sequence.
SEQ ID NO:411 amino acid sequence for 2086 protein from CDC-983 strain prepared using a P4 leader sequence.
SEQ ID NO:412 nucleic acid sequence encoding amino acid sequence for 2086 protein from CDC-983 strain.
SEQ ID NO:413 amino acid sequence for 2086 protein from CDC-983 strain.
SEQ ID NO:414 nucleic acid sequence encoding amino acid sequence for mature 2086 protein from CDC-983 strain when combined with a native leader sequence.
SEQ ID NO:415 amino acid sequence for mature 2086 protein from CDC-983 strain prepared using a native leader sequence.
SEQ ID NO:416 nucleic acid sequence for encoding amino acid sequence for mature 2086 protein from CDC-983 strain when combined with a P4 leader sequence.
SEQ ID NO:417 amino acid sequence for mature 2086 protein from CDC-983 strain prepared using a P4 leader sequence.
SEQ ID NO:418 nucleic acid sequence encoding amino acid sequence for mature 2086 protein from CDC-983 strain.
SEQ ID NO:419 amino acid sequence for mature 2086 protein from CDC-983 strain.
SEQ ID NO:420 nucleic acid sequence encoding amino acid sequence for 2086 protein from M97 250571 strain when combined with a native leader sequence.
SEQ ID NO:421 amino acid sequence for 2086 protein from M97 250571 strain prepared using a native leader sequence.
SEQ ID NO:422 nucleic acid sequence for encoding amino acid sequence for 2086 protein from M97 250571 strain when combined with a P4 leader sequence.
SEQ ID NO:423 amino acid sequence for 2086 protein from M97 250571 strain prepared using a P4 leader sequence.
SEQ ID NO:424 nucleic acid sequence encoding amino acid sequence for 2086 protein from M97 250571 strain.
SEQ ID NO:425 amino acid sequence for 2086 protein from M97 250571 strain.
SEQ ID NO:426 nucleic acid sequence encoding amino acid sequence for mature 2086 protein from M97 250571 strain when combined with a native leader sequence.
SEQ ID NO:427 amino acid sequence for mature 2086 protein from M97 250571 strain prepared using a native leader sequence.
SEQ ID NO:428 nucleic acid sequence for encoding amino acid sequence for mature 2086 protein from M97 250571 strain when combined with a P4 leader sequence.
SEQ ID NO:429 amino acid sequence for mature 2086 protein from M97 250571 strain prepared using a P4 leader sequence.
SEQ ID NO:430 nucleic acid sequence encoding amino acid sequence for mature 2086 protein from M97 250571 strain.
SEQ ID NO:431 amino acid sequence for mature 2086 protein from M97 250571 strain.
SEQ ID NO:432 nucleic acid sequence encoding amino acid sequence for 2086 protein from M98 250716 strain when combined with a native leader sequence.
SEQ ID NO:433 amino acid sequence for 2086 protein from M98 250716 strain prepared using a native leader sequence.
SEQ ID NO:434 nucleic acid sequence for encoding amino acid sequence for 2086 protein from M98 250716 strain when combined with a P4 leader sequence.
SEQ ID NO:435 amino acid sequence for 2086 protein from M98 250716 strain prepared using a P4 leader sequence.
SEQ ID NO:436 nucleic acid sequence encoding amino acid sequence for 2086 protein from M98 250716 strain.
SEQ ID NO:437 amino acid sequence for 2086 protein from M98 250716 strain.
SEQ ID NO:438 nucleic acid sequence encoding amino acid sequence for mature 2086 protein from M98 250716 strain when combined with a native leader sequence.
SEQ ID NO:439 amino acid sequence for mature 2086 protein from M98 250716 strain prepared using a native leader sequence.
SEQ ID NO:440 nucleic acid sequence for encoding amino acid sequence for mature 2086 protein from M98 250716 strain when combined with a P4 leader sequence.
SEQ ID NO:441 amino acid sequence for mature 2086 protein from M98 250716 strain prepared using a P4 leader sequence.
SEQ ID NO:442 nucleic acid sequence encoding amino acid sequence for mature 2086 protein from M98 250716 strain.
SEQ ID NO:443 amino acid sequence for mature 2086 protein from M98 250716 strain.
SEQ ID NOS:444 to 449 amino acid sequences associated with proteins of 2086 Subfamily B.
SEQ ID NOS:450 to 452 amino acid sequences associated with proteins of 2086 Subfamily A.

### DETAILED DESCRIPTION OF THE INVENTION

A new class of antigens with cross-functional bactericidal activity against Neisseria meningitidis serogroup B would obviate the need for a multi-valent PorA approach to immunization against infection. Such an antigen has been unexpectedly identified and is described and claimed herein. The presence of one such antigen was first observed in a complex mixture of soluble outer membrane proteins (sOMPs) from a meningococcal strain. The bactericidal activity of this antigen was followed through a series of fractionation and protein purification steps until the protein mixture of interest contained just a few proteins. The major proteins in this mixture were identified by N-terminal amino acid sequencing and peptide mapping. The protein of interest exhibiting bactericidal activity was identified as ORF2086 protein, a lipidated protein (also more specifically referred to as LP2086). “ORF2086 protein” refers to a protein encoded by open reading frame 2086 (ORF2086) of Neisseria species.

As described herein, new immunogenic composition candidates based on Neisseria species ORF2086 protein (also referred to as “2086 protein” or “ORF2086” protein, used interchangably herein, or P2086 for the non-lipated proteins and LP2086 for the lipidated version of the proteins) isolated from N. meningitidis were identified by combining cell fractionation, differential detergent extraction, protein purification, with the preparation of antisera, and a bactericidal activity assay utilizing multiple strains. As an alternative to potential immunogenic compositions and diagnostics disclosed in the references cited above, this invention relates to compositions and methods of treating and/or preventing meningococcal infection through the use of proteins, immunogenic portions thereof and biological equivalents thereof, as well as genes encoding said polypeptides, portions and equivalents, and antibodies that immunospecifically bind to same.

According to an embodiment of the present invention, immunogenic agents based on 2086 protein, including isolated polypeptides, immunogenic portions thereof and/or biological equivalents thereof were unexpectedly identified as immunogenic candidates based on the ability of said agents to exhibit cross-reactivity or non-strain specificity. In particular, candidates were identified that unexpectedly demonstrate the ability to (1) elicit bactericidal antibodies to multiple neisserial and/or gonococcal strains; (2) react with the surface of multiple strains; (3) confer passive protection against a live challenge; and/or (4) prevent colonization. Accordingly, the present invention provides immunogenic compositions comprising said immunogenic agents, including isolated polypeptides, immunogenic portions thereof, and/or biological equivalents thereof, as well as methods for using same against infection by N. meningitidis. (See Example 1 herein for the methodology used in the identification of the 2086 protein.)

As used herein, the term “non-strain specific” refers to the characteristic of an antigen to elicit an immune response effective against more than one strain of N. meningitidis (e.g., heterologous meningococcal strains). The term “cross-reactive” as it is used herein is used interchangeably with the term “non-strain specific”. The term “immunogenic non-strain specific N. meningitidis antigen,” as used herein, describes an antigen that can be isolated from N. meningitidis, although it can also be isolated from another bacterium (e.g., other neisserial strains, such as gonococcal strains, for example), or prepared using recombinant technology.

The 2086 proteins of the present invention include lipidated and non-lipidated proteins. Further, the present invention also contemplates the use of the immature proteins or preproteins that correspond to each protein as intermediate compounds/compositions.

The present invention also provides antibodies that immunospecifically bind to the foregoing immunogenic agents, according to implementations of the invention. Further, the present invention relates to isolated polynucleotides comprising nucleic acid sequences encoding any of the foregoing. Additionally, the present invention provides compositions and/or immunogenic compositions and their use in preventing, treating and/or diagnosing meningococcal meningitis, in particular serogroup B meningococcal disease, as well as methods for preparing said compositions.

The compositions of the present invention have been shown to be highly immunogenic and capable of eliciting the production of bactericidal antibodies. These antibodies are cross-reactive to serogroup, serotype and serosubtype heterologous meningococcal strains. Accordingly, the present compositions overcome the deficiencies of previous N. meningitidis vaccine attempts by exhibiting the ability to elicit bactericidal antibodies to heterologous neisserial strains. Thus, among other advantages, the present invention provides immunogenic compositions that can be compounded with fewer components to elicit protection comparable to previously used agents. The compositions or immunogenic agents therein (e.g., polypeptides, immunogenic portions or fragments, and biological equivalents, etc., without limitation) can be used alone or in combination with other antigens or agents to elicit immunological protection from meningococcal infection and disease, as well as to elicit immunological protection from infection and/or disease caused by other pathogens. This simplifies the design of an immunogenic composition for use against meningococcal infection by reducing the number of antigens required for protection against multiple strains. In fact, purified 2086 protein will dramatically and unexpectedly reduce the number of proteins required to provide adequate immunogenic coverage of the strains responsible for meningococcal disease. The 2086 protein can be recombinantly expressed in E. coli as a lipoprotein, which is the wild type form of the protein, at levels much higher than in the native meningococci.

Because antibodies directed against the 2086 protein from a single strain were found to kill unrelated (i.e., heterologous) strains, an attempt was made to characterize a large number of heterologous strains for the presence of a “2086 homolog”, and to determine the level of sequence conservation. While about 70% of the strains tested by PCR had 2086 homologs that could be amplified using the primers that amplified the original 2086 gene from strain 8529, the remaining approximately 30% were negative by this test. These remaining approximately 30% were found to contain a 2086 homolog that has only about 60% amino acid sequence homology to the original 8529 derived 2086 gene. Other primers were identified that could amplify a 2086 homolog from these approximately 30% of strains. The N. meningitidis strains tested have been designated as belonging to Subfamily A or Subfamily B depending on which primer set can amplify a 2086 homolog. The details of these experiments are outlined in Example 5 below.

The Presence of a 2086 Protein in Numerous Serosubtypes.

To determine the level of sequence conservation of the 2086 gene between N. meningitidis strains, several representatives from Subfamilies A and B were cloned as full length genes and submitted for DNA sequence analysis. Using primers as disclosed herein, see, for example, Table IV, twenty four serogroup B meningococcal strains were identified, which express different serosubtype antigens and also express a shared protein, P2086. Examples of these sequences are provided herein and are shown as mature DNA sequences (i.e., all lipoprotein signal sequences have been cleaved at the cysteine residue). See, for example, the amino acid sequences of even numbered SEQ ID NOS: 2-252 and the odd numbered SEQ ID NOS:331-443, without limitation.

Although the 2086 protein is not present in large amounts in wild type strains, it is a target for bactericidal antibodies. These antibodies, unlike those produced in response to the PorAs, are capable of killing strains expressing heterologous serosubtypes.

Antibodies to the 2086 protein also passively protect infant rats from challenge with meningococci. (see Table VII) Recombinant expression of 2086 protein enables the use of 2086 protein as an immunogenic composition for the prevention of meningococcal disease. All of the recent meningococcal immunogenic composition candidates in clinical trials have been complex mixtures or outer membrane protein preparations containing many different proteins. The PorA protein, that provides serosubtype specificity, will require the inclusion of 6 to 9 variants in an immunogenic composition to provide about 70-80% coverage of disease related serosubtypes. In contrast, it is clearly demonstrated herein that antisera to a single 2086 protein alone is able to kill representatives of six serosubtypes responsible for about 65% of the disease isolates in western Europe and the United States. Therefore, purified 2086 protein has the potential to reduce the number of proteins required to provide adequate immunogenic composition coverage of the serosubtypes responsible for meningococcal disease.

Proteins, Immunogenic Portions and Biological Equivalents

The 2086 proteins provided by the present invention are isolated proteins. The term “isolated” means altered by the hand of man from the natural state. If an “isolated” composition or substance occurs in nature, it has been changed or removed from its original environment, or both. For example, a polypeptide or a polynucleotide naturally present in a living animal is not “isolated,” but the same polypeptide or polynucleotide separated from the coexisting materials of its natural state is “isolated”, as the term is employed herein. Accordingly, as used herein, the term “isolated protein” encompasses proteins isolated from a natural source and proteins prepared using recombinant technology, as well as such proteins when combined with other antigens and/or additives, such as pharmaceutically acceptable carriers, buffers, adjuvants, etc., for example.

A 2086 protein, immunogenic portion thereof and/or a biological equivalent thereof, according an embodiment of the invention, comprises an amino acid sequence selected from the group consisting of:

ADIGxGLADA (SEQ ID NO:254), wherein x is any amino acid;

IGxGLADALT (SEQ ID NO:255), wherein x is any amino acid;

SLNTGKLKND (SEQ ID NO:256);

SLNTGKLKNDKxSRFDF (SEQ ID NO:257, wherein x is any amino acid);

SGEFQxYKQ (SEQ ID NO:258), wherein x is any amino acid;

IEHLKxPE (SEQ ID NO:259), wherein x is any amino acid; or combinations thereof.

A 2086 Subfamily A protein, immunogenic portion thereof and/or biological equivalent thereof comprises an amino acid sequence selected from the group consisting of the following amino acid sequences, in accordance with an embodiment of the present invention:

| | |
|---|---|
| GGGVAADIGx, | (SEQ ID NO:260) |
| wherein x is any amino acid; | |
| SGEFQIYKQ; | (SEQ ID NO:261) |
| HSAVVALQIE; | (SEQ ID NO:262) |
| EKINNPDKID; | (SEQ ID NO:263) |
| SLINQRSFLV; | (SEQ ID NO:264) |
| SGLGGEHTAF; | (SEQ ID NO:265) |
| GEHTAFNQLP; | (SEQ ID NO:266) |
| SFLVSGLGGEH; | (SEQ ID NO:267) |
| EKINNPDKIDSLINQRSFLVSGLGGEHTAFNQ | (SEQ ID NO:268) |
| LP; | |
| GKAEYHGKAF; | (SEQ ID NO:269) |
| YHGKAFSSDD; | (SEQ ID NO:270) |
| GKAEYHGKAFSSDD; | (SEQ ID NO:271) |
| IEHLKTPEQN; | (SEQ ID NO:272) |
| KTPEQNVELA; | (SEQ ID NO:273) |
| IEHLKTPEQNVELA; | (SEQ ID NO:274) |
| AELKADEKSH; | (SEQ ID NO:275) |
| AVILGDTRYG; | (SEQ ID NO:276) |
| AELKADEKSHAVILGDTRYG; | (SEQ ID NO:277) |
| EEKGTYHLAL; | (SEQ ID NO:278) |
| or combinations thereof. | |

A 2086 Subfamily B protein, immunogenic portion thereof and/or biological equivalent thereof comprises any of the following amino acid sequences, in accordance with an embodiment embodiment of the present invention:

| | |
|---|---|
| LITLESGEFQ; | (SEQ ID NO:279) |
| SALTALQTEQ; | (SEQ ID NO:280) |
| FQVYKQSHSA; | (SEQ ID NO:281) |
| LITLESGEFQVYKQSHSALTALQTEQ; | (SEQ ID NO:282) |
| IGDIAGEHTS; | (SEQ ID NO:283) |
| EHTSFDKLPK; | (SEQ ID NO:284) |
| IGDIAGEHTSFDKLPK; | (SEQ ID NO:285) |
| ATYRGTAFGS; | (SEQ ID NO:286) |
| DDAGGKLTYT; | (SEQ ID NO:287) |
| IDFAAKQGHG; | (SEQ ID NO:288) |
| KIEHLKSPEL; | (SEQ ID NO:289) |
| ATYRGTAFGSDDAGGKLTYTIDFAAKQGHGKIE | (SEQ ID NO:290) |
| HLKSPELNV; | |
| HAVISGSVLY; | (SEQ ID NO:291) |
| KGSYSLGIFG; | (SEQ ID NO:292) |
| VLYNQDEKGS; | (SEQ ID NO:293) |
| HAVISGSVLYNQDEKGSYSLGIFG; | (SEQ ID NO:294) |
| AQEVAGSAEV; | (SEQ ID NO:295) |
| IHHIGLAAKQ; | (SEQ ID NO:296) |
| VETANGIHHI; | (SEQ ID NO:297) |
| AQEVAGSAEVETANGIHHIGLAAKQ; | (SEQ ID NO:298) |
| or | |
| VAGSAEVETANGIHHIGLAAKQ. | (SEQ ID NO:299) |

The 2086 protein comprises the following consensus sequence and/or immunogenic portions thereof in accordance with an embodiment of the present invention.

2086 Protein Consensus Sequence (SEQ ID NO: 300):

CSSG-----GGGVxADIGxGLADALTxPxDxKDKxLxSLTLxxSxxxNxx
LxLxAQGAEKTxxxGD---SLNTGKLKNDKxSRFDFxxxIxVDGxxITLx
SGEFQxYKQxHSAxxALQxExxxxxxxxxxxxxxRxFxxxxxxGEHTxFx
xLPxx-xAxYxGxAFxSDDxxGxLxYxIDFxxKQGxGxIEHLKxPExNVx
LAxxxxKxDEKxHAVIxGxxxYxxxEKGxYxLxxxGxxAQExAGxAxVxx
xxxxHxIxxAxKQ

In the foregoing consensus sequence, the “x” represents any amino acid, the region from amino acid position 5 to amino acid position 9 is any of 0 to 5 amino acids, the region from amino acid position 67 to amino acid position 69 is any of 0 to 3 amino acids, and amino acid position 156 is any of 0 to 1 amino acid. The region from amino acid position 5 to amino acid position 9 preferably comprises 0, 4 or 5 amino acids. The region from amino acid position 67 to amino acid position 69 preferably comprises 0 or 3 amino acids. It should be particularly noted that this consensus sequence illustrates the high variability of the 2086 proteins. By way of theory, without intending to be bound thereto, it is believed that this high variability provides the advantageous and unexpected cross-reactivity.

According to an implementation of the present invention, the 2086 proteins are characterized as being immunogenic, nonpathogenic and non-strain specific. Moreover, according to a further implementation of the present invention, these proteins unexpectedly exhibit immunogenicity while being about 2% to about 40% nonconserved.

As used herein, the term “nonconserved” refers to the number of amino acids that may undergo insertions, substitution and/or deletions as a percentage of the total number of amino acids in a protein. For example, if a protein is 40% nonconserved and has, for example, 263 amino acids, then there are 105 amino acid positions in the protein at which amino acids may undergo substitution. Likewise, if a protein is 10% nonconserved and has, for example, about 280 amino acids, then there are 28 amino acid positions at which amino acids may undergo substitution. The 2086 proteins may also undergo deletion of amino acid residues without compromising the immunogenicity of the proteins.

Further, the 2086 proteins may be divided into subfamilies based upon homology at various regions. For example, without intending to be limited thereto, the consensus sequences for two such subfamilies, Subfamily A and Subfamily B, are provided below:

2086 Subfamily A sequence (SEQ ID 301)

CSSG----GGGVAADIGxGLADALTxPxDxKDKxLxSLTLxxSxxxNxxL
xLxAQGAEKTxxxGD---SLNTGKLKNDKxSRFDFxxxIxVDGQxITLxS
GEFQIYKQxHSAVVALQIEKINNPDKIDSLINQRSFLVSGLGGEHTAFNQ
LPxGKAEYHGKAFSSDDxxGxLxYxIDFxxKQGxGxIEHLKTPEQNVELA
xAELKADEKSHAVILGDTRYGxEEKGTYHLALxGDRAQEIAGxATVKIxE
KVHEIxIAxKQ

The reference “x” is any amino acid.

The region from amino acid position 5 to amino acid position 8 is any of 0 to 4 amino acids.

The region from amino acid position 66 to amino acid position 68 is any of 0 to 3 amino acids.

The region from amino acid position 5 to amino acid position 8 preferably comprises 0 or 4 amino acids. The region from amino acid position 66 to amino acid position 68 preferably comprises 0 or 3 amino acids.

In accordance with an implementation of the present invention, the 2086 protein from Subfamily A comprises an amino acid sequence selected from the group consisting of:

| | |
|---|---|
| KINNPDKIDSLINQ; | (SEQ ID NO:450) |
| DEKSHAVILG; | (SEQ ID NO:451) |
| KIGEKVHEIG; | (SEQ ID NO:452) |

and combinations thereof.

In accordance with a further embodiment of the invention, the 2086 protein from Subfamily A comprises an amino acid sequence that is encoded by a polynucleotide that hybridizes under stringent conditions to any of the polynucleotides that encode any of SEQ ID NOS:450-452. A person of skill in the art would be able to identify and select polynucleotides (i.e., nucleic acid sequences) that encode any of SEQ ID NOS:450-452 and polynucleotides that hybridize under stringent conditions to any of the polynucleotides that encode any of SEQ ID NOS:450-452.

2086 Subfamily B (SEQ ID 302)

CSSGGGG-----VxADIGxGLADALTAPLDHKDKxLxSLTLxxSxxxNxx
LxLxAQGAEKTYGNGDSLNTGKLKNDKVSRFDFIRQIEVDGxLITLESGE
FQVYKQSHSALTALQTEQxQDxExSxKMVAKRxFxIGDIAGEHTSFDKLP
KxxxATYRGTAFGSDDAGGKLTYTIDFAAKQGHGKIEHLKSPELNVxLAx
xYIKPDEKxHAVISGSVLYNQDEKGSYSLGIFGxxAQEVAGSAEVETANG
IHHIGLAAKQ

The reference “x” is any amino acid.

The region from amino acid position 8 to amino acid position 12 is any of 0 to 5 amino acids.

The region from amino acid position 8 to amino acid position 12 preferably comprises 0 or 5 amino acids.

In accordance with an implementation of the present invention, the 2086 protein comprises an amino acid sequence selected from the group consisting of:

| | |
|---|---|
| MVAKRQFRIG; | (SEQ ID NO:444) |
| DIAGEHTSFDKLP; | (SEQ ID NO:445) |
| YTIDFAAKQG; | (SEQ ID NO:446) |
| GKIEHLKSPELNV; | (SEQ ID NO:447) |
| HAVISGSVLYNQ; | (SEQ ID NO:448) |
| AQEVAGSAEV; | (SEQ ID NO:449) |

and combinations thereof.

In accordance with a further embodiment of the invention, the 2086 protein from Subfamily B comprises an amino acid sequence that is encoded by a polynucleotide that hybridizes under stringent conditions to any of the polynucleotides that encode a any of SEQ ID NOS: 444-449. A person of skill in the art would be able to identify and select polynucleotides (i.e., nucleic acid sequences) that encode any of SEQ ID NOS:444-449 and nucleotide sequences that hybridize under stringent conditions to any of the nucleotide sequences that encode any of SEQ ID NOS:444-449.

According to implementations of the present invention, the 2086 protein subfamilies may be further subdivided into clusters. For example, according to an implementation of the present invention, the following clusters are provided: even numbered SEQ ID NOS:2-12; even numbered SEQ ID NOS:14-24; even numbered SEQ ID NOS:26-42; even numbered SEQ ID NOS:50-60; even numbered SEQ ID NOS:62-108; even numbered SEQ ID NOS:110-138; even numbered SEQ ID NOS:140-156; even numbered SEQ ID NOS:158-174; and even numbered SEQ ID NOS: 224-252.

A polypeptide sequence of the invention may be identical to the reference sequence (e.g., even numbered SEQ ID NOS:2-252 or odd numbered SEQ ID NOS:331-443), that is, 100% identical, or it may include a number of amino acid alterations as compared to the reference sequence such that the % identity is less than 100%. Such alterations include at least one amino acid deletion, substitution, including conservative and non-conservative substitution, or insertion. The alterations may occur at the amino- or carboxy-terminal positions of the reference polypeptide sequence or anywhere between those terminal positions, interspersed either individually among the amino acids in the reference amino acid sequence or in one or more contiguous groups within the reference amino acid sequence.

Thus, the invention also provides proteins having sequence identity to the amino acid sequences contained in the Sequence Listing (i.e., even numbered SEQ ID NOS: 2-252 or odd numbered SEQ ID NOS:331-443). Depending on the particular sequence, the degree of sequence identity is preferably greater than 60% (e.g., 60%, 70%, 80%, 90%, 95%, 97%, 99%, 99.9% or more). These homologous proteins include mutants and allelic variants.

In preferred embodiments of the invention, the 2086 proteins or other 2086 polypeptides (e.g., immunological portions and biological equivalents) generate bactericidal antibodies to homologous and at least one heterologous strain of meningococci. Specifically, the antibodies to the 2086 polypeptides passively protect infant rats from challenge, such as intranasal, with meningococci. In further preferred embodiments, the 2086 polypeptides exhibit such protection for infants rats for homologous strains and at least one heterologous strain. The polypeptide may be selected from the Sequence Summary above, as set forth in the even numbered SEQ ID NOS: 2-252 and odd numbered SEQ ID NOS:331-443, or the polypeptide may be any immunological fragment or biological equivalent of the listed polypeptides. Preferably, the polypeptide is selected from any of the even numbered SEQ ID NOS: 2-252 or the odd numbered SEQ ID NOS:331-443 in the Sequence Summary above.

This invention also relates to allelic or other variants of the 2086 polypeptides, which are biological equivalents. Suitable biological equivalents will exhibit the ability to (1) elicit bactericidal antibodies to homologous strains and at least one heterologous neisserial strain and/or gonococcal strain; (2) react with the surface of homologous strains and at least one heterologous neisserial and/or gonococcal strain; (3) confer passive protection against a live challenge; and/or (4) prevent colonization.

Suitable biological equivalents have at least about 60%, preferably at least about 70%, more preferably at least about 75%, even more preferably about 80%, even more preferably about 85%, even more preferably about 90%, even more preferably 95% or even more preferably 98%, or even more preferably 99% similarity to one of the 2086 polypeptides specified herein (i.e., the even numbered SEQ ID NOS: 2-252 and odd numbered SEQ ID NOS:331-443), provided the equivalent is capable of eliciting substantially the same immunogenic properties as one of the 2086 proteins of this invention.

Alternatively, the biological equivalents have substantially the same immunogenic properties of one of the 2086 protein in the even numbered SEQ ID NOS: 2-252 and odd numbered SEQ ID NOS:331-443. According to embodiments of the present invention, the biological equivalents have the same immunogenic properties as the even numbered SEQ ID NOS: 2-252 or odd numbered SEQ ID NOS:331-443.

The biological equivalents are obtained by generating variants and modifications to the proteins of this invention. These variants and modifications to the proteins are obtained by altering the amino acid sequences by insertion, deletion or substitution of one or more amino acids. The amino acid sequence is modified, for example by substitution in order to create a polypeptide having substantially the same or improved qualities. A preferred means of introducing alterations comprises making predetermined mutations of the nucleic acid sequence of the polypeptide by site-directed mutagenesis.

Modifications and changes can be made in the structure of a polypeptide of the present invention and still obtain a molecule having N. meningitidis immunogencity. For example, without limitation, certain amino acids can be substituted for other amino acids, including nonconserved and conserved substitution, in a sequence without appreciable loss of immunogenicity. Because it is the interactive capacity and nature of a polypeptide that defines that polypeptide's biological functional activity, a number of amino acid sequence substitutions can be made in a polypeptide sequence (or, of course, its underlying DNA coding sequence) and nevertheless obtain a polypeptide with like properties. The present invention contemplates any changes to the structure of the polypeptides herein, as well as the nucleic acid sequences encoding said polypeptides, wherein the polypeptide retains immunogenicity. A person of ordinary skill in the art would be readily able to modify the disclosed polypeptides and polynucleotides accordingly, based upon the guidance provided herein.

For example, certain variable regions have been identified where substitution or deletion is permissible The 2086 consensus sequence, as previously discussed, shows conserved and nonconserved regions of the 2086 family of proteins according to an implementation of the present invention.

In making such changes, any techniques known to persons of skill in the art may be utilized. For example, without intending to be limited thereto, the hydropathic index of amino acids can be considered. The importance of the hydropathic amino acid index in conferring interactive biologic function on a polypeptide is generally understood in the art. Kyte etal. 1982. J. Mol. Bio. 157:105-132.

Substitution of like amino acids can also be made on the basis of hydrophilicity, particularly where the biological functional equivalent polypeptide or peptide thereby created is intended for use in immunological embodiments. U.S. Pat. No. 4,554,101, incorporated herein by reference, states that the greatest local average hydrophilicity of a polypeptide, as governed by the hydrophilicity of its adjacent amino acids, correlates with its immunogenicity, i.e. with a biological property of the polypeptide.

Biological equivalents of a polypeptide can also be prepared using site-specific mutagenesis. Site-specific mutagenesis is a technique useful in the preparation of second generation polypeptides, or biologically functional equivalent polypeptides or peptides, derived from the sequences thereof, through specific mutagenesis of the underlying DNA. Such changes can be desirable where amino acid substitutions are desirable. The technique further provides a ready ability to prepare and test sequence variants, for example, incorporating one or more of the foregoing considerations, by introducing one or more nucleotide sequence changes into the DNA. Site-specific mutagenesis allows the production of mutants through the use of specific oligonucleotide sequences which encode the DNA sequence of the desired mutation, as well as a sufficient number of adjacent nucleotides, to provide a primer sequence of sufficient size and sequence complexity to form a stable duplex on both sides of the deletion junction being traversed. Typically, a primer of about 17 to 25 nucleotides in length is preferred, with about 5 to 10 residues on both sides of the junction of the sequence being altered.

In general, the technique of site-specific mutagenesis is well known in the art. As will be appreciated, the technique typically employs a phage vector which can exist in both a single stranded and double stranded form. Typically, site-directed mutagenesis in accordance herewith is performed by first obtaining a single-stranded vector which includes within its sequence a DNA sequence which encodes all or a portion of the N. meningitidis polypeptide sequence selected. An oligonucleotide primer bearing the desired mutated sequence is prepared (e.g., synthetically). This primer is then annealed to the single-stranded vector, and extended by the use of enzymes such as E. coli polymerase I Klenow fragment, in order to complete the synthesis of the mutation-bearing strand. Thus, a heteroduplex is formed wherein one strand encodes the original non-mutated sequence and the second strand bears the desired mutation. This heteroduplex vector is then used to transform appropriate cells such as E. coli cells and clones are selected which include recombinant vectors bearing the mutation. Commercially available kits come with all the reagents necessary, except the oligonucleotide primers.

2086 polypeptides include any protein or polypeptide comprising substantial sequence similarity and/or biological equivalence to a 2086 protein having an amino acid sequence from one of the even numbered SEQ ID NOS 2-252. In addition, a 2086 polypeptide of the invention is not limited to a particular source. Thus, the invention provides for the general detection and isolation of the polypeptides from a variety of sources. Also, the 2086 polypeptides can be prepared recombinantly, as is well within the skill in the art, based upon the guidance provided herein, or in any other synthetic manner, as known in the art.

It is contemplated in the present invention, that a 2086 polypeptide may advantageously be cleaved into fragments for use in further structural or functional analysis, or in the generation of reagents such as 2086-related polypeptides and 2086-specific antibodies. This can be accomplished by treating purified or unpurified N. meningitidis polypeptides with a peptidase such as endoproteinase glu-C (Boehringer, Indianapolis, Ind.). Treatment with CNBr is another method by which peptide fragments may be produced from natural N. meningitidis 2086 polypeptides. Recombinant techniques also can be used to produce specific fragments of a 2086 protein.

“Variant” as the term is used herein, is a polynucleotide or polypeptide that differs from a reference polynucleotide or polypeptide respectively, but retains essential properties. A typical variant of a polynucleotide differs in nucleotide sequence from another, reference polynucleotide. Changes in the nucleotide sequence of the variant may or may not alter the amino acid sequence of a polypeptide encoded by the reference polynucleotide. Nucleotide changes may result in amino acid substitutions, additions, deletions, fusions and truncations in the polypeptide encoded by the reference sequence, as discussed below. A typical variant of a polypeptide differs in amino acid sequence from another, reference polypeptide. Generally, differences are limited so that the sequences of the reference polypeptide and the variant are closely similar overall and, in many regions, identical (i.e., biologically equivalent). A variant and reference polypeptide may differ in amino acid sequence by one or more substitutions, additions, deletions in any combination. A substituted or inserted amino acid residue may or may not be one encoded by the genetic code. A variant of a polynucleotide or polypeptide may be a naturally occurring such as an allelic variant, or it may be a variant that is not known to occur naturally. Non-naturally occurring variants of polynucleotides and polypeptides may be made by mutagenesis techniques or by direct synthesis.

“Identity,” as known in the art, is a relationship between two or more polypeptide sequences or two or more polynucleotide sequences, as determined by comparing the sequences. In the art, “identity” also means the degree of sequence relatedness between polypeptide or polynucleotide sequences, as the case may be, as determined by the match between strings of such sequences. “Identity” and “similarity” can be readily calculated by known methods, including but not limited to those described in Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991; and Carillo, H., and Lipman, D., SIAM J. Applied Math., 48:1073 (1988). Preferred methods to determine identity are designed to give the largest match between the sequences tested. Methods to determine identity and similarity are codified in publicly available computer programs. Preferred computer program methods to determine identity and similarity between two sequences include, but are not limited to, the GCG program package (Devereux, J., et al 1984), BLASTP, BLASTN, and FASTA (Altschul, S. F., et al., 1990). The BLASTX program is publicly available from NCBI and other sources (BLAST Manual, Altschul, S., et al., NCBI NLM NIH Bethesda, Md. 20894; Altschul, S., et al., 1990). The well known Smith Waterman algorithm may also be used to determine identity.

By way of example, without intending to be limited thereto, an amino acid sequence of the present invention may be identical to the reference sequences, even numbered SEQ ID NOS: 2-252 and odd numbered SEQ ID NOS:331-443; that is be 100% identical, or it may include a number of amino acid alterations as compared to the reference sequence such that the % identity is less than 100%. Such alterations are selected from the group consisting of at least one amino acid deletion, substitution, including conservative and non-conservative substitution, or insertion, and wherein said alterations may occur at the amino- or carboxy-terminal positions of the reference polypeptide sequence or anywhere between those terminal positions, interspersed either individually among the amino acids in the reference sequence or in one or more contiguous groups within the reference sequence. The number of amino acid alterations for a given % identity is determined by multiplying the total number of amino acids in even numbered SEQ ID NOS:2-252 and odd numbered SEQ ID NOS:331-443 by the numerical percent of the respective percent identity (divided by 100) and then subtracting that product from said total number of amino acids in any of even numbered SEQ ID NOS:2-252 and odd numbered SEQ ID NOS:331-443, or:
nₐ=Xₐ−(Xₐ·Y),
wherein nₐ is the number of amino acid alterations, xₐ is the total number of amino acids in even numbered SEQ ID NOS:2-252 and odd numbered SEQ ID NOS:331-443, and y is, for instance 0.70 for 70%, 0.80 for 80%, 0.85 for 85% etc., and wherein any non-integer product of x.sub.a and y is rounded down to the nearest integer prior to subtracting it from xₐ.

In preferred embodiments, the polypeptide above is selected from the proteins set forth in the even numbered SEQ ID NOS 2-252, such as mature processed form of a 2086 protein. The 2086 proteins or equivalents, etc. may be lipidated or non-lipidated.

ORF 2086 is expressible in E. coli with the native ORF 2086 signal sequence. However, it is desirable to find means to improve the expression of proteins. According to an embodiment of the present invention, a leader sequence produces a lipidated form of the protein. For example, the following describes the use of the signal sequence of the nontypable Haemophilus influenzae P4 protein to enhance expression.

The processing of bacterial lipoproteins begins with the synthesis of a precursor or prolipoprotein containing a signal sequence, which in turn contains a consensus lipoprotein processing/modification site. This prolipoprotein initially passes through the common Sec system on the inner membrane of Gram negative bacteria or on the membrane in Gram positive bacteria. Once placed in the membrane by the Sec system, the prolipoprotein is cleaved by signal peptidase II at the consensus site and the exposed N-terminal cysteine residue is glycerated and acylated. Hayashi et al. 1990. Lipoproteins in bacteria. J. Bioenerg. Biomembr. June; 22(3):451-71; Oudega et al. 1993. Escherichia coli SecB, SecA, and SecY proteins are required for expression and membrane insertion of the bacteriocin release protein, a small lipoprotein. J. Bacteriol. March; 175(5):1543-7; Sankaran et al. 1995. Modification of bacterial lipoproteins. Methods Enzymol. 250:683-97.

In Gram negative bacteria, transport of the lipidated protein to the outer membrane is mediated by a unique ABC transporter system with membrane specificity depending on a sorting signal at position 2 of the lipoprotein. Yakushi et al. 2000. A new ABC transporter mediating the detachment of lipid modified proteins from membranes. Nat Cell Biol. April; 2(4):212-8.

Fusion with bacterial lipoproteins and their signal sequences has been used to display recombinant proteins on the surface of bacteria. U.S. Pat. Nos. 5,583,038 and 6,130,085. Exchanging lipoprotein signal sequences can increase the production of the lipoprotein. De et al. 2000. Purification and characterization of Streptococcus pneumoniae palmitoylated pneumococcal surface adhesin A expressed in Escherichia coli. Vaccine. March 6; 18(17):1811-21.

Bacterial lipidation of proteins is known to increase or modify the immunological response to proteins. Erdile et al. 1993. Role of attached lipid in immunogenicity of Borrelia burgdorferi OspA. Infect. Immun. January; 61(1):81-90; Snapper et al. 1995. Bacterial lipoproteins may substitute for cytokines in the humoral immune response to T cell-independent type II antigens. J. Immunol. December 15; 155(12):5582-9. However, bacterial lipoprotein expression can be complicated by the stringency of the processing. Pollitt et al. 1986. Effect of amino acid substitutions at the signal peptide cleavage site of the Escherichia coli major outer membrane lipoprotein. J. Biol. Chem. February 5; 261(4):1835-7; Lunn et al. 1987. Effects of prolipoprotein signal peptide mutations on secretion of hybrid prolipo-beta-lactamase in Escherichia coli. J. Biol. Chem. June 15; 262(17):8318-24; Klein et al. 1988. Distinctive properties of signal sequences from bacterial lipoproteins. Protein Eng. April; 2(1):15-20. Bacterial lipoprotein expression is also complicated by other problems such as toxicity and low expression levels. Gomez et al. 1994. Nucleotide The Bacillus subtilis lipoprotein LplA causes cell lysis when expressed in Escherichia coli. Microbiology. August; 140 (Pt 8):1839-45; Hansson et al. 1995. Expression of truncated and full-length forms of the Lyme disease Borrelia outer surface protein A in Escherichia coli. Protein Expr. Purif. February; 6(1):15-24; Yakushi et al. 1997. Lethality of the covalent linkage between mislocalized major outer membrane lipoprotein and the peptidoglycan of Escherichia coli. J. Bacteriol. May; 179(9):2857-62.

The nontypable Haemophilus influenzae bacterium expresses a lipoprotein designated P4 (also known as protein “e”). The recombinant form of the P4 protein is highly expressed in E. coli using the native P4 signal sequence. U.S. Pat. No. 5,955,580. When the native P4 signal sequence is substituted for the native ORF 2086 signal sequence in an expression vector in E. coli, the level of expression of ORF2086 is increased.

This concept of using the heterologous P4 signal sequence to increase expression is extendible to other bacterial lipoproteins. In particular, analysis of bacterial genomes leads to the identification of many ORFs as being of possible interest. Attempting to express each ORF with its native signal sequence in a heterologous host cell, such as E. coli, gives rise to a variety of problems inherent in using a variety of signal sequences, including stability, compatibility and so forth. To minimize these problems, the P4 signal sequence is used to express each ORF of interest. As described above, the P4 signal sequence improves the expression of the heterologous 2086 ORF. An expression vector is constructed by deleting the native signal sequence of the ORF of interest, and ligating the P4 signal sequence to the ORF. A suitable host cell is then transformed, transfected or infected with the expression vector, and expression of the ORF is increased in comparison to expression using the native signal sequence of the ORF.

The non-lipidated form is produced by a protein lacking the original leader sequence or a by a leader sequence which is replaced with a portion of sequence that does not specify a site for fatty acid acylation in a host cell.

The various forms of the 2086 proteins of this invention are referred to herein as “2086” protein, unless otherwise specifically noted. Also “2086 polypeptide” refers to the 2086 proteins as well as immunogenic portions or biological equivalents thereof as noted above, unless otherwise noted.

The full length isolated and purified N. meningitidis 2086 protein has an apparent molecular weight of about 28 to 35 kDa as measured on a 10% to 20% gradient SDS polyacrylamide gel (SDS-PAGE). More specifically, this protein has a molecular weight of about 26,000 to 30,000 daltons as measured by mass spectrometry.

Preferably, the 2086 polypeptides and nucleic acids encoding such polypeptides are used for preventing or ameliorating infection caused by N. meningitidis and/or other species.

Antibodies

The proteins of the invention, including the amino acid sequences of SEQ ID NOS: 2-252 and odd numbered SEQ ID NOS:331-443, their fragments, and analogs thereof, or cells expressing them, are also used as immunogens to produce antibodies immunospecific for the polypeptides of the invention. The invention includes antibodies to immunospecific polypeptides and the use of such antibodies to detect the presence of N. meningitidis, provide passive protection or measure the quantity or concentration of the polypeptides in a cell, a cell or tissue extract, or a biological fluid.

The antibodies of the invention include polyclonal antibodies, monoclonal antibodies, chimeric antibodies, and anti-idiotypic antibodies. Polyclonal antibodies are heterogeneous populations of antibody molecules derived from the sera of animals immunized with an antigen. Monoclonal antibodies are a substantially homogeneous population of antibodies to specific antigens. Monoclonal antibodies may be obtained by methods known to those skilled in the art, e.g., Kohler and Milstein, 1975, Nature 256:495-497 and U.S. Pat. No. 4,376,110. Such antibodies may be of any immunoglobulin class including IgG, IgM, IgE, IgA, GILD and any subclass thereof.

Chimeric antibodies are molecules, different portions of which are derived from different animal species, such as those having variable region derived from a murine monoclonal antibody and a human immunoglobulin constant region. Chimeric antibodies and methods for their production are known in the art (Cabilly et al., 1984, Proc. Natl. Acad. Sci. USA 81:3273-3277; Morrison et al., 1984, Proc. Natl. Acad. Sci. USA 81:6851-6855; Boulianne et al., 1984, Nature 312:643-646; Cabilly et al., European Patent Application 125023 (published Nov. 14, 1984); Taniguchi et al., European Patent Application 171496 (published Feb. 19, 1985); Morrison et al., European Patent Application 173494 (published Mar. 5, 1986); Neuberger et al., PCT Application WO 86/01533 (published Mar. 13, 1986); Kudo et al., European Patent Application 184187 (published Jun. 11, 1986); Morrison et al., European Patent Application 173494 (published Mar. 5, 1986); Sahagan et al., 1986, J. Immunol. 137:1066-1074; Robinson et al., PCT/US86/02269 (published May 7, 1987); Liu et al., 1987, Proc. Natl. Acad. Sci. USA 84:3439-3443; Sun et al., 1987, Proc. Natl. Acad. Sci. USA 84:214-218; Better et al., 1988, Science 240:1041-1043). These references are hereby incorporated by reference in their entirety.

An anti-idiotypic (anti-Id) antibody is an antibody which recognizes unique determinants generally associated with the antigen-binding site of an antibody. An anti-Id antibody is prepared by immunizing an animal of the same species and genetic type (e.g., mouse strain) as the source of the monoclonal antibody with the monoclonal antibody to which an anti-Id is being prepared. The immunized animal will recognize and respond to the idiotypic determinants of the immunizing antibody by producing an antibody to these isotypic determinants (the anti-Id antibody).

Accordingly, monoclonal antibodies generated against the polypeptides of the present invention may be used to induce anti-Id antibodies in suitable animals. Spleen cells from such immunized mice can be used to produce anti-Id hybridomas secreting anti-Id monoclonal antibodies. Further, the anti-Id antibodies can be coupled to a carrier such as keyhole limpet hemocyanin (KLH) and used to immunize additional BALB/c mice. Sera from these mice will contain anti-anti-Id antibodies that have the binding properties of the final mAb specific for an R-PTPase epitope. The anti-Id antibodies thus have their idiotypic epitopes, or “idiotopes” structurally similar to the epitope being evaluated, such as Streptococcus pyogenes polypeptides.

The term “antibody” is also meant to include both intact molecules as well as fragments such as Fab which are capable of binding antigen. Fab fragments lack the Fc fragment of intact antibody, clear more rapidly from the circulation, and may have less non-specific tissue binding than an intact antibody (Wahl et al., 1983, J. Nucl. Med. 24:316-325). It will be appreciated that Fab and other fragments of the antibodies useful in the present invention may be used for the detection and quantitation of N. meningitidis polypeptides according to the methods for intact antibody molecules.

The antibodies of this invention, such as anti-iodiotypic (“anti-Id”) antibodies, can be employed in a method for the treatment or prevention of Neisseria infection in mammalian hosts, which comprises administration of an immunologically effective amount of antibody, specific for a polypeptide as described above. The anti-Id antibody may also be used as an “immunogen” to induce an immune response in yet another animal, producing a so-called anti-anti-Id antibody. The anti-anti-Id may be epitopically identical to the original mAb which induced the anti-Id. Thus, by using antibodies to the idiotypic determinants of a mAb, it is possible to identify other clones expressing antibodies of identical specificity.

The antibodies are used in a variety of ways, e.g., for confirmation that a protein is expressed, or to confirm where a protein is expressed. Labeled antibody (e.g., fluorescent labeling for FACS) can be incubated with intact bacteria and the presence of the label on the bacterial surface confirms the location of the protein, for instance.

Antibodies generated against the polypeptides of the invention can be obtained by administering the polypeptides or epitope-bearing fragments, analogs, or cells to an animal using routine protocols. For preparing monoclonal antibodies, any technique which provides antibodies produced by continuous cell line cultures are used.

Polynucleotides

As with the proteins of the present invention, a polynucleotide of the present invention may comprise a nucleic acid sequence that is identical to any of the reference sequences of even numbered SEQ ID NOS:330-442, that is be 100% identical, or it may include up to a number of nucleotide alterations as compared to the reference sequence. Such alterations are selected from the group consisting of at least one nucleotide deletion, substitution, including transition and transversion, or insertion, and wherein said alterations may occur at the 5′ or 3′ terminal positions of the reference nucleotide sequence or anywhere between those terminal positions, interspersed either individually among the nucleotides in the reference sequence or in one or more contiguous groups within the reference sequence. The number of nucleotide alterations is determined by multiplying the total number of nucleotides in any of even numbered SEQ ID NOS:330-442 by the numerical percent of the respective percent identity (divided by 100) and subtracting that product from said total number of nucleotides in said sequence.

By way of example, without intending to be limited thereto, an isolated N. meningitidis polynucleotide comprising a polynucleotide sequence that has at least 70% identity to any nucleic acid sequence of odd numbered SEQ ID NOS:1-253 and even numbered SEQ ID NOS:330-442; a degenerate variant thereof or a fragment thereof, wherein the polynucleotide sequence may include up to nₙ nucleic acid alterations over the entire polynucleotide region of the nucleic acid sequence of odd numbered SEQ ID NOS:1-253 and even numbered SEQ ID NOS:330-442, wherein nₙ is the maximum number of alterations and is calculated by the formula:
nₙ=xₙ−(xₙ·y),
in which xₙ is the total number of nucleic acids of any of odd numbered SEQ ID NOS:1-253 and even numbered SEQ ID NOS:330-442 and y has a value of 0.70, wherein any non-integer product of xₙ and y is rounded down to the nearest integer prior to subtracting such product from xₙ. Of course, y may also have a value of 0.80 for 80%, 0.85 for 85%, 0.90 for 90% 0.95 for 95%, etc. Alterations of a polynucleotide sequence encoding the polypeptides comprising amino acid sequences of any of even numbered SEQ ID NOS:2-252 and odd numbered SEQ ID NOS:331-443 may create nonsense, missense or frameshift mutations in this coding sequence and thereby alter the polypeptide encoded by the polynucleotide following such alterations.

Certain embodiments of the present invention relate to polynucleotides (herein referred to as the “2086 polynucleotides” or “ORF2086 polynucleotides”) which encode the 2086 proteins and antibodies made against the 2086 proteins. In preferred embodiments, an isolated polynucleotide of the present invention is a polynucleotide comprising a nucleotide sequence having at least about 95% identity to a nucleotide sequence chosen from one of the odd numbered SEQ ID NOS:1-253 or even numbered SEQ ID NOS:330-442, a degenerate variant thereof, or a fragment thereof. As defined herein, a “degenerate variant” is defined as a polynucleotide that differs from the nucleotide sequence shown in the odd numbered SEQ ID NOS:1 and SEQ ID NOS:253 (and fragments thereof) due to degeneracy of the genetic code, but still encodes the same 2086 protein (e.g., the even numbered SEQ ID NOS: 2-252 and odd numbered SEQ ID NOS:331-443) as that encoded by the nucleotide sequence shown in the odd numbered SEQ ID NOS: 1-253 and even numbered SEQ ID NOS:330-442.

In other embodiments, the polynucleotide is a complement to a nucleotide sequence chosen from one of the odd numbered SEQ ID NOS: 1-253 or even numbered SEQ ID NOS:330-442, a degenerate variant thereof, or a fragment thereof. In yet other embodiments, the polynucleotide is selected from the group consisting of DNA, chromosomal DNA, cDNA and RNA and may further comprises heterologous nucleotides. In another embodiment, an isolated polynucleotide hybridizes to a nucleotide sequence chosen from one of SEQ ID NOS: 1-253 or even numbered SEQ ID NOS:330-442, a complement thereof, a degenerate variant thereof, or a fragment thereof, under high stringency hybridization conditions. In yet other embodiments, the polynucleotide hybridizes under intermediate stringency hybridization conditions.

It will be appreciated that the 2086 polynucleotides may be obtained from natural, synthetic or semi-synthetic sources; furthermore, the nucleotide sequence may be a naturally occurring sequence, or it may be related by mutation, including single or multiple base substitutions, deletions, insertions and inversions, to such a naturally occurring sequence, provided always that the nucleic acid molecule comprising such a sequence is capable of being expressed as 2086 immunogenic polypeptide as described above. The nucleic acid molecule may be RNA, DNA, single stranded or double stranded, linear or covalently closed circular form. The nucleotide sequence may have expression control sequences positioned adjacent to it, such control sequences usually being derived from a heterologous source. Generally, recombinant expression of the nucleic acid sequence of this invention will use a stop codon sequence, such as TAA, at the end of the nucleic acid sequence.

The invention also includes polynucleotides capable of hybridizing under reduced stringency conditions, more preferably stringent conditions, and most preferably highly stringent conditions, to polynucleotides described herein. Examples of stringency conditions are shown in the Stringency Conditions Table below: highly stringent conditions are those that are at least as stringent as, for example, conditions A-F; stringent conditions are at least as stringent as, for example, conditions G-L; and reduced stringency conditions are at least as stringent as, for example, conditions M-R.

TABLE I
STRINGENCY CONDITIONS

| | | | Hybridization | |
|---|---|---|---|---|
| Stringency | Polynucleotide | Hybrid Length | Temperature and | Wash Temperature |
| Condition | Hybrid | (bp)^{I} | Buffer^{H} | and Buffer^{H} |
| A | DNA:DNA | >50 | 65EC; 1xSSC -or- | 65EC; |
| | | | 42EC; 1xSSC, 50% | 0.3xSSC |
| | | | formamide | |
| B | DNA:DNA | <50 | T_{B}; 1xSSC | T_{B}; 1xSSC |
| C | DNA:RNA | >50 | 67EC; 1xSSC -or- | 67EC; |
| | | | 45EC; 1xSSC, 50% | 0.3xSSC |
| | | | formamide | |
| D | DNA:RNA | <50 | T_{D}; 1xSSC | T_{D}; 1xSSC |
| E | RNA:RNA | >50 | 70EC; 1xSSC -or- | 70EC; |
| | | | 50EC; 1xSSC, 50% | 0.3xSSC |
| | | | formamide | |
| F | RNA:RNA | <50 | T_{F}; 1xSSC | T_{f}; 1xSSC |
| G | DNA:DNA | >50 | 65EC; 4xSSC -or- | 65EC; 1xSSC |
| | | | 42EC; 4xSSC, 50% | |
| | | | formamide | |
| H | DNA:DNA | <50 | T_{H}; 4xSSC | T_{H}; 4xSSC |
| I | DNA:RNA | >50 | 67EC; 4xSSC -or- | 67EC; 1xSSC |
| | | | 45EC; 4xSSC, 50% | |
| | | | formamide | |
| J | DNA:RNA | <50 | T_{J}; 4xSSC | T_{J}; 4xSSC |
| K | RNA:RNA | >50 | 70EC; 4xSSC -or- | 67EC; 1xSSC |
| | | | 50EC; 4xSSC, 50% | |
| | | | formamide | |
| L | RNA:RNA | <50 | T_{L}; 2xSSC | T_{L}; 2xSSC |
| M | DNA:DNA | >50 | 50EC; 4xSSC -or- | 50EC; 2xSSC |
| | | | 40EC; 6xSSC, 50% | |
| | | | formamide | |
| N | DNA:DNA | <50 | T_{N}; 6xSSC | T_{N}; 6xSSC |
| O | DNA:RNA | >50 | 55EC; 4xSSC -or- | 55EC; 2xSSC |
| | | | 42EC; 6xSSC, 50% | |
| | | | formamide | |
| P | DNA:RNA | <50 | T_{P}; 6xSSC | T_{P}; 6xSSC |
| Q | RNA:RNA | >50 | 60EC; 4xSSC -or- | 60EC; 2xSSC |
| | | | 45EC; 6xSSC, 50% | |
| | | | formamide | |
| R | RNA:RNA | <50 | T_{R}; 4xSSC | T_{R}; 4xSSC |

bp¹: The hybrid length is that anticipated for the hybridized region(s) of the hybridizing polynucleotides. When hybridizing a polynucleotide to a target polynucleotide of unknown sequence, the hybrid length is assumed to be that of the hybridizing polynucleotide. When polynucleotides of known sequence are hybridized, the hybrid length can be determined by aligning the sequences of the polynucleotides and identifying the region or regions of optimal sequence complementarities.
Buffer^{H}: SSPE (1xSSPE is 0.15M NaCl, 10 mM NaH₂PO₄, and 1.25 mM EDTA, pH 7.4) can be substituted for SSC (1xSSC is 0.15M NaCl and 15 mM sodium citrate) in the hybridization and wash buffers; washes are performed for 15 minutes after hybridization is complete.
T_{B} through T_{R}: The hybridization temperature for hybrids anticipated to be less than 50 base pairs in length should be 5-10EC less than the melting temperature (Tₘ) of the hybrid, where Tₘ is determined according to the following equations. For hybrids less than 18 base pairs in length, Tₘ(EC) = 2(# of A + T bases) + 4(# of G + C bases).
For hybrids between 18 and 49 base pairs in length, Tₘ(EC) = 81.5 + 16.6(log₁₀[Na⁺]) + 0.41(% G + C) − (600/N), where N is the number of bases in the hybrid, and ([Na⁺] is the concentration of sodium ions in the hybridization buffer ([Na⁺] for 1xSSC = 0.165 M).

Additional examples of stringency conditions for polynucleotide hybridization are provided in Sambrook, J., E. F. Fritsch, and T. Maniatis, 1989, Molecular Cloning: A Laboratory Manual, Cold Spring Harbor Laboratory Press, Cold Spring Harbor, N.Y., chapters 9 and 11, and Current Protocols in Molecular Biology, 1995, F. M. Ausubel et al., eds., John Wiley & Sons, Inc., sections 2.10 and 6.3-6.4, incorporated herein by reference.

The invention also provides polynucleotides that are fully complementary to these polynucleotides and also provides antisense sequences. The antisense sequences of the invention, also referred to as antisense oligonucleotides, include both internally generated and externally administered sequences that block expression of polynucleotides encoding the polypeptides of the invention. The antisense sequences of the invention comprise, for example, about 15-20 base pairs. The antisense sequences can be designed, for example, to inhibit transcription by preventing promoter binding to an upstream nontranslated sequence or by preventing translation of a transcript encoding a polypeptide of the invention by preventing the ribosome from binding.

The polynucleotides of the invention are prepared in many ways (e.g., by chemical synthesis, from DNA libraries, from the organism itself) and can take various forms (e.g., single-stranded, double-stranded, vectors, probes, primers). The term “polynucleotide” includes DNA and RNA, and also their analogs, such as those containing modified backbones.

According to further implementations of the present invention, the polynucleotides of the present invention comprise a DNA library, such as a cDNA library.

Fusion Proteins

The present invention also relates to fusion proteins. A “fusion protein” refers to a protein encoded by two, often unrelated, fused genes or fragments thereof. For example, fusion proteins comprising various portions of constant region of immunoglobulin molecules together with another immunogenic protein or part thereof. In many cases, employing an immunoglobulin Fc region as a part of a fusion protein is advantageous for use in therapy and diagnosis resulting in, for example, improved pharmacokinetic properties (see, e.g., EP 0 232 262 A1). On the other hand, for some uses it would be desirable to be able to delete the Fc part after the fusion protein has been expressed, detected and purified. The 2086 polynucleotides of the invention are used for the recombinant production of polypeptides of the present invention, the polynucleotide may include the coding sequence for the mature polypeptide, by itself, or the coding sequence for the mature polypeptide in reading frame with other coding sequences, such as those encoding a leader or secretory sequence, a pre-, or pro- or prepro-protein sequence, or other fusion peptide portions. For example, a marker sequence which facilitates purification of a 2086 polypeptide or fused polypeptide can be encoded (see Gentz et al., 1989, incorporated herein by reference in its entirety). Thus, contemplated in an implementation of the present invention is the preparation of polynucleotides encoding fusion polypeptides permitting His-tag purification of expression products. The polynucleotide may also contain non-coding 5′ and 3′ sequences, such as transcribed, non-translated sequences, splicing and polyadenylation signals. Such a fused polypeptide can be produced by a host cell transformed/transfected or infected or infected with a recombinant DNA cloning vehicle as described below and it can be subsequently isolated from the host cell to provide the fused polypeptide substantially free of other host cell. proteins.

Immunogenic Compositions

One aspect of the present invention provides immunogenic compositions which comprise at least one 2086 proteins or a nucleic acid encoding said proteins. The foregoing have the ability to (1) elicit bactericidal antibodies to multiple strains; (2) react with the surface of multiple strains; (3) confer passive protection against a live challenge; and/or (4) prevent colonization.

The formulation of such immunogenic compositions is well known to persons skilled in this field. Immunogenic compositions of the invention preferably include a pharmaceutically acceptable carrier. Suitable pharmaceutically acceptable carriers and/or diluents include any and all conventional solvents, dispersion media, fillers, solid carriers, aqueous solutions, coatings, antibacterial and antifungal agents, isotonic and absorption delaying agents, and the like. Suitable pharmaceutically acceptable carriers include, for example, one or more of water, saline, phosphate buffered saline, dextrose, glycerol, ethanol and the like, as well as combinations thereof. Pharmaceutically acceptable carriers may further comprise minor amounts of auxiliary substances such as wetting or emulsifying agents, preservatives or buffers, which enhance the shelf life or effectiveness of the antibody. The preparation and use of pharmaceutically acceptable carriers is well known in the art. Except insofar as any conventional media or agent is incompatible with the active ingredient, use thereof in the immunogenic compositions of the present invention is contemplated.

Such immunogenic compositions can be administered parenterally, e.g., by injection, either subcutaneously or intramuscularly, as well as orally or intranasally. Methods for intramuscular immunization are described by Wolff et al. and by Sedegah et al. Other modes of administration employ oral formulations, pulmonary formulations, suppositories, and transdermal applications, for example, without limitation. Oral formulations, for example, include such normally employed excipients as, for example, pharmaceutical grades of mannitol, lactose, starch, magnesium stearate, sodium saccharine, cellulose, magnesium carbonate, and the like, without limitation.

The immunogenic compositions of the invention can include one or more adjuvants, including, but not limited to aluminum hydroxide; aluminum phosphate; STIMULON™ QS-21 (Aquila Biopharmaceuticals, Inc., Framingham, Mass.); MPL™ (3-O-deacylated monophosphoryl lipid A; Corixa, Hamilton, Mont.), 529 (an amino alkyl glucosamine phosphate compound, Corixa, Hamilton, Mont.), IL-12 (Genetics Institute, Cambridge, Mass.); GM-CSF (Immunex Corp., Seattle, Wash.); N-acetyl-muramyl--L-theronyl-D-isoglutamine (thr-MDP); N-acetyl-nor-muramyl-L-alanyl-D-isoglutamine (CGP 11637, referred to as nor-MDP); N-acetylmuramyl-L-alanyl-D-isoglutaminyl-L-alanine-2-(1′-2′-dipalmitoyl-sn-glycero-3-hydroxyphos-phoryloxy-ethylamine) (CGP 19835A, referred to as MTP-PE); and cholera toxin. Others which may be used are non-toxic derivatives of cholera toxin, including its A subunit, and/or conjugates or genetically engineered fusions of the N. meningitidis polypeptide with cholera toxin or its B subunit (“CTB”), procholeragenoid, fungal polysaccharides, including schizophyllan, muramyl dipeptide, muramyl dipeptide (“MDP”) derivatives, phorbol esters, the heat labile toxin of E. coli, block polymers or saponins.

In certain preferred embodiments, the proteins of this invention are used in an immunogenic composition for oral administration which includes a mucosal adjuvant and used for the treatment or prevention of N. meningitidis infection in a human host. The mucosal adjuvant can be a cholera toxin; however, preferably, mucosal adjuvants other than cholera toxin which may be used in accordance with the present invention include non-toxic derivatives of a cholera holotoxin, wherein the A subunit is mutagenized, chemically modified cholera toxin, or related proteins produced by modification of the cholera toxin amino acid sequence. For a specific cholera toxin which may be particularly useful in preparing immunogenic compositions of this invention, see the mutant cholera holotoxin E29H, as disclosed in Published International Application WO 00/18434, which is hereby incorporated herein by reference in its entirety. These may be added to, or conjugated with, the polypeptides of this invention. The same techniques can be applied to other molecules with mucosal adjuvant or delivery properties such as Escherichia coli heat labile toxin (LT). Other compounds with mucosal adjuvant or delivery activity may be used such as bile; polycations such as DEAE-dextran and polyornithine; detergents such as sodium dodecyl benzene sulphate; lipid-conjugated materials; antibiotics such as streptomycin; vitamin A; and other compounds that alter the structural or functional integrity of mucosal surfaces. Other mucosally active compounds include derivatives of microbial structures such as MDP; acridine and cimetidine. STIMULON™ QS-21, MPL, and IL-12, as described above, may also be used.

The immunogenic compositions of this invention may be delivered in the form of ISCOMS (immune stimulating complexes), ISCOMS containing CTB, liposomes or encapsulated in compounds such as acrylates or poly(DL-lactide-co-glycoside) to form microspheres of a size suited to adsorption. The proteins of this invention may also be incorporated into oily emulsions.

Multiple Antigens

The immunogenic agents, including proteins, polynucleotides and equivalents of the present invention may be administered as the sole active immunogen in a immunogenic composition, or alternatively, the composition may include other active immunogens, including other Neisseria sp. immunogenic polypeptides, or immunologically-active proteins of one or more other microbial pathogens (e.g. virus, prion, bacterium, or fungus, without limitation) or capsular polysaccharide. The compositions may comprise one or more desired proteins, fragments or pharmaceutical compounds as desired for a chosen indication. In the same manner, the compositions of this invention which employ one or more nucleic acids in the immunogenic composition may also include nucleic acids which encode the same diverse group of proteins, as noted above.

Any multi-antigen or multi-valent immunogenic composition is contemplated by the present invention. For example, the compositions of the present invention may a comprise combinations of two or more 2086 proteins, a combination of 2086 protein with one or more Por A proteins, a combination of 2086 protein with meningococcus serogroup A, C, Y and W135 polysaccharides and/or polysaccharide conjugates, a combination of 2086 protein with meningococcus and pneumococcus combinations, or a combination of any of the foregoing in a form suitable for mucosal delivery. Persons of skill in the art would be readily able to formulate such multi-antigen or multi-valent immunologic compositions.

The present invention also contemplates multi-immunization regimens wherein any composition useful against a pathogen may be combined therein or therewith the compositions of the present invention. For example, without limitation, a patient may be administered the immunogenic composition of the present invention and another immununological composition for immunizing against S. Pneumoniae, as part of a multi-immunization regimen. Persons of skill in the art would be readily able to select immunogenic compositions for use in conjunction with the immunogenic compositions of the present invention for the purposes of developing and implementing multi-immunization regimens.

Specific embodiments of this invention relate to the use of one or more polypeptides of this invention, or nucleic acids encoding such, in a composition or as part of a treatment regimen for the prevention or amelioration of S. pneumonaie infection. One can combine the 2086 polypeptides or 2086 polynucleotides with any immunogenic composition for use against S. pneumonaie infection. One can also combine the 2086 polypeptides or 2086 polynucleotides with any other protein or polysaccharide-based meningococcal vaccine.

The 2086 polypeptides, fragments and equivalents can be used as part of a conjugate immunogenic composition; wherein one or more proteins or polypeptides are conjugated to a carrier in order to generate a composition that has immunogenic properties against several serotypes and/or against several diseases. Alternatively, one of the 2086 polypeptides can be used as a carrier protein for other immunogenic polypeptides.

The present invention also relates to a method of inducing immune responses in a mammal comprising the step of providing to said mammal an immunogenic composition of this invention. The immunogenic composition is a composition which is antigenic in the treated animal or human such that the immunologically effective amount of the polypeptide(s) contained in such composition brings about the desired immune response against N. meningitidis infection. Preferred embodiments relate to a method for the treatment, including amelioration, or prevention of N. meningitidis infection in a human comprising administering to a human an immunologically effective amount of the composition.

The phrase “immunologically effective amount,” as used herein, refers to the administration of that amount to a mammalian host (preferably human), either in a single dose or as part of a series of doses, sufficient to at least cause the immune system of the individual treated to generate a response that reduces the clinical impact of the bacterial infection. This may range from a minimal decrease in bacterial burden to prevention of the infection. Ideally, the treated individual will not exhibit the more serious clinical manifestations of the bacterial infection. The dosage amount can vary depending upon specific conditions of the individual. This amount can be determined in routine trials or otherwise by means known to those skilled in the art.

Another specific aspect of the present invention relates to using as the immunogenic composition a vector or plasmid which expresses an protein of this invention, or an immunogenic portion thereof. Accordingly, a further aspect this invention provides a method of inducing an immune response in a mammal, which comprises providing to a mammal a vector or plasmid expressing at least one isolated 2086 polypeptide. The protein of the present invention can be delivered to the mammal using a live vector, in particular using live recombinant bacteria, viruses or other live agents, containing the genetic material necessary for the expression of the polypeptide or immunogenic portion as a foreign polypeptide.

According to a further implementation of the present invention, a method is provided for diagnosing bacterial meningitis in a mammal comprising: detecting the presence of immune complexes in the mammal or a tissue sample from said mammal, said mammal or tissue sample being contacted with an antibody composition comprising antibodies that immunospecifically bind with at least one polypeptide comprising the amino acid sequence of any of the even numbered SEQ ID NOS: 2-252 and odd numbered SEQ ID NOS:331-443; wherein the mammal or tissue sample is contacted with the antibody composition under conditions suitable for the formation of the immune complexes.

Viral and Non-Viral Vectors

Preferred vectors, particularly for cellular assays in vitro and in vivo, are viral vectors, such as lentiviruses, retroviruses, herpes viruses, adenoviruses, adeno-associated viruses, vaccinia virus, baculovirus, and other recombinant viruses with desirable cellular tropism. Thus, a nucleic acid encoding a 2086 protein or immunogenic fragment thereof can be introduced in vivo, ex vivo, or in vitro using a viral vector or through direct introduction of DNA. Expression in targeted tissues can be effected by targeting the transgenic vector to specific cells, such as with a viral vector or a receptor ligand, or by using a tissue-specific promoter, or both. Targeted gene delivery is described in PCT Publication No. WO 95/28494, which is incorporated herein by reference in its entirety.

Viral vectors commonly used for in vivo or ex vivo targeting and therapy procedures are DNA-based vectors and retroviral vectors. Methods for constructing and using viral vectors are known in the art (e.g., Miller and Rosman, BioTechniques, 1992, 7:980-990). Preferably, the viral vectors are replication-defective, that is, they are unable to replicate autonomously in the target cell. Preferably, the replication defective virus is a minimal virus, i.e., it retains only the sequences of its genome which are necessary for encapsulating the genome to produce viral particles.

DNA viral vectors include an attenuated or defective DNA virus, such as but not limited to herpes simplex virus (HSV), papillomavirus, Epstein Barr virus (EBV), adenovirus, adeno-associated virus (AAV), and the like. Defective viruses, which entirely or almost entirely lack viral genes, are preferred. Defective virus is not infective after introduction into a cell. Use of defective viral vectors allows for administration to cells in a specific, localized area, without concern that the vector can infect other cells. Thus, a specific tissue can be specifically targeted. Examples of particular vectors include, but are not limited to, a defective herpes virus 1 (HSV1) vector (Kaplitt et al., Molec. Cell. Neurosci., 1991, 2:320-330), defective herpes virus vector lacking a glyco-protein L gene, or other defective herpes virus vectors (PCT Publication Nos. WO 94/21807 and WO 92/05263); an attenuated adenovirus vector, such as the vector described by Stratford-Perricaudet et al. (J. Clin. Invest., 1992, 90:626-630; see also La Salle et al. , Science, 1993, 259:988-990); and a defective adeno-associated virus vector (Samulski etal., J. Virol., 1987, 61:3096-3101; Samulski et al., J. Virol., 1989, 63:3822-3828; Lebkowski et al., Mol. Cell. Biol., 1988, 8:3988-3996), each of which is incorporated by reference herein in its entirety.

Various companies produce viral vectors commercially, including, but not limited to, Avigen, Inc. (Alameda, Calif.; AAV vectors), Cell Genesys (Foster City, Calif.; retroviral, adenoviral, AAV vectors, and lentiviral vectors), Clontech (retroviral and baculoviral vectors), Genovo, Inc. (Sharon Hill, Pa.; adenoviral and AAV vectors), Genvec (adenoviral vectors), IntroGene (Leiden, Netherlands; adenoviral vectors), Molecular Medicine (retroviral, adenoviral, AAV, and herpes viral vectors), Norgen (adenoviral vectors), Oxford BioMedica (Oxford, United Kingdom; lentiviral vectors), and Transgene (Strasbourg, France; adenoviral, vaccinia, retroviral, and lentiviral vectors), incorporated by reference herein in its entirety.

Adenovirus vectors. Adenoviruses are eukaryotic DNA viruses that can be modified to efficiently deliver a nucleic acid of this invention to a variety of cell types. Various serotypes of adenovirus exist. Of these serotypes, preference is given, within the scope of the present invention, to using type 2 or type 5 human adenoviruses (Ad 2 or Ad 5) or adenoviruses of animal origin (see PCT Publication No. WO 94/26914). Those adenoviruses of animal origin which can be used within the scope of the present invention include adenoviruses of canine, bovine, murine (example: Mavl, Beard et al., Virology, 1990, 75-81), ovine, porcine, avian, and simian (example: SAV) origin. Preferably, the adenovirus of animal origin is a canine adenovirus, more preferably a CAV2 adenovirus (e.g., Manhattan or A26/61 strain, ATCC VR-800, for example). Various replication defective adenovirus and minimum adenovirus vectors have been described (PCT Publication Nos. WO 94/26914, WO 95/02697, WO 94/28938, WO 94/28152, WO 94/12649, WO 95/02697, WO 96/22378). The replication defective recombinant adenoviruses according to the invention can be prepared by any technique known to the person skilled in the art (Levrero et al., Gene, 1991, 101:195; European Publication No. EP 185 573; Graham, EMBO J., 1984, 3:2917; Graham et al., J. Gen. Virol., 1977, 36:59). Recombinant adenoviruses are recovered and purified using standard molecular biological techniques, which are well known to persons of ordinary skill in the art.

Adeno-associated viruses. The adeno-associated viruses (AAV) are DNA viruses of relatively small size that can integrate, in a stable and site-specific manner, into the genome of the cells which they infect. They are able to infect a wide spectrum of cells without inducing any effects on cellular growth, morphology or differentiation, and they do not appear to be involved in human pathologies. The AAV genome has been cloned, sequenced and characterized. The use of vectors derived from the AAVs for transferring genes in vitro and in vivo has been described (see, PCT Publication Nos. WO 91/18088 and WO 93/09239; U.S. Pat. Nos. 4,797,368 and 5,139,941; European Publication No. EP 488 528). The replication defective recombinant AAVs according to the invention can be prepared by cotransfecting a plasmid containing the nucleic acid sequence of interest flanked by two AAV inverted terminal repeat (ITR) regions, and a plasmid carrying the AAV encapsidation genes (rep and cap genes), into a cell line which is infected with a human helper virus (for example an adenovirus). The AAV recombinants which are produced are then purified by standard techniques.

Retrovirus vectors. In another implementation of the present invention, the nucleic acid can be introduced in a retroviral vector, e.g., as described in U.S. Pat. No. 5,399,346; Mann et al., Cell, 1983, 33:153; U.S. Pat. Nos. 4,650,764 and 4,980,289; Markowitz et al., J. Virol., 1988, 62:1120; U.S. Pat. No. 5,124,263; European Publication Nos. EP 453 242 and EP178 220; Bernstein et al., Genet. Eng., 1985, 7:235; McCormick, BioTechnology, 1985, 3:689; PCT Publication No. WO 95/07358; and Kuo et al., Blood, 1993, 82:845, each of which is incorporated by reference in its entirety. The retroviruses are integrating viruses that infect dividing cells. The retrovirus genome includes two LTRs, an encapsidation sequence and three coding regions (gag, pol and env). In recombinant retroviral vectors, the gag, pol and env genes are generally deleted, in whole or in part, and replaced with a heterologous nucleic acid sequence of interest. These vectors can be constructed from different types of retrovirus, such as, HIV, MoMuLV (“murine Moloney leukaemia virus” MSV (“murine Moloney sarcoma virus”), HaSV (“Harvey sarcoma virus”); SNV (“spleen necrosis virus”); RSV (“Rous sarcoma virus”) and Friend virus. Suitable packaging cell lines have been described in the prior art, in particular the cell line PA317 (U.S. Pat. No. 4,861,719); the PsiCRIP cell line (PCT Publication No. WO 90/02806) and the GP+envAm-12 cell line (PCT Publication No. WO 89/07150). In addition, the recombinant retroviral vectors can contain modifications within the LTRs for suppressing transcriptional activity as well as extensive encapsidation sequences which may include a part of the gag gene (Bender et al., J. Virol., 1987, 61:1639). Recombinant retroviral vectors are purified by standard techniques known to those having ordinary skill in the art.

Retroviral vectors can be constructed to function as infectious particles or to undergo a single round of transfection. In the former case, the virus is modified to retain all of its genes except for those responsible for oncogenic transformation properties, and to express the heterologous gene. Non-infectious viral vectors are manipulated to destroy the viral packaging signal, but retain the structural genes required to package the co-introduced virus engineered to contain the heterologous gene and the packaging signals. Thus, the viral particles that are produced are not capable of producing additional virus.

Retrovirus vectors can also be introduced by DNA viruses, which permits one cycle of retroviral replication and amplifies transfection efficiency (see PCT Publication Nos. WO 95/22617, WO 95/26411, WO 96/39036 and WO 97/19182).

Lentivirus vectors. In another implementation of the present invention, lentiviral vectors can be used as agents for the direct delivery and sustained expression of a transgene in several tissue types, including brain, retina, muscle, liver and blood. The vectors can efficiently transduce dividing and nondividing cells in these tissues, and effect long-term expression of the gene of interest. For a review, see, Naldini, Curr. Opin. Biotechnol., 1998, 9:457-63; see also Zufferey, et al., J. Virol., 1998, 72:9873-80). Lentiviral packaging cell lines are available and known generally in the art. They facilitate the production of high-titer lentivirus vectors for gene therapy. An example is a tetracycline-inducible VSV-G pseudotyped lentivirus packaging cell line that can generate virus particles at titers greater than 106 IU/mL for at least 3 to 4 days (Kafri, et al., J. Virol., 1999, 73: 576-584). The vector produced by the inducible cell line can be concentrated as needed for efficiently transducing non-dividing cells in vitro and in vivo.

Non-viral vectors. In another implementation of the present invention, the vector can be introduced in vivo by lipofection, as naked DNA, or with other transfection facilitating agents (peptides, polymers, etc.). Synthetic cationic lipids can be used to prepare liposomes for in vivo transfection of a gene encoding a marker (Felgner, et. al., Proc. Natl. Acad. Sci. U.S.A., 1987, 84:7413-7417; Felgner and Ringold, Science, 1989, 337:387-388; see Mackey, et al., Proc. Natl. Acad. Sci. U.S.A., 1988, 85:8027-8031; Ulmer et al., Science, 1993, 259:1745-1748). Useful lipid compounds and compositions for transfer of nucleic acids are described in PCT Patent Publication Nos. WO 95/18863 and WO 96/17823, and in U.S. Pat. No. 5,459,127. Lipids may be chemically coupled to other molecules for the purpose of targeting (see Mackey, et. al., supra). Targeted peptides, e.g., hormones or neurotransmitters, and proteins such as antibodies, or non-peptide molecules could be coupled to liposomes chemically.

Other molecules are also useful for facilitating transfection of a nucleic acid in vivo, such as a cationic oligopeptide (e.g., PCT Patent Publication No. WO 95/21931), peptides derived from DNA binding proteins (e.g., PCT Patent Publication No. WO 96/25508), or a cationic polymer (e.g., PCT Patent Publication No. WO 95/21931).

It is also possible to introduce the vector in vivo as a naked DNA plasmid. Naked DNA vectors for vaccine purposes or gene therapy can be introduced into the desired host cells by methods known in the art, e.g., electroporation, microinjection, cell fusion, DEAE dextran, calcium phosphate precipitation, use of a gene gun, or use of a DNA vector transporter (e.g., Wu et al., J. Biol. Chem., 1992, 267:963-967; Wu and Wu, J. Biol. Chem., 1988, 263:14621-14624; Canadian Patent Application No. 2,012,311; Williams et al., Proc. Natl. Acad. Sci. USA, 1991, 88:2726-2730). Receptor-mediated DNA delivery approaches can also be used (Curiel et al., Hum. Gene Ther., 1992, 3:147-154; Wu and Wu, J. Biol. Chem., 1987, 262:4429-4432). U.S. Pat. Nos. 5,580,859 and 5,589,466 disclose delivery of exogenous DNA sequences, free of transfection facilitating agents, in a mammal. Recently, a relatively low voltage, high efficiency in vivo DNA transfer technique, termed electrotransfer, has been described (Mir et al. , C.P. Acad. Sci., 1988, 321:893; PCT Publication Nos. WO 99/01157; WO 99/01158; WO 99/01175). Accordingly, additional embodiments of the present invention relates to a method of inducing an immune response in a human comprising administering to said human an amount of a DNA molecule encoding a 2086 polypeptide of this invention, optionally with a transfection-facilitating agent, where said polypeptide, when expressed, retains immunogenicity and, when incorporated into an immunogenic composition and administered to a human, provides protection without inducing enhanced disease upon subsequent infection of the human with Neisseria sp. pathogen, such as N. meningitidis. Transfection-facilitating agents are known in the art and include bupivicaine, and other local anesthetics (for examples see U.S. Pat. No. 5,739,118) and cationic polyamines (as published in International Patent Application WO 96/10038), which are hereby incorporated by reference.

The present invention also relates to an antibody, which may either be a monoclonal or polyclonal antibody, specific for 2086 polypeptides as described above. Such antibodies may be produced by methods which are well known to those skilled in the art.

Bacterial Expression Systems and Plasmids

This invention also provides a recombinant DNA molecule, such as a vector or plasmid, comprising an expression control sequence having promoter sequences and initiator sequences and a nucleotide sequence which codes for a polypeptide of this invention, the nucleotide sequence being located 3′ to the promoter and initiator sequences. In yet another aspect, the invention provides a recombinant DNA cloning vehicle capable of expressing a 2086 polypeptide comprising an expression control sequence having promoter sequences and initiator sequences, and a nucleotide sequence which codes for a 2086 polypeptide, the nucleotide sequence being located 3′ to the promoter and initiator sequences. In a further aspect, there is provided a host cell containing a recombinant DNA cloning vehicle and/or a recombinant DNA molecule as described above. Suitable expression control sequences and host cell/cloning vehicle combinations are well known in the art, and are described by way of example, in Sambrook et al. (1989).

Once recombinant DNA cloning vehicles and/or host cells expressing a desired a polypeptide of this invention have been constructed by transforming, transfecting or infecting such cloning vehicles or host cells with plasmids containing the corresponding 2086 polynucleotide, cloning vehicles or host cells are cultured under conditions such that the polypeptides are expressed. The polypeptide is then isolated substantially free of contaminating host cell components by techniques well known to those skilled in the art.

The following examples are included to demonstrate preferred embodiments of the invention. It should be appreciated by those skilled in the art that the techniques disclosed in the examples which follow represent techniques discovered by the inventors to function well in the practice of the invention, and thus can be considered to constitute preferred modes for its practice. However, those of skill in the art should, in view of the present disclosure, appreciate that many changes can be made in the specific embodiments which are disclosed and still obtain a like or similar result without departing from the spirit and scope of the invention.

### EXAMPLES

### Example 1

Identification of a Neisserial Membrane Protein Extract Capable of Eliciting Bactericidal Antibodies Against Heterologous Strains:

Referring to Table II below, LOS-depleted outer membrane protein preparations have been shown to elicit bactericidal antibodies. These antibodies are often directed towards the PorA of the respective strain. LOS-depleted outer membrane preparations from serogroup B meningococcal strain 8529 (B:15:P1.7b,3) are unusual in this manner because they unexpectedly elicit bactericidal antibodies to several heterologous strains.

TABLE II
BC Activity of Anti-sOMPS Against Different Strains of N. meningitidis

| | Anti-serum Week 6 | | | | | | |
|---|---|---|---|---|---|---|---|
| | H44/76 | 5315 | H355 | M982 | 880049 | 8529* | NMB |
| | Serosubtype | | | | | | |
| | P1.7,16 | P1.5 | P1.15 | P1.9 | P1.4 | P1.3 | P1.5,2 |
| sOMPs H44/76 25 μg | 1,000 | <50 | <50 | <50 | <50 | 980 | <50 |
| QS-21 20 μg | | | | | | | |
| sOMPs 5315 25 μg | 50 | <50 | <50 | <50 | <50 | 2170 | <50 |
| QS-21 20 μg | | | | | | | |
| sOMPs H355 25 μg | <50 | <50 | 450 | <50 | <50 | 860 | <50 |
| QS-21 20 μg | | | | | | | |
| sOMPs M982 25 μg | 92 | <50 | <50 | 300 | <50 | 1100 | <50 |
| QS-21 20 μg | | | | | | | |
| sOMPs 880049 25 μg | 50 | <50 | <50 | <50 | <50 | 1190 | <50 |
| QS-21 20 μg | | | | | | | |
| sOMPs 8529 25 μg | 1,000 | <50 | 450 | 50 | 215 | >4050 | <50 |
| QS-21 20 μg | | | | | | (81.7) | |
| sOMPs 2996 25 μg | <50 | <50 | <50 | <50 | <50 | 790 | 148 |
| QS-21 20 μg | | | | | | | |
| Whole-cell control serum | 450 | 50 | 100 | 500 | 150 | >1350 | 952 |
| 25 μg 3DMPL 25 μg | | | | | | (66.0) | |

To facilitate the isolation and characterization of the antigen(s) responsible for eliciting heterologous bactericidal antibodies, we sought to identify which detergent optimally extracted the antigen(s)

Strains and Culture Conditions.

N. meningitidis strain 8529 from a frozen vial was streaked onto a GC plate. (The meningococcal strain 8529 was received from The RIVM, Bilthoven, The Netherlands). The plate was incubated at 36C/5% CO₂ for 7.5h. Several colonies were used to inoculate a flask containing 50 mL of modified Frantz medium+GC supplement. The flask was incubated in an air shaker at 36° C. and agitated at 200 RPM for 4.5 h. 5 mL was used to inoculate a Fernbach flask containing 450 mL of modified Frantz medium+GC supplement. The flask was incubated in an air shaker at 36° C. and agitated at 100 RPM for 11 h. The entire 450 mL was used to inoculate 8.5 L of modified Frantz medium+GC supplement in a 10 L fermentor.

Composition of Modified Frantz Medium:

| Glutamic acid | 1.3 g/L |
|---|---|
| Cysteine | 0.02 |
| Sodium phosphate, dibasic, 7 hydrate | 10 |
| Potassium chloride | 0.09 |
| Sodium chloride | 6 |
| Ammonium chloride | 1.25 |
| Dialyzed yeast extract (YE) | 40 ml |
| (25% YE soln. dialyzed against 5 volumes | |
| of dH₂O overnight, then autoclaved) | |
| GC supplement 100X, filter sterilize | |
| Dextrose | 400 g/L |
| Glutamic acid | 10 |
| Cocarboxylase | 0.02 |
| Ferric nitrate | 0.5 |

The following parameters were controlled during fermentation: Temperature=36°C.; pH=7.4; Dissolved Oxygen=20%. Several drops of P-2000 antifoam were added to control foaming. The culture was grown to stationary phase. Cells were harvested by centrifugation at OD650=5.25. A total of 100-300 grams of wet cell paste is typically harvested from ˜8.5L of culture.

Partial Purification of Outer Membrane Protein Fractions from Meningococci which Elicit Heterologous Bactericidal Antibodies:

100 gms wet weight of cells were suspended, to a volume five times the wet weight, with 10 mM HEPES-NaOH, pH 7.4, 1 mM Na2EDTA and lysed by passage through a 110Y microfluidizer equipped with a chamber at ˜18,000 psi. The cell lysate was clarified and the cell envelope isolated by centrifugation at 300,000×g for 1 hour at 10° C. The cell envelopes were washed 2× with the same buffer by suspension with a homogenizer followed by centrifugation as above. The cell envelopes were then extracted with 320 mL of 1% (w/v) Triton X-100 in 10 mM HEPES-NaOH, pH 7.4, 1 mM MgCl₂. Referring to Table III below, results from sequential differential detergent extraction using Triton X-100 and Zwittergent 3-14 followed by immunization of mice, allowed us to determine that the Triton extracts optimally extracted the candidate(s) of interest. This Triton X-100 extract, eliciting bactericidal antibody response against 4 out of five strains listed in table III, was then fractionated by preparative isoelectric focusing (IEF) in a BioRad Rotophor unit. Ampholyte concentrations were 1% pH 3-10 mixed with 1% pH 4-6. As shown in Table III, several fractions were found to elicit a heterologous bactericidal response. The fractions obtained from IEF, which focused in the pH range of 5.5-7.8, elicited a heterologous response to the most strains as determined by the bactericidal assay. The pooled IEF fractions were concentrated and the ampholytes removed by ethanol precipitation. A further purification was achieved by adsorbing some of the proteins obtained in the pH range of about 5.5-7.8 on an anion exchange column and comparing the bactericidal activity obtained after immunizing mice with the adsorbed and unadsorbed proteins. Referring again to Table II, while many proteins were adsorbed to the anion exchange resin, the proteins which were not adsorbed by the column elicited more heterologous bactericidal antibodies.

TABLE III

| | | BC₅₀ Target Strain | | | | |
|---|---|---|---|---|---|---|
| Method | Fraction | H44/76 | 880049 | H355 | 539* | M982 |
| LOS- | sOMPs | 1,000 | 215 | 450 | NC | 50 |
| depleted | | | | | | |
| Detergent | Cytoplasmic Extract | 200 | NT | NT | NT | NT |
| Extractions | TX-100 | >800 | >800 | >800 | >800 | <25 |
| | Zwittergent 3-12 | 400 | >25 | 100 | 400 | <25 |
| | Zwittergent 3-14 | <25 | NT | NT | NT | NT |
| | Zw.3-14 + NaCl | <25 | NT | NT | NT | NT |
| | Sarcosyl | <25 | NT | NT | NT | NT |
| | Zw.3-14 + heat | <25 | NT | NT | NT | NT |
| Preparative | Fractions 1-3 (pH 2.3-3.9) | 50 | NT | NT | NT | NT |
| IEF | Fraction 4 (pH 4.1) | >800 | <25 | 100 | <25 | NT |
| | Fraction 5 (pH 4.3) | >800 | <25 | 100 | 200 | NT |
| | Fraction 6 (pH 4.5) | 400 | NT | NT | NT | NT |
| | Fraction 7 (pH 4.8) | <25 | NT | NT | NT | NT |
| | Fractions 8-9 (pH 5.0-5.3) | <25 | NT | NT | NT | NT |
| | Fractions 10-17 (pH 5.5-7.8) | >800 | 200 | <800 | <800 | NT |
| Anion | Adsorbed | 400 | NT | 100 | 100 | NT |
| Exchange | Unadsorbed | >6,400 | NT | <800 | <800 | NT |

NT: not tested
*Clinical isolate 539 is a homologous strain to 8529, isolated from the same outbreak

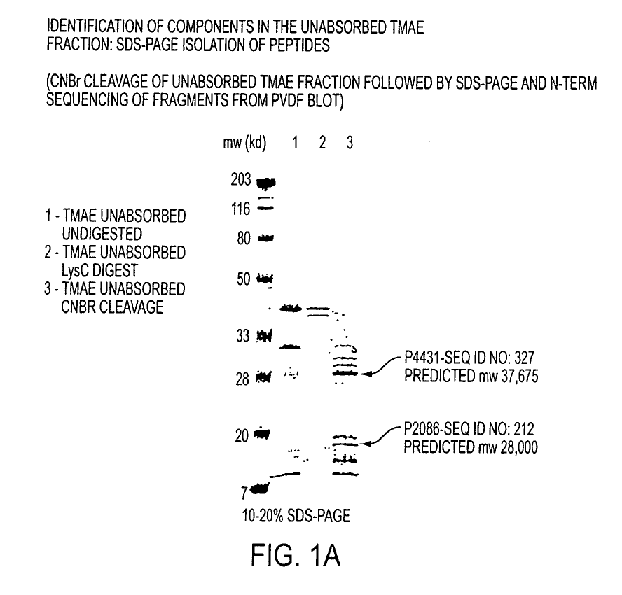

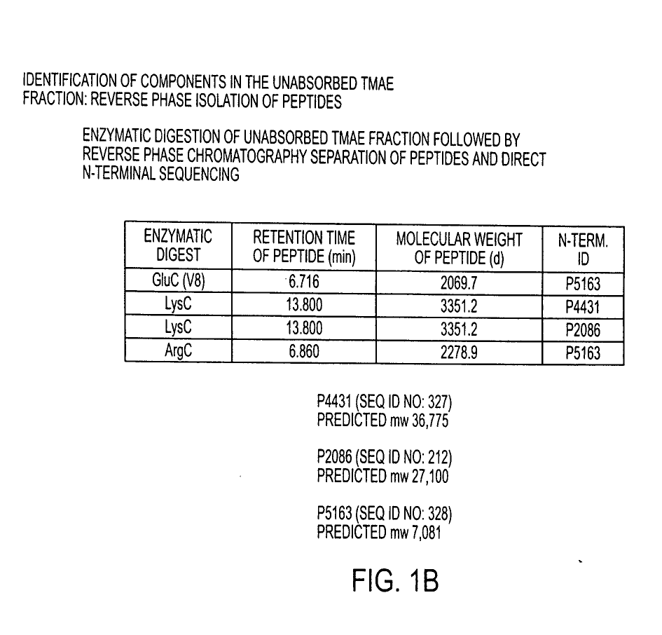

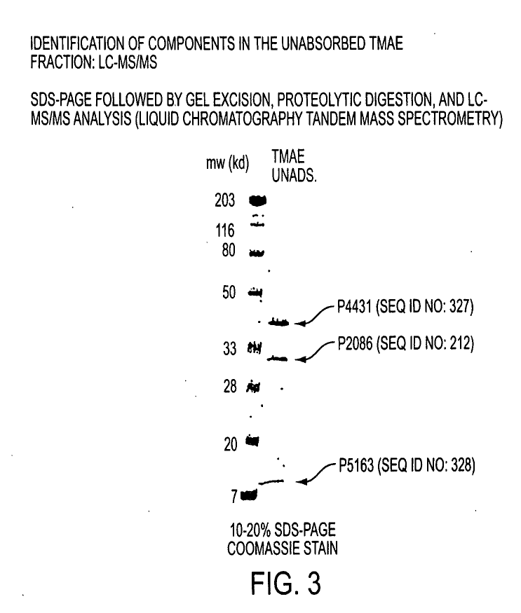

As shown in FIG. 1A, two major proteins were present in the unadsorbed fraction as determined by SDS-PAGE. To identify these proteins, two types of analysis were performed. One analysis was to perform limited proteolytic degradation (See FIG. 1A, and FIG. 1B) followed by isolation of peptides and direct protein sequencing. The other analysis was to perform SDS-PAGE followed by gel excision, proteolytic digestion, and LC-MS/MS (Liquid Chromotography tandem Mass Spectrometry), (see FIG. 3) to obtain mass spectral information on the components of the preparations of interest. (See peptide mapping and sequencing methods described later in this section)

The N. meningitidis A Sanger genomic sequence was analyzed using the methods and algorithms described in Zagursky and Russell, 2001, BioTechniques, 31:636-659. This mining analysis yielded over 12,000 possible Open Reading Frames (ORFs). Both the direct sequence data and the mass spectral data described above indicated that the major components of the unadsorbed fraction were the products of several ORFs present in an analysis of the Sanger database. The three predominant proteins identified by this methodology correspond to ORFs 4431, 5163 and 2086, (see FIGS. 1B and 3).

Although ORF 4431 was the most predominant protein identified in the fractions, mouse antibodies to recombinant lipidated 4431 were not bactericidal and did not provide a protective response in an animal model. Additional analysis of ORF 5163 is in progress.

The second most predominant component of the preparations described herein corresponds to the product of ORF 2086.

Immunogenicity Methods:

Preparation of Antisera:

Except where noted, protein compositions/vaccines were formulated with 25μg of total protein and were adjuvanted with 20 μg QS-21. A 0.2 mL dose was administered by subcutaneous (rump) injection to 6-8 week old female Swiss-Webster mice at week 0 and 4. Bleeds were collected at week 0 and 4, and a final exsanguination bleed was performed on week 6.

Bactericidal Assay:

Bactericidal assays were performed essentially as described (See Mountzouros and Howell, 2000, J. Clin. Microbiol. 38(8):2878-2884). Complement-mediated antibody-dependent bactericidal titers for the SBA were expressed as the reciprocal of the highest dilution of test serum that killed ≧50% of the target cells introduced into the assays (BC₅₀ titer).

Methods Used to Identify 2086 Protein:

Cyanogen Bromide Cleavage and Direct Sequencing of Fragments:

Cyanogen Bromide cleavage of Anion Exchange Unadsorbed Fraction (AEUF). The AEUF was precipitated with 90% cold ethanol and was solubilized with 10 mg/mL cyanogen bromide in 70% formic acid to a protein concentration of 1 mg/mL. The reaction was performed overnight at room temperature in the dark. The cleaved products were dried down by speed vacuum, and the pellet was solubilized with HE/0.1% reduced TX-100. SDS-PAGE followed by N-terminal amino acid sequencing were used to identify the components of this fraction.

Protease Digestion/Reverse Phase/N-Terminal Sequencing to Identify Components:

The AEUF was digested with either GluC (V8), LysC or ArgC. The protein to enzyme ratio was 30 μg protein to 1 μg enzyme. The digestion was carried out at 37° C. overnight. The digested protein mixture (30 μg) was passed over a seven micron Aquapore RF-300 column and was eluted with a gradient of 10-95% acetonitrile in 0.1% trifluoroacetic acid, and peaks were collected manually. A no protein blank was also run, and the peaks from this were subtracted from the sample chromatogram. Peaks occurring only in the sample run were analyzed by mass spectrometer, and those samples giving a clear mass were analyzed for N-terminal amino acid sequencing.

N-Terminal Amino Acid Sequencing:

For bands excised from a blot, the protein sample is transferred from an SDS gel to a PVDF membrane, stained with Amido Black (10% acetic acid, 0.1% amido black in deionized water) and destained in 10% acetic acid. The desired protein band is then excised from all ten lanes using a methanol cleaned scalpel or mini-Exacto knife and placed in the reaction cartridge of the Applied Biosystems 477A Protein Sequencer. For direct sequencing of samples in solution, the Prosorb cartridge is assembled and the PVDF wetted with 60 μL of methanol. The PVDF is rinsed with 50 μL of deionized water and the sample (50 μL) is loaded to the PVDF. After 50 μL of deionized water is used to rinse the sample, the Prosorb PVDF is punched out, dried, and placed in the reaction cartridge of the Applied Biosystems 477A Protein Sequencer. For both methods, the Applied Biosystems N-terminal Sequencer is then run under optimal blot conditions for 12 or more cycles (1 cycle Blank, 1 cycle Standard, and 10 or more cycles for desired residue identification) and PTH-amino acid detection is done on the Applied Biosystems 120A PTH Analyzer. The cycles are collected both on an analog chart recorder and digitally via the instrument software. Amino acid assignment is done using the analog and digital data by comparison of a standard set of PTH-amino acids and their respective retention times on the analyzer (cysteine residues are destroyed during conversion and are not detected). Multiple sequence information can be obtained from a single residue and primary versus secondary assignments are made based on signal intensity.

LC-MS/MS

Protein samples purified by IEF were further analyzed by SDS-polyacrylamide gel electrophoresis. Proteins were visualized by Coomaasie blue staining, and bands of interest were excised manually, then reduced, alkylated and digested with trypsin (Promega, Madison, Wis.) in situ using an automated in-gel tryptic digestion robot (1). After digestion, peptide extracts were concentrated to a final volume of 10-20 μL using a Savant Speed Vac Concentrator (ThermoQuest, Holdbrook, N.Y.).

Peptide extracts were analyzed on an automated microelectrospray reversed phase HPLC. In brief, the microelectrospray interface consisted of a Picofrit fused silica spray needle, 50 cm length by 75 um ID, 8 um orifice diameter (New Objective, Cambridge Mass.) packed with 10 um C18 reversed-phase beads (YMC, Wilmington, N.C.) to a length of 10 cm. The Picofrit needle was mounted in a fiber optic holder (Melles Griot, Irvine, Calif.) held on a home-built base positioned at the front of the mass spectrometer detector. The rear of the column was plumbed through a titanium union to supply an electrical connection for the electrospray interface. The union was connected with a length of fused silica capillary (FSC) tubing to a FAMOS autosampler (LC-Packings, San Francisco, Calif.) that was connected to an HPLC solvent pump (ABI 140C, Perkin-Elmer, Norwalk, Conn.). The HPLC solvent pump delivered a flow of 50 μL /min which was reduced to 250 nL/min using a PEEK microtight splitting tee (Upchurch Scientific, Oak Harbor, Wash.), and then delivered to the autosampler using an FSC transfer line. The LC pump and autosampler were each controlled using their internal user programs. Samples were inserted into plastic autosampler vials, sealed, and injected using a 5 μL sample loop.

Microcapillary HPLC-Mass Spectrometry:

Extracted peptides from in-gel digests were separated by the microelectrospray HPLC system using a 50 minute gradient of 0-50% solvent B (A: 0.1M HoAc, B: 90% MeCN/0.1M HoAc). Peptide analyses were done on a Finnigan LCQ ion trap mass spectrometer (ThermoQuest, San Jose, Calif.) operating at a spray voltage of 1.5 kV, and using a heated capillary temperature of 150 ° C. Data were acquired in automated MS/MS mode using the data acquisition software provided with the instrument. The acquisition method included 1 MS scan (375-1200 m/z) followed by MS/MS scans of the top 3 most abundant ions in the MS scan. The dynamic exclusion and isotope exclusion functions were employed to increase the number of peptide ions that were analyzed (settings: 3 amu=exclusion width, 3 min=exclusion duration, 30 secs=pre-exclusion duration, 3 amu=isotope exclusion width). Automated analysis of MS/MS data was performed using the SEQUEST computer algorithm incorporated into the Finnigan Bioworks data analysis package (ThermoQuest, San Jose, Calif.) using the database of proteins derived from the complete genome of N. meningitidis (from Sanger). The results of the study are illustrated in FIG. 3.

### Example 2

Cloning of Recombinant Lipidated P2086 (rLP2086):

A.) Native Leader Sequence:

Source Materials:

The ORF 2086 gene was amplified by PCR from a clinical isolate of a serogroup B Neisseria meningitidis strain designated 8529. The serogroup, serotype and serosubtype of this strain is shown in parentheses; 8529 (B:15, P1:7b,3). This meningococcal strain was received from The RIVM, Bilthoven, The Netherlands. The mature 2086 protein gene sequence from meningococcal strain 8529 is provided herein as SEQ ID. NO. 212.

PCR Amplification and Cloning Strategy:

A visual inspection of ORF 2086 indicated that this gene had a potential lipoprotein signal sequence. Additional analysis using a proprietary Hidden Markov Model Lipoprotein algorithm confirmed that ORF 2086 contains a lipoprotein signal sequence. In order to recombinantly express P2086 in a more native-like conformation, oligonucleotide primers were designed to amplify the full length gene with the lipoprotein signal sequence intact and were based on an analysis of the Sanger sequence for N. meningitidis A ORF 2086, (5′ primer-CT ATT CTG CAT ATG ACT AGG AGC and 3′ primer-GCGC GGATCC TTA CTG CTT GGC GGC AAG ACC), which are SEQ ID NO. 304 (Compound No. 4624) and SEQ ID NO. 303 (Compound No. 4623), respectively (See also Table IV herein). The 2086 gene was amplified by polymerase chain reaction (PCR) [ABI 2400 thermal cycler, Applied Biosystems, Foster City, Calif.] from N. meningitidis strain 8529. The correct size amplified product was ligated and cloned into pCR2.1-TOPO (Invitrogen). The plasmid DNA was restriction digested with NdeI and BamHI, gel purified and ligated into pET-27b(+) vector (Novagen).

Nucleotide primers described herein, were synthesized on a PerSeptive Biosystems oligonucleotide synthesizer, Applied Biosystems, Foster City Calif., using β-Cyanoethylphosphoramidite chemistry, Applied Biosystems, Foster City Calif. The primers used for PCR amplification of the ORF 2086 gene families are listed in Table IV, which shows non-limiting examples of primers of the present invention.

TABLE IV
PRIMERS

| SEQ | | | |
|---|---|---|---|
| ID | | | |
| NO. | | | Re- |
| (Com- | | | stric- |
| pound | | | tion |
| No.) | Primer | Sequence | sites |
| 303 | Reverse | GCGCGGATCCTTACTGCTTGGCGGCAAGA | BamHI |
| (4623) | | CC | |
| 304 | Forward | CTATTCTGCATATGACTAGGAGC | NdeI |
| (4624) | | | |
| 305 | Forward | AGCAGCGGAGGCGGCGGTGTC | |
| (4625) | | | |
| 306 | Forward | TGCCGATGCACTAACCGCACC | |
| (5005) | | | |
| 307 | Reverse | CGTTTCGCAACCATCTTCCCG | |
| (5007) | | | |
| 308 | Reverse | GAGATCTCACTCACTCATTACTGCTTGGC | BglII |
| (5135) | | GGCAAGACCGATATG | |
| 309 | Forward | GCGGATCCAGCGGAGGGGGTGGTGTCGCC | BamHI |
| (5658) | | | |
| 310 | Reverse | GCGCATGCTTACTGCTTGGCGGCAAGACC | SphI |
| (5660) | | GATATG | |
| 311 | Forward | GCGGATCCAGCGGAGGCGGCGGAAGC | BamHI |
| (6385) | | | |
| 312 | Forward | GCGCAGATCTCATATGAGCAGCGGAGGGG | BglII |
| (6406) | | GTGGTGTCGCCGCCGAYATWGGTGCGGGG | and |
| | | CTTGCCG | NdeI |
| 313 | Forward | CTATTCTGCGTATGACTAG | |
| (6470) | | | |
| 314 | Reverse | GTCCGAACGGTAAATTATCGTG | |
| (6472) | | | |
| 315 | Forward | GCGGATCCAGCGGAGGCGGCGGTGTCGCC | BamHI |
| (6473) | | | |
| 316 | Forward | GAGATCTCATATGAGCAGCGGAGGCGGCG | BglII |
| (6474) | | GAAGC | and |
| | | | NdeI |
| 317 | Forward | GACAGCCTGATAAACC | |
| (6495) | | | |
| 318 | Reverse | GATGCCGATTTCGTGAACC | |
| (6496) | | | |
| 319 | Reverse | GCGCATGCCTACTGTTTGCCGGCGATG | SphI |
| (6543) | | | |
| 320 | Reverse | GAGATCTCACTCACTCACTACTGTTTGCC | BglII |
| (6605) | | GGCGATGCCGATTTC | |
| 321 | Forward | GCGCAGATCTCATATGAGCAGCGGAGGCG | BglII |
| (6721) | | GCGGAAGCGGAGGCGGCGGTGTCACCGCC | and |
| | | GACATAGGCACG | NdeI |

rLP2086 Lipoprotein Expression Utilizing Native Leader Sequence:

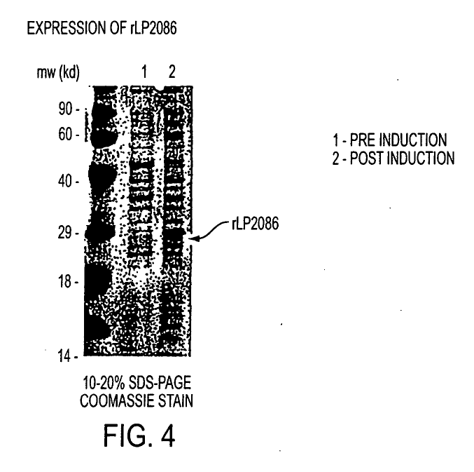

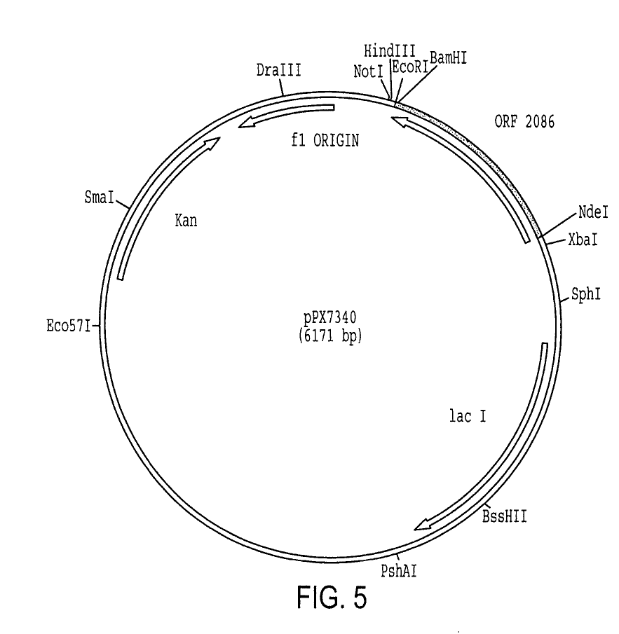

Referring to FIG. 5, plasmid pPX7340 was transformed/transfected or infected into BLR (DE3) pLysS host cells (Life Sciences). One transformant was selected and inoculated into 50 mL of Terrific Broth containing 2% glucose, kanamycin (30 μg/mL), chloramphenicol (30 μg/mL), and tetracycline (12 μg/mL). The OD600 for the overnight culture was 6.0. The overnight culture was diluted out in 1 liter of Terrific Broth with 1% glycerol and the same antibiotics. The starting OD600 was 0.4. After 2 hours the OD600 was 1.6 and a pre-induced sample was taken. Cells equivalent to an OD600=1 were centrifuged and the supernatant was removed. The whole cell pellet was resuspended in 150 μL Tris-EDTA buffer and 150 μL of 2×SDS-PAGE sample buffer. IPTG was added to a final concentration of 1 mM. After 3.5 hours a post-induced sample was taken as described and analyzed on SDS-PAGE (See FIG. 4).

Purification of rLP2086:

The P2086 was solubilized from E. coli following differential detergent extraction. Unlike the P2086 in its native environment, the rLP2086 was not significantly solubilized by Triton X-100 or Zwittergent 3-12. The bulk of the rLP2086 was solubilized with sarcosyl, indicating that it interacts with the outer membrane components of E. coli differently than it does in N. meningitidis. Once solubilized the rLP2086 was purified similarly to the native protein in that many of the contaminating E. coli proteins could be removed by adsorbtion to an anion exchange resin at pH 8. Despite being greater than one half a pH unit above its theoretical pI, the rLP2086 remained unadsorbed at pH 8. Further purification was achieved by adsorbtion of the rLP2086 to a cation exchange resin at pH 4.5.

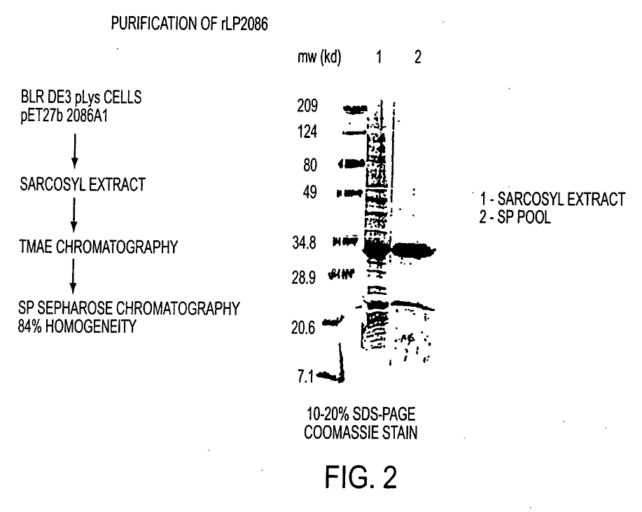

The homogeneity of the rLP2086 is shown in FIG. 2 following SDS-PAGE. The mass of rLP2086 was determined by MALDI-TOF mass spectral analysis to be 27,836. This mass differs from the theoretical mass of 27,100 by 736, which approximates the mass of the N-terminal lipid modification common to bacterial lipoproteins. Both native and rLP2086 appear to be outer membrane lipoproteins. Attempts with N-terminal sequencing were blocked and this is consistent with the terminal modification.

Purification Methods:

Frozen pellets of BLR DE3 pLysS cells expressing P2086 were resuspended in 10 mM HEPES-NaOH/1 mM EDTA/1 μg/mL Pefabloc SC protease inhibitor (Roche) pH 7.4 (HEP) at 20 mL/g wet cell weight and lysed by microfluidizer (Microfluidics Corporation Model 110Y). The cell lysate was centrifuged at 150,000×g for one hour. The pellet was washed twice with HEP and centrifuged twice, and the resulting membrane pellet was frozen overnight. The pellet was solubilized with 10 mM HEPES-NaOH/1 mM MgCl2/1%TX-100 pH 7.4 for 30 minutes, followed by centrifugation at 150,000×g for 30 minutes. This was repeated three times. The membrane pellet was washed as above twice with 50 mM Tris-HCl/5 mM EDTA/1% Zwittergent 3-12 pH 8, followed by two washes each of 50 mM Tris-HCl/5 mM EDTA/1% Zwittergent 3-14 pH 8 and 50 mM Tris-HCl/5 mM EDTA/1% Zwittergent 3-14/0.5M NaCl pH 8.

The rLP2086 was then solubilized with 50 mM Tris-HCl/5 mM EDTA/1% sarcosyl pH 8. This sarcosyl extract was adjusted to 1% Zwittergent 3-14 (Z3-14) and dialyzed twice against a 30 fold excess of 50 mM Tris-HCl/5 mM EDTA/1% Z3-14. The dialyzed rLP2086 extract was precipitated with 90% ethanol to remove remaining sarcosyl, and solubilized with 50 mM Tris-HCl/5 mM EDTA/1% Z3-14 pH 8 (TEZ). Insoluble material was removed by centrifugation, the supernatant was passed over an anion exchange chromatography column, and rLP2086 was collected in the unbound fraction. The unbound material was then dialyzed twice against a 30 fold excess of 25 mM NaAc/1% Z3-14 pH 4.5, and passed over a cation exchange chromatography column. The rLP2086 was eluted with a 0-0.3M NaCl gradient and analyzed by SDS-PAGE (Coomassie stain). The rLP2086 pool was determined to be 84% pure by laser densitometry.

Surface Reactivity and Bactericidal Activity of Antisera to rLP2086 Subfamily B.

Referring to Table VII, antisera to purified rLP2086 from the Subfamily B strain 8529, demonstrated surface reactivity to all ten 2086 Subfamily B strains tested by whole cell ELISA. Bactericidal activity was detected against nine of ten 2086 Subfamily B strains expressing heterologous serosubtype antigens, PorAs. These strains are representative of strains causing serogroup B meningococcal disease throughout western Europe, the Americas, Australia, and New Zealand. The only strain which was not killed in the bactericidal assay, 870227, reacted strongly with the anti-rLP2086 (Subfamily B) sera by whole cell ELISA, indicating that this strain expresses a protein with epitopes in common to P2086.

The 2086 Subfamily A strains listed in Table VII, were also tested for surface reactivity by whole cell ELISA. Two out of three of these strains appeared to have a very low level of reactivity, indicating that some 2086 Subfamily A strains may not be cross-reactive with antibodies raised to rLP2086 Subfamily B. The PCR amplification procedure used to identify the 2086 Subfamily B gene from strain 8529 was also performed on strains 870446, NMB and 6557. No 2086 Subfamily B PCR amplified product was detected.

Immunogenicity Methods:

Preparation of Antisera:

Vaccines were formulated as described previously in Example 1. However, a 10 μg dose was used.

Whole Cell Enzyme-Linked Immunosorbant Assay (ELISA):

N. meningitidis whole cell suspensions were diluted to an optical density of 0.1 at 620 nm in sterile 0.01M phosphate, 0.137M NaCl, 0.002M KCl (PBS). From this suspension, 0.1 mL were added to each well of Nunc Bac T 96 well plates (Cat# 2-69620). Cells were dried on the plates at room temperature for three days, then were covered, inverted and stored at 4° C. Plates were washed three times with wash buffer (0.001M Tris-HCl,0.139M NaCl/KCl,0.1% dodecylpoly(oxyethylereneglycolether)ₙn=23 (Brij-35®, available from ICI Americas, Inc., Wilmington, Del.), pH 7.0-7.4). Dilutions of antisera were prepared in PBS, 0.05% Tween-20/Azide and 0.1 mL was transferred to the coated plates. Plates were incubated for two hours at 37° C. Plates were washed three times in wash buffer. Goat-anti-mouse IgG AP (Southern Biotech) was diluted at 1:1500 in PBS/0.05% Tween-20, 0.1 mL was added to each well, and plates were incubated at 37° C. for two hours. Plates were washed (as above). Substrate solution was prepared by diluting p-nitrophenyl phosphate (Sigma) in 1M diethanolamine/0.5 mM MgCl₂ to 1 mg/mL. Substrate was added to the plate at 0.1 mL per well and incubated at room temperature for one hour. The reaction was stopped with 50 mL /well of 3N NaOH and plates were read at 405 nm with 690 nm reference.

B.) P4 Leader Sequence:

PCR Amplification and Cloning Strategy:

In order to optimize rLP2086 expression, the 2086 gene was cloned behind the P4 signal sequence of nontypable Haemophilus influenzae (Green et al., 1991). Primers utilized for lipoprotein cloning are listed in Table IV and are identified by compound numbers: 5658, 5660, 6473, 6543 and 6385. ORF 2086 was amplified from N. meningitidis B strain 8529 using primers with the following compound numbers 5658 and 5660. ORF 2086 was amplified from N. meningitidis serogroup B strain CDC1573 using primers with the following compound numbers 6385 and 5660. ORF 2086 was amplified from N. meningitidis serogroup B strain 2996 using primers with the following compound numbers 6473 and 6543. The N-terminal (5′) primers were designed to be homologous to the mature region of the 2086 gene (starting at the serine residue at amino acid position 3 just downstream of the cysteine). The restriction site BamHI (GGATTC) was incorporated into the 5′ end of each N-terminal primer and resulted in the insertion of a glycine residue in the mature protein at amino acid position 2. The C-terminal (3′) primers were designed to be homologous to the C-terminal end of the 2086 gene and included the Stop codon as well as an SphI site for cloning purposes. The amplified fragment from each N. meningitidis B strain was cloned into an intermediate vector and screened by sequence analysis.

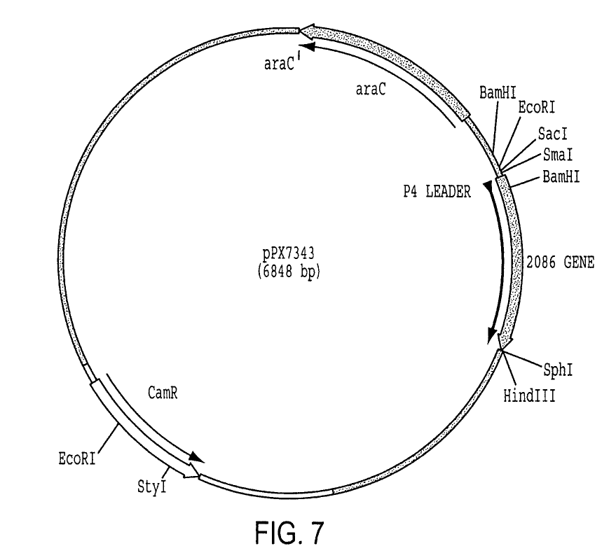

Plasmid DNA from correct clones was digested with BamHI and SphI restriction enzymes (New England Biolabs, (NEB)). A vector designated pLP339 (supplied by applicants' assignee) was chosen as the expression vector. This vector utilizes the pBAD18-Cm backbone (Beckwith et al., 1995) and contains the P4 lipoprotein signal sequence and P4 gene of nontypable Haemophilus influenzae (Green et al., 1991). The pLP339 vector was partially digested with the restriction enzyme BamHI and then subjected to SphI digestion. The amplified 2086 fragments (BamHI/SphI) were each ligated separately into the pLP339 vector (partial BamHI/SphI). This cloning strategy places the mature 2086 gene behind the P4 lipoprotein signal sequence. The BamHI site remains in the cloning junction between the P4 signal sequence and the 2086 gene (See the plasmid construct shown in FIG. 7). The following is an example of the sequence at the BamHI cloning junction:
[P4 signal sequence]-TGT GGA TCC-[remaining 2086 mature nucleic acid sequence]
[P4 signal sequence]-Cys Gly Ser-[remaining 2086 mature amino acid sequence]

Referring to FIG. 7, each amplified fragment was cloned into a modified pBAD18-Cm vector containing the P4 leader sequence. Fermentation was performed on recombinant E. coli BLR pPX7343 which expresses rP4LP2086 (recombinant P4 lipidated 2086) to try to increase the cell density by adding additional glucose. The fermentor was filled with 10 L complete M9 Minimal medium, according to Sambrook, supplemented with 1 % glucose.

The initial concentration of glucose in the fermentor was 45 g/L. The fermentor was inoculated to initial OD of ˜0.25. At ˜OD 25, additional 20 g/L glucose was added. The culture was induced with 1% arabinose at glucose depletion at OD 63.4. The fermentation continued until 3 hours after induction. Samples were saved at t=0, 1, 2, 3 post induction and protein quantified using BSA. At t=3, protein yield is ˜0.35 g/L, and 7% total cellular protein. A total of 895 grams of wet cell paste was harvested from ˜10 L of culture.

Purification of the rP4LP2086 was performed using the same methods as described above in Example 2, section A.

### Example 3

Development Genetics for Non-Lipidated Mature 2086 Protein:

To further evaluate the immunogenicity of the 2086 protein, cloning and expression of the non-lipidated form of P2086 were performed.

PCR Gene Amplification of the ORF 2086:

Oligonucleotides used for PCR amplification of the non-lipidated 2086 gene are listed in the primer table, Table IV. The 2086 gene from strain 8529 can be amplified with primers identified by compound numbers 5135 and 6406 (SEQ ID NOS. 308 and 312, respectively), as indicated in the table. The 2086 gene from strain CDC1573 can be amplified with primers identified by compound numbers 5135 and 6474 (SEQ ID NOS. 308 and 316, respectively). The 2086 gene from strain 2996 can be amplified with primers identified by compound numbers 6406 and 6605 (SEQ ID NOS. 312 and 320, respectively).

Features of these primers include, a synthetic BglII restriction site in each primer, a synthetic NdeI restriction site in compound numbers 6406 and 6474 and termination codons in all three reading frames are present in compound numbers 5135 and 6605. Primer numbers 6406 and 6474 amplify the 2086 gene with an ATG (Met) fused to the second amino terminal codon (ACG) representing a single amino acid substitution (replaces TGC Cys) of the mature 2086 polypeptide.

The PCR cloning vector was TOPO-PCR2.1, Invitrogen, Valencia, Calif.

The vector used to express non-lipidated 2086 protein was pET9a from Novagen, Madison, Wis.

The E. coli cloning strain was Top10, Invitrogen, Carlsbad, Calif.

The E. coli expression strain was BLR(DE3)pLysS, Novagen, Madison, Wis.

The culture media for cloning purposes was Terrific Broth liquid or agar, according to Sambrook et al., with 1% sterile glucose substituted for glycerol, and the appropriate antibiotic (ampicillin or kanamycin).

Plasmid purification was with Qiagen Spin Miniprep Kit (Valencia, Calif.).

Preparation of the Production Strain or Cell Line for Non-Lipidated 2086 Expression:

The 2086 gene was amplified by polymerase chain reaction (PCR) [AmpliTaq and ABI 2400 thermal cycler, Applied Biosystems, Foster City, Calif.] from chromosomal DNA derived from meningococcal strain 8529. The PCR amplification of the 2086 gene utilized two oligonucleotide primers in each reaction identified by compound numbers 6474 and 5135 (SEQ ID NOS. 316 and 308, respectively). The amplified 2086 PCR product was cloned directly into the TOPO-PCR2.1 cloning vector and selected on Terrific Broth agar supplemented with 100 μg/ml ampicillin and 20 μg/ml X-Gal. White colonies were selected and grown. Plasmid DNA was prepared using a Qiagen miniprep kit and the plasmids were screened for the PCR fragment insert. PCR insert plasmids were subjected to DNA sequencing (Big Dye chemistry on an ABI377 sequencer, Applied Biosystems, Foster City, Calif.).

Plasmids exhibiting the correct DNA sequence were digested with BglII restriction enzyme and the BglII fragment was gel purified using a GeneClean II purification kit (Bio101, Carlsbad, Calif.). The purified BglII fragment was cloned into the BamHI site of the expression vector pET9a. The pET9a/2086 clones were selected on Terrific Broth plates supplemented with 30 μg/ml kanamycin. Kanamycin resistant clones were grown and miniprep plasmid DNA was prepared. The plasmids were screened for the appropriate orientation of the 2086 gene in the BamHI site. Correctly oriented plasmids represent a fusion of the T7-antigen to the amino terminus of 2086 gene (rP2086T7). These rP2086T7 gene fusions were transformed into BLR(DE3)pLysS, selected on Terrific Broth/Kan plates, grown in Terrific Broth and induced to express the rP2086T7 fusion protein with 1 mM IPTG (isopropyl P-D-thiogalactopyranoside). The rP2086T7 fusion protein expressed at high levels.

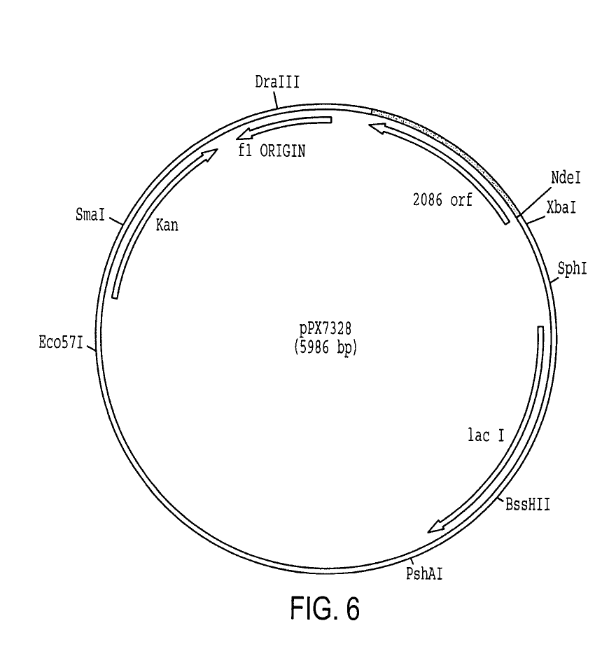

These fusion plasmids were then subjected to a NdeI restriction digest, which deletes the T7-antigen and links the mature 2086 gene directly to the ATG start provided by the vector. These NdeI deleted plasmids were transformed into Top10 cells and selected on Terrific Broth/Kan plates. Candidate clones were grown and miniprep plasmid DNA was prepared. The plasmid DNA was subjected to DNA sequencing to confirm the deletion and the integrity of the 2086 gene sequence. These plasmids are represented by the plasmid map designated pPX7328 (FIG. 6). Plasmids representing the correct DNA sequence were transformed into BLR(DE3)pLysS, selected on Terrific Broth/Kan plates, grown in Terrific Broth and induced to express the 2086 protein with IPTG. The pET9a vector failed to express the mature 2086 protein, in strain BLR(DE3)pLysS, when the T7-Tag was removed.

Production of Non-Lipidated 2086 Protein:

Purified plasmid DNA was used to transform the expression strain BLR(DE3)pLysS. BLR(DE3)pLysS cells carrying the plasmids are resistant to kanamycin and can be induced to express high levels of PorA protein by the addition of 1 mM IPTG. The rP2086T7 fusion protein can be expressed as insoluble inclusion bodies in the E. coli cell line BLR(DE3)pLysS at ˜40% of total protein. This purified fusion protein was used to immunize mice and generated significant levels of bactericidal antibodies against a heterologous meningococcal strain. (See Table V)

2086 Non-Lipidated Gene Mutagenesis:

PCR primer mutagenesis was performed on the 5′ end of the 2086 gene. Expression studies are under way to determine if the T7-Tag can be removed while exhibiting the high expression levels of mature rP2086T7.

Purification of Non-Lipidated rP2086T7:

E. coli BLR(DE3)pLysS cells expressing non-lipidated rP2086T7 were lysed by microfluidizer in 10 mM Hepes-NaOH/5 mM EDTA/1 mM Pefabloc SC pH 7.4. The cell lysate was then centrifuged at 18,000×g for 30 minutes. The inclusion body pellet was washed three times with 50 mM Tris-HCl/5 mM EDTA/1% TritonX-100 pH 8 followed by centrifugation each time at 24,000×g for 30 min. The inclusion body pellet was then washed twice with 50 mM Tris-HCl/5 mM EDTA/1% Zwittergent 3-14 pH 8 followed by centrifugation each time at 24,000×g for 15min. The inclusion body pellet was then solubilized with 50 mM Tris-HCl/5 mM EDTA/4M Urea pH 8 for two hours followed by centrifugation to remove insoluble material. The supernatant (solubilized rP2086T7) was split into four equal samples. One sample was adjusted to 50 mM Tris-HCl/5 mM EDTA/250 mM NaCl/2M Urea pH8 (no detergent), one was adjusted to 50 mM Tris-HCl/5 mM EDTA/250 mM NaCl/2M Urea/1% hydrogenated Triton X-100 pH8 (TX-100), one was adjusted to 50 mM Tris-HCl/5 mM EDTA/250 mM NaCl/2M Urea/1% Zwittergent 3-12 pH8 (Z3-12), and one was adjusted to 50 mM Tris-HCl/5 mM EDTA/250 mM NaCl/2M Urea/1% Zwittergent 3-14 pH8 (Z3-14) using stock solutions. To remove the urea, samples were dialyzed to completion against the respective buffer containing no urea. The samples were then dialyzed to completion against the respective buffer containing no urea and 60 mM NaCl to reduce the NaCl concentration. Insoluble material was removed by centrifugation at 2,000×g for 15 minutes, and the resulting supernatant (refolded rP2086T7) was used for further experiments. Homogeneity of rP2086T7 was found to be 91-95% as determined using Coomassie stained SDS-PAGE and laser densitometry.

Immunogenicity Procedure—As described in Example 2

This purified fusion protein was used to immunize mice and generated significant levels of bactericidal antibodies against a heterologous meningococcal strain. (See Table V below):

TABLE V
Bactericidal titers of mouse antibody raised to rP2086T7

| | | HETEROLOGOUS |
|---|---|---|
| MOUSE SERUM | DESCRIPTION | STRAIN/H44/76 |
| AF780 week 6 | r2086T7, 10 ug | 3200 |
| Week 0 pool | Pre-immune serum | 10 |
| AE203 week 6 | rLP2086, 10 ug (positive | 6400 |
| | control)* | |

(*positive control sera generated by immunization of mice with rLP2086)

### Example 4

Development of Chimeric Clones of ORF 2086

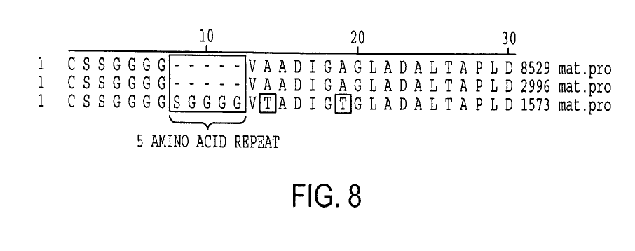

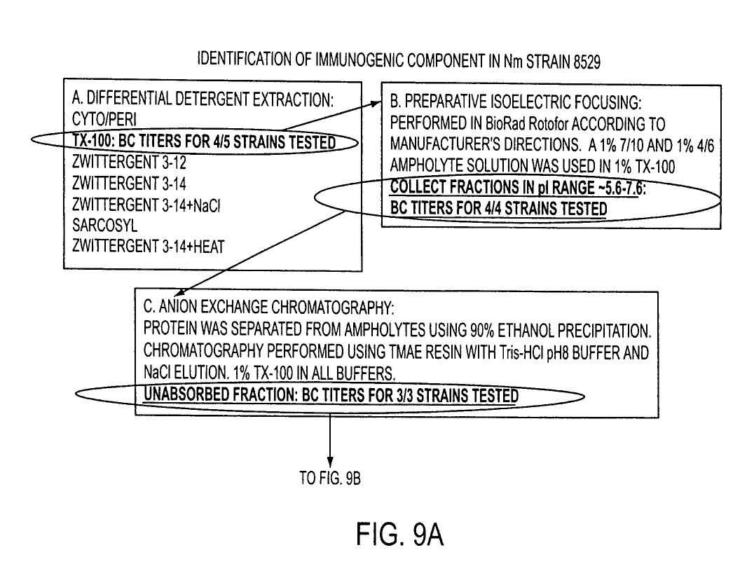

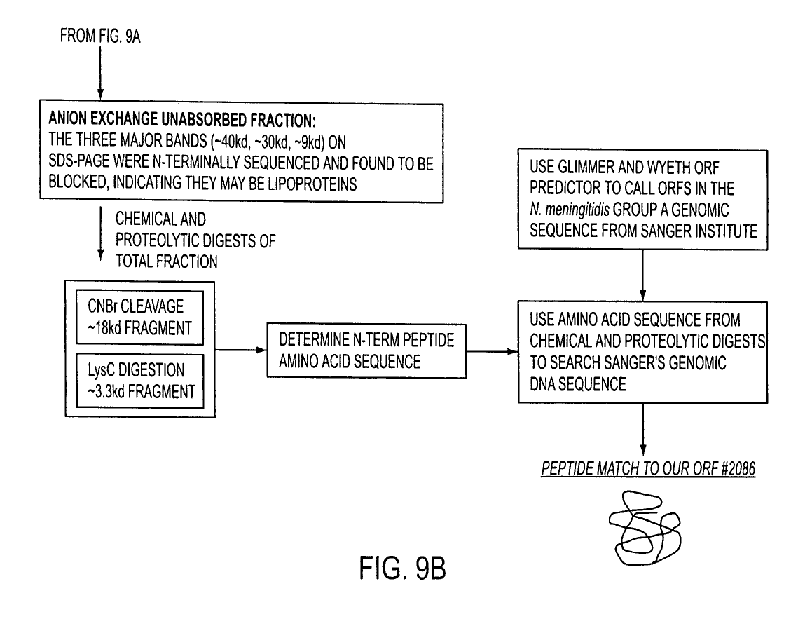

The N-terminal region of the 2086 gene from strain CDC-1573 contains a repeated segment not present in the 2086 gene from strains 8529 and 2996 (see FIG. 8). It appears that this repeated segment is responsible for increased levels of recombinant 2086 protein expression from two E. coli based expression systems (pET and pBAD). The recombinant protein expression level from the CDC-1573 2086 gene was significantly better in the pET and pBAD expression systems as compared to the recombinant expression levels from the 2086 gene with strains 8529 and 2996 using the same systems. The N-terminal region of the 2086 gene from all three strains is relatively homologous, except for this repeated segment. Therefore, it is reasonable to assume that by fusing the CDC-1573 N-terminus to the 2086 genes from strains 8529 and 2996, that the recombinant 2086 protein levels expressed from these genes will increase when using the pET and pBAD systems.

Materials and Methods:

Chromosomal DNA from strains 8529 and 2996 was purified and used as a template for PCR amplification of the chimeric 2086 gene. PCR primers with the compound numbers 6721 and 5135 (SEQ ID NOS. 321 and 308, respectively) were used to amplify the chimeric 2086 gene from strain 8529 and PCR primers with the compound numbers 6721 and 6605 (SEQ ID NOS. 321 and 320, respectively) were used to amplify the chimeric 2086 gene from strain 2996. The PCR products were cloned directly into the PCR2.1 TOPO vector from Invitrogen and then screened by DNA sequence analysis to identify an intact chimeric 2086 gene. That gene was then cleaved from the PCR2.1 vector with BglII and the BglII fragment was inserted into the BamHI site of the pET9a plasmid. Plasmid inserts were screened for the appropriate orientation and then subjected to a NdeI digestion. The linear NdeI fragments were self-ligated to achieve the deletion of a small NdeI fragment containing the T7-tag sequence contributed by the pET9a vector. This deletion directly links the T7 promoter to the 5′ end of the chimeric 2086 gene. The NdeI deleted plasmid was transformed into E. coli strain BL21(DE3) and kanamycin resistant colonies were screened for chimeric 2086 protein expression with IPTG induction.

Initial studies indicate that the chimeric 2086 gene from strain 2996 expresses about twice as much recombinant protein as compared to the native 2996/2086 gene when expressed in the pET9a system. The pBAD system has not been tested yet.

Although only one experiment has been performed, the data indicate that there is an enhanced utility from the chimeric 2086 gene. The generation of CDC-1573 N-terminal fusions to the 2086 genes from strains 8529 and 2996 provides enhanced recombinant 2086 protein expression.

### Example 5

2086 PCR Screening of N. meningitidis Strains:

In order to determine the conservation of the 2086 gene among clinical isolates, PCR amplification was performed on 88 N. meningitidis strains.

Initial PCR identification of ORF 2086 utilized primers listed in Table IV (see Example 2 above) identified by compound numbers: 4623, 4624 and 4625 (SEQ ID NOS. 303, 304 and 305, respectively). These primers were designed based on Sanger's N. meningitidis serogroup A sequence. To facilitate screening a large number of strains, internal primers were designed for the 2086 gene. A total of 88 N. meningitidis strains were screened by PCR with the newly designed internal 2086 primers identified by compound numbers 5005 and 5007 (SEQ ID NOS. 306 and 307). With these primers the applicants were able to identify the 2086 gene from 63 of the 88 (˜70%) N. meningitidis strains, (see Table VI-A).

Expanded regions surrounding the 2086 gene in Sanger's N. meningitidis serogroup A sequence and TIGR's N. meningitidis serogroup B sequence were examined and aligned. Primers were designed to correspond to regions upstream and downstream of the 2086 gene. The purpose was to utilize these primers to amplify greater than full length 2086 genes from a variety of N. meningitidis strains for sequence comparison. PCR amplification of one strain (6557), using Compound Nos. 6470 and 6472 (SEQ ID NOS: 313 and 314, respectively), resulted in a low yield of product. The strain 6557 amplified product was cloned and plasmid DNA was submitted for sequence analysis. Results indicated a new type of 2086 gene with greater sequence variability than had previously been seen. The 2086 gene from strain 6557 was ˜75% identical at the amino acid level to the other strains sequenced. Interestingly, strain 6557 was one of the 30% of strains that had previously tested negative by 2086 PCR screening described above.

Internal primers specific to the C-terminal variable regions within strain 6557 were designed. These primers were used to screen for the more variable 2086 gene in the ˜30% of strains that had previously tested negative by 2086 PCR screening. All available N. meningitidis strains (n=88) were screened by PCR with these newly identified internal 2086 primers (identified by compound numbers 6495 and 6496; SEQ ID NOS. 159 and 160, respectively). Only the ˜30% of N. meningitidis strains that had previously tested negative by PCR for 2086 were PCR positive in this screen. The set of genes amplified from the previously PCR negative (˜30%) strains should represent a new type of 2086 gene or a second family of 2086 genes and herein are designated 2086 Subfamily A. The set of 2086 genes amplified from the ˜70% of strains with the 8529 derived primers are herein designated Subfamily B.

Subfamily A of 2086 genes is exemplified by the odd numbered SEQ ID NOS:1-173 without limitation. Subfamily B of 2086 genes is exemplified, without limitation, by the odd numbered SEQ ID NOS: 175-251

N. meningitidis strains used for PCR amplification studies were selected from the following tables, Table VI-A and Table VI-B. The strains listed in the tables are provided as examples of N. meningitidis strains, without limitation. The strains listed in Table VI-A are classified in 2086 protein Subfamily A and the strains listed in Table VI-B are classified in 2086 protein Subfamily B. The strains listed in each table are grouped by serosubtype. The strains are available from the following four sources as indicated in the table: MPHL-Manchester Public Health Laboratory, Manchester, UK; RIVM, Bilthoven, The Netherlands; University of Iowa, College of Medicine, Department of Microbiology, Iowa City, Iowa; and Walter Reed Army Institute of Research, Washington, D.C.

TABLE VI-A

| Strain | Serosubtype | Source |
|---|---|---|
| M97 251854 | B: 4z, PI: 4 | MPHL |
| M98 250622 | B: 2b, PI: 10 | MPHL |
| M98 250572 | B: 2b, PI: 10 | MPHL |
| M98 250771 | B: 4z, PI: 14 | MPHL |
| M98 250732 | B: 4z, PI: 14 | MPHL |
| M98 250809 | B: 15, PI: 7,16 | MPHL |
| M97 252697 | B: 1, PI: 6 | MPHL |
| M97 252988 | B: 4, PI: 6 | MPHL |
| M97 252976 | B: 4, PI: 6 | MPHL |
| M97 252153 | B: 4, PI: 6 | MPHL |
| M97 253248 | B: 15, PI: 7, NT | MPHL |
| CDC1610 | P1: NT 4(15) | CDC |
| CDC1521 | P1.6,3 2b(4) | CDC |
| CDC1034 | P1.7 4(15) | CDC |
| L8 | P1.7,1 15(4) | Walter Reed |
| CDC1492 | P1.7,1 4(15) | CDC |
| 870446 | P1.12a,13 | RIVM |
| CDC2369 | P1.(9),14 | CDC |
| 6557 | P1.(9),14 | RIVM |
| 2996 | P1.5,2 | RIVM |
| NmB | P1.5,2 | UIOWA |
| L3 | P1.5,2 | Walter Reed |
| B16B6 | P1.5,2 | RIVM |
| CDC1135 | | CDC |
| L5 | P1.NT | Walter Reed |
| L4 | PI.21,16 | Walter Reed |

TABLE VI-B

| Strain | Serosubtype | Source |
|---|---|---|
| M98 250670 | B: 1, PI: 4 | MPHL |
| M98 250024 | B: 1, PI: 4 | MPHL |
| M97 253524 | B: 1, PI: 4 | MPHL |
| M97 252060 | B: 1, PI: 4 | MPHL |
| M97 251870 | B: 4z, PI: 4 | MPHL |
| M97 251836 | B: 4z, PI: 4 | MPHL |
| M97 251830 | B: 4z, PI: 4 | MPHL |
| M97 251905 | B: 4z, PI: 4 | MPHL |
| M97 251898 | B: 4z, PI: 4 | MPHL |
| M97 251885 | B: 4z, PI: 4 | MPHL |
| M97 251876 | B: 4z, PI: 4 | MPHL |
| M97 251994 | B: 4z, PI: 4 | MPHL |
| M97 251985 | B: 4z, PI: 4 | MPHL |
| M97 251957 | B: 4z, PI: 4 | MPHL |
| M97 251926 | B: 4z, PI: 4 | MPHL |
| M97 252045 | B: 4z, PI: 4 | MPHL |
| M97 252038 | B: 4z, PI: 4 | MPHL |
| M97 252026 | B: 4z, PI: 4 | MPHL |
| M97 252010 | B: 4z, PI: 4 | MPHL |
| M97 252098 | B: 4z, PI: 4 | MPHL |
| M97 252083 | B: 4z, PI: 4 | MPHL |
| M97 252078 | B: 4z, PI: 4 | MPHL |
| M98 250735 | B: 4z, PI: 15 | MPHL |
| M98 250797 | B: 4z, PI: 15 | MPHL |
| M98 250768 | B: 4z, PI: 15 | MPHL |
| M98 250716 | B: 2b, PI: 10 | MPHL |
| M98 250699 | B: 4z, PI: 10 | MPHL |
| M98 250393 | B: 4z, PI: 10 | MPHL |
| M98 250173 | B: 4z, PI: 10 | MPHL |
| M97 253462 | B: 4z, PI: 14 | MPHL |
| M98 250762 | B: 15, PI: 7,16 | MPHL |
| M98 250610 | B: 15, PI: 7,16 | MPHL |
| M98 250626 | B: 15, PI: 7,16 | MPHL |
| M97 250571 | B: 15, PI: 16 | MPHL |
| M97 252097 | B: 15, PI: 16 | MPHL |
| M97 253092 | B: 1, PI: 6 | MPHL |
| M97 252029 | B: 15, PI: 7, NT | MPHL |
| M97 251875 | B: 15, PI: 7, NT | MPHL |
| CDC1127 | PI.7,16 4(15) | CDC |
| CDC982 | PI.7,16 4(15) | CDC |
| CDC1359 | PI.7,16 4(15) | CDC |
| CDC798 | PI.7,16 15(4) | CDC |
| CDC1078 | PI.7,16 15(4) | CDC |
| CDC1614 | PI.7,16 15(4) | CDC |
| CDC1658 | PI.7,16 15(4) | CDC |
| H44/76 | PI.7,16 15(4) | RIVM |
| CDC1985 | P1.7,13 4(15) | CDC |
| L6 | P1.7,1 ?(4) | Walter Reed |
| CDC1573 | P1.7,1 4(15) | CDC |
| L7 | P1.7,(9),1 | Walter Reed |
| CDC937 | P1.7,3 | CDC |
| 8529 | P1.7,3 | RIVM |
| 880049 | P1.7b,4 | RIVM |
| CDC2367 | P1.15 4(15) | CDC |
| H355 | P1.19,15 | RIVM |
| CDC1343 | P1.14 4(15) | CDC |
| M982 | P1.22,9 | RIVM |
| 870227 | P1.5c,10 | RIVM |
| B40 | P1.5c,10 | RIVM |
| 5315 | P1.5c,10 | RIVM |
| CDC983 | P1.5,2 | CDC |
| CDC852 | P1.5,2 | CDC |
| 6940 | P1.18,25 (6) | RIVM |

Other strains are readily available as isolates from infected individuals.

### Example 6

Reactivity of rLP2086 Antisera Against Meningococcal Strains:

The following table, Table VII, shows the cross-reactive and cross protection capacity of the rLP2086 as described above. As indicated in the table, the rLP2086 was processed and analyzed using a variety of techniques including whole cell ELISA (WCE) titers, bactericidal assay (BCA) and Infant Rat (IR) assays to determine the bacterial cell surface reactivity of a polyclonal antibody raised against the 2086 protein.

TABLE VII
Reactivity of rLP2086-8529 antisera against multiple
meningococcal strains

| | | | | |
|---|---|---|---|---|
| Strain | Serosubtype | WCE | BC | IR |

2086 Subfamily A

| | | | | |
|---|---|---|---|---|
| 870446 | P1.12a,13 | 808,615 | >800 | |
| NmB | P1.5a,2c | 47,954 | <100 | |
| 6557 | P1.22a,14a | 169,479 | <25 | − |

2086 Subfamily B

| | | | | |
|---|---|---|---|---|
| 880049 | P1.7b,4 | 1,402,767 | 100 | + |
| H44/76 | P1.7,16 | 8,009,507 | >6400 | |
| H355 | P1.19,15 | 10,258,475 | 3,200 | + |
| 6940 | P1.18,25(6) | 5,625,410 | 800 | |
| 870227 | P1.5c,10 | 4,213,324 | <25 | + |
| 252097 | P1.7b,16 | 10,354,512 | >800 | |
| 539/8529 | P1.7b,3 | 11,635,737 | 3,200 | |
| M982 | P1.22,9 | 1,896,800 | 800 | |
| CDC-1573 | P1.7a,1 | 208,259 | 25 | |
| CDC-937 | P1.7b,(3) | 9,151,863 | >800 | |

+ greater than 10 fold reduction in bacteremia
− less than 10 fold reduction in bacteremia

### Example 7

Various constructs for expressing ORF2086 protein were prepared. The following table, Table VIII, is an r2086 construct table which is provided for the purpose of showing examples and illustrating an implementation of the present invention, without limitation thereto.

TABLE VIII
r2086 Construct Summary

| Construct | Promoter | Leader | Expression | Extraction | Vector | % total Protein |
|---|---|---|---|---|---|---|
| pPX7340 | T7 | native | Coomassie | sarcosyl | pET27b | 2.5% processed |
| | | | | soluble | | lipoprotein |
| pPX7341 | T7 | P4 | Coomassie | sarcosyl | pET27b | 5% processed |
| | | | | soluble | | lipoprotein |
| pPX7343 | Arabinose | P4 | Coomassie | sarcosyl | pBAD18 cm | 7-10% processed |
| | | | | soluble | | lipoprotein |
| pPX7325 | T7 | T7-tag | Coomassie | inclusion | pET9a | 40-50% mature |
| | | fusion/ | | bodies | | protein |
| | | mature | | | | |
| pPX7328 | T7 | mature | Coomassie | soluble | pET9a | 10% mature |
| | | | | | | protein |

### Example 8

Further studies with LOS depleted outer membrane proteins identified additional strains producing outer membrane protein(s) other than PorA which were capable of eliciting bactericidal antibodies to strains expressing heterologous serosubtypes. The following describes further studies to identify additional proteins according to one embodiment of the present invention, and specifically outer membrane lipoproteins, which can reduce the number of proteins required in a meningococcal immunogenic composition. These further studies supplement the studies described in the previous examples.

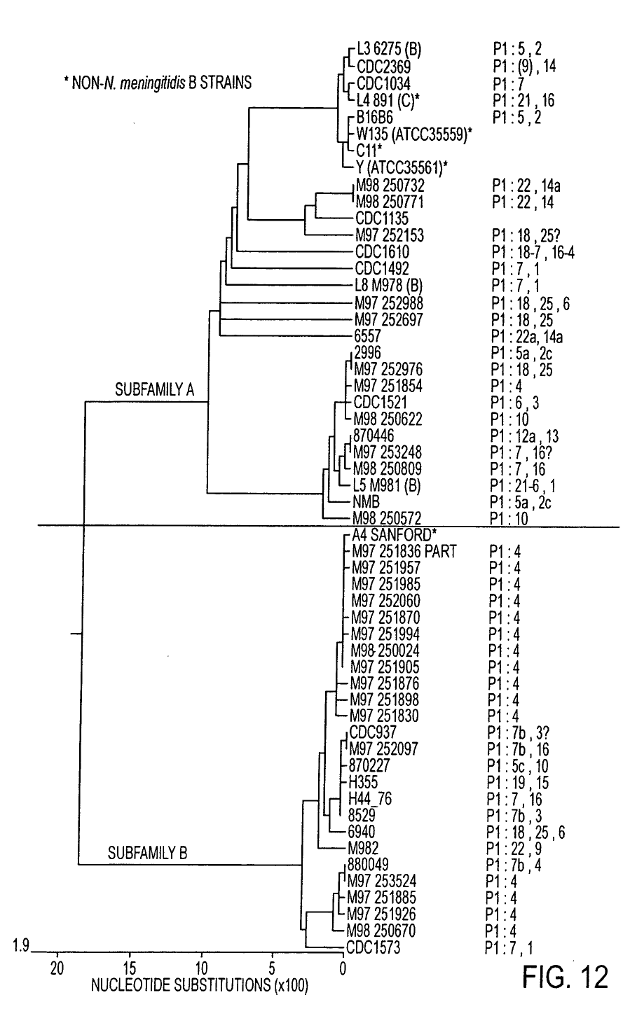

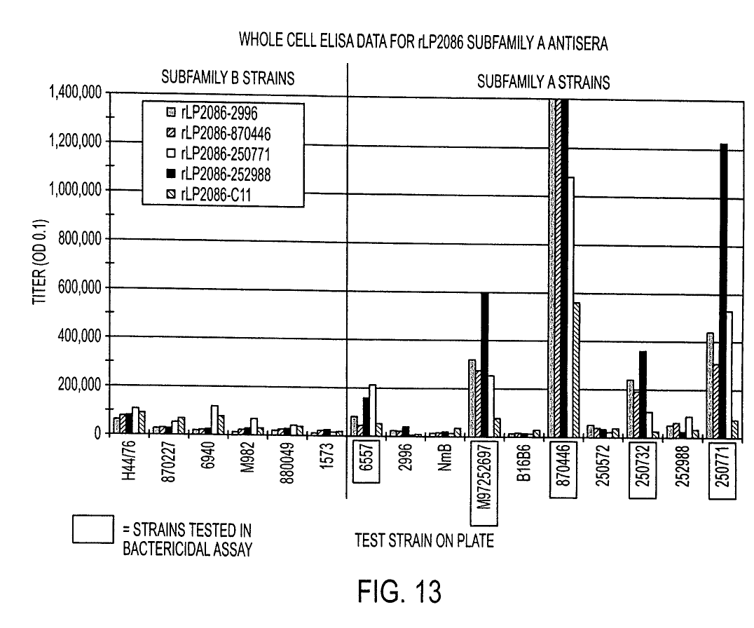

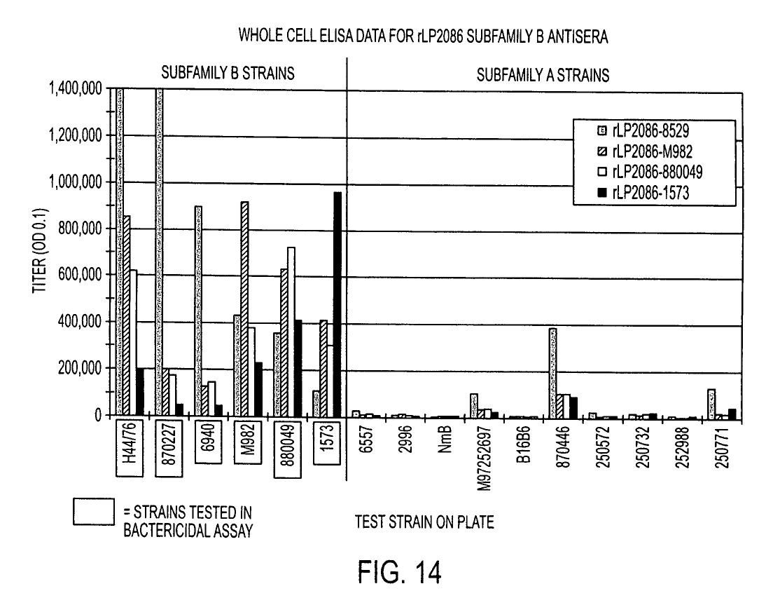

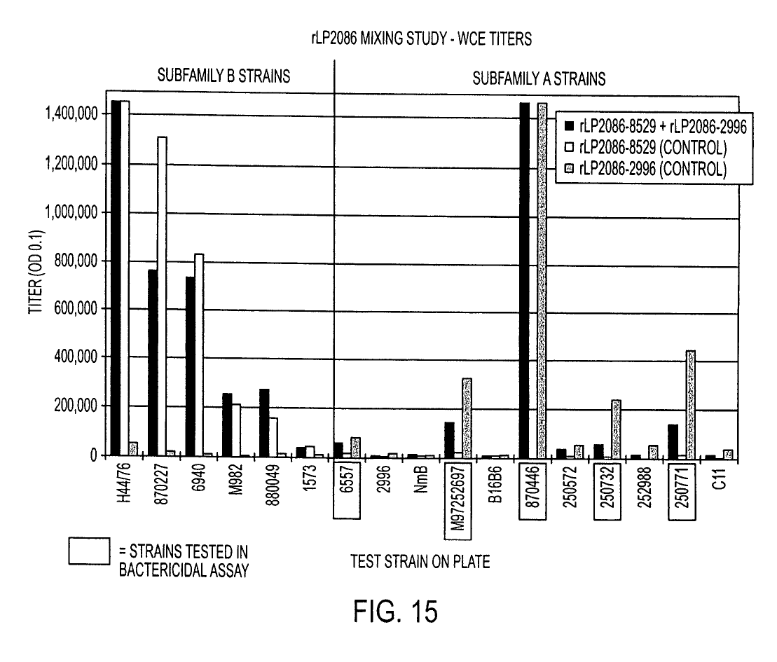

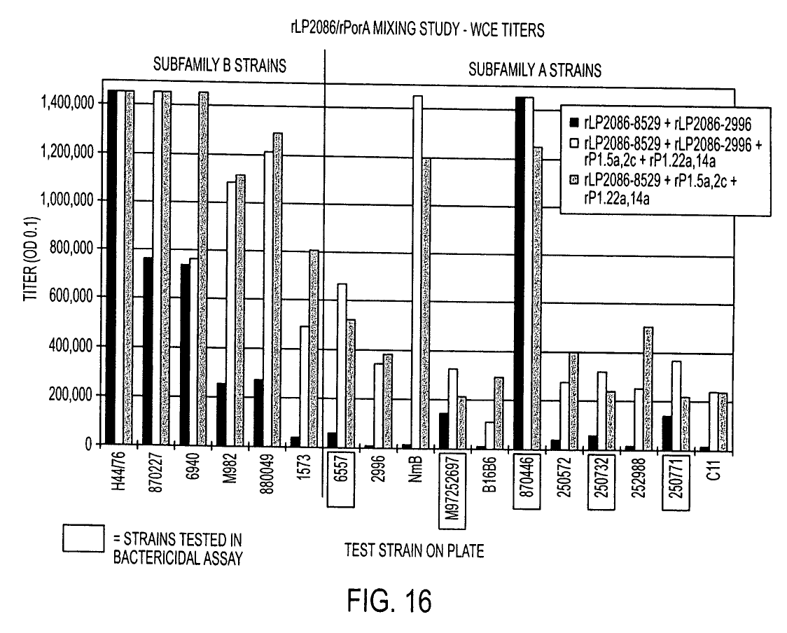

Subcellular fractionation, differential detergent extraction, isoelectric focusing, and ion exchange chromatography were used in conjunction with immunization and bactericidal assays against multiple strains to identify small groups of proteins of interest. Direct sequencing of the main components indicated that the N-termini were blocked. Internal protein sequences were obtained by direct sequencing of polypeptides derived from chemical and proteolytic digests. The genomic sequence of a group A meningococcal strain was downloaded from the Sanger Center and analyzed by our Bioinformatics group using existing and proprietary algorithms to create a searchable database. The peptide sequence data indicated that ORF2086 was of interest. Primers based on this orf were used to PCR the P2086 gene from strain 8529. Analysis of the gene sequence, the fact that the N-terminus was blocked, and its subcellular location indicated that P2086 is a lipidated outer membrane protein(LP2086). rLP2086-8529 and variants from other meningococcal strains were recombinantly expressed as lipoproteins in E. coli using the H. influenzae P4 signal sequence. These recombinant proteins were isolated from E. coli membranes by differential detergent extraction, purified using ion exchange chromatography, and used to immunize mice. Mouse anti-LP2086 sera were able to facilitate bactericidal activity against several different serosubtype strains of N. meningitidis. Further analysis of the P2086 genes from many N. meningitidis strains showed that these sequences fell into two groups designated Subfamily A and Subfamily B. (See FIG. 12) The antisera raised against the Subfamily B proteins were bactericidal against nine strains expressing Subfamily B proteins, and one strain expressing a Subfamily A protein. Subfamily A antisera were bactericidal against Subfamily A strains. A mixture of one rPorA and one rLP2086 elicited complementary antibodies extending vaccine coverage beyond that induced by either protein alone.

These observations lead to the following conclusions. rLP2086 antigens are capable of eliciting bactericidal antibodies against meningococcal strains expressing heterologous PorAs and heterologous P2086 proteins. The P2086 family of antigens may be a useful vaccine or immunogenic either alone or in combination with other neisserial antigens.

The following describes the foregoing study in detail. A complex mixture of soluble outer membrane proteins (sOMPs) was found to elicit PorA independent bactericidal antibody against strains expressing heterologous PorA proteins. A process of differential detergent extraction, isoelectric focusing and ion exchange chromatography followed by mouse immunization was used to follow the immunologically active components.

At each step, sera was assayed for surface reactivity and bactericidal activity against several strains containing serosubtype antigens that are representative of the worldwide epidemiology of meningococcal disease.

This process of separation and immunization was used to identify a novel cross-reactive immunogenic candidate for Group B N. meningitidis.

Generation of PorA deficient strains—The porA chromosomal locus was cloned into plasmid pPX7016 from strain 2996. Within the plasmid the porA promoter, the S/D box and the first 38 N-terminal codons have been deleted and replaced with a self contained KanR expressing cassette. The plasmids were linearized with restriction enzymes and naturally transformed into the serosubtype strains PI:5,2; PI:9; PI:7,16; PI:15; PI:4; PI:3 & PI:10. Kanamycin resistant transformants were selected and screened for the loss of PorA by serosubtype specific monoclonals in an ELISA.

Bactericidal Assay: See Mountzourous, K. T. and Howell, A. P. Detection of Complement-Mediated Antibody-Dependent Bactericidal Activity in a Flourescence-Based Serum Bactericidal Assay for Group B Neisseria meningitidis. J Clin Microbiol. 2000;38:2878-2884.

Whole Cell Enzyme Linked Immonosorbant Assay (ELISA): N. meningitidis whole cell suspensions were diluted to an optical density of 0.1 at 620 nm in sterile 0.01M phosphate, 0.137M NaCl, 0.002M KCl (PBS). From this suspension, 0.1 mL were added to each well of Nunc Bac T 96 well plates (Cat# 2-69620). Cells were dried on the plates at 37° C. overnight, then were covered, inverted and stored at 4° C. Plates were washed three times with wash buffer (0.01M Tris-HCl,0.139M NaCl/KCl,0.1I% Brij-35, pH 7.0-7.4). Dilutions of antisera were prepared in PBS, 0.05% Tween-20/Azide and 0.1 mL was transferred to the coated plates and incubated for two hours at 37° C. Plates were washed three times in wash buffer. Goat-anti-mouse IgG AP (Southern Biotech) was diluted at 1:1500 in PBS/0.05% Tween-20, 0.1 mL was added to each well, and plates were incubated at 37° C. for two hours. Plates were washed (as above). Substrate solution was prepared by diluting p-nitrophenyl phosphate (Sigma) in diethanolamine at 1 mg/ml . Substrate was added to the plate at 0.1 mL per well and incubated at room temperature for one hour. The reaction was stopped with 50 ul/well of 3N NaOH and plates were read at 405 nm with 690 nm reference.

Recombinant PorA Induction: The BLR(DE3)/pET9a strains were grown overnight at 37° C. in HySoy Broth (Sheffield Products) supplemented with Kan-30 and 2% glucose. In the morning the O/N cultures were diluted 1/20 in HySoy Broth Kan-30 and 1% glycerol and grown at 37° C. for 1 hour. These cultures were induced by the addition of IPTG to a final concentration of 1 mM. The cultures were grown for an additional 2-3 hours and then harvested.

Recombinant PorA Purification: The rPorA was solubilized from E. coli inclusion bodies with 8M Urea, and refolded by dialysis against buffer containing no urea. The refolded rPorA was then concentrated by diafiltration and buffer exchanged by G25 column into NaPO4 pH6. The dialyzed rPorA was then run on a cation exchange column (S Fractogel) and eluted with 1M NaCl. The sOMPs from strain 8529 (P1.7-2,3) elicit PorA independent bactericidal activity in mice against strains expressing heterologous serosubtypes. The following table, Table IX, shows the bactericidal activity in the studied strains.

TABLE IX

| Test Strain | Serosubtype | BC₅₀Titer¹ |
|---|---|---|
| 539 | P1.7-2,3 | 1280 |
| 539 PorA- | NST² | 1080 |
| H44/76 | P1.7,16 | 3285 |
| H44/76 PorA- | NST | 2620 |
| H355 | P1.19,15 | >1350 |
| H355 PorA- | NST | >1350 |
| 880049 | P1.7-2,4 | 290 |
| 880049 PorA- | NST | 85 |
| M982 | P1.22,9 | 85 |
| M982 PorA- | NST | <50 |

¹Bactericidal (BC₅₀) titers represented as the reciprocal of the dilution of antisera which reduces viable cell count by 50%. Week 0 normal mouse sera had BC₅₀ titers of <25
²NST = Non Serosubtypable

Preparation of sOMPs: N. meningitidis membranes were extracted with TX- 100, Zwittergent 3-14, and Zwittergent 3-14+0.5M NaCl. The sOMPs referred to above were solubilized in the Zwittergent 3-14/0.5M NaCl extract. The extraction is performed using techniques well known to persons skilled in the art, for example, see U.S. Pat. No. 6,355,253 which is hereby incorporated by reference.

Immunogencity: Female Swiss-Webster mice were immunized with 25 ug total protein adjuvanted with 20 ug QS-21 at week 0 and 4. An exsanguination bleed and data analysis were done at week 6.

The following table, Table X, shows the purification and characterization summary for recombinant lipidated P2086 (rLP2086) for both Subfamily A and Subfamily B.

Subfamily A rLP2086 Purification

TABLE X

| rLP2086 Variant | A.A. Homology (%)¹ | Theoretical pI | Purity (%)² |
|---|---|---|---|
| 870446 | 75 | 6.1 | 80 |
| 2996 | 71 | 5.9 | 95 |
| M97 252988 | 71 | 6.3 | 96 |
| C11 | 68 | 6.4 | 82 |
| M98 250771 | 62 | 6.1 | 83 |

Subfamily B rLP2086 Purification

TABLE XI

| rLP2086 Variant | A.A. Homology (%)¹ | Theoretical pI | Purity (%)² |
|---|---|---|---|
| 8529 | 100 | 7.5 | 96 |
| M982 | 94 | 6.3 | 96 |
| 88049 | 92 | 6.2 | 90 |
| CDC1573 | 87 | 5.6 | 93 |

¹Amino acid homology as compared to P2086 from strain 8529
²Purity as determined by SDS-PAGE and laser densitometry of colloidal Coomassie stained band (Simply Blue stain)

Immunogenicity of a Subfamily B member, rLP2086-8529, tested against homologous and heterologous strains

Table XII below shows immunogenicity of a Subfamily B member, rLP2086-8529, tested against homologous and heterologous strains

TABLE XII

| | P2086 | Target Strain | | Whole Cell | |
|---|---|---|---|---|---|
| Target Strain | Subfamily | Serosubtype | A.A. Homology^{a} | ELISA^{b} Titer | BC₅₀ Titer^{C} |
| 539 | B | P1.7-2,3 | 100 | >1,458,000 | 3,200 |
| H44/76 | B | P1.7,16 | 100 | >1,458,000 | 3,200 |
| H355 | B | P1.19,15 | 100 | >1,458,000 | 3,200 |
| CDC937 | B | P1.7-2,3-4 | 100 | >1,458,000 | >800 |
| M97 252097 | B | P1.7-2,16 | 100 | >1,458,000 | >800 |
| 870227 | B | P1.5-2,10 | 100 | >1,458,000 | <25 |
| 6940 | B | P1.18,25,6 | 97 | 900,162 | >800 |
| M982 | B | P1.22,9 | 94 | 435,909 | 200 |
| 880049 | B | P1.7-2,4 | 92 | 349,912 | 400 |
| CDC1573 | B | P1.7-1,1 | 87 | 102,508 | 25 |
| 870446 | A | P1.12-1,13 | 71 | 389,829 | 800 |
| M98 250771 | A | P1.22,14 | 62 | 139,397 | <25 |
| NmB | A | P1.5-1,2-2 | 71 | <2,000 | <25 |

Vaccination Procedure: 6-8 week old female Swiss-Webster mice were immunized with 10 ug rLP2086-8529 + 20 ug QS-21 at week 0 and week 4. Data analysis was performed on the week 6 exsanguination bleed.
^{a}Amino acid homology of P2086 as compared to rLP2086-8529
^{b}Endpoint titers expressed as the reciprocal of the dilution at absorbance = 0.1
^{C}BC50 titers represented as the reciprocal of the dilution of anti-sera which reduces viable cell count by 50%. Week 0 normal mouse sera had BC50 titers of <10

Table XIII shows immunogenicity of a Subfamily B member, rLP2086-2996, tested against homologous and heterologous strains.

TABLE XIII

| | P2086 | Target Strain | | Whole Cell | |
|---|---|---|---|---|---|
| Target Strain | Subfamily | Serosubtype | A.A. Homology^{a} | ELISA^{b} Titer | BC₅₀ Titer^{C} |
| NmB | A | P1.5-1,2-2 | 99.6 | 8,979 | <25 |
| 870446 | A | P1.12-1,13 | 99 | <1,458,000 | >800 |
| M97 252697 | A | P1.18,25,6 | 98 | 320,732 | >800 |
| 6557 | A | P1.22-1,14-1 | 98 | 17,319 | <25 |
| M98 250732 | A | P1.22,14-1 | 89 | 241,510 | >800 |
| M98 250771 | A | P1.22,14 | 89 | 447,867 | 800 |
| H44/76 | B | P1.7,16 | 72 | 56,386 | <25 |

Vaccination Procedure: 6-8 week old female Swiss-Webster mice were immunized with 10 ug rLP2086-2996 + 20 ug QS-21 at week 0 and week 4. Data analysis was performed on the week 6 exsanguination bleed.
^{a}Amino acid homology of P2086 as compared to rLP2086-2996
^{b}Endpoint titers expressed as the reciprocal of the dilution at absorbance = 0.1
^{C}Bactericidal (BC50) titers represented as the reciprocal of the dilution of antisera which reduces viable cell count by 50%. Week 0 normal mouse sera had BC50 titers of <10.

Table XIV below shows that antisera to rLP2086 and rPorA are complimentary when mixed and assayed for bactericidal activity.

TABLE XIV

| | H44/76 | NMB | 880049 | H355 | 870227 | 6557 |
|---|---|---|---|---|---|---|
| Antisera | (P1.7,16) | (P1.5-1,2-2) | (P1.7-2,4) | (P1.19,15) | (P1.5-2,10) | (P1.22-1,14-1) |
| Anti-rLP2086 + | >3,200 | >800 | 200 | >800 | 200 | 200 |
| three rPorA | | | | | | |
| antisera | | | | | | |
| Controls | | | | | | |
| anti-rLP2086 | 6,400 | <25 | 100 | 3,200 | <25 | <25 |
| Corresponding | — | 1,600 | — | — | 200 | 400 |
| monovalent | | | | | | |
| rPorA antisera | | | | | | |

Vaccination Procedure: 6-8 week old female Swiss-Webster mice were immunized with either 10 ug rLP2086-8529/20 ug QS-21, or 15 ug rPorA/100 ug MPL at week 0 and week 4. Data analysis was performed on the week 6 exsanguination bleed.

a Bactericidal (BC50) titers represented as the reciprocal of the dilution of anti-sera which reduces viable cell count by 50%. Week 0 normal mouse sera had BC50 titers of <10.

The following table, Table XV, shows that mixtures of rLP2086 Subfamilies and two rPorAs elicit bactericidal antibody in mice.

TABLE XV

| | | 6940 | 880049 | | M98 | M98 | M97 | 870446 | NmB | 6557 |
|---|---|---|---|---|---|---|---|---|---|---|
| | H44/76 | SfB | SfB | M982 | 250771 | 250732 | 252697 | SfA | SfA | SfA |
| | SfB^{b} | P1.18 | P1.7- | SfB | SfA^{b} | SfA | SfA | P1.12- | P1.5- | P1.22- |
| Antigen | P1.7,16 | 25,6 | 2,4 | P1.22,9 | P1.22,14 | P1.22,14-1 | P1.18,25,6 | 1,13 | 1,2-2 | 1,14-1 |
| rLP2086- | >800 | >800 | 200 | 400 | 800 | >800 | >800 | >800 | — | <25 |
| 8529 + rLP2086- | | | | | | | | | | |
| 2996 | | | | | | | | | | |
| rLP2086- | >800 | 800 | 100 | 200 | 400 | 400 | >800 | >800 | >800 | 200 |
| 8529 + rLP2086- | | | | | | | | | | |
| 2996 + rP1.5- | | | | | | | | | | |
| 1,2-2 + rP1.22- | | | | | | | | | | |
| 1,14-1 | | | | | | | | | | |
| Monovalent | >800 | >800 | 200 | 400 | 800 | >800 | >800 | >800 | >800 | 800 |
| Controls^{c} | | | | | | | | | | |

Vaccination Procedure: 6-8 week old female Swiss-Webster mice were immunized with 10 ug of each protein+20 ug QS-21 at week 0 and week 4. Data analysis was performed on the week 6 exsanguination bleed.

a Bactericidal (BC50) titers represented as the reciprocal of the dilution of anti-sera which reduces viable cell count by 50%. Week 0 normal mouse sera had BC50 titers of <10.

bSfA—Subfamily A, SfB—Subfamily B

cRelevant monovalent control: rLP2086-8529, rLP2086-2996, rP1.5-1,2-2 or rP1.22-1,14-1 antisera

The following summarizes the results of the above described studies. Anti-rLP2086 antisera is bactericidal against 13/16 test strains. Eleven strains expressing different serosubtypes are killed by anti-P2086 sera. Bactericidal activity of anti-rLP2086 sera is complimentary to anti-rPorA sera. Mixtures of P2086 and PorA elicit complimentary bactericidal antibodies in mice. Differential detergent extraction, purification and immunization in conjunction with a functional antibody assay against many strains can be used to identify new vaccine candidates. P2086 has been identified as a vaccine candidate which elicits bactericidal antibody against strains heterologous in both P2086 and rPorA. Thus, the 2086 family of proteins may be a useful vaccine either alone or in combination with other neisserial antigens.

### Example 9

In accordance with the previous examples, additional meningococcal strains, of varying serogroups, were screened by PCR for the presence of the ORF 2086 gene. Ultimately, one hundred meningococcal strains were screened. The following describes the study and its overall results. These results supplement the data from the previous examples.

Two sets of internal PCR primers specific to the C-terminal variable regions were utilized to discriminate between Subfamily A and B gene sequences. The presence of a PCR amplified product of approximately 350 bp indicated that the 2086 gene sequence was present on the chromosome. All strains yielded a single PCR product of the expected size. The nucleotide sequences of fifty-five full-length ORF 2086 genes were determined, aligned (DNAStar MegAlign) and used to generate a phylogenetic tree. (See FIG. 12).

Nine of these 2086 genes were recombinantly expressed as a rLP2086 lipoprotein in a pBAD arabinose inducible promoter system and three of these genes were recombinantly expressed as a rP2086 non-lipidated protein in an IPTG inducible pET system. These recombinant proteins were expressed in E. coli B. The purified recombinant protein was used to immunize mice and the mouse antisera was assayed for its serum IgG titers and its bactericidal activity against a variety of heterologous meningococcal strains.

ORF 2086 was amplified by PCR from one of the following, whole meningococcal cells, purified chromosomal DNA or plasmid DNA templates.

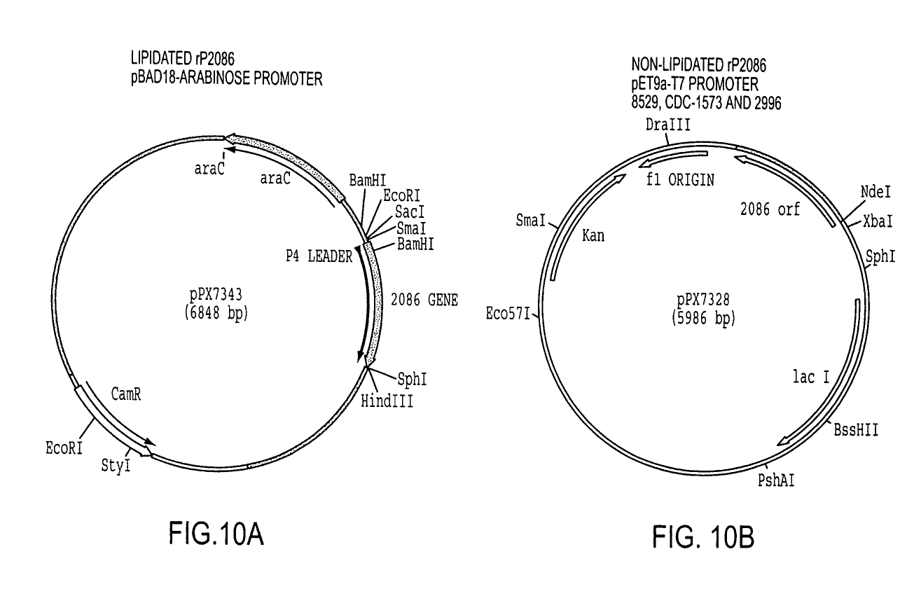

Nine ORF 2086 genes were cloned into the vector pLP339, which fuses the Haemophilus P4 leader sequence to the 5′ end of the ORF 2086 genes. E. coli strain BLR was used as the host strain for recombinant expression of the lipidated form of rP2086 from the pBAD/ORF 2086 clones. (See FIG. 10A) The pBAD arabinose inducible promoter drives the expression the P4 signal/ORF 2086 fusion protein to express a lipidated form of rP2086. Three P2086 genes, lacking a signal sequence, were cloned into a pET9a vector behind the highly active T7 phage promoter. E. coli strain BL21 (DE3) was used as the host strain for recombinant expression of a non-lipidated form of ORF 2086 from the pET9a/ORF 2086 clones. (See FIG. 10B) The DE3 lysogen in E. coli strain BL21 can be induced to express the T7 RNA polymerase under the control of the lacUV5 promoter by addition of IPTG. See, WCE; FEMS Micro. Lett., 48 (1987) 367-371 and BCA; J. Clin. Microbiol., 38 (2000) 2878-2884.

The gene, ORF2086, was cloned and sequenced from fifty-five different N. meningitidis strains. The nucleotide sequences were aligned (DNAStar MegAlign) and used to generate a phylogenetic tree. (See FIG. 12). This tree reveals two distinct subfamilies of the ORF 2086 gene nucleotide sequence. The two subfamilies of genes are similar at their 5′ ends, but contain considerable variation near their 3′ ends. Although there appears to be significant variability, certain key regions of the gene are highly homologous amongst the different strains. These conserved regions may provide functional continuity for the protein and may be indicative of cross-protective epitopes to be exploited as vaccine targets.

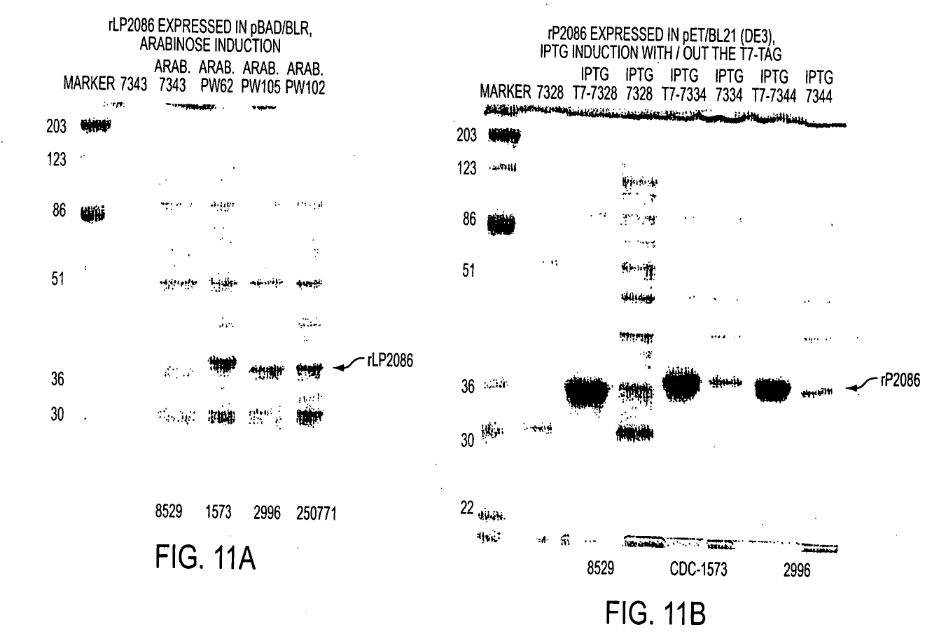

The 2086 gene was cloned from several serogroup B meningococcal strains and expressed with and without the lipidation signal sequence. Referring to FIGS. 11A and 11B, gel photographs show the whole cell lysates of E. coli B expressing the r2086 protein. The non-lipidated form fused to the T7-Tag expressed at the highest level. The T7-Tag sequence may provide stability to the mRNA and significantly enhances the level of polypeptide translated. This fusion protein appears to deposit in inclusion bodies and can be purified and refolded readily with known protocols. The lipidated and non-lipidated forms of P2086 are expressed at approximately 5 to 8% of total cellular protein, with the exception of the T7-Tag fusions, which express rP2086 as approximately 50% of total protein. The non-lipidated form of the protein appears to be soluble and localized in the cytoplasm. The lipidated form of the protein appears to be associated with the membrane fractions and is solubilized with detergent.

The recombinant lipidated 2086 protein from N. meningitidis B strain 8529 consistently elicits greater serum IgG titers than the non-lipidated form (see Table XVI below), which correlates well with the enhanced level of bactericidal activity against both homologous and heterologous meningococcal strains (see Table XVII below). The protein in its native lipidated form may have superior tertiary structure for antigen presentation and/or the, attached lipid may act as an adjuvant stimulating a greater immunogenic response.

TABLE XVI
Immune Response Elicited at Week 6 by WCE using
8529 rP2086 (non-lipidated) vs. 8529 rLP2086 (lipidated)

| Mouse Sera | | | | | | |
|---|---|---|---|---|---|---|
| | Adjuvant | Meningococcal Strains | | | | |
| Antigen (10 ug) | (20 ug) | H44/76 | H355 | 870227 | 880049 | 870446 |
| rP2088 | QS-21 | 273,238 | 212,947 | 102,694 | 69,124 | 21,466 |
| rLP2086 | QS-21 | 5,384,306 | 4,819,061 | 2,930,946 | 1,307,091 | 886,056 |

TABLE XVII
8529 rP2086 Elicits Weaker Bactericidal Activity than 8529 rLP2086

| Mouse Sera | | | | | |
|---|---|---|---|---|---|
| Antigen | Adjuvant | Meningococcal Strains | | | |
| (10 ug) | (20 ug) | H44/76 | H355 | 880049 | NMB |

| | | | | | |
|---|---|---|---|---|---|
| rP2086 | QS-21 | 200 | 100 | <25 | <25 |
| rLP2086 | QS-21 | 6,400 | 3,200 | 100 | <25 |
| Pre- | — | <10 | <10 | <10 | <10 |
| Immune | | | | | |
| Positive | — | 1,600 | 100 | 200 | 1,600 |
| Control | | | | | |

The following is a summary of the results of the study. All N. meningitidis B strains tested appear to have one 2086-like gene. At least two families of the 2086 gene are represented: Subfamily A—about 30% of strains and Subfamily B—about 70% of strains. The 2086 gene has been cloned and sequenced from 55 N. meningitidis strains. Sequences within Subfamily A are ˜86-100% identical at the DNA level. Sequence within Subfamily B are ˜89.5-100% identical at the DNA level. Sequences within Subfamily A vs. Subfamily B ˜60.9%-74% identical at the DNA level. 2086 homologs have been identified by PCR screening in the following:
N. meningitidis A, B, C, W135, Y
N. lactamica
N. gonorrhoeae FA1090
   Several ORF 2086 Genes Have Been Cloned and Recombinantly Expressed

Lipidated versions of P2086 were expressed from nine meningococcal strains.

These recombinant proteins have been purified and used to vaccinate mice.

The resulting antisera is bactericidal.

Non-lipidated versions of P2086 were expressed from three of the above nine strains. rLP2086 consistently elicits a greater immune response than rP2086. rLP2086 also exhibits enhanced bactericidal activity against both homologous and heterologous meningococcal strains.

### Example 10

The following tables, Tables XVIII and XIX, show the characterization of variants of members of the two subfamilies.

TABLE XVIII
Subfamily A rLP2086 Variants -
Characterization

| | | rLP2086- | rLP2086- | rLP2086- | rLP2086- | |
|---|---|---|---|---|---|---|
| | | 252988 | 250771 | 870446 | 2996 | rLP2086-C11 |
| Growth | | HySoy | HySoy | HySoy | HySoy | HySoy |
| Media | | | | | | |
| Solubility | | rTX-100 [symbol] | TX-100 | TX-100 | rTX-100 [symbol] | rTX-100 [symbol] |
| | | Z3-12 | | | Z3-12 | Z3-12 |
| Purification | | TMAE S | HQ Poros | HQ Poros | TMAE | TMAE |
| Steps | | Fractogel | SEC | SEC | SEC | S Fractogel |
| | | SEC | | | | |
| Purity (%) | | 96 | 83 | 80 | 95 | 82 |
| Yield | | 0.2 | 0.7 | 0.8 | 0.5 | 0.1 |
| (mg/g cell | | | | | (fermentor) | |
| pellet) | | | | | | |
| Size | SEC | 134,000 | 155,000 | 132,000 | 163,000 | 126,000 |
| | (Z3- | | | | | |
| | 12) | | | | | |
| | MS | 27,897 | — | — | 27,878 | 28,139 |
| | | (712 lipid) | | | (750 lipid) | (682 lipid) |

| | | | | | |
|---|---|---|---|---|---|
| Thermal | 66° C. | — | NT | 65° C. | 63° C. |
| Denaturation | | | | | |
| Transition | | | | | |
| Midpoint | | | | | |
| (T_{M}) ° C. | | | | | |
| Protein | 2.7 mg | 1 mg (Z3-12) | 5.0 mg | 44 mg | 1.1 mg |
| Available | | | | | |
| (mg) | | | | | |
| 8529 | 71 | 62 | 71 | 72 | 68 |
| Sequence | | | | | |
| Homology | | | | | |
| (%) | | | | | |

TABLE XIX
Subfamily B rLP2086 Variants -
Characterization

| | | rLP2086- | rLP2086- | rLP2086- | rLP2086- |
|---|---|---|---|---|---|
| | | 8529 | M982 | 880049 | CDC1573 |
| Growth Media | | Apollon | Apollon | HySoy | HySoy |
| | | (Sanford) | | | |
| Solubility | | 4M Urea [symbol] | rTX-100 [symbol] | rTX-100 [symbol] | rTX-100 |
| | | Z3-12 | Z3-12 | Z3-12 | |
| Purification | | TMAE | TMAE | TMAE | TMAE |
| Steps | | S Fractogel | S Fractogel | S Fractogel | SEC |
| Purity (%) | | 96 | 96 | 90 | 93 |
| Yield | | 0.2 | 1.6 | 0.4 | 1.0 |
| (mg/g cell | | (fermentor) | (fermentor) | | |
| pellet) | | | | | |
| Size | SEC | 95,000 | 110,000 | 100,000 | 120,000 |
| | (Z3-12) | | 150,000 | | |
| | MS | 27,785 | 27,719 | 28,044 | 28,385 |
| | | (822 lipid) | (711 lipid) | (819 lipid) | (823 lipid) |

| | | | | |
|---|---|---|---|---|
| Thermal | 70° C. | 75° C. | 62° C. | NT |
| Denaturation | | | | |
| Transition | | | | |
| Midpoint (T_{M}) | | | | |
| ° C. | | | | |
| Protein | Urea - 34 mg | Pool 1 - | 3.6 mg | 4.9 mg |
| Available | Sarc - 36 mg | 47 mg | | |
| (mg) | | Pool 2 - | | |
| | | 17 mg | | |
| 8529 Sequence | 100 | 94 | 92 | 87 |
| Homology (%) | | | | |

Table XX below provides the results of fluorescent serum bactericidal assays for the 2086 Subfamily A.

TABLE XX

| | | | | | M98 | M97 |
|---|---|---|---|---|---|---|
| Description | 250771 | 870446 | 6557 | NMB | 250732 | 252697 |
| rLP2086-252988, | >800 | >800 | <25 | — | >800 | >800 |
| 10 μg | (99%)* | (99%)* | | | (99%)* | (93%)* |
| rLP2086-C11, | 200 | >880 | <25 | — | 200 | 400 |
| 10 μg | | (91%)* | | | | |
| rLP2086-250771, | >800 | >800 | <25 | — | >800 | >800 |
| 10 μg | (92%)* | (99%)* | | | (96%)* | (84%)* |
| rLP2086-870446, | 400 | >800 | <25 | — | 400 | 400 |
| 10 μg | | (99%)* | | | | |
| rLP2086-2996, | 800 | >800 | <25 | — | >800 | >800 |
| 10 μg | | (99%)* | | | (93%)* | (72%)* |
| rLP2086-8529 + rLP2086- | 800 | >800 | <25 | — | >800 | >800 |
| 2996, | | (99%)* | | | (80%)* | (72%)* |
| 10 μg | | | | | | |
| rLP2086-8529 + rP1.22a, | — | 800 | 200 | >800 | — | — |
| 14a + rP1.5a, | | | | (98%)* | | |
| 2c, 10 μg | | | | | | |
| rLP2086-8529 + rLP2086- | 400 | >800 | 200 | >800 | 400 | >800 |
| 2996 + rP1.22a, 14a + rP1.5a, | | (99%)* | | (99%)* | | (88%)* |
| 2c, 10 μg | | | | | | |
| NMB/rLP2086- | — | 100 | — | 400 | — | — |
| 8529 | | | | | | |
| vesicles, 20 μg | | | | | | |
| rP1.22a,14a, 10 μg | 25 | — | 800 | — | 100 | — |
| rP1.5a,2c, 10 μg | — | — | — | >800 | — | — |
| | | | | (99%)* | | |
| rLP2086-8529, | — | 800 | — | — | — | — |
| 10 μg | | | | | | |
| rP1.22a,14a, 25 μg | 200 | — | — | — | 800 | — |
| rP1.18,25.6, 5 μg | — | — | — | — | — | |
| nP1.22,9 (M982), | — | — | 100 | — | — | — |
| 25 μg | | | | | | |
| pre-immune mouse serum (negative | <10 | <10 | <10 | <10 | <10 | <10 |
| control) | | | | | | |
| | 800 | 400 | 800 | 1600 | ** | ** |

Notes:
*Percentage indicates the % BC activity at the 1:800 dilution.
** Positive control not available.
— serum not tested

### Example 11

The following further demonstrates that P2086 is expressed in neisserial strains and provides additional specific examples of P2086 expression in several strains.

Cell lysates were prepared with cells from plate cultures resuspended in SDS sample buffer and heated at 98° C. for four minutes. Samples were loaded at ˜30-50 ug total protein per well on 10-20% pre-cast gels (ICN) and run at 175V. The gels were transferred to a nitrocellulose membrane, which was then blocked for 30 min. with 5% powdered milk in Tris-buffered saline (Blotto). The primary antibody used was a pool of polyclonal antisera raised against individual rLP2086 variants in mice.

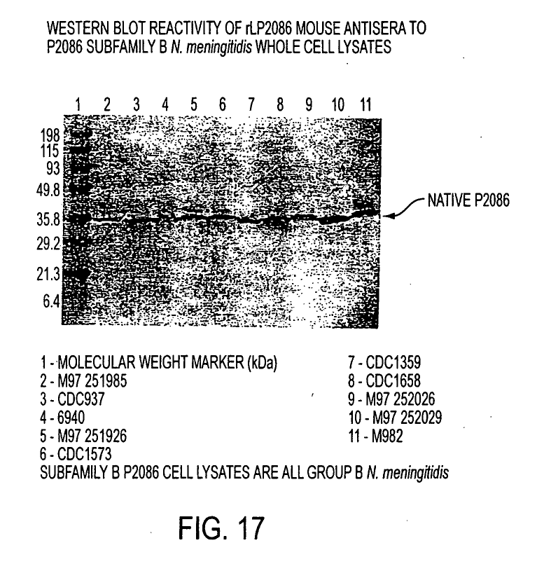

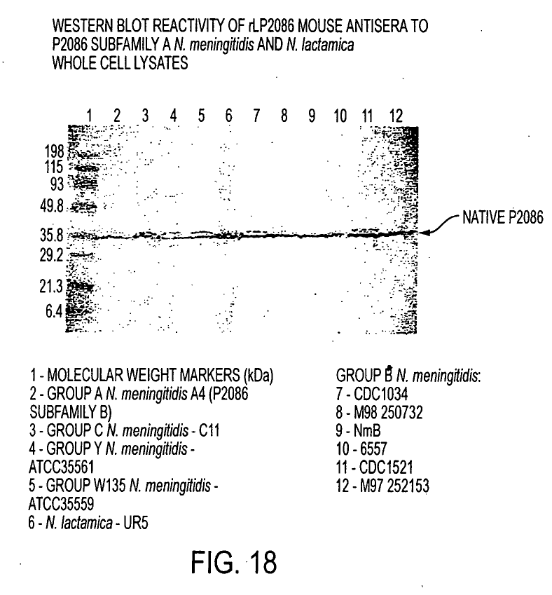

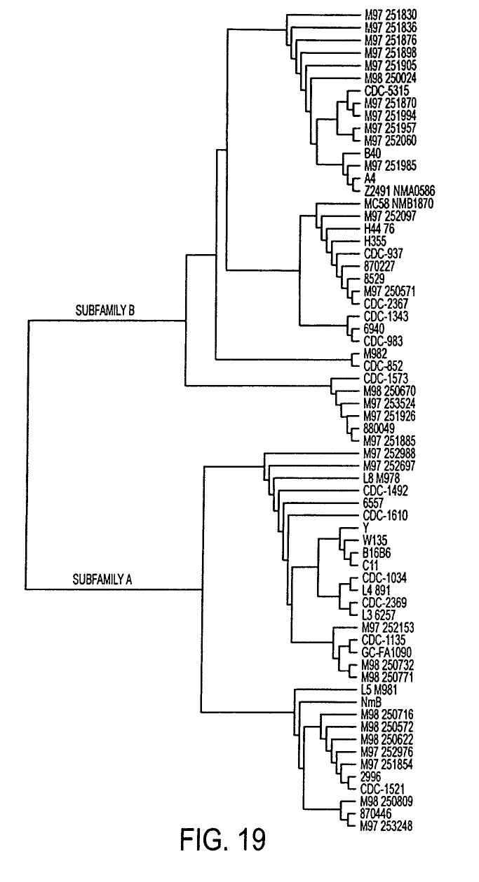

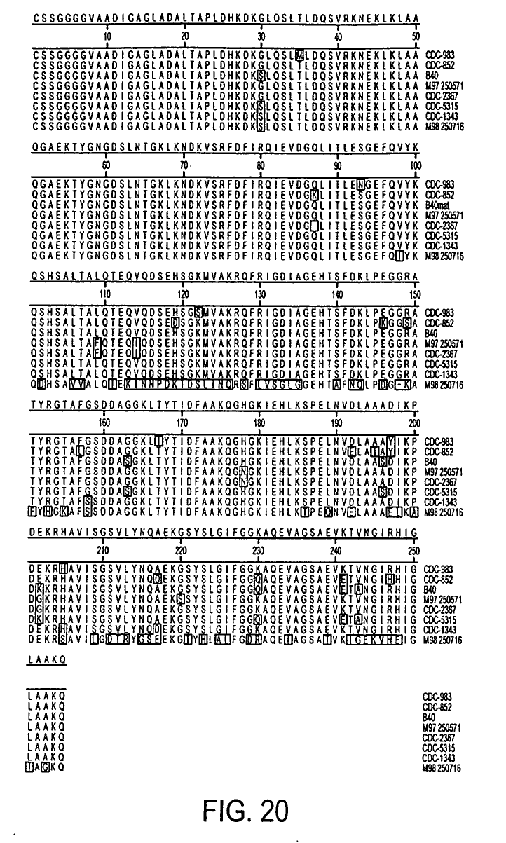

Referring to FIGS. 17 and 18, a Western Blot shows the reactivity of rLP2086 mouse antisera to P2086 Subfamily A and B whole cell lysates. For the Subfamily A cell lysate blot, the antisera used were raised against rLP2086-2996, -870446 and -250771 with rLP2086-250771 diluted at 1/500 in Blotto and the others diluted at 1/1000 in Blotto. For the Subfamily B cell lysate blot, the antisera used were raised against rLP2086-8529 (diluted 1/1000 in Blotto), -CDC1573. -M982 and -880049 (these three diluted 1/500 in Blotto). The primary antisera and blot were incubated at 4° C. overnight. The blot was washed, a goat-anti-mouseAP secondary was added at 1/500 in Blotto, and the blot was incubated for 30min. at room temperature. After washing, the blot was developed using the BCIP/NBT Membrane Phosphatase Substrate System (KPL).

### BIBLIOGRAPHY

References referred to herein above are noted below and are incorporated herein by reference in their entirety:

1. 1997. Case definitions for Infectious Conditions Under Public Health Surveillance. CDC.

2. 1995 Sambrook, J. and D. W. Russell. 1995. Current Protocols in Molecular Biology. John Wiley & Sons, Inc., New York.

3. 1994. Griffin, A. M. and Griffin, H. G., ed., Computer Analysis of Sequence Data, Part I. Humana Press, New Jersey.

4. 1993. Smith, D. W. ed., Biocomputing: Informatics and Genome Projects. Academic Press, New York

5. 1991. Gribskov, M. and Devereux, J., ed. Sequence Analysis Primer. Stockton Press, New York.

6. 1988. Lesk, A. M., ed. Computational Molecular Biology. Oxford University Press, New York.

7. Abdillahi, H., and J. T. Poolman. 1988. Neisseria meningitidis group B serosubtyping using monoclonal antibodies in whole-cell ELISA. Microbial Pathogenesis 4(1):27-32.

8. Achtman, M. 1995. Epidemic spread and antigenic variability of Neisseria meningitidis. Trends in Microbiology 3(5): 186-92.

9. Alm, R. A., L. S. Ling, D. T. Moir, B. L. King, E. D. Brown, P. C. Doig, D. R. Smith, B. Noonan, B. C. Guild, B. L. dejonge, G. Carmel, P. J. Tummino, A. Caruso, M. Uria-Nickelsen, D. M. Mills, C. Ives, R. Gibson, D. Merberg, S. D. Mills, Q. Jiang, D. E. Taylor, G. F. Vovis, and T. J. Trust. 1999. Genomic-sequence comparison of two unrelated isolates of the human gastric pathogen Helicobacter pylori [published erratum appears in Nature 1999 Feb. 25;397(6721):719]. Nature. 397:176-80.

10. Altschul, S. F., T. L. Madden, A. A. Schaffer, J. Zhang, Z. Zhang, W. Miller, and D. J. Lipman. 1997. Gapped BLAST and PSI-BLAST: a new generation of protein database search programs. Nucleic Acids Res. 25:3389-402.

11. Anderson, T. F. 1951. Techniques for the preservation of three-dimensional structure in preparing specimens for the electron microscope. Trans N Y Acad Sci. 13:130-134.

12. Ambrosch, F., G. Wiedermann, P. Crooy, and A. M. George. 1983. Immunogenicity and side-effects of a new tetravalent meningococcal polysaccharide vaccine. Bulletin of the World Health Organization 61(2):317-23.

13. Benson, G. 1999. Tandem repeats finder: a program to analyze DNA sequences. Nucleic Acids Res. 27:573-80.

14. Carillo, H., D. Lipman, and J. Siam. 1988. Applied Math 48:1073.

15. Chen, C. C., and P. P. Cleary. 1989. Cloning and expression of the streptococcal C5a peptidase gene in Escherichia coli: linkage to the type 12 M protein gene. Infect. Immun. 57:1740-1745.

16. Chmouryguina, I., A. Suvorov, P. Ferrieri, and P. P. Cleary. 1996. Conservation of the C5a peptidase genes in group A and B streptococci. Infect. Immun. 64:2387-2390.

17. Cockerill, F. R., 3rd, R. L. Thompson, J. M. Musser, P. M. Schlievert, J. Talbot, K. E. Holley, W. S. Harmsen, D. M. Ilstrup, P. C. Kohner, M. H. Kim, B. Frankfort, J. M. Manahan, J. M. Steckelberg, F. Roberson, and W. R. Wilson. 1998. Molecular, serological, and clinical features of 16 consecutive cases of invasive streptococcal disease. Southeastern Minnesota Streptococcal Working Group. Clin Infect Dis. 26:1448-58.

18. Courtney, H. S., Y. Li, J. B. Dale, and D. L. Hasty. 1994. Cloning, sequencing, and expression of a fibronectin/fibrinogen-binding protein from group A streptococci. Infect Immun. 62:3937-46.

19. Cserzo, M., E. Wallin, I. Simon, G. von Heijne, and A. Elofsson. 1997. Prediction of transmembrane alpha-helices in prokaryotic membrane proteins: the dense alignment surface method. Protein Engineering. 10:673-6.

20. Cunningham, M. W., and A. Quinn. 1997. Imunological crossreactivity between the class I epitope of streptococcal M protein and myosin. Adv Exp Med Biol. 418:887-92.

21. Dale, J. B., R. W. Baird, H. S. Courtney, D. L. Hasty, and M. S. Bronze. 1994. Passive protection of mice against group A streptococcal pharyngeal infection by lipoteichoic acid. J Infect Dis. 169:319-23.

22. Dale, J. B., M. Simmons, E. C. Chiang, and E. Y. Chiang. 1996. Recombinant, octavalent group A streptococcal M protein vaccine. Vaccine. 14:944-8.

23. Dale, J. B., R. G. Washburn, M. B. Marques, and M. R. Wessels. 1996. Hyaluronate capsule and surface M protein in resistance to opsonization of group A streptococci. Infect Immun. 64:1495-501.

24. Eddy, S. R. 1996. Hidden Markov models. Cur Opin Struct Bio. 6:361-5.

25. Ellen, R. P., and R. J. Gibbons. 1972. M protein-associated adherence of Streptococcus pyogenes to epithelial surfaces: prerequisite for virulence. Infect Immun. 5:826-830.

26. Eng, J. K., A. L. McCormack, and J. R. Yates, 3rd. 1994. An approach to correlate tandem mass-spectral data of peptides with amino-acid-sequences in a protein database. Am Soc Mass Spectrometry. 5:976-89.

27. Fischetti, V. A., V. Pancholi, and O. Schneewind. 1990. Conservation of a hexapeptide sequence in the anchor region of surface proteins from gram-positive cocci. Mol Microbiol. 4:1603-5.

28. Fogg, G. C., and M. G. Caparon. 1997. Constitutive expression of fibronectin binding in Streptococcus pyogenes as a result of anaerobic activation of rofA. J Bacteriol. 179:6172-80.

29. Foster, T. J., and M. Hook. 1998. Surface protein adhesins of Staphylococcus aureus. Trends Microbiol. 6:484-8.

30. Fraser, C. M., S. Casjens, W. M. Huang, G. G. Sutton, R. Clayton, R. Lathigra, O. White, K. A. Ketchum, R. Dodson, E. K. Hickey, M. Gwinn, B. Dougherty, J. F. Tomb, R. D. Fleischmann, D. Richardson, J. Peterson, A. R. Kerlavage, J. Quackenbush, S. Salzberg, M. Hanson, R. van Vugt, N. Palmer, M. D. Adams, J. 31.

Gocayne, J. C. Venter, and et al. 1997. Genomic sequence of a Lyme disease spirochaete, Borrelia burgdorferi [see comments]. Nature. 390:580-6.

32. Goldschneider, I., E. C. Gotschlich, and M. S. Artenstein. 1969. Human immunity to the meningococcus. I. The role of humoral antibodies. Journal of Experimental Medicine 129(6): 1307-26.

33. Goldschneider, I., E. C. Gotschlich, and M. S. Artenstein. 1969. Human immunity to the meningococcus. II. Development of natural immunity; Journal of Experimental Medicine 129(6): 1327-48.

34. Gotschlich, E. C., I. Goldschneider, and M. S. Artenstein. 1969. Human immunity to the meningococcus. IV. Immunogenicity of group A and group C meningococcal polysaccharides in human volunteers. Journal of Experimental Medicine 129(6): 1367-84.

35. Gotschlich, E. C., I. Goldschneider, and M. S. Artenstein. 1969. Human immunity to the meningococcus. V. The effect of immunization with meningococcal group C polysaccharide on the carrier state. Journal of Experimental Medicine 129(6): 1385-95.

36. Green, B. A., Farley, J. E., Quinn-Dey, T., Deich, R. A., and Zlotnick, G. W. 1991. The e (P4) Outer Membrane Protein of Haemophilus influenzae: Biologic Activity of Anti-e Serum and Cloning and Sequencing of the Structural Gene. Infect. Immun. 59: 3191-3198.

37. Guzman, L-M, Belin, D., Carson, M. J., Beckwith, J. 1995. Tight Regulation, Modulation, and High-Level Expression by Vectors Containing the Arabinose PBAD Promoter. J. Bacteriology 177:4121-4130.

38. Hacker, J., G. Blum-Oehler, I. Muhldorfer, and H. Tschape. 1997. Pathogenicity islands of virulent bacteria: structure, function and impact on microbial evolution. Mol Microbiol. 23:1089-97.

39. Hanski, E., and M. Caparon. 1992. Protein F, a fibronectin-binding protein, is an adhesion of the group A streptococcus Streptococcus pyogenes. Proc Natl Acad Sci., USA. 89:6172-76.

40. Hanski, E., P. A. Horwitz, and M. G. Caparon. 1992. Expression of protein F, the fibronectin-binding protein of Streptococcus pyogenes JRS4, in heterologous streptococcal and enterococcal strains promotes their adherence to respiratory epithelial cells. Infect Immun. 60:5119-5125.

41. Hernandez-Sanchez, J., J. G. Valadez, J. V. Herrera, C. Ontiveros, and G. Guarneros. 1998. lambda bar minigene-mediated inhibition of protein synthesis involves accumulation of peptidyl-tRNA and starvation for tRNA. EMBO Journal. 17:3758-65.

42. Huang, T. T., H. Malke, and J. J. Ferretti. 1989. The streptokinase gene of group A streptococci: cloning, expression in Escherichia coli, and sequence analysis. Mol Microbiol. 3:197-205.

43. Hynes, W. L., A. R. Dixon, S. L. Walton, and L. J. Aridgides. 2000. The extracellular hyaluronidase gene (hylA) of Streptococcus pyogenes. FEMS Microbial Lett. 184:109-12.

44. Hynes, W. L., L. Hancock, and J. J. Ferretti. 1995. Analysis of a second bacteriophage hyaluronidase gene from Streptococcus pyogenes: evidence for a third hyaluronidase involved in extracellular enzymatic activity. Infect Immun. 63:3015-20.

45. Isberg, R. R., and G. Tran Van Nhieu. 1994. Binding and internalization of microorganisms by integrin receptors. Trends Microbio. 2:10-4.

46. Jones, K. F., and V. A. Fischetti. 1988. The importance of the location of antibody binding on the M6 protein for opsonization and phagocytosis of group A M6 streptococci. J Exp Med. 167:1114-23.

47. Kihlberg, B. M., M. Collin, A. Olsen, and L. Bjorck. 1999. Protein H, an antiphagocytic surface protein in Streptococcus pyogenes. Infect Immun. 67:1708-14.

48. Koebnik, R. 1995. Proposal for a peptidoglycan-associating alpha-helical motif in the C-terminal regions of some bacterial cell-surface proteins [letter; comment]. Molecular Microbiology. 16:1269-70.

49. Kuipers, O. P., H. J. Boot, and W. M. de Vos. 1991. Improved site-directed mutagenesis method using PCR. Nucleic Acids Res. 19:4558.

50. Kyte, J., and R. F. Doolittle. 1982. A simple method for displaying the hydropathic character of a protein. Journal of Molecular Biology 157:105-132.

51. Landt, O., H. P. Grunert, and U. Hahn. 1990. A general method for rapid site-directed mutagenesis using the polymerase chain reaction. Gene 96:125-128.

52. Loessner, M. J., S. Gaeng, and S. Scherer. 1999. Evidence for a holin-like protein gene fully embedded out of frame in the endolysin gene of Staphylococcus aureus bacteriophage 187. J Bacteriol. 181:4452-60.

53. Lukashin, A. V., and M. Borodovsky. 1998. GeneMark.hmm: new solutions for gene finding. Nucleic Acids Res. 26:1107-15.

54. Lukomski, S., C. A. Montgomery, J. Rurangirwa, R. S. Geske, J. P. Barrish, G. J. Adams, and.J. M. Musser. 1999. Extracellular cysteine protease produced by Streptococcus pyogenes participates in the pathogenesis of invasive skin infection and dissemination in mice. Infect Immun. 67:1779-88.

55. Madore, D. V. 1998. Characterization of immune response as an indicator of Haemophilus influenzae type b vaccine efficacy. Pediatr Infect Dis J. 17:S207-10.

56. Matsuka, Y. V., S. Pillai, S. Gubba, J. M. Musser, and S. B. Olmsted. 1999. Fibrinogen cleavage by the Streptococcus pyogenes extracellular cysteine protease and generation of antibodies that inhibit enzyme proteolytic activity. nfect Immun. 67:4326-33.

57. Mazmanian, S. K., G. Liu, H. Ton-That, and 0. Schneewind. 1999. Staphylococcus aureus sortase, an enzyme that anchors surface proteins to the cell wall. Science. 285:760-3.

58. McAtee, C. P., K. E. Fry, and D. E. Berg. 1998. Identification of potential diagnostic and vaccine candidates of Helicobacter pylori by “proteome” technologies. Helicobacter. 3:163-9.

59. McAtee, C. P., M. Y. Lim, K. Fung, M. Velligan, K. Fry, T. Chow, and D. E. Berg. 1998. Identification of potential diagnostic and vaccine candidates of Helicobacter pylori by two-dimensional gel electrophoresis, sequence analysis, and serum profiling. Clin Diagn Lab Immunol. 5:537-42.

60. McAtee, C. P., M. Y. Lim, K. Fung, M. Velligan, K. Fry, T. P. Chow, and D. E. Berg. 1998. Characterization of a Helicobacter pylori vaccine candidate by proteome techniques. J Chromatogr B Biomed Sci Appl. 714:325-33.

61. Mejlhede, N., J. F. Atkins, and J. Neuhard. 1999. Ribosomal −1 frameshifting during decoding of Bacillus subtilis cdd occurs at the sequence CGA AAG. J. Bacteriol. 181:2930-7.

62. Molinari, G., S. R. Talay, P. Valentin-Weigand, M. Rohde, and G. S. Chhatwal. 1997. The fibronectin-binding protein of Streptococcus pyogenes, SfbI, is involved in the internalization of group A streptococci by epithelial cells. Infect Immun. 65:1357-63.

63. Mountzouros, K. T., and A. P. Howell. 2000. Detection of complement-mediated antibody-dependent bactericidal activity in a fluorescence-based serum bactericidal assay for group B Neisseria meningitidis. J. Clin. Microbiol. 38(8):2878-2884.

64. Nakai, K., and M. Kanehisa. 1991. Expert system for predicting protein localization sites in gram-negative bacteria. Proteins. 11:95-110.

65. Navarre, W. W., and O. Schneewind. 1999. Surface proteins of gram-positive bacteria and mechanisms of their targeting to the cell wall envelope. Microbial Mol Biol Rev. 63:174-229.

66. Nielsen, H., J. Engelbrecht, S. Brunak, and G. von Heijne. 1997. Identification of prokaryotic and eukaryotic signal peptides and prediction of their cleavage sites. Protein Engineering. 10:1-6.

67. Nizet, V., B. Beall, D. J. Bast, V. Datta, L. Kilburn, D. E. Low, and J. C. De Azavedo. 2000. Genetic locus for streptolysin S production by group A streptococcus. Infect Immun. 68:4245-54.

68. Nordstrand, A., W. M. McShan, J. J. Ferretti, S. E. Holm, and M. Norgren. 2000. Allele substitution of the streptokinase gene reduces the nephritogenic capacity of group A streptococcal strain NZ131. Infect Immun. 68:1019-25.

69. Olmsted, S. B., S. L. Erlandsen, G. M. Dunny, and C. L. Wells. 1993. High-resolution visualization by field emission scanning electron microscopy of Enterococcus faecalis surface proteins encoded by the pheromone-inducible conjugative plasmid pCF10. J Bacteriol. 175:6229-37.

70. Park, J., and S. A. Teichmann. 1998. DIVCLUS: an automatic method in the GEANFAMMER package that finds homologous domains in single- and multi-domain proteins. Bioinformatics. 14:144-50.

71. Parkhill, J., M. Achtman, K. D. James, S. D. Bentley, C. Churcher, S. R. Klee, G. Morelli, D. Basham, D. Brown, T. Chillingworth, R. M. Davies, P. Davis, K. Devlin, T. Feltwell, N. Hamlin, S. Holroyd, K. Jagels, S. Leather, S. Moule, K. Mungall, M. A. Quail, M. A. Rajandream, K. M. Rutherford, M. Simmonds, J. Skelton, S. Whitehead, B. G. Spratt, and B. G. Barrell. 2000. Complete DNA sequence of a serogroup A strain of Neisseria meningitidis Z2491 [see comments]. Nature. 404:502-6.

72. Pierschbacher, M. D., and E. Ruoslahti. 1987. Influence of stereochemistry of the sequence Arg-Gly-Asp-Xaa on binding specificity in cell adhesion. J Biol Chem. 262:17294-8.

73. Pizza, M., V. Scarlato, V. Masignani, M. M. Giuliani, B. Arico, M. Comanducci, G. T. Jennings, L. Baldi, E. Bartolini, B. Capecchi, C. L. Galeotti, E. Luzzi, R. Manetti, E. Marchetti, M. Mora, S. Nuti, G. Ratti, L. Santini, S. Savino, M. Scarselli, E. Storni, P. Zuo, M. Broeker, E. Hundt, B. Knapp, E. Blair, T. Mason, H. Tettelin, D. W. Hood, A. C. Jeffries, N. J. Saunders, D. M. Granoff, J. C. Venter, E. R. Moxon, G. Grandi, and R. Rappuoli. 2000. Identification of vaccine candidates against serogroup B meningococcus by whole-genome sequencing. Science 287(5459): 1816-20.

74. Podbielski, A., A. Flosdorff, and J. Weber-Heynemann. 1995. The group A streptococcal virR49 gene controls expression of four structural vir regulon genes. Infect Immun. 63:9-20.

75. Poolman, J. T. 1996. Bacterial outer membrane protein vaccines. The meningococcal example. Advances in Experimental Medicine & Biology 397:73-7.

76. Proft, T., S. Louise Moffatt, C. J. Berkahn, and J. D. Fraser. 1999. Identification and Characterization of Novel Superantigens from Streptococcus pyogenes. J Exp Med. 189:89-102.

77. Pugsley, A. P. 1993. The complete general secretory pathway in gram-negative bacteria. Microbiol Rev. 57:50-108.

78. Quinn, A., K. Ward, V. A. Fischetti, M. Hemric, and M. W. Cunningham. 1998. Immunological relationship between the class I epitope of streptococcal M protein and myosin. Infect Immun. 66:4418-24.

79. Reda, K. B., V. Kapur, D. Goela, J. G. Lamphear, J. M. Musser, and R. R. Rich. 1996. Phylogenetic distribution of streptococcal superantigen SSA allelic variants provides evidence for horizontal transfer of ssa within Streptococcus pyogenes. Infect Immun. 64:1161-5.

80. Sambrook, J., and D. W. Russell. 2001. Molecular cloning a laboratory manual, Third ed, vol. 3. Cold Spring Harbor Laboratory Press, Cold Spring Harbor, N.Y.

81. Salzberg, S. L., A. L. Delcher, S. Kasif, and O. White. 1998. Microbial gene identification using interpolated Markov models. Nucleic Acids Res. 26:544-8.

82. Saukkonen, K., H. Abdillahi, J. T. Poolman, and M. Leinonen. 1987. Protective efficacy of monoclonal antibodies to class 1 and class 3 outer membrane proteins of Neisseria meningitidis B:15:P1.16 in infant rat infection model: new prospects for vaccine development. Microbial Pathogenes is 3(4):261-7.

83. Sedegah et al. 1994. Immunology. 91, 9866-9870.

84. Sonnenberg, M. G., and J. T. Belisle. 1997. Definition of Mycobacterium tuberculosis culture filtrate proteins by two-dimensional polyacrylamide gel electrophoresis, N-terminal amino acid sequencing, and electrospray mass spectrometry. Infect Immun. 65:4515-24.

85. Sonnhammer, E. L., S. R. Eddy, and R. Durbin. 1997. Pfam: a comprehensive database of protein domain families based on seed alignments. Proteins. 28:405-20.

86. Stevens, D. L. 1995. Streptococcal toxic-shock syndrome: spectrum of disease, pathogenesis, and new concepts in treatment. Emerg Infect Dis. 1:69-78.

87. Stockbauer, K. E., L. Magoun, M. Liu, E. H. Burns, Jr., S. Gubba, S. Renish, X. Pan, S. C. Bodary, E. Baker, J. Coburn, J. M. Leong, and J. M. Musser. 1999. A natural variant of the cysteine protease virulence factor of group A streptococcus with an arginine-glycine-aspartic acid (RGD) motif preferentially binds human integrins alphavbeta3 and alphaIlbbeta3 Proc Natl Acad Sci., USA. 96:242-7.

88. Tettelin, H., N. J. Saunders, J. Heidelberg, A. C. Jeffries, K. E. Nelson, J. A. Eisen, K. A. Ketchum, D. W. Hood, J. F. Peden, R. J. Dodson, W. C. Nelson, M. L. Gwinn, R. DeBoy, J. D. Peterson, E. K. Hickey, D. H. Haft, S. L. Salzberg, O. White, R. D. Fleischmann, B. A. Dougherty, T. Mason, A. Ciecko, D. S. Parksey, E. Blair, H. Cittone, E. B. Clark, M. D. Cotton, T. R. Utterback, H. Khouri, H. Qin, J. Vamathevan, J. Gill, V. Scarlato, V. Masignani, M. Pizza, G. Grandi, L. Sun, H. O. Smith, C. M. Fraser, E. R. Moxon, R. Rappuoli, and J. C. Venter. 2000. Complete genome sequence of Neisseria meningitidis serogroup B strain MC58. Science 287(5459):1809-15.

89. Ton-That, H., G. Liu, S. K. Mazmanian, K. F. Faull, and O. Schneewind. 1999. Purification and characterization of sortase, the transpeptidase that cleaves surface proteins of Staphylococcus aureus at the LPXTG motif. Proc Natl Acad Sci USA. 96:12424-12429.

90. von Heinje, G. 1987. Sequence Analysis in Molecular Biology. Academic Press, New York.

91. Weldingh, K., I. Rosenkrands, S. Jacobsen, P. B. Rasmussen, M. J. Elhay, and P. Andersen. 1998. Two-dimensional electrophoresis for analysis of Mycobacterium tuberculosis culture filtrate and purification and characterization of six novel proteins. Infect Immun. 66:3492-500.

92. Wolff et al. 1990. Science. 247, 1465-1468.

93. Yutsudo, T., K. Okumura, M. Iwasaki, A. Hara, S. Kamitani, W. Minamide, H. Igarashi, and Y. Hinuma. 1994. The gene encoding a new mitogenic factor in a Streptococcus pyogenes strain is distributed only in group A streptococci. Infection and Immunity. 62:4000-4004.

94. Zagursky, R. J. and D. Russell. 2001. Bioinformatics: Use in Bacterial Vaccine Discovery. BioTechniques. 31:636-659.

The invention now being fully described, it will be apparent to one of ordinary skill in the art that many changes and modifications can be made thereto without departing from the spirit or scope of the invention as set forth herein. The foregoing describes the preferred embodiments of the present invention along with a number of possible alternatives. These embodiments, however, are merely for example and the invention is not restricted thereto.

What is claimed is:

1. A protein comprising an amino acid sequence selected from the group consisting of SEQ ID NOS: 84, 96, 102, 108, 114, 132, 138, 234 and combinations thereof.

2. A composition comprising a protein according to claim 1.

3. The composition of claim 2, wherein the composition additionally comprises a non-ORF2086 protein of N. meningitidis.

4. The composition of claim 2, further comprising:
a protein antigen from N. meningitidis;
a polysaccharide antigen from N. meningitidis; and/or
a polysaccharide antigen from S. pneumoniae.

5. The composition of claim 2, further comprising a pharmaceutically acceptable carrier.

6. The composition of claim 5 for use as a medicament.

7. The composition of claim 5, wherein said composition is a medicament.

8. The composition of claim 5 for use as an immunogenic composition.

9. The composition of claim 5, wherein said composition is an immunogenic composition.

10. A fusion protein comprising an ORF2086 protein and another immunogenic protein or part thereof.

11. A protein comprising the amino acid sequence of SEQ ID NO:114.

12. A protein comprising the amino acid sequence of SEQ ID NO:234.


SEQUENCE LISTING (329 sequences)

SEQ ID NO: 1 - DNA, 765 bp, Neisseria meningitidis
tgcagcagcggaggcggcggtgtcgccgccgacatcggcgcggggcttgccgatgcactaaccacaccgctcgaccataaagacaaaagtttgcagtctttgacgctggatcagtccgtcaggaaaaacgagaaactgaagctggcggcacaaggtgcggaaaaaacttatggaaacggcgacagcctcaatacgggcaaattgaagaacgacaaggtcagccgcttcgactttatccgtcaaatcgaagtggacggacaaaccatcacgctggcaagcggcgaatttcaaatatacaaacagaaccactccgccgtcgttgccctacagattgaaaaaatcaacaaccccgacaaaatcgacagcctgataaaccaacgctccttccttgtcagcggtttgggcggagaacataccgccttcaaccaactgcctgacggcaaagccgagtatcacggcaaagcattcagctccgacgacccgaacggcaggctgcactactccattgattttaccaaaaaacagggttacggcagaatcgaacacctgaaaacgcccgagcagaatgtcgagcttgcctccgccgaactcaaagcagatgaaaaatcacacgccgtcattttgggcgacacgcgctacggcggcgaagaaaaaggcacttaccacctcgcccttttcggcgaccgcgcccaagaaatcgccggctcggcaaccgtgaagataagggaaaaggttcacgaaatcggcatcgccggcaaacagtag

SEQ ID NO: 2 - protein, 254 aa, Neisseria meningitidis
CSSGGGGVAADIGAGLADALTTPLDHKDKSLQSLTLDQSVRKNEKLKLAAQGAEKTYGNGDSLNTGKLKNDKVSRFDFIRQIEVDGQTITLASGEFQIYKQNHSAVVALQIEKINNPDKIDSLINQRSFLVSGLGGEHTAFNQLPDGKAEYHGKAFSSDDPNGRLHYSIDFTKKQGYGRIEHLKTPEQNVELASAELKADEKSHAVILGDTRYGGEEKGTYHLALFGDRAQEIAGSATVKIREKVHEIGIAGKQ

SEQ ID NO: 3 - DNA, 768 bp, Neisseria meningitidis
tgcggatccagcggaggcggcggtgtcgccgccgacatcggcgcggggcttgccgatgcactaaccacaccgctcgaccataaagacaaaagtttgcagtctttgacgctggatcagtccgtcaggaaaaacgagaaactgaagctggcggcacaaggtgcggaaaaaacttatggaaacggcgacagcctcaatacgggcaaattgaagaacgacaaggtcagccgcttcgactttatccgtcaaatcgaagtggacggacaaaccatcacgctggcaagcggcgaatttcaaatatacaaacagaaccactccgccgtcgttgccctacagattgaaaaaatcaacaaccccgacaaaatcgacagcctgataaaccaacgctccttccttgtcagcggtttgggcggagaacataccgccttcaaccaactgcctgacggcaaagccgagtatcacggcaaagcattcagctccgacgacccgaacggcaggctgcactactccattgattttaccaaaaaacagggttacggcagaatcgaacacctgaaaacgcccgagcagaatgtcgagcttgcctccgccgaactcaaagcagatgaaaaatcacacgccgtcattttgggcgacacgcgctacggcggcgaagaaaaaggcacttaccacctcgcccttttcggcgaccgcgcccaagaaatcgccggctcggcaaccgtgaagataagggaaaaggttcacgaaatcggcatcgccggcaaacagtag

SEQ ID NO: 4 - protein, 255 aa, Neisseria meningitidis
CGSSGGGGVAADIGAGLADALTTPLDHKDKSLQSLTLDQSVRKNEKLKLAAQGAEKTYGNGDSLNTGKLKNDKVSRFDFIRQIEVDGQTITLASGEFQIYKQNHSAVVALQIEKINNPDKIDSLINQRSFLVSGLGGEHTAFNQLPDGKAEYHGKAFSSDDPNGRLHYSIDFTKKQGYGRIEHLKTPEQNVELASAELKADEKSHAVILGDTRYGGEEKGTYHLALFGDRAQEIAGSATVKIREKVHEIGIAGKQ

SEQ ID NO: 5 - DNA, 765 bp, Neisseria meningitidis
atgagcagcggaggcggcggtgtcgccgccgacatcggcgcggggcttgccgatgcactaaccacaccgctcgaccataaagacaaaagtttgcagtctttgacgctggatcagtccgtcaggaaaaacgagaaactgaagctggcggcacaaggtgcggaaaaaacttatggaaacggcgacagcctcaatacgggcaaattgaagaacgacaaggtcagccgcttcgactttatccgtcaaatcgaagtggacggacaaaccatcacgctggcaagcggcgaatttcaaatatacaaacagaaccactccgccgtcgttgccctacagattgaaaaaatcaacaaccccgacaaaatcgacagcctgataaaccaacgctccttccttgtcagcggtttgggcggagaacataccgccttcaaccaactgcctgacggcaaagccgagtatcacggcaaagcattcagctccgacgacccgaacggcaggctgcactactccattgattttaccaaaaaacagggttacggcagaatcgaacacctgaaaacgcccgagcagaatgtcgagcttgcctccgccgaactcaaagcagatgaaaaatcacacgccgtcattttgggcgacacgcgctacggcggcgaagaaaaaggcacttaccacctcgcccttttcggcgaccgcgcccaagaaatcgccggctcggcaaccgtgaagataagggaaaaggttcacgaaatcggcatcgccggcaaacagtag

SEQ ID NO: 6 - protein, 254 aa, Neisseria meningitidis
MSSGGGGVAADIGAGLADALTTPLDHKDKSLQSLTLDQSVRKNEKLKLAAQGAEKTYGNGDSLNTGKLKNDKVSRFDFIRQIEVDGQTITLASGEFQIYKQNHSAVVALQIEKINNPDKIDSLINQRSFLVSGLGGEHTAFNQLPDGKAEYHGKAFSSDDPNGRLHYSIDFTKKQGYGRIEHLKTPEQNVELASAELKADEKSHAVILGDTRYGGEEKGTYHLALFGDRAQEIAGSATVKIREKVHEIGIAGKQ

SEQ ID NO: 7 - DNA, 765 bp, Neisseria meningitidis
tgcagcagcggaggcggcggtgtcgccgccgacatcggcgcggggcttgccgatgcactaaccacaccgctcgaccataaagacaaaagtttgcagtctttgacgctggatcagtccgtcaggaaaaacgagaaactgaagctggcggcacaaggtgcggaaaaaacttatggaaacggcgacagcctcaatacgggcaaattgaagaacgacaaggtcagccgcttcgactttatccgtcaaatcgaagtggacggacaaaccatcacgctggcaagcggcgaatttcaaatatacaaacagaaccactccgccgtcgttgccctacagattgaaaaaatcaacaaccccgacaaaatcgacagcctgataaaccaacgctccttccttgtcagcggtttgggcggagaacataccgccttcaaccaactgcctgacggcaaagccgagtatcacggcaaagcattcagctccgacgacccgaacggcaggctgcactactccattgattttaccaaaaaacagggttacggcagaatcgaacacctgaaaacgcccgagcagaatgtcgagcttgcctccgccgaactcaaagcagatgaaaaatcacacgccgtcattttgggcgacacgcgctacggcggcgaagaaaaaggcacttaccacctcgcccttttcggcgaccgcgcccaagaaatcgccggcccggcaaccgtgaagataagggaaaaggttcacgaaatcggcatcgccagcaaacagtag

SEQ ID NO: 8 - protein, 254 aa, Neisseria meningitidis
CSSGGGGVAADIGAGLADALTTPLDHKDKSLQSLTLDQSVRKNEKLKLAAQGAEKTYGNGDSLNTGKLKNDKVSRFDFIRQIEVDGQTITLASGEFQIYKQNHSAVVALQIEKINNPDKIDSLINQRSFLVSGLGGEHTAFNQLPDGKAEYHGKAFSSDDPNGRLHYSIDFTKKQGYGRIEHLKTPEQNVELASAELKADEKSHAVILGDTRYGGEEKGTYHLALFGDRAQEIAGPATVKIREKVHEIGIASKQ

SEQ ID NO: 9 - DNA, 768 bp, Neisseria meningitidis
tgcggatccagcggaggcggcggtgtcgccgccgacatcggcgcggggcttgccgatgcactaaccacaccgctcgaccataaagacaaaagtttgcagtctttgacgctggatcagtccgtcaggaaaaacgagaaactgaagctggcggcacaaggtgcggaaaaaacttatggaaacggcgacagcctcaatacgggcaaattgaagaacgacaaggtcagccgcttcgactttatccgtcaaatcgaagtggacggacaaaccatcacgctggcaagcggcgaatttcaaatatacaaacagaaccactccgccgtcgttgccctacagattgaaaaaatcaacaaccccgacaaaatcgacagcctgataaaccaacgctccttccttgtcagcggtttgggcggagaacataccgccttcaaccaactgcctgacggcaaagccgagtatcacggcaaagcattcagctccgacgacccgaacggcaggctgcactactccattgattttaccaaaaaacagggttacggcagaatcgaacacctgaaaacgcccgagcagaatgtcgagcttgcctccgccgaactcaaagcagatgaaaaatcacacgccgtcattttgggcgacacgcgctacggcggcgaagaaaaaggcacttaccacctcgcccttttcggcgaccgcgcccaagaaatcgccggcccggcaaccgtgaagataagggaaaaggttcacgaaatcggcatcgccagcaaacagtag

SEQ ID NO: 10 - protein, 255 aa, Neisseria meningitidis
CGSSGGGGVAADIGAGLADALTTPLDHKDKSLQSLTLDQSVRKNEKLKLAAQGAEKTYGNGDSLNTGKLKNDKVSRFDFIRQIEVDGQTITLASGEFQIYKQNHSAVVALQIEKINNPDKIDSLINQRSFLVSGLGGEHTAFNQLPDGKAEYHGKAFSSDDPNGRLHYSIDFTKKQGYGRIEHLKTPEQNVELASAELKADEKSHAVILGDTRYGGEEKGTYHLALFGDRAQEIAGPATVKIREKVHEIGIASKQ

SEQ ID NO: 11 - DNA, 765 bp, Neisseria meningitidis
atgagcagcggaggcggcggtgtcgccgccgacatcggcgcggggcttgccgatgcactaaccacaccgctcgaccataaagacaaaagtttgcagtctttgacgctggatcagtccgtcaggaaaaacgagaaactgaagctggcggcacaaggtgcggaaaaaacttatggaaacggcgacagcctcaatacgggcaaattgaagaacgacaaggtcagccgcttcgactttatccgtcaaatcgaagtggacggacaaaccatcacgctggcaagcggcgaatttcaaatatacaaacagaaccactccgccgtcgttgccctacagattgaaaaaatcaacaaccccgacaaaatcgacagcctgataaaccaacgctccttccttgtcagcggtttgggcggagaacataccgccttcaaccaactgcctgacggcaaagccgagtatcacggcaaagcattcagctccgacgacccgaacggcaggctgcactactccattgattttaccaaaaaacagggttacggcagaatcgaacacctgaaaacgcccgagcagaatgtcgagcttgcctccgccgaactcaaagcagatgaaaaatcacacgccgtcattttgggcgacacgcgctacggcggcgaagaaaaaggcacttaccacctcgcccttttcggcgaccgcgcccaagaaatcgccggcccggcaaccgtgaagataagggaaaaggttcacgaaatcggcatcgccagcaaacagtag

SEQ ID NO: 12 - protein, 254 aa, Neisseria meningitidis
MSSGGGGVAADIGAGLADALTTPLDHKDKSLQSLTLDQSVRKNEKLKLAAQGAEKTYGNGDSLNTGKLKNDKVSRFDFIRQIEVDGQTITLASGEFQIYKQNHSAVVALQIEKINNPDKIDSLINQRSFLVSGLGGEHTAFNQLPDGKAEYHGKAFSSDDPNGRLHYSIDFTKKQGYGRIEHLKTPEQNVELASAELKADEKSHAVILGDTRYGGEEKGTYHLALFGDRAQEIAGPATVKIREKVHEIGIASKQ

SEQ ID NO: 13 - DNA, 765 bp, Neisseria meningitidis
tgcagcagcggaggcggcggtgtcgccgccgacatcggcgcggggcttgccgatgcactaaccgcaccgctcgaccataaagacaaaagtttgcagtctttgacgctggatcagtccgtcaggaaaaacgagaaactgaagctggcggcacaaggtgcggaaaaaacttatggaaacggcgacagcctcaatacgggcaaattgaagaacgacaaggtcagccgcttcgactttatccgtcaaatcggagtggacggacaaaccatcacgctggcaagcggcgaatttcaaatatacaaacagaaccactccgccgtcgttgccctacagattgaaaaaatcaacaaccccgacaaaatcgacagcctgataaaccaacgctccttccttgtcagcggtttgggcggagaacataccgccttcaaccaactgcctgacggcaaagccgagtatcacggcaaagcattcagctccgacgacccgaacggcaggctgcactactccattgattttaccaaaaaacagggttacggcagaatcgaacacctgaaaacgcccgagcagaatgtcgagcttgcctccgccgaactcaaagcagatgaaaaatcacacgccgtcattttgggcgacacgcgctacggcggcgaagaaaaaggcacttaccacctcgcccttttcggcgaccgcgcccaagaaatcgccggctcggcaaccgtgaagataagggaaaaggttcacgaaatcggcatcgccggcaaacagtag

SEQ ID NO: 14 - protein, 254 aa, Neisseria meningitidis
CSSGGGGVAADIGAGLADALTAPLDHKDKSLQSLTLDQSVRKNEKLKLAAQGAEKTYGNGDSLNTGKLKNDKVSRFDFIRQIGVDGQTITLASGEFQIYKQNHSAVVALQIEKINNPDKIDSLINQRSFLVSGLGGEHTAFNQLPDGKAEYHGKAFSSDDPNGRLHYSIDFTKKQGYGRIEHLKTPEQNVELASAELKADEKSHAVILGDTRYGGEEKGTYHLALFGDRAQEIAGSATVKIREKVHEIGIAGKQ

SEQ ID NO: 15 - DNA, 768 bp, Neisseria meningitidis
tgcggatccagcggaggcggcggtgtcgccgccgacatcggcgcggggcttgccgatgcactaaccgcaccgctcgaccataaagacaaaagtttgcagtctttgacgctggatcagtccgtcaggaaaaacgagaaactgaagctggcggcacaaggtgcggaaaaaacttatggaaacggcgacagcctcaatacgggcaaattgaagaacgacaaggtcagccgcttcgactttatccgtcaaatcggagtggacggacaaaccatcacgctggcaagcggcgaatttcaaatatacaaacagaaccactccgccgtcgttgccctacagattgaaaaaatcaacaaccccgacaaaatcgacagcctgataaaccaacgctccttccttgtcagcggtttgggcggagaacataccgccttcaaccaactgcctgacggcaaagccgagtatcacggcaaagcattcagctccgacgacccgaacggcaggctgcactactccattgattttaccaaaaaacagggttacggcagaatcgaacacctgaaaacgcccgagcagaatgtcgagcttgcctccgccgaactcaaagcagatgaaaaatcacacgccgtcattttgggcgacacgcgctacggcggcgaagaaaaaggcacttaccacctcgcccttttcggcgaccgcgcccaagaaatcgccggctcggcaaccgtgaagataagggaaaaggttcacgaaatcggcatcgccggcaaacagtag

SEQ ID NO: 16 - protein, 255 aa, Neisseria meningitidis
CGSSGGGGVAADIGAGLADALTAPLDHKDKSLQSLTLDQSVRKNEKLKLAAQGAEKTYGNGDSLNTGKLKNDKVSRFDFIRQIGVDGQTITLASGEFQIYKQNHSAVVALQIEKINNPDKIDSLINQRSFLVSGLGGEHTAFNQLPDGKAEYHGKAFSSDDPNGRLHYSIDFTKKQGYGRIEHLKTPEQNVELASAELKADEKSHAVILGDTRYGGEEKGTYHLALFGDRAQEIAGSATVKIREKVHEIGIAGKQ

SEQ ID NO: 17 - DNA, 765 bp, Neisseria meningitidis
atgagcagcggaggcggcggtgtcgccgccgacatcggcgcggggcttgccgatgcactaaccgcaccgctcgaccataaagacaaaagtttgcagtctttgacgctggatcagtccgtcaggaaaaacgagaaactgaagctggcggcacaaggtgcggaaaaaacttatggaaacggcgacagcctcaatacgggcaaattgaagaacgacaaggtcagccgcttcgactttatccgtcaaatcggagtggacggacaaaccatcacgctggcaagcggcgaatttcaaatatacaaacagaaccactccgccgtcgttgccctacagattgaaaaaatcaacaaccccgacaaaatcgacagcctgataaaccaacgctccttccttgtcagcggtttgggcggagaacataccgccttcaaccaactgcctgacggcaaagccgagtatcacggcaaagcattcagctccgacgacccgaacggcaggctgcactactccattgattttaccaaaaaacagggttacggcagaatcgaacacctgaaaacgcccgagcagaatgtcgagcttgcctccgccgaactcaaagcagatgaaaaatcacacgccgtcattttgggcgacacgcgctacggcggcgaagaaaaaggcacttaccacctcgcccttttcggcgaccgcgcccaagaaatcgccggctcggcaaccgtgaagataagggaaaaggttcacgaaatcggcatcgccggcaaacagtag

SEQ ID NO: 18 - protein, 254 aa, Neisseria meningitidis
MSSGGGGVAADIGAGLADALTAPLDHKDKSLQSLTLDQSVRKNEKLKLAAQGAEKTYGNGDSLNTGKLKNDKVSRFDFIRQIGVDGQTITLASGEFQIYKQNHSAVVALQIEKINNPDKIDSLINQRSFLVSGLGGEHTAFNQLPDGKAEYHGKAFSSDDPNGRLHYSIDFTKKQGYGRIEHLKTPEQNVELASAELKADEKSHAVILGDTRYGGEEKGTYHLALFGDRAQEIAGSATVKIREKVHEIGIAGKQ

SEQ ID NO: 19 - DNA, 765 bp, Neisseria meningitidis
tgcagcagcagaggcggcggtgtcgccgccgacatcggcgcggggcttgccgatgcactaaccgcaccgctcgaccataaagacaaaagtttgcagtctttgacgctggatcagtccgtcaggaaaaacgagaaactgaagctggcggcacaaggtgcggaaaaaacttatggaaacggcgacagcctcaatacgggcaaattgaagaacgacaaggtcagccgcttcgactttatccgtcaaatcgaagtggacggacaaaccatcacgctggcaagcggcgaatttcaaatatacaaacagaaccactccgccgtcgttgccctacagattgaaaaaatcaacaaccccgacaaaatcgacagcctgataaaccaacgctccttccttgtcagcggtttgggcggagaacataccgccttcaaccaactgcctgacggcaaagccgagtatcacggcaaagcattcagctccgacgacccgaacggcaggctgcactactccattgattttaccaaaaaacagggttacggcagaatcgaacacctgaaaacgcccgagcagaatgtcgagcttgcctccgccgaactcaaagcagatgaaaaatcacacgccgtcattttgggcgacacgcgctacggcggcgaagaaaaaggcacttaccacctcgcccttttcggcgaccgcgcccaagaaatcgccggctcggcaaccgtgaagataagggaaaaggttcacgaaatcggcatcgccggcaaacagtag

SEQ ID NO: 20 - protein, 254 aa, Neisseria meningitidis
CSSRGGGVAADIGAGLADALTAPLDHKDKSLQSLTLDQSVRKNEKLKLAAQGAEKTYGNGDSLNTGKLKNDKVSRFDFIRQIEVDGQTITLASGEFQIYKQNHSAVVALQIEKINNPDKIDSLINQRSFLVSGLGGEHTAFNQLPDGKAEYHGKAFSSDDPNGRLHYSIDFTKKQGYGRIEHLKTPEQNVELASAELKADEKSHAVILGDTRYGGEEKGTYHLALFGDRAQEIAGSATVKIREKVHEIGIAGKQ

SEQ ID NO: 21 - DNA, 768 bp, Neisseria meningitidis
tgcggatccagcagaggcggcggtgtcgccgccgacatcggcgcggggcttgccgatgcactaaccgcaccgctcgaccataaagacaaaagtttgcagtctttgacgctggatcagtccgtcaggaaaaacgagaaactgaagctggcggcacaaggtgcggaaaaaacttatggaaacggcgacagcctcaatacgggcaaattgaagaacgacaaggtcagccgcttcgactttatccgtcaaatcgaagtggacggacaaaccatcacgctggcaagcggcgaatttcaaatatacaaacagaaccactccgccgtcgttgccctacagattgaaaaaatcaacaaccccgacaaaatcgacagcctgataaaccaacgctccttccttgtcagcggtttgggcggagaacataccgccttcaaccaactgcctgacggcaaagccgagtatcacggcaaagcattcagctccgacgacccgaacggcaggctgcactactccattgattttaccaaaaaacagggttacggcagaatcgaacacctgaaaacgcccgagcagaatgtcgagcttgcctccgccgaactcaaagcagatgaaaaatcacacgccgtcattttgggcgacacgcgctacggcggcgaagaaaaaggcacttaccacctcgcccttttcggcgaccgcgcccaagaaatcgccggctcggcaaccgtgaagataagggaaaaggttcacgaaatcggcatcgccggcaaacagtag

SEQ ID NO: 22 - protein, 255 aa, Neisseria meningitidis
CGSSRGGGVAADIGAGLADALTAPLDHKDKSLQSLTLDQSVRKNEKLKLAAQGAEKTYGNGDSLNTGKLKNDKVSRFDFIRQIEVDGQTITLASGEFQIYKQNHSAVVALQIEKINNPDKIDSLINQRSFLVSGLGGEHTAFNQLPDGKAEYHGKAFSSDDPNGRLHYSIDFTKKQGYGRIEHLKTPEQNVELASAELKADEKSHAVILGDTRYGGEEKGTYHLALFGDRAQEIAGSATVKIREKVHEIGIAGKQ

SEQ ID NO: 23 - DNA, 765 bp, Neisseria meningitidis
atgagcagcagaggcggcggtgtcgccgccgacatcggcgcggggcttgccgatgcactaaccgcaccgctcgaccataaagacaaaagtttgcagtctttgacgctggatcagtccgtcaggaaaaacgagaaactgaagctggcggcacaaggtgcggaaaaaacttatggaaacggcgacagcctcaatacgggcaaattgaagaacgacaaggtcagccgcttcgactttatccgtcaaatcgaagtggacggacaaaccatcacgctggcaagcggcgaatttcaaatatacaaacagaaccactccgccgtcgttgccctacagattgaaaaaatcaacaaccccgacaaaatcgacagcctgataaaccaacgctccttccttgtcagcggtttgggcggagaacataccgccttcaaccaactgcctgacggcaaagccgagtatcacggcaaagcattcagctccgacgacccgaacggcaggctgcactactccattgattttaccaaaaaacagggttacggcagaatcgaacacctgaaaacgcccgagcagaatgtcgagcttgcctccgccgaactcaaagcagatgaaaaatcacacgccgtcattttgggcgacacgcgctacggcggcgaagaaaaaggcacttaccacctcgcccttttcggcgaccgcgcccaagaaatcgccggctcggcaaccgtgaagataagggaaaaggttcacgaaatcggcatcgccggcaaacagtag

SEQ ID NO: 24 - protein, 254 aa, Neisseria meningitidis
MSSRGGGVAADIGAGLADALTAPLDHKDKSLQSLTLDQSVRKNEKLKLAAQGAEKTYGNGDSLNTGKLKNDKVSRFDFIRQIEVDGQTITLASGEFQIYKQNHSAVVALQIEKINNPDKIDSLINQRSFLVSGLGGEHTAFNQLPDGKAEYHGKAFSSDDPNGRLHYSIDFTKKQGYGRIEHLKTPEQNVELASAELKADEKSHAVILGDTRYGGEEKGTYHLALFGDRAQEIAGSATVKIREKVHEIGIAGKQ

SEQ ID NO: 25 - DNA, 765 bp, Neisseria meningitidis
tgcagcagcggaggcggcggtgtcgccgccgacatcggcgcggggcttgccgatgcactaaccgcaccgttcgaccataaagacaaaagtttgcagtctttgacgctggatcagtccgtcaggaaaaacgagaaactgaagctggcggcacaaggtgcggaaaaaacttatggaaacggcgacagcctcaatacgggcaaattgaagaacgacaaggtcagccgcttcgactttatccgtcaaatcgaagtggacgggcagctcattaccttggagagcggagagttccaaatatacaaacaggaccactccgccgtcgttgccctacagattgaaaaaatcaacaaccccgacaaaatcgacagcctgataaaccaacgctccttccttgtcagcggtttgggtggagaacataccgccttcaaccaactgcccagcggcaaagccgagtatcacggcaaagcattcagctccgacgacccgaacggcaggctgcactactccattgattttaccaaaaaacagggttacggcagaatcgaacacctgaaaacgcccgagcagaatgtcgagcttgcctccgccgaactcaaagcagatgaaaaatcacacgccgtcattttgggcgacacgcgctacggcggcgaagaaaaaggcacttaccacctcgcccttttcggcgaccgcgcccaagaaatcgccggctcggcaaccgtgaagataagggaaaaggttcacgaaatcggcatcgccggcaaacagtag

SEQ ID NO: 26 - protein, 254 aa, Neisseria meningitidis
CSSGGGGVAADIGAGLADALTAPFDHKDKSLQSLTLDQSVRKNEKLKLAAQGAEKTYGNGDSLNTGKLKNDKVSRFDFIRQIEVDGQLITLESGEFQIYKQDHSAVVALQIEKINNPDKIDSLINQRSFLVSGLGGEHTAFNQLPSGKAEYHGKAFSSDDPNGRLHYSIDFTKKQGYGRIEHLKTPEQNVELASAELKADEKSHAVILGDTRYGGEEKGTYHLALFGDRAQEIAGSATVKIREKVHEIGIAGKQ

SEQ ID NO: 27 - DNA, 768 bp, Neisseria meningitidis
tgcggatccagcggaggcggcggtgtcgccgccgacatcggcgcggggcttgccgatgcactaaccgcaccgttcgaccataaagacaaaagtttgcagtctttgacgctggatcagtccgtcaggaaaaacgagaaactgaagctggcggcacaaggtgcggaaaaaacttatggaaacggcgacagcctcaatacgggcaaattgaagaacgacaaggtcagccgcttcgactttatccgtcaaatcgaagtggacgggcagctcattaccttggagagcggagagttccaaatatacaaacaggaccactccgccgtcgttgccctacagattgaaaaaatcaacaaccccgacaaaatcgacagcctgataaaccaacgctccttccttgtcagcggtttgggtggagaacataccgccttcaaccaactgcccagcggcaaagccgagtatcacggcaaagcattcagctccgacgacccgaacggcaggctgcactactccattgattttaccaaaaaacagggttacggcagaatcgaacacctgaaaacgcccgagcagaatgtcgagcttgcctccgccgaactcaaagcagatgaaaaatcacacgccgtcattttgggcgacacgcgctacggcggcgaagaaaaaggcacttaccacctcgcccttttcggcgaccgcgcccaagaaatcgccggctcggcaaccgtgaagataagggaaaaggttcacgaaatcggcatcgccggcaaacagtag

SEQ ID NO: 28 - protein, 255 aa, Neisseria meningitidis
CGSSGGGGVAADIGAGLADALTAPFDHKDKSLQSLTLDQSVRKNEKLKLAAQGAEKTYGNGDSLNTGKLKNDKVSRFDFIRQIEVDGQLITLESGEFQIYKQDHSAVVALQIEKINNPDKIDSLINQRSFLVSGLGGEHTAFNQLPSGKAEYHGKAFSSDDPNGRLHYSIDFTKKQGYGRIEHLKTPEQNVELASAELKADEKSHAVILGDTRYGGEEKGTYHLALFGDRAQEIAGSATVKIREKVHEIGIAGKQ

SEQ ID NO: 29 - DNA, 765 bp, Neisseria meningitidis
atgagcagcggaggcggcggtgtcgccgccgacatcggcgcggggcttgccgatgcactaaccgcaccgttcgaccataaagacaaaagtttgcagtctttgacgctggatcagtccgtcaggaaaaacgagaaactgaagctggcggcacaaggtgcggaaaaaacttatggaaacggcgacagcctcaatacgggcaaattgaagaacgacaaggtcagccgcttcgactttatccgtcaaatcgaagtggacgggcagctcattaccttggagagcggagagttccaaatatacaaacaggaccactccgccgtcgttgccctacagattgaaaaaatcaacaaccccgacaaaatcgacagcctgataaaccaacgctccttccttgtcagcggtttgggtggagaacataccgccttcaaccaactgcccagcggcaaagccgagtatcacggcaaagcattcagctccgacgacccgaacggcaggctgcactactccattgattttaccaaaaaacagggttacggcagaatcgaacacctgaaaacgcccgagcagaatgtcgagcttgcctccgccgaactcaaagcagatgaaaaatcacacgccgtcattttgggcgacacgcgctacggcggcgaagaaaaaggcacttaccacctcgcccttttcggcgaccgcgcccaagaaatcgccggctcggcaaccgtgaagataagggaaaaggttcacgaaatcggcatcgccggcaaacagtag

SEQ ID NO: 30 - protein, 254 aa, Neisseria meningitidis
MSSGGGGVAADIGAGLADALTAPFDHKDKSLQSLTLDQSVRKNEKLKLAAQGAEKTYGNGDSLNTGKLKNDKVSRFDFIRQIEVDGQLITLESGEFQIYKQDHSAVVALQIEKINNPDKIDSLINQRSFLVSGLGGEHTAFNQLPSGKAEYHGKAFSSDDPNGRLHYSIDFTKKQGYGRIEHLKTPEQNVELASAELKADEKSHAVILGDTRYGGEEKGTYHLALFGDRAQEIAGSATVKIREKVHEIGIAGKQ

SEQ ID NO: 31 - DNA, 765 bp, Neisseria meningitidis
tgcagcagcggaggcggcggtgtcgccgccgacatcggcgcggggcttgccgatgcactaaccgcaccgctcgaccataaagacaaaagtttgcagtctttgacgctggatcagtccgtcaggaaaaacgagaaactgaagctggcggcacaaggtgcggaaaaaacttatggaaacggcgacagcctcaatacgggcaaattgaagaacgacaaggtcagccgcttcgactttatccgtcaaatcgaagtggacgggcagctcattaccttggagagcggagagttccaaatatacaaacaggaccactccgccgtcgttgccctacagattgaaaaaatcaacaaccccgacaaaatcgacagcctgataaaccaacgctccttccttgtcagcggtttgggtggagaacataccgccttcaaccaactgcccagcggcaaagccgagtatcacggcaaagcattcagctccgacgacccgaacggcaggctgcactactccattgattttaccaaaaaacagggttacggcagaatcgaacacctgaaaacgcccgagcagaatgtcgagcttgcctccgccgaactcaaagcagatgaaaaatcacacgccgtcattttgggcgacacgcgctacggcggcgaagaaaaaggcacttaccacctcgcccttttcggcgaccgcgcccaagaaatcgccggctcggcaaccgtgaagataagggaaaaggttcacgaaatcggcatcgccggcaaacagtag

SEQ ID NO: 32 - protein, 254 aa, Neisseria meningitidis
CSSGGGGVAADIGAGLADALTAPLDHKDKSLQSLTLDQSVRKNEKLKLAAQGAEKTYGNGDSLNTGKLKNDKVSRFDFIRQIEVDGQLITLESGEFQIYKQDHSAVVALQIEKINNPDKIDSLINQRSFLVSGLGGEHTAFNQLPSGKAEYHGKAFSSDDPNGRLHYSIDFTKKQGYGRIEHLKTPEQNVELASAELKADEKSHAVILGDTRYGGEEKGTYHLALFGDRAQEIAGSATVKIREKVHEIGIAGKQ

SEQ ID NO: 33 - DNA, 768 bp, Neisseria meningitidis
tgcggatccagcggaggcggcggtgtcgccgccgacatcggcgcggggcttgccgatgcactaaccgcaccgctcgaccataaagacaaaagtttgcagtctttgacgctggatcagtccgtcaggaaaaacgagaaactgaagctggcggcacaaggtgcggaaaaaacttatggaaacggcgacagcctcaatacgggcaaattgaagaacgacaaggtcagccgcttcgactttatccgtcaaatcgaagtggacgggcagctcattaccttggagagcggagagttccaaatatacaaacaggaccactccgccgtcgttgccctacagattgaaaaaatcaacaaccccgacaaaatcgacagcctgataaaccaacgctccttccttgtcagcggtttgggtggagaacataccgccttcaaccaactgcccagcggcaaagccgagtatcacggcaaagcattcagctccgacgacccgaacggcaggctgcactactccattgattttaccaaaaaacagggttacggcagaatcgaacacctgaaaacgcccgagcagaatgtcgagcttgcctccgccgaactcaaagcagatgaaaaatcacacgccgtcattttgggcgacacgcgctacggcggcgaagaaaaaggcacttaccacctcgcccttttcggcgaccgcgcccaagaaatcgccggctcggcaaccgtgaagataagggaaaaggttcacgaaatcggcatcgccggcaaacagtag

SEQ ID NO: 34 - protein, 255 aa, Neisseria meningitidis
CGSSGGGGVAADIGAGLADALTAPLDHKDKSLQSLTLDQSVRKNEKLKLAAQGAEKTYGNGDSLNTGKLKNDKVSRFDFIRQIEVDGQLITLESGEFQIYKQDHSAVVALQIEKINNPDKIDSLINQRSFLVSGLGGEHTAFNQLPSGKAEYHGKAFSSDDPNGRLHYSIDFTKKQGYGRIEHLKTPEQNVELASAELKADEKSHAVILGDTRYGGEEKGTYHLALFGDRAQEIAGSATVKIREKVHEIGIAGKQ

SEQ ID NO: 35 - DNA, 765 bp, Neisseria meningitidis
atgagcagcggaggcggcggtgtcgccgccgacatcggcgcggggcttgccgatgcactaaccgcaccgctcgaccataaagacaaaagtttgcagtctttgacgctggatcagtccgtcaggaaaaacgagaaactgaagctggcggcacaaggtgcggaaaaaacttatggaaacggcgacagcctcaatacgggcaaattgaagaacgacaaggtcagccgcttcgactttatccgtcaaatcgaagtggacgggcagctcattaccttggagagcggagagttccaaatatacaaacaggaccactccgccgtcgttgccctacagattgaaaaaatcaacaaccccgacaaaatcgacagcctgataaaccaacgctccttccttgtcagcggtttgggtggagaacataccgccttcaaccaactgcccagcggcaaagccgagtatcacggcaaagcattcagctccgacgacccgaacggcaggctgcactactccattgattttaccaaaaaacagggttacggcagaatcgaacacctgaaaacgcccgagcagaatgtcgagcttgcctccgccgaactcaaagcagatgaaaaatcacacgccgtcattttgggcgacacgcgctacggcggcgaagaaaaaggcacttaccacctcgcccttttcggcgaccgcgcccaagaaatcgccggctcggcaaccgtgaagataagggaaaaggttcacgaaatcggcatcgccggcaaacagtag

SEQ ID NO: 36 - protein, 254 aa, Neisseria meningitidis
MSSGGGGVAADIGAGLADALTAPLDHKDKSLQSLTLDQSVRKNEKLKLAAQGAEKTYGNGDSLNTGKLKNDKVSRFDFIRQIEVDGQLITLESGEFQIYKQDHSAVVALQIEKINNPDKIDSLINQRSFLVSGLGGEHTAFNQLPSGKAEYHGKAFSSDDPNGRLHYSIDFTKKQGYGRIEHLKTPEQNVELASAELKADEKSHAVILGDTRYGGEEKGTYHLALFGDRAQEIAGSATVKIREKVHEIGIAGKQ

SEQ ID NO: 37 - DNA, 765 bp, Neisseria meningitidis; identical to SEQ ID NO: 31

SEQ ID NO: 38 - protein, 254 aa, Neisseria meningitidis; identical to SEQ ID NO: 32

SEQ ID NO: 39 - DNA, 768 bp, Neisseria meningitidis; identical to SEQ ID NO: 33

SEQ ID NO: 40 - protein, 255 aa, Neisseria meningitidis; identical to SEQ ID NO: 34

SEQ ID NO: 41 - DNA, 765 bp, Neisseria meningitidis; identical to SEQ ID NO: 35

SEQ ID NO: 42 - protein, 254 aa, Neisseria meningitidis; identical to SEQ ID NO: 36

SEQ ID NO: 43 - DNA, 765 bp, Neisseria meningitidis; identical to SEQ ID NO: 31

SEQ ID NO: 44 - protein, 254 aa, Neisseria meningitidis; identical to SEQ ID NO: 32

SEQ ID NO: 45 - DNA, 768 bp, Neisseria meningitidis; identical to SEQ ID NO: 33

SEQ ID NO: 46 - protein, 255 aa, Neisseria meningitidis; identical to SEQ ID NO: 34

SEQ ID NO: 47 - DNA, 765 bp, Neisseria meningitidis; identical to SEQ ID NO: 35

SEQ ID NO: 48 - protein, 254 aa, Neisseria meningitidis; identical to SEQ ID NO: 36

SEQ ID NO: 49 - DNA, 786 bp, Neisseria meningitidis
tgcagcagcggaagcggaagcggaggcggcggtgtcgccgccgacatcggcacagggcttgccgatgcactaactgcgccgctcgaccataaagacaaaggtttgaaatccctgacattggaagactccatttcccaaaacggaacactgaccctgtcggcacaaggtgcggaaaaaactttcaaagtcggcgacaaagacaacagtctcaatacaggcaaattgaagaacgacaaaatcagccgcttcgactttgtgcaaaaaatcgaagtggacggacaaaccatcacgctggcaagcggcgaatttcaaatatacaaacaggaccactccgccgtcgttgccctacagattgaaaaaatcaacaaccccgacaaaatcgacagcctgataaaccaacgctccttccttgtcagcggtttgggcggagaacataccgccttcaaccaactgcccagcggcaaagccgagtatcacggcaaagcattcagctccgacgatgccggcggaaaactgacctataccatagattttgccgccaaacagggacacggcaaaatcgaacacctgaaaacacccgagcagaatgtcgagcttgcctccgccgaactcaaagcagatgaaaaatcacacgccgtcattttgggcgacacgcgctacggcagcgaagaaaaaggcacttaccacctcgctcttttcggcgaccgagcccaagaaatcgccggctcggcaaccgtgaagataagggaaaaggttcacgaaatcggcatcgccggcaaacagtag

SEQ ID NO: 50 - protein, 261 aa, Neisseria meningitidis
CSSGSGSGGGGVAADIGTGLADALTAPLDHKDKGLKSLTLEDSISQNGTLTLSAQGAEKTFKVGDKDNSLNTGKLKNDKISRFDFVQKIEVDGQTITLASGEFQIYKQDHSAVVALQIEKINNPDKIDSLINQRSFLVSGLGGEHTAFNQLPSGKAEYHGKAFSSDDAGGKLTYTIDFAAKQGHGKIEHLKTPEQNVELASAELKADEKSHAVILGDTRYGSEEKGTYHLALFGDRAQEIAGSATVKIREKVHEIGIAGKQ

SEQ ID NO: 51 - DNA, 789 bp, Neisseria meningitidis
tgcggatccagcggaagcggaagcggaggcggcggtgtcgccgccgacatcggcacagggcttgccgatgcactaactgcgccgctcgaccataaagacaaaggtttgaaatccctgacattggaagactccatttcccaaaacggaacactgaccctgtcggcacaaggtgcggaaaaaactttcaaagtcggcgacaaagacaacagtctcaatacaggcaaattgaagaacgacaaaatcagccgcttcgactttgtgcaaaaaatcgaagtggacggacaaaccatcacgctggcaagcggcgaatttcaaatatacaaacaggaccactccgccgtcgttgccctacagattgaaaaaatcaacaaccccgacaaaatcgacagcctgataaaccaacgctccttccttgtcagcggtttgggcggagaacataccgccttcaaccaactgcccagcggcaaagccgagtatcacggcaaagcattcagctccgacgatgccggcggaaaactgacctataccatagattttgccgccaaacagggacacggcaaaatcgaacacctgaaaacacccgagcagaatgtcgagcttgcctccgccgaactcaaagcagatgaaaaatcacacgccgtcattttgggcgacacgcgctacggcagcgaagaaaaaggcacttaccacctcgctcttttcggcgaccgagcccaagaaatcgccggctcggcaaccgtgaagataagggaaaaggttcacgaaatcggcatcgccggcaaacagtag

SEQ ID NO: 52 - protein, 262 aa, Neisseria meningitidis
CGSSGSGSGGGGVAADIGTGLADALTAPLDHKDKGLKSLTLEDSISQNGTLTLSAQGAEKTFKVGDKDNSLNTGKLKNDKISRFDFVQKIEVDGQTITLASGEFQIYKQDHSAVVALQIEKINNPDKIDSLINQRSFLVSGLGGEHTAFNQLPSGKAEYHGKAFSSDDAGGKLTYTIDFAAKQGHGKIEHLKTPEQNVELASAELKADEKSHAVILGDTRYGSEEKGTYHLALFGDRAQEIAGSATVKIREKVHEIGIAGKQ

SEQ ID NO: 53 - DNA, 786 bp, Neisseria meningitidis
atgagcagcggaagcggaagcggaggcggcggtgtcgccgccgacatcggcacagggcttgccgatgcactaactgcgccgctcgaccataaagacaaaggtttgaaatccctgacattggaagactccatttcccaaaacggaacactgaccctgtcggcacaaggtgcggaaaaaactttcaaagtcggcgacaaagacaacagtctcaatacaggcaaattgaagaacgacaaaatcagccgcttcgactttgtgcaaaaaatcgaagtggacggacaaaccatcacgctggcaagcggcgaatttcaaatatacaaacaggaccactccgccgtcgttgccctacagattgaaaaaatcaacaaccccgacaaaatcgacagcctgataaaccaacgctccttccttgtcagcggtttgggcggagaacataccgccttcaaccaactgcccagcggcaaagccgagtatcacggcaaagcattcagctccgacgatgccggcggaaaactgacctataccatagattttgccgccaaacagggacacggcaaaatcgaacacctgaaaacacccgagcagaatgtcgagcttgcctccgccgaactcaaagcagatgaaaaatcacacgccgtcattttgggcgacacgcgctacggcagcgaagaaaaaggcacttaccacctcgctcttttcggcgaccgagcccaagaaatcgccggctcggcaaccgtgaagataagggaaaaggttcacgaaatcggcatcgccggcaaacagtag

SEQ ID NO: 54 - protein, 261 aa, Neisseria meningitidis
MSSGSGSGGGGVAADIGTGLADALTAPLDHKDKGLKSLTLEDSISQNGTLTLSAQGAEKTFKVGDKDNSLNTGKLKNDKISRFDFVQKIEVDGQTITLASGEFQIYKQDHSAVVALQIEKINNPDKIDSLINQRSFLVSGLGGEHTAFNQLPSGKAEYHGKAFSSDDAGGKLTYTIDFAAKQGHGKIEHLKTPEQNVELASAELKADEKSHAVILGDTRYGSEEKGTYHLALFGDRAQEIAGSATVKIREKVHEIGIAGKQ

SEQ ID NO: 55 - DNA, 786 bp, Neisseria meningitidis; identical to SEQ ID NO: 49

SEQ ID NO: 56 - protein, 261 aa, Neisseria meningitidis; identical to SEQ ID NO: 50

SEQ ID NO: 57 - DNA, 789 bp, Neisseria meningitidis; identical to SEQ ID NO: 51

SEQ ID NO: 58 - protein, 262 aa, Neisseria meningitidis; identical to SEQ ID NO: 52

SEQ ID NO: 59 - DNA, 786 bp, Neisseria meningitidis; identical to SEQ ID NO: 53

SEQ ID NO: 60 - protein, 261 aa, Neisseria meningitidis; identical to SEQ ID NO: 54

SEQ ID NO: 61 - DNA, 789 bp, Neisseria meningitidis
tgcagcagcggaggcggcggaagcggaggcggcggtgtcgccgccgacatcggcacggggcttgccgatgcactaactgcgccgctcgaccataaagacaaaggtttgaaatccctgacattggaagactctattccccaaaacggaacactaaccctgtcggcacaaggtgcggaaaaaactttcaaagccggcgacaaagacaacagcctcaacacgggcaaactgaagaacgacaaaatcagccgcttcgactttgtgcaaaaaatcgaagtggacggacaaaccatcacgctggcaagcggcgaatttcaaatatacaaacaggaccactccgccgtcgttgccctacagattgaaaaaatcaacaaccccgacaaaatcgacagcctgataaaccaacgctccttccttgtcagcggtttgggcggagaacataccgccttcaaccaactgcccggcggcaaagccgagtatcacggcaaagcattcagctccgacgacccgaacggcaggctgcactactccattgattttaccaaaaaacagggttacggcggaatcgaacacctgaaaacacccgagcaaaatgtcgagcttgcctccgccgaactcaaagcagatgaaaaatcacacgccgtcattttgggcgacacgcgctacggcagcgaagaaaaaggcacttaccacctcgcccttttcggcgaccgcgcccaagaaatcgccggctcggcaaccgtgaagataggggaaaaggttcacgaaatcggcatcgccggcaaacagtag

SEQ ID NO: 62 - protein, 262 aa, Neisseria meningitidis
CSSGGGGSGGGGVAADIGTGLADALTAPLDHKDKGLKSLTLEDSIPQNGTLTLSAQGAEKTFKAGDKDNSLNTGKLKNDKISRFDFVQKIEVDGQTITLASGEFQIYKQDHSAVVALQIEKINNPDKIDSLINQRSFLVSGLGGEHTAFNQLPGGKAEYHGKAFSSDDPNGRLHYSIDFTKKQGYGGIEHLKTPEQNVELASAELKADEKSHAVILGDTRYGSEEKGTYHLALFGDRAQEIAGSATVKIGEKVHEIGIAGKQ

SEQ ID NO: 63 - DNA, 792 bp, Neisseria meningitidis
tgcggatccagcggaggcggcggaagcggaggcggcggtgtcgccgccgacatcggcacggggcttgccgatgcactaactgcgccgctcgaccataaagacaaaggtttgaaatccctgacattggaagactctattccccaaaacggaacactaaccctgtcggcacaaggtgcggaaaaaactttcaaagccggcgacaaagacaacagcctcaacacgggcaaactgaagaacgacaaaatcagccgcttcgactttgtgcaaaaaatcgaagtggacggacaaaccatcacgctggcaagcggcgaatttcaaatatacaaacaggaccactccgccgtcgttgccctacagattgaaaaaatcaacaaccccgacaaaatcgacagcctgataaaccaacgctccttccttgtcagcggtttgggcggagaacataccgccttcaaccaactgcccggcggcaaagccgagtatcacggcaaagcattcagctccgacgacccgaacggcaggctgcactactccattgattttaccaaaaaacagggttacggcggaatcgaacacctgaaaacacccgagcaaaatgtcgagcttgcctccgccgaactcaaagcagatgaaaaatcacacgccgtcattttgggcgacacgcgctacggcagcgaagaaaaaggcacttaccacctcgcccttttcggcgaccgcgcccaagaaatcgccggctcggcaaccgtgaagataggggaaaaggttcacgaaatcggcatcgccggcaaacagtag

SEQ ID NO: 64 - protein, 263 aa, Neisseria meningitidis
CGSSGGGGSGGGGVAADIGTGLADALTAPLDHKDKGLKSLTLEDSIPQNGTLTLSAQGAEKTFKAGDKDNSLNTGKLKNDKISRFDFVQKIEVDGQTITLASGEFQIYKQDHSAVVALQIEKINNPDKIDSLINQRSFLVSGLGGEHTAFNQLPGGKAEYHGKAFSSDDPNGRLHYSIDFTKKQGYGGIEHLKTPEQNVELASAELKADEKSHAVILGDTRYGSEEKGTYHLALFGDRAQEIAGSATVKIGEKVHEIGIAGKQ

SEQ ID NO: 65 - DNA, 789 bp, Neisseria meningitidis
atgagcagcggaggcggcggaagcggaggcggcggtgtcgccgccgacatcggcacggggcttgccgatgcactaactgcgccgctcgaccataaagacaaaggtttgaaatccctgacattggaagactctattccccaaaacggaacactaaccctgtcggcacaaggtgcggaaaaaactttcaaagccggcgacaaagacaacagcctcaacacgggcaaactgaagaacgacaaaatcagccgcttcgactttgtgcaaaaaatcgaagtggacggacaaaccatcacgctggcaagcggcgaatttcaaatatacaaacaggaccactccgccgtcgttgccctacagattgaaaaaatcaacaaccccgacaaaatcgacagcctgataaaccaacgctccttccttgtcagcggtttgggcggagaacataccgccttcaaccaactgcccggcggcaaagccgagtatcacggcaaagcattcagctccgacgacccgaacggcaggctgcactactccattgattttaccaaaaaacagggttacggcggaatcgaacacctgaaaacacccgagcaaaatgtcgagcttgcctccgccgaactcaaagcagatgaaaaatcacacgccgtcattttgggcgacacgcgctacggcagcgaagaaaaaggcacttaccacctcgcccttttcggcgaccgcgcccaagaaatcgccggctcggcaaccgtgaagataggggaaaaggttcacgaaatcggcatcgccggcaaacagtag

SEQ ID NO: 66 - protein, 262 aa, Neisseria meningitidis
MSSGGGGSGGGGVAADIGTGLADALTAPLDHKDKGLKSLTLEDSIPQNGTLTLSAQGAEKTFKAGDKDNSLNTGKLKNDKISRFDFVQKIEVDGQTITLASGEFQIYKQDHSAVVALQIEKINNPDKIDSLINQRSFLVSGLGGEHTAFNQLPGGKAEYHGKAFSSDDPNGRLHYSIDFTKKQGYGGIEHLKTPEQNVELASAELKADEKSHAVILGDTRYGSEEKGTYHLALFGDRAQEIAGSATVKIGEKVHEIGIAGKQ

SEQ ID NO: 67 - DNA, 774 bp, Neisseria meningitidis
tgcagcagcggaggcggcggtgtcgccgccgacatcggcgcggggcttgccgatgcactaaccgcaccgctcgaccataaagacaaaggtttgaaatccctgacattggaagactccatttcccaaaacggaacactgaccctgtcggcacaaggtgcggaaaaaactttcaaagtcggcgacaaagacaacagtctcaatacaggcaaattgaagaacgacaaaatcagccgcttcgactttgtgcaaaaaatcgaagtggacggacaaaccatcacgctggcaagcggcgaatttcaaatatacaaacagaaccactccgccgtcgttgccctacagattgaaaaaatcaacaaccccgacaaaatcgacagcctgataaaccaacgctccttccttgtcagcggtttgggcggagaacataccgccttcaaccaactgcccggcggcaaagccgagtatcacggcaaagcattcagctccgacgatgccggcggaaaactgacctataccatagattttgccgccaaacagggacacggcaaaatcgaacacctgaaaacacccgagcaaaatgtcgagcttgccgccgccgaactcaaagcagatgaaaaatcacacgccgtcattttgggcgacacgcgctacggcagcgaagaaaaaggcacttaccacctcgcccttttcggcgaccgcgctcaagaaatcgccggctcggcaaccgtgaagataggagaaaaggttcacgaaatcagcatcgccggcaaacagtag

SEQ ID NO: 68 - protein, 257 aa, Neisseria meningitidis
CSSGGGGVAADIGAGLADALTAPLDHKDKGLKSLTLEDSISQNGTLTLSAQGAEKTFKVGDKDNSLNTGKLKNDKISRFDFVQKIEVDGQTITLASGEFQIYKQNHSAVVALQIEKINNPDKIDSLINQRSFLVSGLGGEHTAFNQLPGGKAEYHGKAFSSDDAGGKLTYTIDFAAKQGHGKIEHLKTPEQNVELAAAELKADEKSHAVILGDTRYGSEEKGTYHLALFGDRAQEIAGSATVKIGEKVHEISIAGKQ

SEQ ID NO: 69 - DNA, 777 bp, Neisseria meningitidis
tgcggatccagcggaggcggcggtgtcgccgccgacatcggcgcggggcttgccgatgcactaaccgcaccgctcgaccataaagacaaaggtttgaaatccctgacattggaagactccatttcccaaaacggaacactgaccctgtcggcacaaggtgcggaaaaaactttcaaagtcggcgacaaagacaacagtctcaatacaggcaaattgaagaacgacaaaatcagccgcttcgactttgtgcaaaaaatcgaagtggacggacaaaccatcacgctggcaagcggcgaatttcaaatatacaaacagaaccactccgccgtcgttgccctacagattgaaaaaatcaacaaccccgacaaaatcgacagcctgataaaccaacgctccttccttgtcagcggtttgggcggagaacataccgccttcaaccaactgcccggcggcaaagccgagtatcacggcaaagcattcagctccgacgatgccggcggaaaactgacctataccatagattttgccgccaaacagggacacggcaaaatcgaacacctgaaaacacccgagcaaaatgtcgagcttgccgccgccgaactcaaagcagatgaaaaatcacacgccgtcattttgggcgacacgcgctacggcagcgaagaaaaaggcacttaccacctcgcccttttcggcgaccgcgctcaagaaatcgccggctcggcaaccgtgaagataggagaaaaggttcacgaaatcagcatcgccggcaaacagtag

SEQ ID NO: 70 - protein, 258 aa, Neisseria meningitidis
CGSSGGGGVAADIGAGLADALTAPLDHKDKGLKSLTLEDSISQNGTLTLSAQGAEKTFKVGDKDNSLNTGKLKNDKISRFDFVQKIEVDGQTITLASGEFQIYKQNHSAVVALQIEKINNPDKIDSLINQRSFLVSGLGGEHTAFNQLPGGKAEYHGKAFSSDDAGGKLTYTIDFAAKQGHGKIEHLKTPEQNVELAAAELKADEKSHAVILGDTRYGSEEKGTYHLALFGDRAQEIAGSATVKIGEKVHEISIAGKQ

SEQ ID NO: 71 - DNA, 774 bp, Neisseria meningitidis
atgagcagcggaggcggcggtgtcgccgccgacatcggcgcggggcttgccgatgcactaaccgcaccgctcgaccataaagacaaaggtttgaaatccctgacattggaagactccatttcccaaaacggaacactgaccctgtcggcacaaggtgcggaaaaaactttcaaagtcggcgacaaagacaacagtctcaatacaggcaaattgaagaacgacaaaatcagccgcttcgactttgtgcaaaaaatcgaagtggacggacaaaccatcacgctggcaagcggcgaatttcaaatatacaaacagaaccactccgccgtcgttgccctacagattgaaaaaatcaacaaccccgacaaaatcgacagcctgataaaccaacgctccttccttgtcagcggtttgggcggagaacataccgccttcaaccaactgcccggcggcaaagccgagtatcacggcaaagcattcagctccgacgatgccggcggaaaactgacctataccatagattttgccgccaaacagggacacggcaaaatcgaacacctgaaaacacccgagcaaaatgtcgagcttgccgccgccgaactcaaagcagatgaaaaatcacacgccgtcattttgggcgacacgcgctacggcagcgaagaaaaaggcacttaccacctcgcccttttcggcgaccgcgctcaagaaatcgccggctcggcaaccgtgaagataggagaaaaggttcacgaaatcagcatcgccggcaaacagtag

SEQ ID NO: 72 - protein, 257 aa, Neisseria meningitidis
MSSGGGGVAADIGAGLADALTAPLDHKDKGLKSLTLEDSISQNGTLTLSAQGAEKTFKVGDKDNSLNTGKLKNDKISRFDFVQKIEVDGQTITLASGEFQIYKQNHSAVVALQIEKINNPDKIDSLINQRSFLVSGLGGEHTAFNQLPGGKAEYHGKAFSSDDAGGKLTYTIDFAAKQGHGKIEHLKTPEQNVELAAAELKADEKSHAVILGDTRYGSEEKGTYHLALFGDRAQEIAGSATVKIGEKVHEISIAGKQ

SEQ ID NO: 73 - DNA, 765 bp, Neisseria meningitidis
tgcagcagcggaggcggcggtgtcgccgccgacatcggcgcggggcttgccgatgcactaaccgcaccgctcgaccataaagacaaaagtttgcagtctttgacgctggatcagtccgtcaggaaaaacgagaaactgaagctggcggcacaaggtgcggaaaaaacttatggaaacggcgacagcctcaatacgggcaaattgaagaacgacaaggtcagccgcttcgactttatccgtcaaatcgaagtggacgggcagctcattaccttggagagcggagagttccaaatatacaaacaggaccactccgccgtcgttgccctacagattgaaaaaatcaacaaccccgacaaaatcgacagcctgataaaccaacgctccttccttgtcagcggtttgggtggagaacataccgccttcaaccaactgcccagcggcaaagccgagtatcacggcaaagcattcagctccgacgatgctggcggaaaactgacctataccatagatttcgccgccaaacagggacacggcaaaatcgaacacttgaaaacacccgagcaaaatgtcgagcttgcctccgccgaactcaaagcagatgaaaaatcacacgccgtcattttgggcgacacgcgctacggcggcgaagaaaaaggcacttaccacctcgcccttctcggcgaccgcgcccaagaaatcgccggctcggcaaccgtgaagataagggaaaaggttcacgaaatcggcattgccggcaaacagtag

SEQ ID NO: 74 - protein, 254 aa, Neisseria meningitidis
CSSGGGGVAADIGAGLADALTAPLDHKDKSLQSLTLDQSVRKNEKLKLAAQGAEKTYGNGDSLNTGKLKNDKVSRFDFIRQIEVDGQLITLESGEFQIYKQDHSAVVALQIEKINNPDKIDSLINQRSFLVSGLGGEHTAFNQLPSGKAEYHGKAFSSDDAGGKLTYTIDFAAKQGHGKIEHLKTPEQNVELASAELKADEKSHAVILGDTRYGGEEKGTYHLALLGDRAQEIAGSATVKIREKVHEIGIAGKQ

SEQ ID NO: 75 - DNA, 768 bp, Neisseria meningitidis
tgcggatccagcggaggcggcggtgtcgccgccgacatcggcgcggggcttgccgatgcactaaccgcaccgctcgaccataaagacaaaagtttgcagtctttgacgctggatcagtccgtcaggaaaaacgagaaactgaagctggcggcacaaggtgcggaaaaaacttatggaaacggcgacagcctcaatacgggcaaattgaagaacgacaaggtcagccgcttcgactttatccgtcaaatcgaagtggacgggcagctcattaccttggagagcggagagttccaaatatacaaacaggaccactccgccgtcgttgccctacagattgaaaaaatcaacaaccccgacaaaatcgacagcctgataaaccaacgctccttccttgtcagcggtttgggtggagaacataccgccttcaaccaactgcccagcggcaaagccgagtatcacggcaaagcattcagctccgacgatgctggcggaaaactgacctataccatagatttcgccgccaaacagggacacggcaaaatcgaacacttgaaaacacccgagcaaaatgtcgagcttgcctccgccgaactcaaagcagatgaaaaatcacacgccgtcattttgggcgacacgcgctacggcggcgaagaaaaaggcacttaccacctcgcccttctcggcgaccgcgcccaagaaatcgccggctcggcaaccgtgaagataagggaaaaggttcacgaaatcggcattgccggcaaacagtag

SEQ ID NO: 76 - protein, 255 aa, Neisseria meningitidis
CGSSGGGGVAADIGAGLADALTAPLDHKDKSLQSLTLDQSVRKNEKLKLAAQGAEKTYGNGDSLNTGKLKNDKVSRFDFIRQIEVDGQLITLESGEFQIYKQDHSAVVALQIEKINNPDKIDSLINQRSFLVSGLGGEHTAFNQLPSGKAEYHGKAFSSDDAGGKLTYTIDFAAKQGHGKIEHLKTPEQNVELASAELKADEKSHAVILGDTRYGGEEKGTYHLALLGDRAQEIAGSATVKIREKVHEIGIAGKQ

SEQ ID NO: 77 - DNA, 765 bp, Neisseria meningitidis
atgagcagcggaggcggcggtgtcgccgccgacatcggcgcggggcttgccgatgcactaaccgcaccgctcgaccataaagacaaaagtttgcagtctttgacgctggatcagtccgtcaggaaaaacgagaaactgaagctggcggcacaaggtgcggaaaaaacttatggaaacggcgacagcctcaatacgggcaaattgaagaacgacaaggtcagccgcttcgactttatccgtcaaatcgaagtggacgggcagctcattaccttggagagcggagagttccaaatatacaaacaggaccactccgccgtcgttgccctacagattgaaaaaatcaacaaccccgacaaaatcgacagcctgataaaccaacgctccttccttgtcagcggtttgggtggagaacataccgccttcaaccaactgcccagcggcaaagccgagtatcacggcaaagcattcagctccgacgatgctggcggaaaactgacctataccatagatttcgccgccaaacagggacacggcaaaatcgaacacttgaaaacacccgagcaaaatgtcgagcttgcctccgccgaactcaaagcagatgaaaaatcacacgccgtcattttgggcgacacgcgctacggcggcgaagaaaaaggcacttaccacctcgcccttctcggcgaccgcgcccaagaaatcgccggctcggcaaccgtgaagataagggaaaaggttcacgaaatcggcattgccggcaaacagtag

SEQ ID NO: 78 - protein, 254 aa, Neisseria meningitidis
MSSGGGGVAADIGAGLADALTAPLDHKDKSLQSLTLDQSVRKNEKLKLAAQGAEKTYGNGDSLNTGKLKNDKVSRFDFIRQIEVDGQLITLESGEFQIYKQDHSAVVALQIEKINNPDKIDSLINQRSFLVSGLGGEHTAFNQLPSGKAEYHGKAFSSDDAGGKLTYTIDFAAKQGHGKIEHLKTPEQNVELASAELKADEKSHAVILGDTRYGGEEKGTYHLALLGDRAQEIAGSATVKIREKVHEIGIAGKQ

SEQ ID NO: 79 - DNA, 765 bp, Neisseria meningitidis
tgcagcagcggaggcggcggtgtcgccgccgacatcggcgcggggcttgccgatgcactaaccgcaccgctcgaccataaagacaaaagtttgcagtctttgacgctggatcagtccgtcaggaaaaacgagaaactgaagctggcggcacaaggtgcggaaaaaacttatggaaacggcgacagcctcaatacgggcaaattgaagaacgacaaggtcagccgcttcgactttatccgtcaaatcgaagtggacgggcagctcattaccttggagagcggagagttccaaatatacaaacaggaccactccgccgtcgttgccctacagattgaaaaaatcaacaaccccgacaaaatcgacagcctgataaaccaacgctccttccttgtcagcggtttgggtggagaacataccgccttcaaccaactgcccagcggcaaagccgagtatcacggcaaagcattcagctccgacgatgctggcggaaaactgacctataccatagatttcgccgccaaacagggacacggcaaaatcgaacacttgaaaacacccgagcaaaatgtcgagcttgcctccgccgaactcaaagcagatgaaaaatcacacgccgtcattttgggcgacacgcgctacggcggcgaagaaaaaggcacttaccacctcgcccttttcggcgaccgcgcccaagaaatcgccggctcggcaaccgtgaagataagggaaaaggttcacgaaatcggcatcgccggcaaacagtag

SEQ ID NO: 80 - protein, 254 aa, Neisseria meningitidis
CSSGGGGVAADIGAGLADALTAPLDHKDKSLQSLTLDQSVRKNEKLKLAAQGAEKTYGNGDSLNTGKLKNDKVSRFDFIRQIEVDGQLITLESGEFQIYKQDHSAVVALQIEKINNPDKIDSLINQRSFLVSGLGGEHTAFNQLPSGKAEYHGKAFSSDDAGGKLTYTIDFAAKQGHGKIEHLKTPEQNVELASAELKADEKSHAVILGDTRYGGEEKGTYHLALFGDRAQEIAGSATVKIREKVHEIGIAGKQ

SEQ ID NO: 81 - DNA, 768 bp, Neisseria meningitidis
tgcggatccagcggaggcggcggtgtcgccgccgacatcggcgcggggcttgccgatgcactaaccgcaccgctcgaccataaagacaaaagtttgcagtctttgacgctggatcagtccgtcaggaaaaacgagaaactgaagctggcggcacaaggtgcggaaaaaacttatggaaacggcgacagcctcaatacgggcaaattgaagaacgacaaggtcagccgcttcgactttatccgtcaaatcgaagtggacgggcagctcattaccttggagagcggagagttccaaatatacaaacaggaccactccgccgtcgttgccctacagattgaaaaaatcaacaaccccgacaaaatcgacagcctgataaaccaacgctccttccttgtcagcggtttgggtggagaacataccgccttcaaccaactgcccagcggcaaagccgagtatcacggcaaagcattcagctccgacgatgctggcggaaaactgacctataccatagatttcgccgccaaacagggacacggcaaaatcgaacacttgaaaacacccgagcaaaatgtcgagcttgcctccgccgaactcaaagcagatgaaaaatcacacgccgtcattttgggcgacacgcgctacggcggcgaagaaaaaggcacttaccacctcgcccttttcggcgaccgcgcccaagaaatcgccggctcggcaaccgtgaagataagggaaaaggttcacgaaatcggcatcgccggcaaacagtag

SEQ ID NO: 82 - protein, 255 aa, Neisseria meningitidis
CGSSGGGGVAADIGAGLADALTAPLDHKDKSLQSLTLDQSVRKNEKLKLAAQGAEKTYGNGDSLNTGKLKNDKVSRFDFIRQIEVDGQLITLESGEFQIYKQDHSAVVALQIEKINNPDKIDSLINQRSFLVSGLGGEHTAFNQLPSGKAEYHGKAFSSDDAGGKLTYTIDFAAKQGHGKIEHLKTPEQNVELASAELKADEKSHAVILGDTRYGGEEKGTYHLALFGDRAQEIAGSATVKIREKVHEIGIAGKQ

SEQ ID NO: 83 - DNA, 765 bp, Neisseria meningitidis
atgagcagcggaggcggcggtgtcgccgccgacatcggcgcggggcttgccgatgcactaaccgcaccgctcgaccataaagacaaaagtttgcagtctttgacgctggatcagtccgtcaggaaaaacgagaaactgaagctggcggcacaaggtgcggaaaaaacttatggaaacggcgacagcctcaatacgggcaaattgaagaacgacaaggtcagccgcttcgactttatccgtcaaatcgaagtggacgggcagctcattaccttggagagcggagagttccaaatatacaaacaggaccactccgccgtcgttgccctacagattgaaaaaatcaacaaccccgacaaaatcgacagcctgataaaccaacgctccttccttgtcagcggtttgggtggagaacataccgccttcaaccaactgcccagcggcaaagccgagtatcacggcaaagcattcagctccgacgatgctggcggaaaactgacctataccatagatttcgccgccaaacagggacacggcaaaatcgaacacttgaaaacacccgagcaaaatgtcgagcttgcctccgccgaactcaaagcagatgaaaaatcacacgccgtcattttgggcgacacgcgctacggcggcgaagaaaaaggcacttaccacctcgcccttttcggcgaccgcgcccaagaaatcgccggctcggcaaccgtgaagataagggaaaaggttcacgaaatcggcatcgccggcaaacagtag

SEQ ID NO: 84 - protein, 254 aa, Neisseria meningitidis
MSSGGGGVAADIGAGLADALTAPLDHKDKSLQSLTLDQSVRKNEKLKLAAQGAEKTYGNGDSLNTGKLKNDKVSRFDFIRQIEVDGQLITLESGEFQIYKQDHSAVVALQIEKINNPDKIDSLINQRSFLVSGLGGEHTAFNQLPSGKAEYHGKAFSSDDAGGKLTYTIDFAAKQGHGKIEHLKTPEQNVELASAELKADEKSHAVILGDTRYGGEEKGTYHLALFGDRAQEIAGSATVKIREKVHEIGIAGKQ

SEQ ID NO: 85 - DNA, 765 bp, Neisseria meningitidis; identical to SEQ ID NO: 79

SEQ ID NO: 86 - protein, 254 aa, Neisseria meningitidis; identical to SEQ ID NO: 80

SEQ ID NO: 87 - DNA, 768 bp, Neisseria meningitidis; identical to SEQ ID NO: 81

SEQ ID NO: 88 - protein, 255 aa, Neisseria meningitidis; identical to SEQ ID NO: 82

SEQ ID NO: 89 - DNA, 765 bp, Neisseria meningitidis; identical to SEQ ID NO: 83

SEQ ID NO: 90 - protein, 254 aa, Neisseria meningitidis; identical to SEQ ID NO: 84

SEQ ID NO: 91 - DNA, 765 bp, Neisseria meningitidis; identical to SEQ ID NO: 79

SEQ ID NO: 92 - protein, 254 aa, Neisseria meningitidis; identical to SEQ ID NO: 80

SEQ ID NO: 93 - DNA, 768 bp, Neisseria meningitidis; identical to SEQ ID NO: 81

SEQ ID NO: 94 - protein, 255 aa, Neisseria meningitidis; identical to SEQ ID NO: 82

SEQ ID NO: 95 - DNA, 765 bp, Neisseria meningitidis; identical to SEQ ID NO: 83

SEQ ID NO: 96 - protein, 254 aa, Neisseria meningitidis; identical to SEQ ID NO: 84

SEQ ID NO: 97 - DNA, 765 bp, Neisseria meningitidis
tgcagcagcggaggcggcggtgtcgccgccgacatcggcgcggggcttgccgatgcactaaccgcaccgctcgaccataaagacaaaagtttgcagtctttgacgctggatcagtccgtcaggaaaaacgagaaactgaagctggcggcacaaggtgcggaaaaaacttatggaaacggcgacagcctcaatacgggcaaattgaagaacgacaaggtcagccgcttcgactttatccgtcaaatcgaagtggacgggcagctcattaccttggagagcggagagttccaaatatacaaacaggaccactccgccgtcgttgccctacagattgaaaaaatcaacaaccccgacaaaatcgacagcctgataaaccaacgctccttccttgtcagcggtttgggtggagaacataccgccttcaaccaactgcccagcggcaaagccgagtatcacggcaaagcattcagctccgacgatgctggcggaaaactgacctataccatagatttcgccgccaaacagggacacggcaaaatcgaacacttgaaaacacccgagcaaaatgtcgagcttgcctccgccgaactcaaagcagatgaaaaatcacacgccgtcattttgggcgacacgcgctacggcggcgaagaaaaaggcacttaccacctcgcccttttcggcgaccgcgcccaagaaatcgccggctcggcaaccgtgaagataagggaaaaggttcacgaaatcggcatcgccggcaaacagtaa

SEQ ID NO: 98 - protein, 254 aa, Neisseria meningitidis; identical to SEQ ID NO: 80

SEQ ID NO: 99 - DNA, 768 bp, Neisseria meningitidis
tgcggatccagcggaggcggcggtgtcgccgccgacatcggcgcggggcttgccgatgcactaaccgcaccgctcgaccataaagacaaaagtttgcagtctttgacgctggatcagtccgtcaggaaaaacgagaaactgaagctggcggcacaaggtgcggaaaaaacttatggaaacggcgacagcctcaatacgggcaaattgaagaacgacaaggtcagccgcttcgactttatccgtcaaatcgaagtggacgggcagctcattaccttggagagcggagagttccaaatatacaaacaggaccactccgccgtcgttgccctacagattgaaaaaatcaacaaccccgacaaaatcgacagcctgataaaccaacgctccttccttgtcagcggtttgggtggagaacataccgccttcaaccaactgcccagcggcaaagccgagtatcacggcaaagcattcagctccgacgatgctggcggaaaactgacctataccatagatttcgccgccaaacagggacacggcaaaatcgaacacttgaaaacacccgagcaaaatgtcgagcttgcctccgccgaactcaaagcagatgaaaaatcacacgccgtcattttgggcgacacgcgctacggcggcgaagaaaaaggcacttaccacctcgcccttttcggcgaccgcgcccaagaaatcgccggctcggcaaccgtgaagataagggaaaaggttcacgaaatcggcatcgccggcaaacagtaa

SEQ ID NO: 100 - protein, 255 aa, Neisseria meningitidis; identical to SEQ ID NO: 82

SEQ ID NO: 101 - DNA, 765 bp, Neisseria meningitidis
atgagcagcggaggcggcggtgtcgccgccgacatcggcgcggggcttgccgatgcactaaccgcaccgctcgaccataaagacaaaagtttgcagtctttgacgctggatcagtccgtcaggaaaaacgagaaactgaagctggcggcacaaggtgcggaaaaaacttatggaaacggcgacagcctcaatacgggcaaattgaagaacgacaaggtcagccgcttcgactttatccgtcaaatcgaagtggacgggcagctcattaccttggagagcggagagttccaaatatacaaacaggaccactccgccgtcgttgccctacagattgaaaaaatcaacaaccccgacaaaatcgacagcctgataaaccaacgctccttccttgtcagcggtttgggtggagaacataccgccttcaaccaactgcccagcggcaaagccgagtatcacggcaaagcattcagctccgacgatgctggcggaaaactgacctataccatagatttcgccgccaaacagggacacggcaaaatcgaacacttgaaaacacccgagcaaaatgtcgagcttgcctccgccgaactcaaagcagatgaaaaatcacacgccgtcattttgggcgacacgcgctacggcggcgaagaaaaaggcacttaccacctcgcccttttcggcgaccgcgcccaagaaatcgccggctcggcaaccgtgaagataagggaaaaggttcacgaaatcggcatcgccggcaaacagtaa

SEQ ID NO: 102 - protein, 254 aa, Neisseria meningitidis; identical to SEQ ID NO: 84

SEQ ID NO: 103 - DNA, 765 bp, Neisseria meningitidis
tgcagcagcggaggcggcggtgtcgccgccgacatcggcgcggggcttgccgatgcactaaccgcaccgctcgaccataaagacaaaagtttgcagtctttgacgctggatcagtccgtcaggaaaaacgagaaactgaagctggcggcacaaggtgcggaaaaaacttatggaaacggcgacagccttaatacgggcaaattgaagaacgacaaggtcagccgtttcgactttatccgtcaaatcgaagtggacgggcagctcattaccttggagagcggagagttccaaatatacaaacaggaccactccgccgtcgttgccctacagattgaaaaaatcaacaaccccgacaaaatcgacagcctgataaaccaacgctccttccttgtcagcggtttgggtggagaacataccgccttcaaccaactgcccagcggcaaagccgagtatcacggcaaagcattcagctccgacgatgctggcggaaaactgacctataccatagatttcgccgccaaacagggacacggcaaaatcgaacacttgaaaacacccgagcaaaatgtcgagcttgcctccgccgaactcaaagcagatgaaaaatcacacgccgtcattttgggcgacacgcgctacggcggcgaagaaaaaggcacttaccacctcgcccttttcggcgaccgcgcccaagaaatcgccggctcggcaaccgtgaagataagggaaaaggttcacgaaatcggcatcgccggcaaacagtag

SEQ ID NO: 104 - protein, 254 aa, Neisseria meningitidis; identical to SEQ ID NO: 80

SEQ ID NO: 105 - DNA, 768 bp, Neisseria meningitidis
tgcggatccagcggaggcggcggtgtcgccgccgacatcggcgcggggcttgccgatgcactaaccgcaccgctcgaccataaagacaaaagtttgcagtctttgacgctggatcagtccgtcaggaaaaacgagaaactgaagctggcggcacaaggtgcggaaaaaacttatggaaacggcgacagccttaatacgggcaaattgaagaacgacaaggtcagccgtttcgactttatccgtcaaatcgaagtggacgggcagctcattaccttggagagcggagagttccaaatatacaaacaggaccactccgccgtcgttgccctacagattgaaaaaatcaacaaccccgacaaaatcgacagcctgataaaccaacgctccttccttgtcagcggtttgggtggagaacataccgccttcaaccaactgcccagcggcaaagccgagtatcacggcaaagcattcagctccgacgatgctggcggaaaactgacctataccatagatttcgccgccaaacagggacacggcaaaatcgaacacttgaaaacacccgagcaaaatgtcgagcttgcctccgccgaactcaaagcagatgaaaaatcacacgccgtcattttgggcgacacgcgctacggcggcgaagaaaaaggcacttaccacctcgcccttttcggcgaccgcgcccaagaaatcgccggctcggcaaccgtgaagataagggaaaaggttcacgaaatcggcatcgccggcaaacagtag

SEQ ID NO: 106 - protein, 255 aa, Neisseria meningitidis; identical to SEQ ID NO: 82

SEQ ID NO: 107 - DNA, 765 bp, Neisseria meningitidis
atgagcagcggaggcggcggtgtcgccgccgacatcggcgcggggcttgccgatgcactaaccgcaccgctcgaccataaagacaaaagtttgcagtctttgacgctggatcagtccgtcaggaaaaacgagaaactgaagctggcggcacaaggtgcggaaaaaacttatggaaacggcgacagccttaatacgggcaaattgaagaacgacaaggtcagccgtttcgactttatccgtcaaatcgaagtggacgggcagctcattaccttggagagcggagagttccaaatatacaaacaggaccactccgccgtcgttgccctacagattgaaaaaatcaacaaccccgacaaaatcgacagcctgataaaccaacgctccttccttgtcagcggtttgggtggagaacataccgccttcaaccaactgcccagcggcaaagccgagtatcacggcaaagcattcagctccgacgatgctggcggaaaactgacctataccatagatttcgccgccaaacagggacacggcaaaatcgaacacttgaaaacacccgagcaaaatgtcgagcttgcctccgccgaactcaaagcagatgaaaaatcacacgccgtcattttgggcgacacgcgctacggcggcgaagaaaaaggcacttaccacctcgcccttttcggcgaccgcgcccaagaaatcgccggctcggcaaccgtgaagataagggaaaaggttcacgaaatcggcatcgccggcaaacagtag

SEQ ID NO: 108 - protein, 254 aa, Neisseria meningitidis; identical to SEQ ID NO: 84

SEQ ID NO: 109 - DNA, 765 bp, Neisseria meningitidis
tgcagcagcggaggcggcggtgtcgccgccgacatcggcgcggggcttgccgatgcactaaccgcaccgctcgaccataaagacaaaagtttgcagtctttgacgctggatcagtccgtcaggaaaaacgagaaactgaagctggcggcacaaggtgcggagaaaacttatggaaacggcgacagcctcaatacgggcaaattgaagaacgacaaggtcagccgcttcgactttatccgtcaaatcgaagtggacgggcagctcattaccttggagagcggagagttccaaatatacaaacaggaccactccgccgtcgttgccctacagattgaaaaaatcaacaaccccgacaaaatcgacagcctgataaaccaacgctccttccttgtcagcggtttgggcggagaacataccgccttcaaccaactgcctgacggcaaagccgagtatcacggcaaagcattcagctccgacgatgctggcggaaaactgacctataccatagatttcgccgccaaacagggacacggcaaaatcgaacacctgaaaacacccgagcaaaatgtcgagcttgccgccgccgaactcaaagcagatgaaaaatcacacgccgtcattttgggcgacacgcgctacggcagcgaagaaaaaggcacttaccacctcgcccttttcggcgaccgcgcccaagaaatcgccggctcggcaaccgtgaagataggggaaaaggttcacgaaatcggcatcgccggcaaacagtag

SEQ ID NO: 110 - protein, 254 aa, Neisseria meningitidis
CSSGGGGVAADIGAGLADALTAPLDHKDKSLQSLTLDQSVRKNEKLKLAAQGAEKTYGNGDSLNTGKLKNDKVSRFDFIRQIEVDGQLITLESGEFQIYKQDHSAVVALQIEKINNPDKIDSLINQRSFLVSGLGGEHTAFNQLPDGKAEYHGKAFSSDDAGGKLTYTIDFAAKQGHGKIEHLKTPEQNVELAAAELKADEKSHAVILGDTRYGSEEKGTYHLALFGDRAQEIAGSATVKIGEKVHEIGIAGKQ

SEQ ID NO: 111 - DNA, 768 bp, Neisseria meningitidis
tgcggatccagcggaggcggcggtgtcgccgccgacatcggcgcggggcttgccgatgcactaaccgcaccgctcgaccataaagacaaaagtttgcagtctttgacgctggatcagtccgtcaggaaaaacgagaaactgaagctggcggcacaaggtgcggagaaaacttatggaaacggcgacagcctcaatacgggcaaattgaagaacgacaaggtcagccgcttcgactttatccgtcaaatcgaagtggacgggcagctcattaccttggagagcggagagttccaaatatacaaacaggaccactccgccgtcgttgccctacagattgaaaaaatcaacaaccccgacaaaatcgacagcctgataaaccaacgctccttccttgtcagcggtttgggcggagaacataccgccttcaaccaactgcctgacggcaaagccgagtatcacggcaaagcattcagctccgacgatgctggcggaaaactgacctataccatagatttcgccgccaaacagggacacggcaaaatcgaacacctgaaaacacccgagcaaaatgtcgagcttgccgccgccgaactcaaagcagatgaaaaatcacacgccgtcattttgggcgacacgcgctacggcagcgaagaaaaaggcacttaccacctcgcccttttcggcgaccgcgcccaagaaatcgccggctcggcaaccgtgaagataggggaaaaggttcacgaaatcggcatcgccggcaaacagtag

SEQ ID NO: 112 - protein, 255 aa, Neisseria meningitidis
CGSSGGGGVAADIGAGLADALTAPLDHKDKSLQSLTLDQSVRKNEKLKLAAQGAEKTYGNGDSLNTGKLKNDKVSRFDFIRQIEVDGQLITLESGEFQIYKQDHSAVVALQIEKINNPDKIDSLINQRSFLVSGLGGEHTAFNQLPDGKAEYHGKAFSSDDAGGKLTYTIDFAAKQGHGKIEHLKTPEQNVELAAAELKADEKSHAVILGDTRYGSEEKGTYHLALFGDRAQEIAGSATVKIGEKVHEIGIAGKQ

SEQ ID NO: 113 - DNA, 765 bp, Neisseria meningitidis
atgagcagcggaggcggcggtgtcgccgccgacatcggcgcggggcttgccgatgcactaaccgcaccgctcgaccataaagacaaaagtttgcagtctttgacgctggatcagtccgtcaggaaaaacgagaaactgaagctggcggcacaaggtgcggagaaaacttatggaaacggcgacagcctcaatacgggcaaattgaagaacgacaaggtcagccgcttcgactttatccgtcaaatcgaagtggacgggcagctcattaccttggagagcggagagttccaaatatacaaacaggaccactccgccgtcgttgccctacagattgaaaaaatcaacaaccccgacaaaatcgacagcctgataaaccaacgctccttccttgtcagcggtttgggcggagaacataccgccttcaaccaactgcctgacggcaaagccgagtatcacggcaaagcattcagctccgacgatgctggcggaaaactgacctataccatagatttcgccgccaaacagggacacggcaaaatcgaacacctgaaaacacccgagcaaaatgtcgagcttgccgccgccgaactcaaagcagatgaaaaatcacacgccgtcattttgggcgacacgcgctacggcagcgaagaaaaaggcacttaccacctcgcccttttcggcgaccgcgcccaagaaatcgccggctcggcaaccgtgaagataggggaaaaggttcacgaaatcggcatcgccggcaaacagtag

SEQ ID NO: 114 - protein, 254 aa, Neisseria meningitidis
MSSGGGGVAADIGAGLADALTAPLDHKDKSLQSLTLDQSVRKNEKLKLAAQGAEKTYGNGDSLNTGKLKNDKVSRFDFIRQIEVDGQLITLESGEFQIYKQDHSAVVALQIEKINNPDKIDSLINQRSFLVSGLGGEHTAFNQLPDGKAEYHGKAFSSDDAGGKLTYTIDFAAKQGHGKIEHLKTPEQNVELAAAELKADEKSHAVILGDTRYGSEEKGTYHLALFGDRAQEIAGSATVKIGEKVHEIGIAGKQ

SEQ ID NO: 115 - DNA, 765 bp, Neisseria meningitidis
tgcagcagcggaggcggcggtgtcgccgccgacatcggcgcggggcttgccgatgcactaaccgcaccgctcgaccataaagacaaaagtttgcagtctttgacgctggatcagtccgtcaggaaaaacgagaaactgaagctggcggcacaaggtgcggaaaaaacttatggaaacggcgacagcctcaatacgggcaaattgaagaacgacaaggtcagccgcttcgactttatccgtcaaatcgaagtggacgggcagctcattaccttggagagcggagagttccaaatatacaaacaggaccactccgccgtcgttgccctacagattgaaaaaatcaacaaccccgacaaaatcgacagcctgataaaccaacgctccttccttgtcagcggtttgggcggagaacataccgccttcaaccaactgcctgacggcaaagccgagtatcacggcaaagcattcagctccgacgatgctggcggaaaactgacctataccatagatttcgccgccaaacagggacacggcaaaatcgaacacctgaaaacacccgagcaaaatgtcgagcttgccgccgccgaactcaaagcagatgaaaaatcacacgccgtcattttgggcgacacgcgctacggcagcgaagaaaaaggcacttaccacctcgcccttttcggcgaccgcgcccaagaaatcgccggctcggcaaccgtgaagataggggaaaaggttcacgaaatcggcatcgccggcaaacagtag

SEQ ID NO: 116 - protein, 254 aa, Neisseria meningitidis; identical to SEQ ID NO: 110

SEQ ID NO: 117 - DNA, 768 bp, Neisseria meningitidis
tgcggatccagcggaggcggcggtgtcgccgccgacatcggcgcggggcttgccgatgcactaaccgcaccgctcgaccataaagacaaaagtttgcagtctttgacgctggatcagtccgtcaggaaaaacgagaaactgaagctggcggcacaaggtgcggaaaaaacttatggaaacggcgacagcctcaatacgggcaaattgaagaacgacaaggtcagccgcttcgactttatccgtcaaatcgaagtggacgggcagctcattaccttggagagcggagagttccaaatatacaaacaggaccactccgccgtcgttgccctacagattgaaaaaatcaacaaccccgacaaaatcgacagcctgataaaccaacgctccttccttgtcagcggtttgggcggagaacataccgccttcaaccaactgcctgacggcaaagccgagtatcacggcaaagcattcagctccgacgatgctggcggaaaactgacctataccatagatttcgccgccaaacagggacacggcaaaatcgaacacctgaaaacacccgagcaaaatgtcgagcttgccgccgccgaactcaaagcagatgaaaaatcacacgccgtcattttgggcgacacgcgctacggcagcgaagaaaaaggcacttaccacctcgcccttttcggcgaccgcgcccaagaaatcgccggctcggcaaccgtgaagataggggaaaaggttcacgaaatcggcatcgccggcaaacagtag

SEQ ID NO: 118 - protein, 255 aa, Neisseria meningitidis; identical to SEQ ID NO: 112

SEQ ID NO: 119 - DNA, 765 bp, Neisseria meningitidis
atgagcagcggaggcggcggtgtcgccgccgacatcggcgcggggcttgccgatgcactaaccgcaccgctcgaccataaagacaaaagtttgcagtctttgacgctggatcagtccgtcaggaaaaacgagaaactgaagctggcggcacaaggtgcggaaaaaacttatggaaacggcgacagcctcaatacgggcaaattgaagaacgacaaggtcagccgcttcgactttatccgtcaaatcgaagtggacgggcagctcattaccttggagagcggagagttccaaatatacaaacaggaccactccgccgtcgttgccctacagattgaaaaaatcaacaaccccgacaaaatcgacagcctgataaaccaacgctccttccttgtcagcggtttgggcggagaacataccgccttcaaccaactgcctgacggcaaagccgagtatcacggcaaagcattcagctccgacgatgctggcggaaaactgacctataccatagatttcgccgccaaacagggacacggcaaaatcgaacacctgaaaacacccgagcaaaatgtcgagcttgccgccgccgaactcaaagcagatgaaaaatcacacgccgtcattttgggcgacacgcgctacggcagcgaagaaaaaggcacttaccacctcgcccttttcggcgaccgcgcccaagaaatcgccggctcggcaaccgtgaagataggggaaaaggttcacgaaatcggcatcgccggcaaacagtag

SEQ ID NO: 120 - protein, 254 aa, Neisseria meningitidis; identical to SEQ ID NO: 114

SEQ ID NO: 121 - DNA, 765 bp, Neisseria meningitidis; identical to SEQ ID NO: 115

SEQ ID NO: 122 - protein, 254 aa, Neisseria meningitidis; identical to SEQ ID NO: 110

SEQ ID NO: 123 - DNA, 768 bp, Neisseria meningitidis; identical to SEQ ID NO: 117

SEQ ID NO: 124 - protein, 255 aa, Neisseria meningitidis; identical to SEQ ID NO: 112

SEQ ID NO: 125 - DNA, 765 bp, Neisseria meningitidis; identical to SEQ ID NO: 119

SEQ ID NO: 126 - protein, 254 aa, Neisseria meningitidis; identical to SEQ ID NO: 114

SEQ ID NO: 127 - DNA, 765 bp, Neisseria meningitidis; identical to SEQ ID NO: 115

SEQ ID NO: 128 - protein, 254 aa, Neisseria meningitidis; identical to SEQ ID NO: 110

SEQ ID NO: 129 - DNA, 768 bp, Neisseria meningitidis; identical to SEQ ID NO: 117

SEQ ID NO: 130 - protein, 255 aa, Neisseria meningitidis; identical to SEQ ID NO: 112

SEQ ID NO: 131 - DNA, 765 bp, Neisseria meningitidis; identical to SEQ ID NO: 119

SEQ ID NO: 132 - protein, 254 aa, Neisseria meningitidis; identical to SEQ ID NO: 114

SEQ ID NO: 133 - DNA, 765 bp, Neisseria meningitidis
tgcagcagcggaggcggcggtgtcgccgccgacatcggcgcggggcttgccgatgcactaaccgcaccgctcgaccataaagacaaaagtttgcagtctttgacgctggatcagtccgtcaggaaaaacgagaaactgaagctggcggcacaaggtgcggaaaaaacttatggaaacggcgacagcctcaatacgggcaaattgaagaacgacaaggtcagccgcttcgactttatccgtcaaatcgaagtggacgggcagctcattaccttggagagcggagagttccaaatatacaaacaggaccactccgccgtcgttgccctacagattgaaaaaatcaacaaccccgacaaaatcgacagcctgataaaccaacgctccttccttgtcagcggtttgggcggagaacataccgccttcaaccaactgcctgacggcaaagccgagtatcacggcaaagcattcagctccgacgatgctggcggaaaactgacctataccatagatttcgccgccaaacagggacacggcaaaatcgaacacctgaaaacacccgagcaaaatgtcgagcttgccgccgccgaactcaaagcagatgaaaaatcacacgccgtcattttgggcgacacgcgctacggcagcgaagaaaaaggcacttaccacctcgcccttttcggcgaccgcgcccaagaaatcgccggctcggcaaccgtgaagataggggaaaaggttcacgaaatcggcatcgccggcaaacagtaa

SEQ ID NO: 134 - protein, 254 aa, Neisseria meningitidis; identical to SEQ ID NO: 110

SEQ ID NO: 135 - DNA, 768 bp, Neisseria meningitidis
tgcggatccagcggaggcggcggtgtcgccgccgacatcggcgcggggcttgccgatgcactaaccgcaccgctcgaccataaagacaaaagtttgcagtctttgacgctggatcagtccgtcaggaaaaacgagaaactgaagctggcggcacaaggtgcggaaaaaacttatggaaacggcgacagcctcaatacgggcaaattgaagaacgacaaggtcagccgcttcgactttatccgtcaaatcgaagtggacgggcagctcattaccttggagagcggagagttccaaatatacaaacaggaccactccgccgtcgttgccctacagattgaaaaaatcaacaaccccgacaaaatcgacagcctgataaaccaacgctccttccttgtcagcggtttgggcggagaacataccgccttcaaccaactgcctgacggcaaagccgagtatcacggcaaagcattcagctccgacgatgctggcggaaaactgacctataccatagatttcgccgccaaacagggacacggcaaaatcgaacacctgaaaacacccgagcaaaatgtcgagcttgccgccgccgaactcaaagcagatgaaaaatcacacgccgtcattttgggcgacacgcgctacggcagcgaagaaaaaggcacttaccacctcgcccttttcggcgaccgcgcccaagaaatcgccggctcggcaaccgtgaagataggggaaaaggttcacgaaatcggcatcgccggcaaacagtaa

SEQ ID NO: 136 - protein, 255 aa, Neisseria meningitidis; identical to SEQ ID NO: 112

SEQ ID NO: 137 - DNA, 765 bp, Neisseria meningitidis
atgagcagcggaggcggcggtgtcgccgccgacatcggcgcggggcttgccgatgcactaaccgcaccgctcgaccataaagacaaaagtttgcagtctttgacgctggatcagtccgtcaggaaaaacgagaaactgaagctggcggcacaaggtgcggaaaaaacttatggaaacggcgacagcctcaatacgggcaaattgaagaacgacaaggtcagccgcttcgactttatccgtcaaatcgaagtggacgggcagctcattaccttggagagcggagagttccaaatatacaaacaggaccactccgccgtcgttgccctacagattgaaaaaatcaacaaccccgacaaaatcgacagcctgataaaccaacgctccttccttgtcagcggtttgggcggagaacataccgccttcaaccaactgcctgacggcaaagccgagtatcacggcaaagcattcagctccgacgatgctggcggaaaactgacctataccatagatttcgccgccaaacagggacacggcaaaatcgaacacctgaaaacacccgagcaaaatgtcgagcttgccgccgccgaactcaaagcagatgaaaaatcacacgccgtcattttgggcgacacgcgctacggcagcgaagaaaaaggcacttaccacctcgcccttttcggcgaccgcgcccaagaaatcgccggctcggcaaccgtgaagataggggaaaaggttcacgaaatcggcatcgccggcaaacagtaa

SEQ ID NO: 138 - protein, 254 aa, Neisseria meningitidis; identical to SEQ ID NO: 114

SEQ ID NO: 139 - DNA, 765 bp, Neisseria meningitidis
tgcagcagcggaggcggcggtgtcgccgccgacatcggcgcggggcttgccgatgcactaaccgcaccgctcgaccataaagacaaaagtttgcagtctttgacgctggatcagtccgtcaggaaaaacgagaaactgaagctggcggcacaaggtgcggaaaaaacttatggaaacggcgacagccttaatacgggcaaattgaagaacgacaaggtcagccgtttcgactttatccgtcaaatcgaagtggacgggcagctcattaccttggagagcggagagttccaaatatacaaacaggaccactccgccgtcgttgccctacagattgaaaaaatcaacaaccccgacaaaatcgacagcctgataaaccaacgctccttccttgtcagcggtttgggtggagaacataccgccttcaaccaactgcccagcggcaaagccgagtatcacggcaaagcattcagctccgacgatgctggcggaaaactgacctataccatagatttcgccaccaaacagggacacggcaaaatcgaacacttgaaaacacccgagcaaaatgtcgagcttgccgccgccgaactcaaagcagatgaaaaatcacacgccgtcattttgggcgacacgcgctacggcagcgaagaaaaaggcacttaccacctcgcccttttcggcgaccgcgcccaagaaatcgccggctcggcaaccgtgaagataggggaaaaggttcacgaaatcggcatcgccggcaaacagtag

SEQ ID NO: 140 - protein, 254 aa, Neisseria meningitidis
CSSGGGGVAADIGAGLADALTAPLDHKDKSLQSLTLDQSVRKNEKLKLAAQGAEKTYGNGDSLNTGKLKNDKVSRFDFIRQIEVDGQLITLESGEFQIYKQDHSAVVALQIEKINNPDKIDSLINQRSFLVSGLGGEHTAFNQLPSGKAEYHGKAFSSDDAGGKLTYTIDFATKQGHGKIEHLKTPEQNVELAAAELKADEKSHAVILGDTRYGSEEKGTYHLALFGDRAQEIAGSATVKIGEKVHEIGIAGKQ

SEQ ID NO: 141 - DNA, 768 bp, Neisseria meningitidis
tgcggatccagcggaggcggcggtgtcgccgccgacatcggcgcggggcttgccgatgcactaaccgcaccgctcgaccataaagacaaaagtttgcagtctttgacgctggatcagtccgtcaggaaaaacgagaaactgaagctggcggcacaaggtgcggaaaaaacttatggaaacggcgacagccttaatacgggcaaattgaagaacgacaaggtcagccgtttcgactttatccgtcaaatcgaagtggacgggcagctcattaccttggagagcggagagttccaaatatacaaacaggaccactccgccgtcgttgccctacagattgaaaaaatcaacaaccccgacaaaatcgacagcctgataaaccaacgctccttccttgtcagcggtttgggtggagaacataccgccttcaaccaactgcccagcggcaaagccgagtatcacggcaaagcattcagctccgacgatgctggcggaaaactgacctataccatagatttcgccaccaaacagggacacggcaaaatcgaacacttgaaaacacccgagcaaaatgtcgagcttgccgccgccgaactcaaagcagatgaaaaatcacacgccgtcattttgggcgacacgcgctacggcagcgaagaaaaaggcacttaccacctcgcccttttcggcgaccgcgcccaagaaatcgccggctcggcaaccgtgaagataggggaaaaggttcacgaaatcggcatcgccggcaaacagtag

SEQ ID NO: 142 - protein, 255 aa, Neisseria meningitidis
CGSSGGGGVAADIGAGLADALTAPLDHKDKSLQSLTLDQSVRKNEKLKLAAQGAEKTYGNGDSLNTGKLKNDKVSRFDFIRQIEVDGQLITLESGEFQIYKQDHSAVVALQIEKINNPDKIDSLINQRSFLVSGLGGEHTAFNQLPSGKAEYHGKAFSSDDAGGKLTYTIDFATKQGHGKIEHLKTPEQNVELAAAELKADEKSHAVILGDTRYGSEEKGTYHLALFGDRAQEIAGSATVKIGEKVHEIGIAGKQ

SEQ ID NO: 143 - DNA, 765 bp, Neisseria meningitidis
atgagcagcggaggcggcggtgtcgccgccgacatcggcgcggggcttgccgatgcactaaccgcaccgctcgaccataaagacaaaagtttgcagtctttgacgctggatcagtccgtcaggaaaaacgagaaactgaagctggcggcacaaggtgcggaaaaaacttatggaaacggcgacagccttaatacgggcaaattgaagaacgacaaggtcagccgtttcgactttatccgtcaaatcgaagtggacgggcagctcattaccttggagagcggagagttccaaatatacaaacaggaccactccgccgtcgttgccctacagattgaaaaaatcaacaaccccgacaaaatcgacagcctgataaaccaacgctccttccttgtcagcggtttgggtggagaacataccgccttcaaccaactgcccagcggcaaagccgagtatcacggcaaagcattcagctccgacgatgctggcggaaaactgacctataccatagatttcgccaccaaacagggacacggcaaaatcgaacacttgaaaacacccgagcaaaatgtcgagcttgccgccgccgaactcaaagcagatgaaaaatcacacgccgtcattttgggcgacacgcgctacggcagcgaagaaaaaggcacttaccacctcgcccttttcggcgaccgcgcccaagaaatcgccggctcggcaaccgtgaagataggggaaaaggttcacgaaatcggcatcgccggcaaacagtag

SEQ ID NO: 144 - protein, 254 aa, Neisseria meningitidis
MSSGGGGVAADIGAGLADALTAPLDHKDKSLQSLTLDQSVRKNEKLKLAAQGAEKTYGNGDSLNTGKLKNDKVSRFDFIRQIEVDGQLITLESGEFQIYKQDHSAVVALQIEKINNPDKIDSLINQRSFLVSGLGGEHTAFNQLPSGKAEYHGKAFSSDDAGGKLTYTIDFATKQGHGKIEHLKTPEQNVELAAAELKADEKSHAVILGDTRYGSEEKGTYHLALFGDRAQEIAGSATVKIGEKVHEIGIAGKQ

SEQ ID NO: 145 - DNA, 765 bp, Neisseria meningitidis; identical to SEQ ID NO: 139

SEQ ID NO: 146 - protein, 254 aa, Neisseria meningitidis; identical to SEQ ID NO: 140

SEQ ID NO: 147 - DNA, 768 bp, Neisseria meningitidis; identical to SEQ ID NO: 141

SEQ ID NO: 148 - protein, 255 aa, Neisseria meningitidis; identical to SEQ ID NO: 142

SEQ ID NO: 149 - DNA, 765 bp, Neisseria meningitidis; identical to SEQ ID NO: 143

SEQ ID NO: 150 - protein, 254 aa, Neisseria meningitidis; identical to SEQ ID NO: 144

SEQ ID NO: 151 - DNA, 765 bp, Neisseria meningitidis; identical to SEQ ID NO: 139

SEQ ID NO: 152 - protein, 254 aa, Neisseria meningitidis; identical to SEQ ID NO: 140

SEQ ID NO: 153 - DNA, 768 bp, Neisseria meningitidis; identical to SEQ ID NO: 141

SEQ ID NO: 154 - protein, 255 aa, Neisseria meningitidis; identical to SEQ ID NO: 142

SEQ ID NO: 155 - DNA, 765 bp, Neisseria meningitidis; identical to SEQ ID NO: 143

SEQ ID NO: 156 - protein, 254 aa, Neisseria meningitidis; identical to SEQ ID NO: 144

SEQ ID NO: 157 - DNA, 765 bp, Neisseria meningitidis
tgcagcagcggaggcggcggtgtcgccgccgacatcggcgcggggcttgccgatgcactaaccgcaccgctcgactataaagacaaaagtttgcagtctttgacgctggatcagtccgtcaggaaaaacgagaaactgaagctggcggcacaaggtgcggaaaaaacttatggaaacggcgacagcctcaatacgggcaaattgaagaacgacaaggtcagccgcttcgactttatccgtcaaatcgaagtggacgggcagctcattaccttggagagcggagagttccaaatatacaaacaggaccactccgccgtcgttgccctatagattgaaaaaatcaacaaccccgacaaaatcgacagcctgataaaccaacgctccttccttgtcagcggtttgggtggagaacataccgccttcaaccaactgcccggcggcaaagccgagtatcacggcaaagcattcagctccgacgatgctggcggaaaactgacctataccatagatttcgccgccaaacagggacacggcaaaatcgaacacctgaaaacacccgagcaaaatgtcgagcttgccgccgccgaactcaaagcagatgaaaaatcacacgccgtcattttgggcgacacgcgctacggcagcgaagaaaaaggcacttaccacctcgcccttttcggcgaccgcgctcaagaaatcgccggctcggcaaccgtgaagataggggaaaaggttcacgaaatcggcatcgccggcaaacagtag

SEQ ID NO: 158 - protein, 253 aa, Neisseria meningitidis
CSSGGGGVAADIGAGLADALTAPLDYKDKSLQSLTLDQSVRKNEKLKLAAQGAEKTYGNGDSLNTGKLKNDKVSRFDFIRQIEVDGQLITLESGEFQIYKQDHSAVVALIEKINNPDKIDSLINQRSFLVSGLGGEHTAFNQLPGGKAEYHGKAFSSDDAGGKLTYTIDFAAKQGHGKIEHLKTPEQNVELAAAELKADEKSHAVILGDTRYGSEEKGTYHLALFGDRAQEIAGSATVKIGEKVHEIGIAGKQ

SEQ ID NO: 159 - DNA, 768 bp, Neisseria meningitidis
tgcggatccagcggaggcggcggtgtcgccgccgacatcggcgcggggcttgccgatgcactaaccgcaccgctcgactataaagacaaaagtttgcagtctttgacgctggatcagtccgtcaggaaaaacgagaaactgaagctggcggcacaaggtgcggaaaaaacttatggaaacggcgacagcctcaatacgggcaaattgaagaacgacaaggtcagccgcttcgactttatccgtcaaatcgaagtggacgggcagctcattaccttggagagcggagagttccaaatatacaaacaggaccactccgccgtcgttgccctatagattgaaaaaatcaacaaccccgacaaaatcgacagcctgataaaccaacgctccttccttgtcagcggtttgggtggagaacataccgccttcaaccaactgcccggcggcaaagccgagtatcacggcaaagcattcagctccgacgatgctggcggaaaactgacctataccatagatttcgccgccaaacagggacacggcaaaatcgaacacctgaaaacacccgagcaaaatgtcgagcttgccgccgccgaactcaaagcagatgaaaaatcacacgccgtcattttgggcgacacgcgctacggcagcgaagaaaaaggcacttaccacctcgcccttttcggcgaccgcgctcaagaaatcgccggctcggcaaccgtgaagataggggaaaaggttcacgaaatcggcatcgccggcaaacagtag

SEQ ID NO: 160 - protein, 254 aa, Neisseria meningitidis
CGSSGGGGVAADIGAGLADALTAPLDYKDKSLQSLTLDQSVRKNEKLKLAAQGAEKTYGNGDSLNTGKLKNDKVSRFDFIRQIEVDGQLITLESGEFQIYKQDHSAVVALIEKINNPDKIDSLINQRSFLVSGLGGEHTAFNQLPGGKAEYHGKAFSSDDAGGKLTYTIDFAAKQGHGKIEHLKTPEQNVELAAAELKADEKSHAVILGDTRYGSEEKGTYHLALFGDRAQEIAGSATVKIGEKVHEIGIAGKQ

SEQ ID NO: 161 - DNA, 765 bp, Neisseria meningitidis
atgagcagcggaggcggcggtgtcgccgccgacatcggcgcggggcttgccgatgcactaaccgcaccgctcgactataaagacaaaagtttgcagtctttgacgctggatcagtccgtcaggaaaaacgagaaactgaagctggcggcacaaggtgcggaaaaaacttatggaaacggcgacagcctcaatacgggcaaattgaagaacgacaaggtcagccgcttcgactttatccgtcaaatcgaagtggacgggcagctcattaccttggagagcggagagttccaaatatacaaacaggaccactccgccgtcgttgccctatagattgaaaaaatcaacaaccccgacaaaatcgacagcctgataaaccaacgctccttccttgtcagcggtttgggtggagaacataccgccttcaaccaactgcccggcggcaaagccgagtatcacggcaaagcattcagctccgacgatgctggcggaaaactgacctataccatagatttcgccgccaaacagggacacggcaaaatcgaacacctgaaaacacccgagcaaaatgtcgagcttgccgccgccgaactcaaagcagatgaaaaatcacacgccgtcattttgggcgacacgcgctacggcagcgaagaaaaaggcacttaccacctcgcccttttcggcgaccgcgctcaagaaatcgccggctcggcaaccgtgaagataggggaaaaggttcacgaaatcggcatcgccggcaaacagtag

SEQ ID NO: 162 - protein, 253 aa, Neisseria meningitidis
MSSGGGGVAADIGAGLADALTAPLDYKDKSLQSLTLDQSVRKNEKLKLAAQGAEKTYGNGDSLNTGKLKNDKVSRFDFIRQIEVDGQLITLESGEFQIYKQDHSAVVALIEKINNPDKIDSLINQRSFLVSGLGGEHTAFNQLPGGKAEYHGKAFSSDDAGGKLTYTIDFAAKQGHGKIEHLKTPEQNVELAAAELKADEKSHAVILGDTRYGSEEKGTYHLALFGDRAQEIAGSATVKIGEKVHEIGIAGKQ

SEQ ID NO: 163 - DNA, 765 bp, Neisseria meningitidis
tgcagcagcggaggcggcggtgtcgccgccgacatcggcgcggggcttgccgatgcactaaccgcaccgctcgaccataaagacaaaagtttgcagtctttgacgctggatcagtccgtcaggaaaaacgagaaactgaagctggcggcacaaggtgcggaaaaaacttatggaaacggcgacagcctcaatacgggcaaattgaagaacgacaaggtcagccgcttcgactttatccgtcaaatcgaagtggacgggcagctcattaccttggagagcggagagttccaaatatacaaacaggaccactccgccgtcgttgccctacagattgaaaaaatcaacaaccccgacaaaatcgacagcctgataaaccgacgctccttccttgtcagcggtttgggcggagaacataccgccttcaaccaactgcctgacggcaaagccgagtatcacggcaaagcattcagctccgacgatgctggcggaaaactgacctataccatagatttcgccgccaaacagggacacggcaaaatcgaacacctgaaaacacccgagcaaaatgtcgagcttgccgccgccgaactcaaagcagatgaaaaatcacacgccgtcattttgggcgacacgcgctacggcagcgaagaaaaaggcacttaccacctcgcccttttcggcgaccgcgcccaagaaatcgccggctcggcaaccgtgaagataggggaaaaggttcacgaaatcggcatcgccggcaaacagtag

SEQ ID NO: 164 - protein, 254 aa, Neisseria meningitidis
CSSGGGGVAADIGAGLADALTAPLDHKDKSLQSLTLDQSVRKNEKLKLAAQGAEKTYGNGDSLNTGKLKNDKVSRFDFIRQIEVDGQLITLESGEFQIYKQDHSAVVALQIEKINNPDKIDSLINRRSFLVSGLGGEHTAFNQLPDGKAEYHGKAFSSDDAGGKLTYTIDFAAKQGHGKIEHLKTPEQNVELAAAELKADEKSHAVILGDTRYGSEEKGTYHLALFGDRAQEIAGSATVKIGEKVHEIGIAGKQ

SEQ ID NO: 165 - DNA, 768 bp, Neisseria meningitidis
tgcggatccagcggaggcggcggtgtcgccgccgacatcggcgcggggcttgccgatgcactaaccgcaccgctcgaccataaagacaaaagtttgcagtctttgacgctggatcagtccgtcaggaaaaacgagaaactgaagctggcggcacaaggtgcggaaaaaacttatggaaacggcgacagcctcaatacgggcaaattgaagaacgacaaggtcagccgcttcgactttatccgtcaaatcgaagtggacgggcagctcattaccttggagagcggagagttccaaatatacaaacaggaccactccgccgtcgttgccctacagattgaaaaaatcaacaaccccgacaaaatcgacagcctgataaaccgacgctccttccttgtcagcggtttgggcggagaacataccgccttcaaccaactgcctgacggcaaagccgagtatcacggcaaagcattcagctccgacgatgctggcggaaaactgacctataccatagatttcgccgccaaacagggacacggcaaaatcgaacacctgaaaacacccgagcaaaatgtcgagcttgccgccgccgaactcaaagcagatgaaaaatcacacgccgtcattttgggcgacacgcgctacggcagcgaagaaaaaggcacttaccacctcgcccttttcggcgaccgcgcccaagaaatcgccggctcggcaaccgtgaagataggggaaaaggttcacgaaatcggcatcgccggcaaacagtag

SEQ ID NO: 166 - protein, 255 aa, Neisseria meningitidis
CGSSGGGGVAADIGAGLADALTAPLDHKDKSLQSLTLDQSVRKNEKLKLAAQGAEKTYGNGDSLNTGKLKNDKVSRFDFIRQIEVDGQLITLESGEFQIYKQDHSAVVALQIEKINNPDKIDSLINRRSFLVSGLGGEHTAFNQLPDGKAEYHGKAFSSDDAGGKLTYTIDFAAKQGHGKIEHLKTPEQNVELAAAELKADEKSHAVILGDTRYGSEEKGTYHLALFGDRAQEIAGSATVKIGEKVHEIGIAGKQ

SEQ ID NO: 167 - DNA, 765 bp, Neisseria meningitidis
atgagcagcggaggcggcggtgtcgccgccgacatcggcgcggggcttgccgatgcactaaccgcaccgctcgaccataaagacaaaagtttgcagtctttgacgctggatcagtccgtcaggaaaaacgagaaactgaagctggcggcacaaggtgcggaaaaaacttatggaaacggcgacagcctcaatacgggcaaattgaagaacgacaaggtcagccgcttcgactttatccgtcaaatcgaagtggacgggcagctcattaccttggagagcggagagttccaaatatacaaacaggaccactccgccgtcgttgccctacagattgaaaaaatcaacaaccccgacaaaatcgacagcctgataaaccgacgctccttccttgtcagcggtttgggcggagaacataccgccttcaaccaactgcctgacggcaaagccgagtatcacggcaaagcattcagctccgacgatgctggcggaaaactgacctataccatagatttcgccgccaaacagggacacggcaaaatcgaacacctgaaaacacccgagcaaaatgtcgagcttgccgccgccgaactcaaagcagatgaaaaatcacacgccgtcattttgggcgacacgcgctacggcagcgaagaaaaaggcacttaccacctcgcccttttcggcgaccgcgcccaagaaatcgccggctcggcaaccgtgaagataggggaaaaggttcacgaaatcggcatcgccggcaaacagtag

SEQ ID NO: 168 - protein, 254 aa, Neisseria meningitidis
MSSGGGGVAADIGAGLADALTAPLDHKDKSLQSLTLDQSVRKNEKLKLAAQGAEKTYGNGDSLNTGKLKNDKVSRFDFIRQIEVDGQLITLESGEFQIYKQDHSAVVALQIEKINNPDKIDSLINRRSFLVSGLGGEHTAFNQLPDGKAEYHGKAFSSDDAGGKLTYTIDFAAKQGHGKIEHLKTPEQNVELAAAELKADEKSHAVILGDTRYGSEEKGTYHLALFGDRAQEIAGSATVKIGEKVHEIGIAGKQ

SEQ ID NO: 169 - DNA, 765 bp, Neisseria meningitidis
tgcagcagcggaggcggcggtgtcgccgccgacatcggcgcggggcttgccgatgcactaaccgcaccgctcgaccataaagacaaaagtttgcagtctttgacgctggatcagtccgtcaggaaaaacgagaaactgaagctggcggcacaaggtgcggaaaaaacttatggaaacggcgacagcctcaatacgggcaaattgaagaacgacaaggtcagccgcttcgactttatccgtcaaatcgaagtggacgggcagctcattaccttggagagcggagagttccaaatatacaaacaggaccactccgccgtcgttgccctacagattgaaaaaatcaacaaccccgacaaaatcgacagcctgataaaccaacgctccttccttgtcagcggtttgggcggagaacataccgccttcaaccaactgcctgacggcaaagccgagtatcacggcaaagcattcagctccgacgatgctggcggaaaactgacctataccatagatttcgccgccaaacagggacacggcaaaatcgaacacctgaaaacacccgagcaaaatgtcgagcttgccgccgccgaactcaaagcagatgaaaaatcacacgccgtcattttgggcgacacgcgctacggcagcgaagaaaaaggcacttaccacctcgcccttttcggcgaccgcgcccaagaaatcgccggctcggcaaccgtggagataggggaaaaggttcacgaaatcggcatcgccggcaaacagtag

SEQ ID NO: 170 - protein, 254 aa, Neisseria meningitidis
CSSGGGGVAADIGAGLADALTAPLDHKDKSLQSLTLDQSVRKNEKLKLAAQGAEKTYGNGDSLNTGKLKNDKVSRFDFIRQIEVDGQLITLESGEFQIYKQDHSAVVALQIEKINNPDKIDSLINQRSFLVSGLGGEHTAFNQLPDGKAEYHGKAFSSDDAGGKLTYTIDFAAKQGHGKIEHLKTPEQNVELAAAELKADEKSHAVILGDTRYGSEEKGTYHLALFGDRAQEIAGSATVEIGEKVHEIGIAGKQ

SEQ ID NO: 171 - DNA, 768 bp, Neisseria meningitidis
tgcggatccagcggaggcggcggtgtcgccgccgacatcggcgcggggcttgccgatgcactaaccgcaccgctcgaccataaagacaaaagtttgcagtctttgacgctggatcagtccgtcaggaaaaacgagaaactgaagctggcggcacaaggtgcggaaaaaacttatggaaacggcgacagcctcaatacgggcaaattgaagaacgacaaggtcagccgcttcgactttatccgtcaaatcgaagtggacgggcagctcattaccttggagagcggagagttccaaatatacaaacaggaccactccgccgtcgttgccctacagattgaaaaaatcaacaaccccgacaaaatcgacagcctgataaaccaacgctccttccttgtcagcggtttgggcggagaacataccgccttcaaccaactgcctgacggcaaagccgagtatcacggcaaagcattcagctccgacgatgctggcggaaaactgacctataccatagatttcgccgccaaacagggacacggcaaaatcgaacacctgaaaacacccgagcaaaatgtcgagcttgccgccgccgaactcaaagcagatgaaaaatcacacgccgtcattttgggcgacacgcgctacggcagcgaagaaaaaggcacttaccacctcgcccttttcggcgaccgcgcccaagaaatcgccggctcggcaaccgtggagataggggaaaaggttcacgaaatcggcatcgccggcaaacagtag

SEQ ID NO: 172 - protein, 255 aa, Neisseria meningitidis
CGSSGGGGVAADIGAGLADALTAPLDHKDKSLQSLTLDQSVRKNEKLKLAAQGAEKTYGNGDSLNTGKLKNDKVSRFDFIRQIEVDGQLITLESGEFQIYKQDHSAVVALQIEKINNPDKIDSLINQRSFLVSGLGGEHTAFNQLPDGKAEYHGKAFSSDDAGGKLTYTIDFAAKQGHGKIEHLKTPEQNVELAAAELKADEKSHAVILGDTRYGSEEKGTYHLALFGDRAQEIAGSATVEIGEKVHEIGIAGKQ

SEQ ID NO: 173 - DNA, 765 bp, Neisseria meningitidis
atgagcagcggaggcggcggtgtcgccgccgacatcggcgcggggcttgccgatgcactaaccgcaccgctcgaccataaagacaaaagtttgcagtctttgacgctggatcagtccgtcaggaaaaacgagaaactgaagctggcggcacaaggtgcggaaaaaacttatggaaacggcgacagcctcaatacgggcaaattgaagaacgacaaggtcagccgcttcgactttatccgtcaaatcgaagtggacgggcagctcattaccttggagagcggagagttccaaatatacaaacaggaccactccgccgtcgttgccctacagattgaaaaaatcaacaaccccgacaaaatcgacagcctgataaaccaacgctccttccttgtcagcggtttgggcggagaacataccgccttcaaccaactgcctgacggcaaagccgagtatcacggcaaagcattcagctccgacgatgctggcggaaaactgacctataccatagatttcgccgccaaacagggacacggcaaaatcgaacacctgaaaacacccgagcaaaatgtcgagcttgccgccgccgaactcaaagcagatgaaaaatcacacgccgtcattttgggcgacacgcgctacggcagcgaagaaaaaggcacttaccacctcgcccttttcggcgaccgcgcccaagaaatcgccggctcggcaaccgtggagataggggaaaaggttcacgaaatcggcatcgccggcaaacagtag

SEQ ID NO: 174 - protein, 254 aa, Neisseria meningitidis
MSSGGGGVAADIGAGLADALTAPLDHKDKSLQSLTLDQSVRKNEKLKLAAQGAEKTYGNGDSLNTGKLKNDKVSRFDFIRQIEVDGQLITLESGEFQIYKQDHSAVVALQIEKINNPDKIDSLINQRSFLVSGLGGEHTAFNQLPDGKAEYHGKAFSSDDAGGKLTYTIDFAAKQGHGKIEHLKTPEQNVELAAAELKADEKSHAVILGDTRYGSEEKGTYHLALFGDRAQEIAGSATVEIGEKVHEIGIAGKQ

SEQ ID NO: 175 - DNA, 768 bp, Neisseria meningitidis
tgcagcagcggaggcggcggtgtcgccgccgacatcggcgcggtgcttgccgatgcactaaccgcaccgctcgaccataaagacaaaagtttgcagtctttgacgctggatcagtccgtcaggaaaaacgagaaactgaagctggcggcacaaggtgcggaaaaaacttatggaaacggcgacagcctcaatacgggcaaattgaagaacgacaaggtcagccgcttcgactttatccgtcaaatcgaagtggacgggcagctcattaccttggagagcggagagttccaagtgtacaaacaaagccattccgccttaaccgcccttcagaccgagcaagtacaagattcggagcattcagggaagatggttgcgaaacgccagttcagaatcggcgatatagcgggtgaacatacatcttttgacaagcttcccgaaggcggcagggcgacatatcgcgggacggcattcggttcagacgatgccagtggaaaactgacctacaccatagatttcgccgccaagcagggacacggcaaaatcgaacatttgaaatcgccagaactcaatgttgacctggccgcctccgatatcaagccggataaaaaacgccatgccgtcatcagcggttccgtcctttacaaccaagccgagaaaggcagttactctctaggcatctttggcgggcaagcccaggaagttgccggcagcgcagaagtggaaaccgcaaacggcatacgccatatcggtcttgccgccaagcagtaa

SEQ ID NO: 176 - protein, 255 aa, Neisseria meningitidis
CSSGGGGVAADIGAVLADALTAPLDHKDKSLQSLTLDQSVRKNEKLKLAAQGAEKTYGNGDSLNTGKLKNDKVSRFDFIRQIEVDGQLITLESGEFQVYKQSHSALTALQTEQVQDSEHSGKMVAKRQFRIGDIAGEHTSFDKLPEGGRATYRGTAFGSDDASGKLTYTIDFAAKQGHGKIEHLKSPELNVDLAASDIKPDKKRHAVISGSVLYNQAEKGSYSLGIFGGQAQEVAGSAEVETANGIRHIGLAAKQ

SEQ ID NO: 177 - DNA, 771 bp, Neisseria meningitidis
tgcggatccagcggaggcggcggtgtcgccgccgacatcggcgcggtgcttgccgatgcactaaccgcaccgctcgaccataaagacaaaagtttgcagtctttgacgctggatcagtccgtcaggaaaaacgagaaactgaagctggcggcacaaggtgcggaaaaaacttatggaaacggcgacagcctcaatacgggcaaattgaagaacgacaaggtcagccgcttcgactttatccgtcaaatcgaagtggacgggcagctcattaccttggagagcggagagttccaagtgtacaaacaaagccattccgccttaaccgcccttcagaccgagcaagtacaagattcggagcattcagggaagatggttgcgaaacgccagttcagaatcggcgatatagcgggtgaacatacatcttttgacaagcttcccgaaggcggcagggcgacatatcgcgggacggcattcggttcagacgatgccagtggaaaactgacctacaccatagatttcgccgccaagcagggacacggcaaaatcgaacatttgaaatcgccagaactcaatgttgacctggccgcctccgatatcaagccggataaaaaacgccatgccgtcatcagcggttccgtcctttacaaccaagccgagaaaggcagttactctctaggcatctttggcgggcaagcccaggaagttgccggcagcgcagaagtggaaaccgcaaacggcatacgccatatcggtcttgccgccaagcagtaa

SEQ ID NO: 178 - protein, 256 aa, Neisseria meningitidis
CGSSGGGGVAADIGAVLADALTAPLDHKDKSLQSLTLDQSVRKNEKLKLAAQGAEKTYGNGDSLNTGKLKNDKVSRFDFIRQIEVDGQLITLESGEFQVYKQSHSALTALQTEQVQDSEHSGKMVAKRQFRIGDIAGEHTSFDKLPEGGRATYRGTAFGSDDASGKLTYTIDFAAKQGHGKIEHLKSPELNVDLAASDIKPDKKRHAVISGSVLYNQAEKGSYSLGIFGGQAQEVAGSAEVETANGIRHIGLAAKQ

SEQ ID NO: 179 - DNA, 768 bp, Neisseria meningitidis
atgagcagcggaggcggcggtgtcgccgccgacatcggcgcggtgcttgccgatgcactaaccgcaccgctcgaccataaagacaaaagtttgcagtctttgacgctggatcagtccgtcaggaaaaacgagaaactgaagctggcggcacaaggtgcggaaaaaacttatggaaacggcgacagcctcaatacgggcaaattgaagaacgacaaggtcagccgcttcgactttatccgtcaaatcgaagtggacgggcagctcattaccttggagagcggagagttccaagtgtacaaacaaagccattccgccttaaccgcccttcagaccgagcaagtacaagattcggagcattcagggaagatggttgcgaaacgccagttcagaatcggcgatatagcgggtgaacatacatcttttgacaagcttcccgaaggcggcagggcgacatatcgcgggacggcattcggttcagacgatgccagtggaaaactgacctacaccatagatttcgccgccaagcagggacacggcaaaatcgaacatttgaaatcgccagaactcaatgttgacctggccgcctccgatatcaagccggataaaaaacgccatgccgtcatcagcggttccgtcctttacaaccaagccgagaaaggcagttactctctaggcatctttggcgggcaagcccaggaagttgccggcagcgcagaagtggaaaccgcaaacggcatacgccatatcggtcttgccgccaagcagtaa

SEQ ID NO: 180 - protein, 255 aa, Neisseria meningitidis
MSSGGGGVAADIGAVLADALTAPLDHKDKSLQSLTLDQSVRKNEKLKLAAQGAEKTYGNGDSLNTGKLKNDKVSRFDFIRQIEVDGQLITLESGEFQVYKQSHSALTALQTEQVQDSEHSGKMVAKRQFRIGDIAGEHTSFDKLPEGGRATYRGTAFGSDDASGKLTYTIDFAAKQGHGKIEHLKSPELNVDLAASDIKPDKKRHAVISGSVLYNQAEKGSYSLGIFGGQAQEVAGSAEVETANGIRHIGLAAKQ

SEQ ID NO: 181 - DNA, 747 bp, Neisseria meningitidis
tgcagcagcggagggggtggtgtcgccgccgacatcggtgcggggcttgccgatgcactaaccgcaccgctcgaccataaagacaaaggtttgcagtctttgacgctggatcagtccgtcaggaaaaacgagaaactgaagctggcggcacaaggtgcggaaaaaacttatggaaacggtgacagcctcaatacgggcaaattgaagaacgacaaggtcagccgtttcgactttatccgccaaatcgaagtggacgggcagctcattaccttggagagtggagagttccaagtatacaaacaaagccattccgccttaaccgcctttcagaccgagcaaatacaagattcggagcattccgggaagatggttgcgaaacgccagttcagaatcggcgacatagcgggcgaacatacatcttttgacaagcttcccgaaggcggcagggcgacatatcgcgggacggcgttcggttcagacgatgccggcggaaaactgacctacaccatagatttcgccgccaagcagggaaacggcaaaatcgaacatttgaaatcgccagaactcaatgtcgacctggccgccgccgatatcaagccggatggaaaacgccatgccgtcatcagcggttccgtcctttacaaccaagccgagaaaggcagttactccctcggtatctttggcggaaaagcccaggaagttgccggcagcgcggaagtgaaaaccgtaaacggcatacgccatatc

SEQ ID NO: 182 - protein, 249 aa, Neisseria meningitidis
CSSGGGGVAADIGAGLADALTAPLDHKDKGLQSLTLDQSVRKNEKLKLAAQGAEKTYGNGDSLNTGKLKNDKVSRFDFIRQIEVDGQLITLESGEFQVYKQSHSALTAFQTEQIQDSEHSGKMVAKRQFRIGDIAGEHTSFDKLPEGGRATYRGTAFGSDDAGGKLTYTIDFAAKQGNGKIEHLKSPELNVDLAAADIKPDGKRHAVISGSVLYNQAEKGSYSLGIFGGKAQEVAGSAEVKTVNGIRHI

SEQ ID NO: 183 - DNA, 750 bp, Neisseria meningitidis
tgcggatccagcggagggggtggtgtcgccgccgacatcggtgcggggcttgccgatgcactaaccgcaccgctcgaccataaagacaaaggtttgcagtctttgacgctggatcagtccgtcaggaaaaacgagaaactgaagctggcggcacaaggtgcggaaaaaacttatggaaacggtgacagcctcaatacgggcaaattgaagaacgacaaggtcagccgtttcgactttatccgccaaatcgaagtggacgggcagctcattaccttggagagtggagagttccaagtatacaaacaaagccattccgccttaaccgcctttcagaccgagcaaatacaagattcggagcattccgggaagatggttgcgaaacgccagttcagaatcggcgacatagcgggcgaacatacatcttttgacaagcttcccgaaggcggcagggcgacatatcgcgggacggcgttcggttcagacgatgccggcggaaaactgacctacaccatagatttcgccgccaagcagggaaacggcaaaatcgaacatttgaaatcgccagaactcaatgtcgacctggccgccgccgatatcaagccggatggaaaacgccatgccgtcatcagcggttccgtcctttacaaccaagccgagaaaggcagttactccctcggtatctttggcggaaaagcccaggaagttgccggcagcgcggaagtgaaaaccgtaaacggcatacgccatatc

SEQ ID NO: 184 - protein, 250 aa, Neisseria meningitidis
CGSSGGGGVAADIGAGLADALTAPLDHKDKGLQSLTLDQSVRKNEKLKLAAQGAEKTYGNGDSLNTGKLKNDKVSRFDFIRQIEVDGQLITLESGEFQVYKQSHSALTAFQTEQIQDSEHSGKMVAKRQFRIGDIAGEHTSFDKLPEGGRATYRGTAFGSDDAGGKLTYTIDFAAKQGNGKIEHLKSPELNVDLAAADIKPDGKRHAVISGSVLYNQAEKGSYSLGIFGGKAQEVAGSAEVKTVNGIRHI

SEQ ID NO: 185 - DNA, 747 bp, Neisseria meningitidis
atgagcagcggagggggtggtgtcgccgccgacatcggtgcggggcttgccgatgcactaaccgcaccgctcgaccataaagacaaaggtttgcagtctttgacgctggatcagtccgtcaggaaaaacgagaaactgaagctggcggcacaaggtgcggaaaaaacttatggaaacggtgacagcctcaatacgggcaaattgaagaacgacaaggtcagccgtttcgactttatccgccaaatcgaagtggacgggcagctcattaccttggagagtggagagttccaagtatacaaacaaagccattccgccttaaccgcctttcagaccgagcaaatacaagattcggagcattccgggaagatggttgcgaaacgccagttcagaatcggcgacatagcgggcgaacatacatcttttgacaagcttcccgaaggcggcagggcgacatatcgcgggacggcgttcggttcagacgatgccggcggaaaactgacctacaccatagatttcgccgccaagcagggaaacggcaaaatcgaacatttgaaatcgccagaactcaatgtcgacctggccgccgccgatatcaagccggatggaaaacgccatgccgtcatcagcggttccgtcctttacaaccaagccgagaaaggcagttactccctcggtatctttggcggaaaagcccaggaagttgccggcagcgcggaagtgaaaaccgtaaacggcatacgccatatc

SEQ ID NO: 186 - protein, 249 aa, Neisseria meningitidis
MSSGGGGVAADIGAGLADALTAPLDHKDKGLQSLTLDQSVRKNEKLKLAAQGAEKTYGNGDSLNTGKLKNDKVSRFDFIRQIEVDGQLITLESGEFQVYKQSHSALTAFQTEQIQDSEHSGKMVAKRQFRIGDIAGEHTSFDKLPEGGRATYRGTAFGSDDAGGKLTYTIDFAAKQGNGKIEHLKSPELNVDLAAADIKPDGKRHAVISGSVLYNQAEKGSYSLGIFGGKAQEVAGSAEVKTVNGIRHI

SEQ ID NO: 187 - DNA, 747 bp, Neisseria meningitidis; identical to SEQ ID NO: 181

SEQ ID NO: 188 - protein, 249 aa, Neisseria meningitidis; identical to SEQ ID NO: 182

SEQ ID NO: 189 - DNA, 750 bp, Neisseria meningitidis; identical to SEQ ID NO: 183

SEQ ID NO: 190 - protein, 250 aa, Neisseria meningitidis; identical to SEQ ID NO: 184

SEQ ID NO: 191 - DNA, 747 bp, Neisseria meningitidis; identical to SEQ ID NO: 185

SEQ ID NO: 192 - protein, 249 aa, Neisseria meningitidis; identical to SEQ ID NO: 186

SEQ ID NO: 193 - DNA, 768 bp, Neisseria meningitidis
tgcagcagcggagggggtggtgtcgccgccgacatcggtgcggggcttgccgatgcactaaccgcaccgctcgaccataaagacaaaggtttgcagtctttgacgctggatcagtccgtcaggaaaaacgagaaactgaagctggcggcacaaggtgcggaaaaaacttatggaaacggtgacagcctcaatacgggcaaattgaagaacgacaaggtcagccgtttcgactttatccgccaaatcgaagtggacgggcagctcattaccttggagagtggagagttccaagtatacaaacaaagccattccgccttaaccgcctttcagaccgagcaaatacaagattcggagcattccgggaagatggttgcgaaacgccagttcagaatcggcgacatagcgggcgaacatacatcttttgacaagcttcccgaaggcggcagggcgacatatcgcgggacggcgttcggttcagacgatgccggcggaaaactgacctacaccatagatttcgccgccaagcagggaaacggcaaaatcgaacatttgaaatcgccagaactcaatgtcgacctggccgccgccgatatcaagccggatggaaaacgccatgccgtcatcagcggttccgtcctttacaaccaagccgagaaaggcagttactccctcggtatctttggcggaaaagcccaggaagttgccggcagcgcggaagtgaaaaccgtaaacggcatacgccatatcggccttgccgccaagcaataa

SEQ ID NO: 194 - protein, 255 aa, Neisseria meningitidis
CSSGGGGVAADIGAGLADALTAPLDHKDKGLQSLTLDQSVRKNEKLKLAAQGAEKTYGNGDSLNTGKLKNDKVSRFDFIRQIEVDGQLITLESGEFQVYKQSHSALTAFQTEQIQDSEHSGKMVAKRQFRIGDIAGEHTSFDKLPEGGRATYRGTAFGSDDAGGKLTYTIDFAAKQGNGKIEHLKSPELNVDLAAADIKPDGKRHAVISGSVLYNQAEKGSYSLGIFGGKAQEVAGSAEVKTVNGIRHIGLAAKQ

SEQ ID NO: 195 - DNA, 771 bp, Neisseria meningitidis
tgcggatccagcggagggggtggtgtcgccgccgacatcggtgcggggcttgccgatgcactaaccgcaccgctcgaccataaagacaaaggtttgcagtctttgacgctggatcagtccgtcaggaaaaacgagaaactgaagctggcggcacaaggtgcggaaaaaacttatggaaacggtgacagcctcaatacgggcaaattgaagaacgacaaggtcagccgtttcgactttatccgccaaatcgaagtggacgggcagctcattaccttggagagtggagagttccaagtatacaaacaaagccattccgccttaaccgcctttcagaccgagcaaatacaagattcggagcattccgggaagatggttgcgaaacgccagttcagaatcggcgacatagcgggcgaacatacatcttttgacaagcttcccgaaggcggcagggcgacatatcgcgggacggcgttcggttcagacgatgccggcggaaaactgacctacaccatagatttcgccgccaagcagggaaacggcaaaatcgaacatttgaaatcgccagaactcaatgtcgacctggccgccgccgatatcaagccggatggaaaacgccatgccgtcatcagcggttccgtcctttacaaccaagccgagaaaggcagttactccctcggtatctttggcggaaaagcccaggaagttgccggcagcgcggaagtgaaaaccgtaaacggcatacgccatatcggccttgccgccaagcaataa

SEQ ID NO: 196 - protein, 256 aa, Neisseria meningitidis
CGSSGGGGVAADIGAGLADALTAPLDHKDKGLQSLTLDQSVRKNEKLKLAAQGAEKTYGNGDSLNTGKLKNDKVSRFDFIRQIEVDGQLITLESGEFQVYKQSHSALTAFQTEQIQDSEHSGKMVAKRQFRIGDIAGEHTSFDKLPEGGRATYRGTAFGSDDAGGKLTYTIDFAAKQGNGKIEHLKSPELNVDLAAADIKPDGKRHAVISGSVLYNQAEKGSYSLGIFGGKAQEVAGSAEVKTVNGIRHIGLAAKQ

SEQ ID NO: 197 - DNA, 768 bp, Neisseria meningitidis
atgagcagcggagggggtggtgtcgccgccgacatcggtgcggggcttgccgatgcactaaccgcaccgctcgaccataaagacaaaggtttgcagtctttgacgctggatcagtccgtcaggaaaaacgagaaactgaagctggcggcacaaggtgcggaaaaaacttatggaaacggtgacagcctcaatacgggcaaattgaagaacgacaaggtcagccgtttcgactttatccgccaaatcgaagtggacgggcagctcattaccttggagagtggagagttccaagtatacaaacaaagccattccgccttaaccgcctttcagaccgagcaaatacaagattcggagcattccgggaagatggttgcgaaacgccagttcagaatcggcgacatagcgggcgaacatacatcttttgacaagcttcccgaaggcggcagggcgacatatcgcgggacggcgttcggttcagacgatgccggcggaaaactgacctacaccatagatttcgccgccaagcagggaaacggcaaaatcgaacatttgaaatcgccagaactcaatgtcgacctggccgccgccgatatcaagccggatggaaaacgccatgccgtcatcagcggttccgtcctttacaaccaagccgagaaaggcagttactccctcggtatctttggcggaaaagcccaggaagttgccggcagcgcggaagtgaaaaccgtaaacggcatacgccatatcggccttgccgccaagcaataa

SEQ ID NO: 198 - protein, 255 aa, Neisseria meningitidis
MSSGGGGVAADIGAGLADALTAPLDHKDKGLQSLTLDQSVRKNEKLKLAAQGAEKTYGNGDSLNTGKLKNDKVSRFDFIRQIEVDGQLITLESGEFQVYKQSHSALTAFQTEQIQDSEHSGKMVAKRQFRIGDIAGEHTSFDKLPEGGRATYRGTAFGSDDAGGKLTYTIDFAAKQGNGKIEHLKSPELNVDLAAADIKPDGKRHAVISGSVLYNQAEKGSYSLGIFGGKAQEVAGSAEVKTVNGIRHIGLAAKQ

SEQ ID NO: 199 - DNA, 768 bp, Neisseria meningitidis; identical to SEQ ID NO: 193

SEQ ID NO: 200 - protein, 255 aa, Neisseria meningitidis; identical to SEQ ID NO: 194

SEQ ID NO: 201 - DNA, 771 bp, Neisseria meningitidis; identical to SEQ ID NO: 195

SEQ ID NO: 202 - protein, 256 aa, Neisseria meningitidis; identical to SEQ ID NO: 196

SEQ ID NO: 203 - DNA, 768 bp, Neisseria meningitidis; identical to SEQ ID NO: 197

SEQ ID NO: 204 - protein, 255 aa, Neisseria meningitidis; identical to SEQ ID NO: 198

SEQ ID NO: 205 - DNA, 768 bp, Neisseria meningitidis; identical to SEQ ID NO: 193

SEQ ID NO: 206 - protein, 255 aa, Neisseria meningitidis; identical to SEQ ID NO: 194

SEQ ID NO: 207 - DNA, 771 bp, Neisseria meningitidis; identical to SEQ ID NO: 195

SEQ ID NO: 208 - protein, 256 aa, Neisseria meningitidis; identical to SEQ ID NO: 196

SEQ ID NO: 209 - DNA, 768 bp, Neisseria meningitidis; identical to SEQ ID NO: 197

SEQ ID NO: 210 - protein, 255 aa, Neisseria meningitidis; identical to SEQ ID NO: 198

SEQ ID NO: 211 - DNA, 768 bp, Neisseria meningitidis
tgcagcagcggagggggtggtgtcgccgccgacatcggtgcggggcttgccgatgcactaaccgcaccgctcgaccataaagacaaaggtttgcagtctttgacgctggatcagtccgtcaggaaaaacgagaaactgaagctggcggcacaaggtgcggaaaaaacttatggaaacggtgacagcctcaatacgggcaaattgaagaacgacaaggtcagccgtttcgactttatccgccaaatcgaagtggacgggcagctcattaccttggagagtggagagttccaagtatacaaacaaagccattccgccttaaccgcctttcagaccgagcaaatacaagattcggagcattccgggaagatggttgcgaaacgccagttcagaatcggcgacatagcgggcgaacatacatcttttgacaagcttcccgaaggcggcagggcgacatatcgcgggacggcgttcggttcagacgatgccggcggaaaactgacctacaccatagatttcgccgccaagcagggaaacggcaaaatcgaacatttgaaatcgccagaactcaatgtcgacctggccgccgccgatatcaagccggatggaaaacgccatgccgtcatcagcggttccgtcctttacaaccaagccgagaaaggcagttactccctcggtatctttggcggaaaagcccaggaagttgccggcagcgcggaagtgaaaaccgtaaacggcatacgccatatcggtcttgccgccaagcagtaa

SEQ ID NO: 212 - protein, 255 aa, Neisseria meningitidis; identical to SEQ ID NO: 194

SEQ ID NO: 213 - DNA, 771 bp, Neisseria meningitidis
tgcggatccagcggagggggtggtgtcgccgccgacatcggtgcggggcttgccgatgcactaaccgcaccgctcgaccataaagacaaaggtttgcagtctttgacgctggatcagtccgtcaggaaaaacgagaaactgaagctggcggcacaaggtgcggaaaaaacttatggaaacggtgacagcctcaatacgggcaaattgaagaacgacaaggtcagccgtttcgactttatccgccaaatcgaagtggacgggcagctcattaccttggagagtggagagttccaagtatacaaacaaagccattccgccttaaccgcctttcagaccgagcaaatacaagattcggagcattccgggaagatggttgcgaaacgccagttcagaatcggcgacatagcgggcgaacatacatcttttgacaagcttcccgaaggcggcagggcgacatatcgcgggacggcgttcggttcagacgatgccggcggaaaactgacctacaccatagatttcgccgccaagcagggaaacggcaaaatcgaacatttgaaatcgccagaactcaatgtcgacctggccgccgccgatatcaagccggatggaaaacgccatgccgtcatcagcggttccgtcctttacaaccaagccgagaaaggcagttactccctcggtatctttggcggaaaagcccaggaagttgccggcagcgcggaagtgaaaaccgtaaacggcatacgccatatcggtcttgccgccaagcagtaa

SEQ ID NO: 214 - protein, 256 aa, Neisseria meningitidis; identical to SEQ ID NO: 196

SEQ ID NO: 215 - DNA, 768 bp, Neisseria meningitidis
atgagcagcggagggggtggtgtcgccgccgacatcggtgcggggcttgccgatgcactaaccgcaccgctcgaccataaagacaaaggtttgcagtctttgacgctggatcagtccgtcaggaaaaacgagaaactgaagctggcggcacaaggtgcggaaaaaacttatggaaacggtgacagcctcaatacgggcaaattgaagaacgacaaggtcagccgtttcgactttatccgccaaatcgaagtggacgggcagctcattaccttggagagtggagagttccaagtatacaaacaaagccattccgccttaaccgcctttcagaccgagcaaatacaagattcggagcattccgggaagatggttgcgaaacgccagttcagaatcggcgacatagcgggcgaacatacatcttttgacaagcttcccgaaggcggcagggcgacatatcgcgggacggcgttcggttcagacgatgccggcggaaaactgacctacaccatagatttcgccgccaagcagggaaacggcaaaatcgaacatttgaaatcgccagaactcaatgtcgacctggccgccgccgatatcaagccggatggaaaacgccatgccgtcatcagcggttccgtcctttacaaccaagccgagaaaggcagttactccctcggtatctttggcggaaaagcccaggaagttgccggcagcgcggaagtgaaaaccgtaaacggcatacgccatatcggtcttgccgccaagcagtaa

SEQ ID NO: 216 - protein, 255 aa, Neisseria meningitidis; identical to SEQ ID NO: 198

SEQ ID NO: 217 - DNA, 768 bp, Neisseria meningitidis
tgcagcagcggagggggcggtgtcgccgccgacatcggtgcggggcttgccgatgcactaaccgcaccgctcgaccataaagacaaaggtttgcagtctttgacgctggatcagtccgtcaggaaaaacgagaaactgaagctggcggcacaaggtgcggaaaaaacttatggaaacggcgacagcctcaatacgggcaaattgaagaacgacaaggtcagccgcttcgactttatccgtcaaatcgaagtggacaggcagctcattaccttggagagcggagagttccaagtgtacaaacaaagccattccgccttaaccgcccttcagaccgagcaagtacaagactcggagcattccgggaagatggttgcgaaacgtcagttcagaatcggcgacatagcgggtgaacatacatcttttgacaagcttcccgaaggcggcagggcgacatatcgcgggacggcgttcggttcagacgatgccggcggaaaactgatttacaccatagatttcgccgctaagcagggacacggtaaaatcgaacatttgaaatcgccagaactcaatgtcgacctggccgccgccgatatcaagccggatgaaaaacaccatgccgtcatcagcggttccgtcctttacaaccaagccgagaaaggcagttactccctcggtatctttggcggaaaagcccaggaagttgccggcagcgcggaagtgaaaaccgtaaacggtatacgccatatcggccttgccgccaagcaataa

SEQ ID NO: 218 - protein, 255 aa, Neisseria meningitidis
CSSGGGGVAADIGAGLADALTAPLDHKDKGLQSLTLDQSVRKNEKLKLAAQGAEKTYGNGDSLNTGKLKNDKVSRFDFIRQIEVDRQLITLESGEFQVYKQSHSALTALQTEQVQDSEHSGKMVAKRQFRIGDIAGEHTSFDKLPEGGRATYRGTAFGSDDAGGKLIYTIDFAAKQGHGKIEHLKSPELNVDLAAADIKPDEKHHAVISGSVLYNQAEKGSYSLGIFGGKAQEVAGSAEVKTVNGIRHIGLAAKQ

SEQ ID NO: 219 - DNA, 771 bp, Neisseria meningitidis
tgcggatccagcggagggggcggtgtcgccgccgacatcggtgcggggcttgccgatgcactaaccgcaccgctcgaccataaagacaaaggtttgcagtctttgacgctggatcagtccgtcaggaaaaacgagaaactgaagctggcggcacaaggtgcggaaaaaacttatggaaacggcgacagcctcaatacgggcaaattgaagaacgacaaggtcagccgcttcgactttatccgtcaaatcgaagtggacaggcagctcattaccttggagagcggagagttccaagtgtacaaacaaagccattccgccttaaccgcccttcagaccgagcaagtacaagactcggagcattccgggaagatggttgcgaaacgtcagttcagaatcggcgacatagcgggtgaacatacatcttttgacaagcttcccgaaggcggcagggcgacatatcgcgggacggcgttcggttcagacgatgccggcggaaaactgatttacaccatagatttcgccgctaagcagggacacggtaaaatcgaacatttgaaatcgccagaactcaatgtcgacctggccgccgccgatatcaagccggatgaaaaacaccatgccgtcatcagcggttccgtcctttacaaccaagccgagaaaggcagttactccctcggtatctttggcggaaaagcccaggaagttgccggcagcgcggaagtgaaaaccgtaaacggtatacgccatatcggccttgccgccaagcaataa

SEQ ID NO: 220 - protein, 256 aa, Neisseria meningitidis
CGSSGGGGVAADIGAGLADALTAPLDHKDKGLQSLTLDQSVRKNEKLKLAAQGAEKTYGNGDSLNTGKLKNDKVSRFDFIRQIEVDRQLITLESGEFQVYKQSHSALTALQTEQVQDSEHSGKMVAKRQFRIGDIAGEHTSFDKLPEGGRATYRGTAFGSDDAGGKLIYTIDFAAKQGHGKIEHLKSPELNVDLAAADIKPDEKHHAVISGSVLYNQAEKGSYSLGIFGGKAQEVAGSAEVKTVNGIRHIGLAAKQ

SEQ ID NO: 221 - DNA, 768 bp, Neisseria meningitidis
atgagcagcggagggggcggtgtcgccgccgacatcggtgcggggcttgccgatgcactaaccgcaccgctcgaccataaagacaaaggtttgcagtctttgacgctggatcagtccgtcaggaaaaacgagaaactgaagctggcggcacaaggtgcggaaaaaacttatggaaacggcgacagcctcaatacgggcaaattgaagaacgacaaggtcagccgcttcgactttatccgtcaaatcgaagtggacaggcagctcattaccttggagagcggagagttccaagtgtacaaacaaagccattccgccttaaccgcccttcagaccgagcaagtacaagactcggagcattccgggaagatggttgcgaaacgtcagttcagaatcggcgacatagcgggtgaacatacatcttttgacaagcttcccgaaggcggcagggcgacatatcgcgggacggcgttcggttcagacgatgccggcggaaaactgatttacaccatagatttcgccgctaagcagggacacggtaaaatcgaacatttgaaatcgccagaactcaatgtcgacctggccgccgccgatatcaagccggatgaaaaacaccatgccgtcatcagcggttccgtcctttacaaccaagccgagaaaggcagttactccctcggtatctttggcggaaaagcccaggaagttgccggcagcgcggaagtgaaaaccgtaaacggtatacgccatatcggccttgccgccaagcaataa

SEQ ID NO: 222 - protein, 255 aa, Neisseria meningitidis
MSSGGGGVAADIGAGLADALTAPLDHKDKGLQSLTLDQSVRKNEKLKLAAQGAEKTYGNGDSLNTGKLKNDKVSRFDFIRQIEVDRQLITLESGEFQVYKQSHSALTALQTEQVQDSEHSGKMVAKRQFRIGDIAGEHTSFDKLPEGGRATYRGTAFGSDDAGGKLIYTIDFAAKQGHGKIEHLKSPELNVDLAAADIKPDEKHHAVISGSVLYNQAEKGSYSLGIFGGKAQEVAGSAEVKTVNGIRHIGLAAKQ

SEQ ID NO: 223 - DNA, 768 bp, Neisseria meningitidis
tgcagcagcggagggggcggtgtcgccgccgacatcggtgcggggcttgccgatgcactaaccgcaccgctcgaccataaagacaaaggtttgcagtctttaacgctggatcagtccgtcaggaaaaacgagaaactgaagctggcggcacaaggtgcggaaaaaacttatggaaacggcgacagccttaatacgggcaaattgaagaacgacaaggtcagccgcttcgactttatccgtcaaatcgaagtggacgggaagctcattaccttggagagcggagagttccaagtgtacaaacaaagccattccgccttaaccgcccttcagaccgagcaagtacaagactcggaggattccgggaagatggttgcgaaacgccagttcagaatcggcgacatagcgggcgaacatacatcttttgacaagcttcccaaaggcggcagtgcgacatatcgcgggacggcgttcggttcagacgatgctggcggaaaactgacctatactatagatttcgccgccaagcagggacacggcaaaatcgaacatttgaaatcgcccgaactcaatgtcgagcttgccaccgcctatatcaagccggatgaaaaacgccatgccgttatcagcggttccgtcctttacaaccaagacgagaaaggcagttactccctcggtatctttggcgggcaagcccaggaagttgccggcagcgcggaagtggaaaccgcaaacggcatacaccatatcggtcttgccgccaagcagtaa

SEQ ID NO: 224 - protein, 255 aa, Neisseria meningitidis
CSSGGGGVAADIGAGLADALTAPLDHKDKGLQSLTLDQSVRKNEKLKLAAQGAEKTYGNGDSLNTGKLKNDKVSRFDFIRQIEVDGKLITLESGEFQVYKQSHSALTALQTEQVQDSEDSGKMVAKRQFRIGDIAGEHTSFDKLPKGGSATYRGTAFGSDDAGGKLTYTIDFAAKQGHGKIEHLKSPELNVELATAYIKPDEKRHAVISGSVLYNQDEKGSYSLGIFGGQAQEVAGSAEVETANGIHHIGLAAKQ

SEQ ID NO: 225 - DNA, 771 bp, Neisseria meningitidis
tgcggatccagcggagggggcggtgtcgccgccgacatcggtgcggggcttgccgatgcactaaccgcaccgctcgaccataaagacaaaggtttgcagtctttaacgctggatcagtccgtcaggaaaaacgagaaactgaagctggcggcacaaggtgcggaaaaaacttatggaaacggcgacagccttaatacgggcaaattgaagaacgacaaggtcagccgcttcgactttatccgtcaaatcgaagtggacgggaagctcattaccttggagagcggagagttccaagtgtacaaacaaagccattccgccttaaccgcccttcagaccgagcaagtacaagactcggaggattccgggaagatggttgcgaaacgccagttcagaatcggcgacatagcgggcgaacatacatcttttgacaagcttcccaaaggcggcagtgcgacatatcgcgggacggcgttcggttcagacgatgctggcggaaaactgacctatactatagatttcgccgccaagcagggacacggcaaaatcgaacatttgaaatcgcccgaactcaatgtcgagcttgccaccgcctatatcaagccggatgaaaaacgccatgccgttatcagcggttccgtcctttacaaccaagacgagaaaggcagttactccctcggtatctttggcgggcaagcccaggaagttgccggcagcgcggaagtggaaaccgcaaacggcatacaccatatcggtcttgccgccaagcagtaa

SEQ ID NO: 226 - protein, 256 aa, Neisseria meningitidis
CGSSGGGGVAADIGAGLADALTAPLDHKDKGLQSLTLDQSVRKNEKLKLAAQGAEKTYGNGDSLNTGKLKNDKVSRFDFIRQIEVDGKLITLESGEFQVYKQSHSALTALQTEQVQDSEDSGKMVAKRQFRIGDIAGEHTSFDKLPKGGSATYRGTAFGSDDAGGKLTYTIDFAAKQGHGKIEHLKSPELNVELATAYIKPDEKRHAVISGSVLYNQDEKGSYSLGIFGGQAQEVAGSAEVETANGIHHIGLAAKQ

SEQ ID NO: 227 - DNA, 768 bp, Neisseria meningitidis
atgagcagcggagggggcggtgtcgccgccgacatcggtgcggggcttgccgatgcactaaccgcaccgctcgaccataaagacaaaggtttgcagtctttaacgctggatcagtccgtcaggaaaaacgagaaactgaagctggcggcacaaggtgcggaaaaaacttatggaaacggcgacagccttaatacgggcaaattgaagaacgacaaggtcagccgcttcgactttatccgtcaaatcgaagtggacgggaagctcattaccttggagagcggagagttccaagtgtacaaacaaagccattccgccttaaccgcccttcagaccgagcaagtacaagactcggaggattccgggaagatggttgcgaaacgccagttcagaatcggcgacatagcgggcgaacatacatcttttgacaagcttcccaaaggcggcagtgcgacatatcgcgggacggcgttcggttcagacgatgctggcggaaaactgacctatactatagatttcgccgccaagcagggacacggcaaaatcgaacatttgaaatcgcccgaactcaatgtcgagcttgccaccgcctatatcaagccggatgaaaaacgccatgccgttatcagcggttccgtcctttacaaccaagacgagaaaggcagttactccctcggtatctttggcgggcaagcccaggaagttgccggcagcgcggaagtggaaaccgcaaacggcatacaccatatcggtcttgccgccaagcagtaa

SEQ ID NO: 228 - protein, 255 aa, Neisseria meningitidis
MSSGGGGVAADIGAGLADALTAPLDHKDKGLQSLTLDQSVRKNEKLKLAAQGAEKTYGNGDSLNTGKLKNDKVSRFDFIRQIEVDGKLITLESGEFQVYKQSHSALTALQTEQVQDSEDSGKMVAKRQFRIGDIAGEHTSFDKLPKGGSATYRGTAFGSDDAGGKLTYTIDFAAKQGHGKIEHLKSPELNVELATAYIKPDEKRHAVISGSVLYNQDEKGSYSLGIFGGQAQEVAGSAEVETANGIHHIGLAAKQ

SEQ ID NO: 229 - DNA, 768 bp, Neisseria meningitidis
tgcagcagcggaggcggcggtgtcgccgccgacatcggcgcggggcttgccgatgcactaaccgcaccgctcgaccataaagacaaaagtttgcagtctttgacgctggatcagtccgtcaggaaaaacgagaaactgaagctggcggcacaaggtgcggaaaaaacttatggaaacggcgacagccttaatacgggcaaattgaagaacgacaaggtcagccgtttcgactttatccgtcaaatcgaagtggacgggcagctcattaccttggagagcggagagttccaagtgtacaaacaaagccattccgccttaaccgcccttcagaccgagcaagaacaagatccagagcattccgggaagatggttgcgaaacgccggttcaaaatcggcgacatagcgggcgaacatacatcttttgacaagcttcccaaagacgtcatggcgacatatcgcgggacggcgttcggttcagacgatgccggcggaaaactgacctatactatagattttgctgccaaacagggacacggcaaaatcgaacatttgaaatcgcccgaactcaatgtcgagcttgccaccgcctatatcaagccggatgaaaaacaccatgccgtcatcagcggttccgtcctttacaatcaagacgagaaaggcagttactccctcggtatctttggcgggcaagcccaggaagttgccggcagcgcggaagtggaaaccgcaaacggcatacaccatatcggtcttgccgccaagcaataa

SEQ ID NO: 230 - protein, 255 aa, Neisseria meningitidis
CSSGGGGVAADIGAGLADALTAPLDHKDKSLQSLTLDQSVRKNEKLKLAAQGAEKTYGNGDSLNTGKLKNDKVSRFDFIRQIEVDGQLITLESGEFQVYKQSHSALTALQTEQEQDPEHSGKMVAKRRFKIGDIAGEHTSFDKLPKDVMATYRGTAFGSDDAGGKLTYTIDFAAKQGHGKIEHLKSPELNVELATAYIKPDEKHHAVISGSVLYNQDEKGSYSLGIFGGQAQEVAGSAEVETANGIHHIGLAAKQ

SEQ ID NO: 231 - DNA, 771 bp, Neisseria meningitidis
tgcggatccagcggaggcggcggtgtcgccgccgacatcggcgcggggcttgccgatgcactaaccgcaccgctcgaccataaagacaaaagtttgcagtctttgacgctggatcagtccgtcaggaaaaacgagaaactgaagctggcggcacaaggtgcggaaaaaacttatggaaacggcgacagccttaatacgggcaaattgaagaacgacaaggtcagccgtttcgactttatccgtcaaatcgaagtggacgggcagctcattaccttggagagcggagagttccaagtgtacaaacaaagccattccgccttaaccgcccttcagaccgagcaagaacaagatccagagcattccgggaagatggttgcgaaacgccggttcaaaatcggcgacatagcgggcgaacatacatcttttgacaagcttcccaaagacgtcatggcgacatatcgcgggacggcgttcggttcagacgatgccggcggaaaactgacctatactatagattttgctgccaaacagggacacggcaaaatcgaacatttgaaatcgcccgaactcaatgtcgagcttgccaccgcctatatcaagccggatgaaaaacaccatgccgtcatcagcggttccgtcctttacaatcaagacgagaaaggcagttactccctcggtatctttggcgggcaagcccaggaagttgccggcagcgcggaagtggaaaccgcaaacggcatacaccatatcggtcttgccgccaagcaataa

SEQ ID NO: 232 - protein, 256 aa, Neisseria meningitidis
CGSSGGGGVAADIGAGLADALTAPLDHKDKSLQSLTLDQSVRKNEKLKLAAQGAEKTYGNGDSLNTGKLKNDKVSRFDFIRQIEVDGQLITLESGEFQVYKQSHSALTALQTEQEQDPEHSGKMVAKRRFKIGDIAGEHTSFDKLPKDVMATYRGTAFGSDDAGGKLTYTIDFAAKQGHGKIEHLKSPELNVELATAYIKPDEKHHAVISGSVLYNQDEKGSYSLGIFGGQAQEVAGSAEVETANGIHHIGLAAKQ

SEQ ID NO: 233 - DNA, 768 bp, Neisseria meningitidis
atgagcagcggaggcggcggtgtcgccgccgacatcggcgcggggcttgccgatgcactaaccgcaccgctcgaccataaagacaaaagtttgcagtctttgacgctggatcagtccgtcaggaaaaacgagaaactgaagctggcggcacaaggtgcggaaaaaacttatggaaacggcgacagccttaatacgggcaaattgaagaacgacaaggtcagccgtttcgactttatccgtcaaatcgaagtggacgggcagctcattaccttggagagcggagagttccaagtgtacaaacaaagccattccgccttaaccgcccttcagaccgagcaagaacaagatccagagcattccgggaagatggttgcgaaacgccggttcaaaatcggcgacatagcgggcgaacatacatcttttgacaagcttcccaaagacgtcatggcgacatatcgcgggacggcgttcggttcagacgatgccggcggaaaactgacctatactatagattttgctgccaaacagggacacggcaaaatcgaacatttgaaatcgcccgaactcaatgtcgagcttgccaccgcctatatcaagccggatgaaaaacaccatgccgtcatcagcggttccgtcctttacaatcaagacgagaaaggcagttactccctcggtatctttggcgggcaagcccaggaagttgccggcagcgcggaagtggaaaccgcaaacggcatacaccatatcggtcttgccgccaagcaataa

SEQ ID NO: 234 - protein, 255 aa, Neisseria meningitidis
MSSGGGGVAADIGAGLADALTAPLDHKDKSLQSLTLDQSVRKNEKLKLAAQGAEKTYGNGDSLNTGKLKNDKVSRFDFIRQIEVDGQLITLESGEFQVYKQSHSALTALQTEQEQDPEHSGKMVAKRRFKIGDIAGEHTSFDKLPKDVMATYRGTAFGSDDAGGKLTYTIDFAAKQGHGKIEHLKSPELNVELATAYIKPDEKHHAVISGSVLYNQDEKGSYSLGIFGGQAQEVAGSAEVETANGIHHIGLAAKQ

SEQ ID NO: 235 - DNA, 768 bp, Neisseria meningitidis; identical to SEQ ID NO: 229

SEQ ID NO: 236 - protein, 255 aa, Neisseria meningitidis; identical to SEQ ID NO: 230

SEQ ID NO: 237 - DNA, 771 bp, Neisseria meningitidis; identical to SEQ ID NO: 231

SEQ ID NO: 238 - protein, 256 aa, Neisseria meningitidis; identical to SEQ ID NO: 232

SEQ ID NO: 239 - DNA, 768 bp, Neisseria meningitidis; identical to SEQ ID NO: 233

SEQ ID NO: 240 - protein, 255 aa, Neisseria meningitidis; identical to SEQ ID NO: 234

SEQ ID NO: 241 - DNA, 765 bp, Neisseria meningitidis
tgcagcagcggaggcggcggtgtcgccgccgacatcggcgcggggcttgccgatgcactaaccgcaccgctcgaccataaagacaaaagtttgcagtctttgacgctggatcagtccgtcaggaaaaacgagaaactgaagctggcggcacaaggtgcggaaaaaacttatggaaacggcgacagccttaatacgggcaaattgaagaacgacaaggtcagccgtttcgactttatccgtcaaatcgaagtggacgggcagctcattaccttggagagcggagagttccaagtgtacaaacaaagccattccgccttaaccgcccttcagaccgagcaagaacaagatccagagcattccgggaagatggttgcgaaacgccggttcaaaatcggcgacatagcgggcgaacatacatcttttgacaagcttcccaaagacgtcatggcgacatatcgcgggacggcgttcggttcagacgatgccggcggaaaactgacctatactatagattttgctgccaaacagggacacggcaaaatcgaacatttgaaatcgcccgaactcaatgtcgagcttgccaccgcctatatcaagccggatgaaaaacaccatgccgtcatcagcggttccgtcctttacaatcaagacgagaaaggcagttactccctcggtatctttggcgggcaagcccaggaagttgccggcagcgcggaagtggaaaccgcaaacggcatacaccatatcggtcttgccgccaagcaa

SEQ ID NO: 242 - protein, 255 aa, Neisseria meningitidis; identical to SEQ ID NO: 230

SEQ ID NO: 243 - DNA, 768 bp, Neisseria meningitidis
tgcggatccagcggaggcggcggtgtcgccgccgacatcggcgcggggcttgccgatgcactaaccgcaccgctcgaccataaagacaaaagtttgcagtctttgacgctggatcagtccgtcaggaaaaacgagaaactgaagctggcggcacaaggtgcggaaaaaacttatggaaacggcgacagccttaatacgggcaaattgaagaacgacaaggtcagccgtttcgactttatccgtcaaatcgaagtggacgggcagctcattaccttggagagcggagagttccaagtgtacaaacaaagccattccgccttaaccgcccttcagaccgagcaagaacaagatccagagcattccgggaagatggttgcgaaacgccggttcaaaatcggcgacatagcgggcgaacatacatcttttgacaagcttcccaaagacgtcatggcgacatatcgcgggacggcgttcggttcagacgatgccggcggaaaactgacctatactatagattttgctgccaaacagggacacggcaaaatcgaacatttgaaatcgcccgaactcaatgtcgagcttgccaccgcctatatcaagccggatgaaaaacaccatgccgtcatcagcggttccgtcctttacaatcaagacgagaaaggcagttactccctcggtatctttggcgggcaagcccaggaagttgccggcagcgcggaagtggaaaccgcaaacggcatacaccatatcggtcttgccgccaagcaa

SEQ ID NO: 244 - protein, 256 aa, Neisseria meningitidis; identical to SEQ ID NO: 232

SEQ ID NO: 245 - DNA, 765 bp, Neisseria meningitidis
atgagcagcggaggcggcggtgtcgccgccgacatcggcgcggggcttgccgatgcactaaccgcaccgctcgaccataaagacaaaagtttgcagtctttgacgctggatcagtccgtcaggaaaaacgagaaactgaagctggcggcacaaggtgcggaaaaaacttatggaaacggcgacagccttaatacgggcaaattgaagaacgacaaggtcagccgtttcgactttatccgtcaaatcgaagtggacgggcagctcattaccttggagagcggagagttccaagtgtacaaacaaagccattccgccttaaccgcccttcagaccgagcaagaacaagatccagagcattccgggaagatggttgcgaaacgccggttcaaaatcggcgacatagcgggcgaacatacatcttttgacaagcttcccaaagacgtcatggcgacatatcgcgggacggcgttcggttcagacgatgccggcggaaaactgacctatactatagattttgctgccaaacagggacacggcaaaatcgaacatttgaaatcgcccgaactcaatgtcgagcttgccaccgcctatatcaagccggatgaaaaacaccatgccgtcatcagcggttccgtcctttacaatcaagacgagaaaggcagttactccctcggtatctttggcgggcaagcccaggaagttgccggcagcgcggaagtggaaaccgcaaacggcatacaccatatcggtcttgccgccaagcaa

SEQ ID NO: 246 - protein, 255 aa, Neisseria meningitidis; identical to SEQ ID NO: 234

SEQ ID NO: 247 - DNA, 783 bp, Neisseria meningitidis
tgcagcagcggaggcggcggaagcggaggcggcggtgtcaccgccgacatcggcacggggcttgccgatgcactaactgcgccgctcgaccataaagacaaaggcttgaaatccctgacattggaagactccatttcccaaaacggaacactgaccctgtcggcacaaggtgcggaaaaaacttatggaaacggcgacagccttaatacgggcaaattgaagaacgacaaggtcagccgtttcgactttatccgtcaaatcgaagtggacgggcagctcattaccttggagagcggagagttccaagtgtacaaacaaagccattccgccttaaccgcccttcagaccgagcaagaacaagatccagagcattccgagaagatggttgcgaaacgccggttcagaatcggcgacatagcgggcgaacatacatcttttgacaagcttcccaaagacgtcatggcgacatatcgcgggacggcgttcggttcagacgatgccggcggaaaactgacctatactatagattttgctgccaaacagggacacggcaaaatcgaacatttgaaatcgccggaactcaatgtcgatctggccgtcgcctatatcaagccggatgaaaaacaccatgccgtcatcagcggttccgttctttacaaccaagacgagaaaggcagttactccctcggtatctttggcgaaaaagcccaggaagttgccggcagcgcggaagtggaaaccgcaaacggcatacaccatatcggtcttgccgccaagcagtaa

SEQ ID NO: 248 - protein, 260 aa, Neisseria meningitidis
CSSGGGGSGGGGVTADIGTGLADALTAPLDHKDKGLKSLTLEDSISQNGTLTLSAQGAEKTYGNGDSLNTGKLKNDKVSRFDFIRQIEVDGQLITLESGEFQVYKQSHSALTALQTEQEQDPEHSEKMVAKRRFRIGDIAGEHTSFDKLPKDVMATYRGTAFGSDDAGGKLTYTIDFAAKQGHGKIEHLKSPELNVDLAVAYIKPDEKHHAVISGSVLYNQDEKGSYSLGIFGEKAQEVAGSAEVETANGIHHIGLAAKQ

SEQ ID NO: 249 - DNA, 786 bp, Neisseria meningitidis
tgcggatccagcggaggcggcggaagcggaggcggcggtgtcaccgccgacatcggcacggggcttgccgatgcactaactgcgccgctcgaccataaagacaaaggcttgaaatccctgacattggaagactccatttcccaaaacggaacactgaccctgtcggcacaaggtgcggaaaaaacttatggaaacggcgacagccttaatacgggcaaattgaagaacgacaaggtcagccgtttcgactttatccgtcaaatcgaagtggacgggcagctcattaccttggagagcggagagttccaagtgtacaaacaaagccattccgccttaaccgcccttcagaccgagcaagaacaagatccagagcattccgagaagatggttgcgaaacgccggttcagaatcggcgacatagcgggcgaacatacatcttttgacaagcttcccaaagacgtcatggcgacatatcgcgggacggcgttcggttcagacgatgccggcggaaaactgacctatactatagattttgctgccaaacagggacacggcaaaatcgaacatttgaaatcgccggaactcaatgtcgatctggccgtcgcctatatcaagccggatgaaaaacaccatgccgtcatcagcggttccgttctttacaaccaagacgagaaaggcagttactccctcggtatctttggcgaaaaagcccaggaagttgccggcagcgcggaagtggaaaccgcaaacggcatacaccatatcggtcttgccgccaagcagtaa

SEQ ID NO: 250 - protein, 261 aa, Neisseria meningitidis
CGSSGGGGSGGGGVTADIGTGLADALTAPLDHKDKGLKSLTLEDSISQNGTLTLSAQGAEKTYGNGDSLNTGKLKNDKVSRFDFIRQIEVDGQLITLESGEFQVYKQSHSALTALQTEQEQDPEHSEKMVAKRRFRIGDIAGEHTSFDKLPKDVMATYRGTAFGSDDAGGKLTYTIDFAAKQGHGKIEHLKSPELNVDLAVAYIKPDEKHHAVISGSVLYNQDEKGSYSLGIFGEKAQEVAGSAEVETANGIHHIGLAAKQ

SEQ ID NO: 251 - DNA, 783 bp, Neisseria meningitidis
atgagcagcggaggcggcggaagcggaggcggcggtgtcaccgccgacatcggcacggggcttgccgatgcactaactgcgccgctcgaccataaagacaaaggcttgaaatccctgacattggaagactccatttcccaaaacggaacactgaccctgtcggcacaaggtgcggaaaaaacttatggaaacggcgacagccttaatacgggcaaattgaagaacgacaaggtcagccgtttcgactttatccgtcaaatcgaagtggacgggcagctcattaccttggagagcggagagttccaagtgtacaaacaaagccattccgccttaaccgcccttcagaccgagcaagaacaagatccagagcattccgagaagatggttgcgaaacgccggttcagaatcggcgacatagcgggcgaacatacatcttttgacaagcttcccaaagacgtcatggcgacatatcgcgggacggcgttcggttcagacgatgccggcggaaaactgacctatactatagattttgctgccaaacagggacacggcaaaatcgaacatttgaaatcgccggaactcaatgtcgatctggccgtcgcctatatcaagccggatgaaaaacaccatgccgtcatcagcggttccgttctttacaaccaagacgagaaaggcagttactccctcggtatctttggcgaaaaagcccaggaagttgccggcagcgcggaagtggaaaccgcaaacggcatacaccatatcggtcttgccgccaagcagtaa

SEQ ID NO: 252 - protein, 260 aa, Neisseria meningitidis
MSSGGGGSGGGGVTADIGTGLADALTAPLDHKDKGLKSLTLEDSISQNGTLTLSAQGAEKTYGNGDSLNTGKLKNDKVSRFDFIRQIEVDGQLITLESGEFQVYKQSHSALTALQTEQEQDPEHSEKMVAKRRFRIGDIAGEHTSFDKLPKDVMATYRGTAFGSDDAGGKLTYTIDFAAKQGHGKIEHLKSPELNVDLAVAYIKPDEKHHAVISGSVLYNQDEKGSYSLGIFGEKAQEVAGSAEVETANGIHHIGLAAKQ

SEQ ID NO: 253 - DNA, 390 bp, Neisseria meningitidis
gacagcctgataaaccaacgctccttccttgtcagcggtttgggcggagaacataccgccttcaaccaactgcctgacggcaaagccgagtatcacggcaaagcattcagctccgacgatgctggcggaaaactgacctataccatagatttcgccgccaaacagggacacggcaaaatcgaacacctgaaaacacccgagcaaaatgtcgagcttgccgccgccgaactcaaagcagatgaaaaatcacacgccgtcattttgggcgacacgcgctacggcagcgaagaaaaaggcacttaccacctcgcccttttcggcgaccgcgcccaagaaatcgccggctcggcaaccgtgaagataggggaaaaggttcacgaaatcggcatc

SEQ ID NO: 254 - protein, 10 aa, Neisseria meningitidis
  MISC_FEATURE (5)..(5): Xaa equals any of the naturally occurring L-amino acids
ADIGXGLADA

SEQ ID NO: 255 - protein, 10 aa, Neisseria meningitidis
  MISC_FEATURE (3)..(3): Xaa equals any of the naturally occurring L-amino acids
IGXGLADALT

SEQ ID NO: 256 - protein, 10 aa, Neisseria meningitidis
SLNTGKLKND

SEQ ID NO: 257 - protein, 17 aa, Neisseria meningitidis
  MISC_FEATURE (12)..(12): Xaa equals any of the naturally occurring L-amino acids
SLNTGKLKNDKXSRFDF

SEQ ID NO: 258 - protein, 9 aa, Neisseria meningitidis
  MISC_FEATURE (6)..(6): Xaa equals any of the naturally occurring L-amino acids
SGEFQXYKQ

SEQ ID NO: 259 - protein, 8 aa, Neisseria meningitidis
  MISC_FEATURE (6)..(6): Xaa equals any of the naturally occurring L-amino acids
IEHLKXPE

SEQ ID NO: 260 - protein, 10 aa, Neisseria meningitidis
  MISC_FEATURE (10)..(10): Xaa equals any of the naturally occurring L-amino acids
GGGVAADIGX

SEQ ID NO: 261 - protein, 9 aa, Neisseria meningitidis
SGEFQIYKQ

SEQ ID NO: 262 - protein, 10 aa, Neisseria meningitidis
HSAVVALQIE

SEQ ID NO: 263 - protein, 10 aa, Neisseria meningitidis
EKINNPDKID

SEQ ID NO: 264 - protein, 10 aa, Neisseria meningitidis
SLINQRSFLV

SEQ ID NO: 265 - protein, 10 aa, Neisseria meningitidis
SGLGGEHTAF

SEQ ID NO: 266 - protein, 10 aa, Neisseria meningitidis
GEHTAFNQLP

SEQ ID NO: 267 - protein, 11 aa, Neisseria meningitidis
SFLVSGLGGEH

SEQ ID NO: 268 - protein, 34 aa, Neisseria meningitidis
EKINNPDKIDSLINQRSFLVSGLGGEHTAFNQLP

SEQ ID NO: 269 - protein, 10 aa, Neisseria meningitidis
GKAEYHGKAF

SEQ ID NO: 270 - protein, 10 aa, Neisseria meningitidis
YHGKAFSSDD

SEQ ID NO: 271 - protein, 14 aa, Neisseria meningitidis
GKAEYHGKAFSSDD

SEQ ID NO: 272 - protein, 10 aa, Neisseria meningitidis
IEHLKTPEQN

SEQ ID NO: 273 - protein, 10 aa, Neisseria meningitidis
KTPEQNVELA

SEQ ID NO: 274 - protein, 14 aa, Neisseria meningitidis
IEHLKTPEQNVELA

SEQ ID NO: 275 - protein, 10 aa, Neisseria meningitidis
AELKADEKSH

SEQ ID NO: 276 - protein, 10 aa, Neisseria meningitidis
AVILGDTRYG

SEQ ID NO: 277 - protein, 20 aa, Neisseria meningitidis
AELKADEKSHAVILGDTRYG

SEQ ID NO: 278 - protein, 10 aa, Neisseria meningitidis
EEKGTYHLAL

SEQ ID NO: 279 - protein, 10 aa, Neisseria meningitidis
LITLESGEFQ

SEQ ID NO: 280 - protein, 10 aa, Neisseria meningitidis
SALTALQTEQ

SEQ ID NO: 281 - protein, 10 aa, Neisseria meningitidis
FQVYKQSHSA

SEQ ID NO: 282 - protein, 26 aa, Neisseria meningitidis
LITLESGEFQVYKQSHSALTALQTEQ

SEQ ID NO: 283 - protein, 10 aa, Neisseria meningitidis
IGDIAGEHTS

SEQ ID NO: 284 - protein, 10 aa, Neisseria meningitidis
EHTSFDKLPK

SEQ ID NO: 285 - protein, 16 aa, Neisseria meningitidis
IGDIAGEHTSFDKLPK

SEQ ID NO: 286 - protein, 10 aa, Neisseria meningitidis
ATYRGTAFGS

SEQ ID NO: 287 - protein, 10 aa, Neisseria meningitidis
DDAGGKLTYT

SEQ ID NO: 288 - protein, 10 aa, Neisseria meningitidis
IDFAAKQGHG

SEQ ID NO: 289 - protein, 10 aa, Neisseria meningitidis
KIEHLKSPEL

SEQ ID NO: 290 - protein, 42 aa, Neisseria meningitidis
ATYRGTAFGSDDAGGKLTYTIDFAAKQGHGKIEHLKSPELNV

SEQ ID NO: 291 - protein, 10 aa, Neisseria meningitidis
HAVISGSVLY

SEQ ID NO: 292 - protein, 10 aa, Neisseria meningitidis
KGSYSLGIFG

SEQ ID NO: 293 - protein, 10 aa, Neisseria meningitidis
VLYNQDEKGS

SEQ ID NO: 294 - protein, 24 aa, Neisseria meningitidis
HAVISGSVLYNQDEKGSYSLGIFG

SEQ ID NO: 295 - protein, 10 aa, Neisseria meningitidis
AQEVAGSAEV

SEQ ID NO: 296 - protein, 10 aa, Neisseria meningitidis
IHHIGLAAKQ

SEQ ID NO: 297 - protein, 10 aa, Neisseria meningitidis
VETANGIHHI

SEQ ID NO: 298 - protein, 25 aa, Neisseria meningitidis
AQEVAGSAEVETANGIHHIGLAAKQ

SEQ ID NO: 299 - protein, 22 aa, Neisseria meningitidis
VAGSAEVETANGIHHIGLAAKQ

SEQ ID NO: 300 - protein, 254 aa, Neisseria meningitidis
  MISC_FEATURE (9)..(9): Xaa equals any of the naturally occurring L-amino acids
  MISC_FEATURE (14)..(14): Xaa equals any of the naturally occurring L-amino acids
  MISC_FEATURE (22)..(22): Xaa equals any of the naturally occurring L-amino acids
  MISC_FEATURE (24)..(24): Xaa equals any of the naturally occurring L-amino acids
  MISC_FEATURE (26)..(26): Xaa equals any of the naturally occurring L-amino acids
  MISC_FEATURE (30)..(30): Xaa equals any of the naturally occurring L-amino acids
  MISC_FEATURE (32)..(32): Xaa equals any of the naturally occurring L-amino acids
  MISC_FEATURE (37)..(37): Xaa equals any of the naturally occurring L-amino acids
  MISC_FEATURE (38)..(38): Xaa equals any of the naturally occurring L-amino acids
  MISC_FEATURE (40)..(40): Xaa equals any of the naturally occurring L-amino acids
  MISC_FEATURE (41)..(41): Xaa equals any of the naturally occurring L-amino acids
  MISC_FEATURE (42)..(42): Xaa equals any of the naturally occurring L-amino acids
  MISC_FEATURE (44)..(44): Xaa equals any of the naturally occurring L-amino acids
  MISC_FEATURE (45)..(45): Xaa equals any of the naturally occurring L-amino acids
  MISC_FEATURE (47)..(47): Xaa equals any of the naturally occurring L-amino acids
  MISC_FEATURE (49)..(49): Xaa equals any of the naturally occurring L-amino acids
  MISC_FEATURE (57)..(57): Xaa equals any of the naturally occurring L-amino acids
  MISC_FEATURE (58)..(58): Xaa equals any of the naturally occurring L-amino acids
  MISC_FEATURE (59)..(59): Xaa equals any of the naturally occurring L-amino acids
  MISC_FEATURE (73)..(73): Xaa equals any of the naturally occurring L-amino acids
  MISC_FEATURE (79)..(79): Xaa equals any of the naturally occurring L-amino acids
  MISC_FEATURE (80)..(80): Xaa equals any of the naturally occurring L-amino acids
  MISC_FEATURE (81)..(81): Xaa equals any of the naturally occurring L-amino acids
  MISC_FEATURE (83)..(83): Xaa equals any of the naturally occurring L-amino acids
  MISC_FEATURE (87)..(87): Xaa equals any of the naturally occurring L-amino acids
  MISC_FEATURE (88)..(88): Xaa equals any of the naturally occurring L-amino acids
  MISC_FEATURE (92)..(92): Xaa equals any of the naturally occurring L-amino acids
  MISC_FEATURE (98)..(98): Xaa equals any of the naturally occurring L-amino acids
  MISC_FEATURE (102)..(102): Xaa equals any of the naturally occurring L-amino acids
  MISC_FEATURE (106)..(106): Xaa equals any of the naturally occurring L-amino acids
  MISC_FEATURE (107)..(107): Xaa equals any of the naturally occurring L-amino acids
  MISC_FEATURE (111)..(111): Xaa equals any of the naturally occurring L-amino acids
  MISC_FEATURE (113)..(113): Xaa equals any of the naturally occurring L-amino acids
  MISC_FEATURE (114)..(114): Xaa equals any of the naturally occurring L-amino acids
  MISC_FEATURE (115)..(115): Xaa equals any of the naturally occurring L-amino acids
  MISC_FEATURE (116)..(116): Xaa equals any of the naturally occurring L-amino acids
  MISC_FEATURE (117)..(117): Xaa equals any of the naturally occurring L-amino acids
  MISC_FEATURE (118)..(118): Xaa equals any of the naturally occurring L-amino acids
  MISC_FEATURE (119)..(119): Xaa equals any of the naturally occurring L-amino acids
  MISC_FEATURE (120)..(120): Xaa equals any of the naturally occurring L-amino acids
  MISC_FEATURE (121)..(121): Xaa equals any of the naturally occurring L-amino acids
  MISC_FEATURE (122)..(122): Xaa equals any of the naturally occurring L-amino acids
  MISC_FEATURE (123)..(123): Xaa equals any of the naturally occurring L-amino acids
  MISC_FEATURE (124)..(124): Xaa equals any of the naturally occurring L-amino acids
  MISC_FEATURE (125)..(125): Xaa equals any of the naturally occurring L-amino acids
  MISC_FEATURE (126)..(126): Xaa equals any of the naturally occurring L-amino acids
  MISC_FEATURE (128)..(128): Xaa equals any of the naturally occurring L-amino acids
  MISC_FEATURE (130)..(130): Xaa equals any of the naturally occurring L-amino acids
  MISC_FEATURE (131)..(131): Xaa equals any of the naturally occurring L-amino acids
  MISC_FEATURE (132)..(132): Xaa equals any of the naturally occurring L-amino acids
  MISC_FEATURE (133)..(133): Xaa equals any of the naturally occurring L-amino acids
  MISC_FEATURE (134)..(134): Xaa equals any of the naturally occurring L-amino acids
  MISC_FEATURE (135)..(135): Xaa equals any of the naturally occurring L-amino acids
  MISC_FEATURE (140)..(140): Xaa equals any of the naturally occurring L-amino acids
  MISC_FEATURE (142)..(142): Xaa equals any of the naturally occurring L-amino acids
  MISC_FEATURE (143)..(143): Xaa equals any of the naturally occurring L-amino acids
  MISC_FEATURE (146)..(146): Xaa equals any of the naturally occurring L-amino acids
  MISC_FEATURE (147)..(147): Xaa equals any of the naturally occurring L-amino acids
  MISC_FEATURE (148)..(148): Xaa equals any of the naturally occurring L-amino acids
  MISC_FEATURE (150)..(150): Xaa equals any of the naturally occurring L-amino acids
  MISC_FEATURE (152)..(152): Xaa equals any of the naturally occurring L-amino acids
  MISC_FEATURE (154)..(154): Xaa equals any of the naturally occurring L-amino acids
  MISC_FEATURE (157)..(157): Xaa equals any of the naturally occurring L-amino acids
  MISC_FEATURE (161)..(161): Xaa equals any of the naturally occurring L-amino acids
  MISC_FEATURE (162)..(162): Xaa equals any of the naturally occurring L-amino acids
  MISC_FEATURE (164)..(164): Xaa equals any of the naturally occurring L-amino acids
  MISC_FEATURE (166)..(166): Xaa equals any of the naturally occurring L-amino acids
  MISC_FEATURE (168)..(168): Xaa equals any of the naturally occurring L-amino acids
  MISC_FEATURE (172)..(172): Xaa equals any of the naturally occurring L-amino acids
  MISC_FEATURE (173)..(173): Xaa equals any of the naturally occurring L-amino acids
  MISC_FEATURE (177)..(177): Xaa equals any of the naturally occurring L-amino acids
  MISC_FEATURE (179)..(179): Xaa equals any of the naturally occurring L-amino acids
  MISC_FEATURE (185)..(185): Xaa equals any of the naturally occurring L-amino acids
  MISC_FEATURE (188)..(188): Xaa equals any of the naturally occurring L-amino acids
  MISC_FEATURE (191)..(191): Xaa equals any of the naturally occurring L-amino acids
  MISC_FEATURE (194)..(194): Xaa equals any of the naturally occurring L-amino acids
  MISC_FEATURE (195)..(195): Xaa equals any of the naturally occurring L-amino acids
  MISC_FEATURE (196)..(196): Xaa equals any of the naturally occurring L-amino acids
  MISC_FEATURE (197)..(197): Xaa equals any of the naturally occurring L-amino acids
  MISC_FEATURE (199)..(199): Xaa equals any of the naturally occurring L-amino acids
  MISC_FEATURE (203)..(203): Xaa equals any of the naturally occurring L-amino acids
  MISC_FEATURE (208)..(208): Xaa equals any of the naturally occurring L-amino acids
  MISC_FEATURE (210)..(210): Xaa equals any of the naturally occurring L-amino acids
  MISC_FEATURE (211)..(211): Xaa equals any of the naturally occurring L-amino acids
  MISC_FEATURE (212)..(212): Xaa equals any of the naturally occurring L-amino acids
  MISC_FEATURE (214)..(214): Xaa equals any of the naturally occurring L-amino acids
  MISC_FEATURE (215)..(215): Xaa equals any of the naturally occurring L-amino acids
  MISC_FEATURE (216)..(216): Xaa equals any of the naturally occurring L-amino acids
  MISC_FEATURE (220)..(220): Xaa equals any of the naturally occurring L-amino acids
  MISC_FEATURE (222)..(222): Xaa equals any of the naturally occurring L-amino acids
  MISC_FEATURE (224)..(224): Xaa equals any of the naturally occurring L-amino acids
  MISC_FEATURE (225)..(225): Xaa equals any of the naturally occurring L-amino acids
  MISC_FEATURE (226)..(226): Xaa equals any of the naturally occurring L-amino acids
  MISC_FEATURE (228)..(228): Xaa equals any of the naturally occurring L-amino acids
  MISC_FEATURE (229)..(229): Xaa equals any of the naturally occurring L-amino acids
  MISC_FEATURE (233)..(233): Xaa equals any of the naturally occurring L-amino acids
  MISC_FEATURE (236)..(236): Xaa equals any of the naturally occurring L-amino acids
  MISC_FEATURE (238)..(238): Xaa equals any of the naturally occurring L-amino acids
  MISC_FEATURE (240)..(240): Xaa equals any of the naturally occurring L-amino acids
  MISC_FEATURE (241)..(241): Xaa equals any of the naturally occurring L-amino acids
  MISC_FEATURE (242)..(242): Xaa equals any of the naturally occurring L-amino acids
  MISC_FEATURE (243)..(243): Xaa equals any of the naturally occurring L-amino acids
  MISC_FEATURE (244)..(244): Xaa equals any of the naturally occurring L-amino acids
  MISC_FEATURE (245)..(245): Xaa equals any of the naturally occurring L-amino acids
  MISC_FEATURE (247)..(247): Xaa equals any of the naturally occurring L-amino acids
  MISC_FEATURE (249)..(249): Xaa equals any of the naturally occurring L-amino acids
  MISC_FEATURE (250)..(250): Xaa equals any of the naturally occurring L-amino acids
  MISC_FEATURE (252)..(252): Xaa equals any of the naturally occurring L-amino acids
CSSGGGGVXADIGXGLADALTXPXDXKDKXLXSLTLXXSXXXNXXLXLXAQGAEKTXXXGDSLNTGKLKNDKXSRFDFXXXIXVDGXXITLXSGEFQXYKQXHSAXXALQXEXXXXXXXXXXXXXXRXFXXXXXXGEHTXFXXLPXXXAXYXGXAFXSDDXXGXLXYXIDFXXKQGXGXIEHLKXPEXNVXLAXXXXKXDEKXHAVIXGXXXYXXXEKGXYXLXXXGXXAQEXAGXAXVXXXXXXHXIXXAXKQ

SEQ ID NO: 301 - protein, 254 aa, Neisseria meningitidis
  MISC_FEATURE (14)..(14): Xaa equals any of the naturally occurring L-amino acids
  MISC_FEATURE (22)..(22): Xaa equals any of the naturally occurring L-amino acids
  MISC_FEATURE (24)..(24): Xaa equals any of the naturally occurring L-amino acids
  MISC_FEATURE (26)..(26): Xaa equals any of the naturally occurring L-amino acids
  MISC_FEATURE (30)..(30): Xaa equals any of the naturally occurring L-amino acids
  MISC_FEATURE (32)..(32): Xaa equals any of the naturally occurring L-amino acids
  MISC_FEATURE (37)..(37): Xaa equals any of the naturally occurring L-amino acids
  MISC_FEATURE (38)..(38): Xaa equals any of the naturally occurring L-amino acids
  MISC_FEATURE (40)..(40): Xaa equals any of the naturally occurring L-amino acids
  MISC_FEATURE (41)..(41): Xaa equals any of the naturally occurring L-amino acids
  MISC_FEATURE (42)..(42): Xaa equals any of the naturally occurring L-amino acids
  MISC_FEATURE (44)..(44): Xaa equals any of the naturally occurring L-amino acids
  MISC_FEATURE (45)..(45): Xaa equals any of the naturally occurring L-amino acids
  MISC_FEATURE (47)..(47): Xaa equals any of the naturally occurring L-amino acids
  MISC_FEATURE (49)..(49): Xaa equals any of the naturally occurring L-amino acids
  MISC_FEATURE (57)..(57): Xaa equals any of the naturally occurring L-amino acids
  MISC_FEATURE (58)..(58): Xaa equals any of the naturally occurring L-amino acids
  MISC_FEATURE (59)..(59): Xaa equals any of the naturally occurring L-amino acids
  MISC_FEATURE (73)..(73): Xaa equals any of the naturally occurring L-amino acids
  MISC_FEATURE (79)..(79): Xaa equals any of the naturally occurring L-amino acids
  MISC_FEATURE (80)..(80): Xaa equals any of the naturally occurring L-amino acids
  MISC_FEATURE (81)..(81): Xaa equals any of the naturally occurring L-amino acids
  MISC_FEATURE (83)..(83): Xaa equals any of the naturally occurring L-amino acids
  MISC_FEATURE (88)..(88): Xaa equals any of the naturally occurring L-amino acids
  MISC_FEATURE (92)..(92): Xaa equals any of the naturally occurring L-amino acids
  MISC_FEATURE (102)..(102): Xaa equals any of the naturally occurring L-amino acids
  MISC_FEATURE (146)..(146): Xaa equals any of the naturally occurring L-amino acids
  MISC_FEATURE (161)..(161): Xaa equals any of the naturally occurring L-amino acids
  MISC_FEATURE (162)..(162): Xaa equals any of the naturally occurring L-amino acids
  MISC_FEATURE (164)..(164): Xaa equals any of the naturally occurring L-amino acids
  MISC_FEATURE (166)..(166): Xaa equals any of the naturally occurring L-amino acids
  MISC_FEATURE (168)..(168): Xaa equals any of the naturally occurring L-amino acids
  MISC_FEATURE (172)..(172): Xaa equals any of the naturally occurring L-amino acids
  MISC_FEATURE (173)..(173): Xaa equals any of the naturally occurring L-amino acids
  MISC_FEATURE (177)..(177): Xaa equals any of the naturally occurring L-amino acids
  MISC_FEATURE (179)..(179): Xaa equals any of the naturally occurring L-amino acids
  MISC_FEATURE (194)..(194): Xaa equals any of the naturally occurring L-amino acids
  MISC_FEATURE (215)..(215): Xaa equals any of the naturally occurring L-amino acids
  MISC_FEATURE (226)..(226): Xaa equals any of the naturally occurring L-amino acids
  MISC_FEATURE (236)..(236): Xaa equals any of the naturally occurring L-amino acids
  MISC_FEATURE (242)..(242): Xaa equals any of the naturally occurring L-amino acids
  MISC_FEATURE (249)..(249): Xaa equals any of the naturally occurring L-amino acids
  MISC_FEATURE (252)..(252): Xaa equals any of the naturally occurring L-amino acids
CSSGGGGVAADIGXGLADALTXPXDXKDKXLXSLTLXXSXXXNXXLXLXAQGAEKTXXXGDSLNTGKLKNDKXSRFDFXXXIXVDGQXITLXSGEFQIYKQXHSAVVALQIEKINNPDKIDSLINQRSFLVSGLGGEHTAFNQLPXGKAEYHGKAFSSDDXXGXLXYXIDFXXKQGXGXIEHLKTPEQNVELAXAELKADEKSHAVILGDTRYGXEEKGTYHLALXGDRAQEIAGXATVKIXEKVHEIXIAXKQ

SEQ ID NO: 302 - protein, 255 aa, Neisseria meningitidis
  MISC_FEATURE (9)..(9): Xaa equals any of the naturally occurring L-amino acids
  MISC_FEATURE (14)..(14): Xaa equals any of the naturally occurring L-amino acids
  MISC_FEATURE (30)..(30): Xaa equals any of the naturally occurring L-amino acids
  MISC_FEATURE (32)..(32): Xaa equals any of the naturally occurring L-amino acids
  MISC_FEATURE (37)..(37): Xaa equals any of the naturally occurring L-amino acids
  MISC_FEATURE (38)..(38): Xaa equals any of the naturally occurring L-amino acids
  MISC_FEATURE (40)..(40): Xaa equals any of the naturally occurring L-amino acids
  MISC_FEATURE (41)..(41): Xaa equals any of the naturally occurring L-amino acids
  MISC_FEATURE (42)..(42): Xaa equals any of the naturally occurring L-amino acids
  MISC_FEATURE (44)..(44): Xaa equals any of the naturally occurring L-amino acids
  MISC_FEATURE (45)..(45): Xaa equals any of the naturally occurring L-amino acids
  MISC_FEATURE (47)..(47): Xaa equals any of the naturally occurring L-amino acids
  MISC_FEATURE (49)..(49): Xaa equals any of the naturally occurring L-amino acids
  MISC_FEATURE (87)..(87): Xaa equals any of the naturally occurring L-amino acids
  MISC_FEATURE (114)..(114): Xaa equals any of the naturally occurring L-amino acids
  MISC_FEATURE (117)..(117): Xaa equals any of the naturally occurring L-amino acids
  MISC_FEATURE (119)..(119): Xaa equals any of the naturally occurring L-amino acids
  MISC_FEATURE (121)..(121): Xaa equals any of the naturally occurring L-amino acids
  MISC_FEATURE (128)..(128): Xaa equals any of the naturally occurring L-amino acids
  MISC_FEATURE (130)..(130): Xaa equals any of the naturally occurring L-amino acids
  MISC_FEATURE (147)..(147): Xaa equals any of the naturally occurring L-amino acids
  MISC_FEATURE (148)..(148): Xaa equals any of the naturally occurring L-amino acids
  MISC_FEATURE (149)..(149): Xaa equals any of the naturally occurring L-amino acids
  MISC_FEATURE (192)..(192): Xaa equals any of the naturally occurring L-amino acids
  MISC_FEATURE (195)..(195): Xaa equals any of the naturally occurring L-amino acids
  MISC_FEATURE (196)..(196): Xaa equals any of the naturally occurring L-amino acids
  MISC_FEATURE (204)..(204): Xaa equals any of the naturally occurring L-amino acids
  MISC_FEATURE (229)..(229): Xaa equals any of the naturally occurring L-amino acids
  MISC_FEATURE (230)..(230): Xaa equals any of the naturally occurring L-amino acids
CSSGGGGVXADIGXGLADALTAPLDHKDKXLXSLTLXXSXXXNXXLXLXAQGAEKTYGNGDSLNTGKLKNDKVSRFDFIRQIEVDGXLITLESGEFQVYKQSHSALTALQTEQXQDXEXSXKMVAKRXFXIGDIAGEHTSFDKLPKXXXATYRGTAFGSDDAGGKLTYTIDFAAKQGHGKIEHLKSPELNVXLAXXYIKPDEKXHAVISGSVLYNQDEKGSYSLGIFGXXAQEVAGSAEVETANGIHHIGLAAKQ

SEQ ID NO: 303 - DNA, 31 bp, Artificial Sequence
  Primer
gcgcggatccttactgcttggcggcaagacc

SEQ ID NO: 304 - DNA, 23 bp, Artificial Sequence
  Primer
ctattctgcatatgactaggagc

SEQ ID NO: 305 - DNA, 21 bp, Artificial Sequence
  Primer
agcagcggaggcggcggtgtc

SEQ ID NO: 306 - DNA, 21 bp, Artificial Sequence
  Primer
tgccgatgcactaaccgcacc

SEQ ID NO: 307 - DNA, 21 bp, Artificial Sequence
  Primer
cgtttcgcaaccatcttcccg

SEQ ID NO: 308 - DNA, 44 bp, Artificial Sequence
  Primer
gagatctcactcactcattactgcttggcggcaagaccgatatg

SEQ ID NO: 309 - DNA, 29 bp, Artificial Sequence
  Primer
gcggatccagcggagggggtggtgtcgcc

SEQ ID NO: 310 - DNA, 35 bp, Artificial Sequence
  Primer
gcgcatgcttactgcttggcggcaagaccgatatg

SEQ ID NO: 311 - DNA, 26 bp, Artificial Sequence
  Primer
gcggatccagcggaggcggcggaagc

SEQ ID NO: 312 - DNA, 65 bp, Artificial Sequence
  Primer
  misc_feature (46)..(46): “Y” equals C or T
  misc_feature (49)..(49): “W” equals A or T
gcgcagatctcatatgagcagcggagggggtggtgtcgccgccgayatwggtgcggggcttgccg

SEQ ID NO: 313 - DNA, 19 bp, Artificial Sequence
  Primer
ctattctgcgtatgactag

SEQ ID NO: 314 - DNA, 22 bp, Artificial Sequence
  Primer
gtccgaacggtaaattatcgtg

SEQ ID NO: 315 - DNA, 29 bp, Artificial Sequence
  Primer
gcggatccagcggaggcggcggtgtcgcc

SEQ ID NO: 316 - DNA, 34 bp, Artificial Sequence
  Primer
gagatctcatatgagcagcggaggcggcggaagc

SEQ ID NO: 317 - DNA, 16 bp, Artificial Sequence
  Primer
gacagcctgataaacc

SEQ ID NO: 318 - DNA, 19 bp, Artificial Sequence
  Primer
gatgccgatttcgtgaacc

SEQ ID NO: 319 - DNA, 27 bp, Artificial Sequence
  Primer
gcgcatgcctactgtttgccggcgatg

SEQ ID NO: 320 - DNA, 44 bp, Artificial Sequence
  Primer
gagatctcactcactcactactgtttgccggcgatgccgatttc

SEQ ID NO: 321 - DNA, 70 bp, Artificial Sequence
  Primer
gcgcagatctcatatgagcagcggaggcggcggaagcggaggcggcggtgtcaccgccgacataggcacg

SEQ ID NO: 322 - DNA, 59 bp, Artificial Sequence
  Leader
atgaaaacaacgttaaaaatgaccgcacttgcggctctttctgcttttgttttagctgg

SEQ ID NO: 323 - DNA, 75 bp, Artificial Sequence
  Leader
atgactaggagcaaacctgtgaaccgaactgccttctgctgcctttccctgaccgccgccctgattctgaccgcc

SEQ ID NO: 324 - DNA, 75 bp, Artificial Sequence
  Leader
atgactaggagcaaacctgtgaaccgaactgccttctgctgcttttctctgaccgccgccctgattctgaccgcc

SEQ ID NO: 325 - DNA, 75 bp, Artificial Sequence
  Leader
atgactaggagcaaacctgtgaaccgaactaccttctgttgcctttctctgaccgccgccctgattctgaccgcc

SEQ ID NO: 326 - DNA, 75 bp, Artificial Sequence
  Leader
atgactaggagtaaacctgtgaatcgaactgccttctgctgcctttctctgaccactgccctgattctgaccgcc

SEQ ID NO: 327 - protein, 335 aa, Neisseria meningitidis
CSPAADSNHPSGQNAPANTESDGKNITLLNASYDVARDFYKEYNPLFIKTYQSEHPGTSVSIQQSHGGSSKQALSVANGLQADVVTMNQSSDIDLLEKKGLVEKGWQQALPDHAAPYTSTMVFLVRKNNPKQIRDWNDLAKDGVNIVIANPKTSGNGRYAFLGAYGYGLKTTNGNEQEAQKLVASILKNTPVFENGGRAATTTFTQRNIGDVLITFENEANYVSKKLTQGQFEIVYPSYTISAESPVAVVNSVVAKKGTQKTARAYLEYLWSEPAQELAASLYLRPRNPEVLARHKADFPDLDTFSPEEKFGGWDNIMKTYFADGGIFDRLTAQK

SEQ ID NO: 328 - protein, 68 aa, Neisseria meningitidis
CAGTWEGAKQDTARNLDKTQAAAERAAEQTGNAVEKGWDKTKEAVKKGGNAVGRGISHLGGKIENATE

SEQ ID NO: 329 - protein, 21 aa, Neisseria meningitidis
CMKTTLKMTALAALSAFVLAG